(19) United States
(12) Patent Application Publication
(10) Pub. No.: US 2023/0158179 A1
(43) Pub. Date: May 25, 2023

(54) RADIOPHARMACEUTICAL CONJUGATE COMPOSITIONS AND USES THEREOF

(71) Applicant: RayzeBio, Inc., San Diego, CA (US)

(72) Inventors: Deborah CHARYCH, San Diego, CA (US); Junjie LIU, San Diego, CA (US); Ken SONG, San Diego, CA (US); Alain NONCOVICH, San Diego, CA (US); Derek COLE, San Diego, CA (US)

(21) Appl. No.: 17/989,479

(22) Filed: Nov. 17, 2022

Related U.S. Application Data
(63) Continuation of application No. PCT/US2021/061186, filed on Nov. 30, 2021.
(60) Provisional application No. 63/119,555, filed on Nov. 30, 2020.

(51) Int. Cl. A61K 51/08

(52) U.S. Cl. CPC A61K 51/088

(57) ABSTRACT

Provided herein are radiopharmaceutical conjugate compositions and uses thereof. In one aspect, provided herein are conjugates that comprise a monocyclic peptide of 5 to 40 amino acid residues and a metal chelator configured to bind with a radionuclide. In some embodiments, the monocyclic peptide is cyclized by a non-disulfide bond. In some embodiments, the monocyclic peptide does not comprise any non-disulfide bond. The monocyclic peptide can be configured to bind with a structure on a cell. Further provided herein are methods of treating cancer by administering the described conjugates and compositions.

### CROSS-REFERENCE

This application is a continuation of International Application No. PCT/US2021/061186, filed Nov. 30, 2021, which claims the benefit of U.S. Provisional Application No. 63/119,555, filed on Nov. 30, 2020, each of which are incorporated herein by reference in their entirety.

### SEQUENCE LISTING

The instant application contains a Sequence Listing which has been submitted electronically in XML format and is hereby incorporated by reference in its entirety. Said XML copy, created on Jan. 9, 2023, is named 59541-707_301_SL.xml and is 81,156 bytes in size.

### BACKGROUND

In the United States, cancer is the leading cause of death for those under 65 years of age, and it accounted for about 21% of all death in 2018. Traditional radiotherapies such as external beam radiation therapy have been used for decades as a standard-of-care treatment for diagnosed cancer patients. While some patients respond to external beam radiation therapy, many others do not. Further, metastasis and circulating tumor cells can spread and remain in the bloodstream or bodily fluids after standard-of-care treatment and lead to resistance to therapy. The presence of cancer cells in various parts of the body reduces the therapeutic efficacy of traditional radiotherapies. Accordingly, strategies for targeted radiotherapies are being developed, and there remains a need for targeted radiotherapies that have the desired affinity, stability, and exertion profile.

### SUMMARY

In one aspect, the present disclosure relates to radiopharmaceutical conjugates that comprise a peptide such as a monocyclic peptide, a metal chelator, optionally a linker, and optionally an alpha-emitting radionuclide such as ²²⁵Ac bound to the metal chelator. In some embodiments, the peptide consists of 5 to 40, 10 to 20, or 10 to 15 amino acid residues. In some embodiments, the peptide does not contain or is not cyclized by typical disulfide bond such as is formed by L-cysteines. The conjugates of the present disclosures provide several advantages over existing radiopharmaceutical agents. For example, by adjusting and having a suitable number and type of amino acid residues in the peptide, an optimum balance of peptide stability, receptor binding, tumor penetration, and elimination profile can be achieved. Traditionally, radiopharmaceutical agents utilize a small molecule or an antibody to target receptors. Small molecule ligands tend to have low target specificity; and on the other hand, the antibody-based conjugates have low tumor penetration. In addition, the elimination half-life for small molecule ligand-based conjugates tends to be very short and the elimination half-life for antibody-based conjugates can be too long. It is revealed by the present disclosure that peptide (such as macrocyclic peptide)-based conjugates are ideal for radiopharmaceuticals. In some embodiments, conjugates of the present disclosure can be superior to existing cyclic peptide-based conjugates (such as DOTA-TATE) in terms of stability and other properties. In another embodiment, peptides with disulfide bonds can be susceptible to reduction reactions, leading to destabilization followed by metabolism of the peptides that contain a disulfide bond. Thus, in some cases, the conjugates that comprise a peptide that is not cyclized by a disulfide bond is more proteolytically, thermally and pH stable than a corresponding conjugate that comprises a disulfide-bridged peptide. The resulting conjugate has a more optimal pharmacodynamic and therefore, pharmacological profile.

In one aspect, disclosed herein is a conjugate comprising: a monocyclic peptide of 5 to 40 amino acid residues; a metal chelator configured to bind with a radionuclide; and optionally a radionuclide. In one aspect, disclosed herein is a conjugate comprising: a monocyclic peptide of 5 to 40 amino acid residues; a metal chelator configured to bind with a radionuclide; and optionally a radionuclide. In one aspect, disclosed herein is a conjugate comprising: a monocyclic peptide of 5 to 40 amino acid residues, wherein the monocyclic peptide is cyclized by a non-disulfide bond; and a metal chelator configured to bind with a radionuclide. In one aspect, disclosed herein is a conjugate comprising: a monocyclic peptide of 6 to 40 amino acid residues; and a metal chelator configured to bind with a radionuclide, provided that the monocyclic peptide does not comprise a disulfide bond. In some embodiments, the monocyclic peptide consists of 5 to 20 amino acid residues. In some embodiments, the monocyclic peptide consists of 10 to 20 amino acid residues. In some embodiments, the monocyclic peptide consists of 5, 6, 7, 8, or 9 amino acid residues. In some embodiments, the monocyclic peptide consists of 10, 11, 12, 13, 14, or 15 amino acid residues. In some embodiments, the peptide binds to a structure on a cell. In some embodiments, the conjugate comprises ²²⁵Ac. In some embodiments, the conjugate comprises ¹⁷⁷Lu.

In one aspect, disclosed herein is a conjugate comprising: a cyclic binding peptide that binds a cell-surface protein, wherein the cyclic binding peptide is cyclized by a non-disulfide bond and consists of 5-40 amino acid residues; and a metal chelator configured to bind with a radionuclide, wherein the cell-surface protein is a transmembrane glycoprotein. In one aspect, disclosed herein is a conjugate comprising: a binding peptide that binds a cell-surface protein; a metal chelator configured to bind with a radionuclide; and a linker that covalently attaches the binding peptide to the metal chelator; wherein the cell-surface protein is an oncofetal antigen, a tight junction protein, an adhesion protein, a transporter receptor, or a tyrosine kinase receptor (TKR). In some embodiments, the peptide consists of 5 to 20 amino acid residues. In some embodiments, the peptide consists of 10 to 20 amino acid residues. In some embodiments, the binding peptide consists of 5, 6, 7, 8, 9, 10, 11, 12, 13, 14, or 15 amino acid residues. In some embodiments, the peptide binds to a structure on a cell. In some embodiments, the conjugate comprises ²²⁵Ac. In some embodiments, the conjugate comprises ¹⁷⁷Lu.

In one aspect, disclosed herein is a conjugate comprising: a targeting moiety that comprises a monocyclic peptide of 5 to 40 amino acid residues, a metal chelator configured to bind with a radionuclide, and an alpha particle-emitting radionuclide bound to the metal chelator, provided that the monocyclic peptide does not comprise a disulfide bond. In some embodiments, the alpha particle-emitting radionuclide is actinium-225, astatine-211, thorium-227, or radium-223. In some embodiments, the alpha particle-emitting radionuclide is actinium-225. In some embodiments, the monocyclic peptide consists of 10, 11, 12, 13, 14, or 15 amino acid residues. In some embodiments, the monocyclic peptide consists of 5, 6, 7, 8, 9, 10, 11, or 12 amino acid residues. In some embodiments, the targeting moiety binds an oncofetal antigen, a tight junction protein, an adhesion protein, an eph-related tyrosine kinase receptor (TKR), or a transporter protein. In some embodiments, the targeting moiety binds a transmembrane glycoprotein. In some embodiments, the targeting moiety binds prostate-specific membrane antigen (PSMA), tumor-associated calcium signal transducer 2 (Trop-2), folate receptor 1 (FOLR1), nectin cell adhesion molecule 4 (Nectin-4), carcinoembryonic antigen-related cell adhesion molecule 5 (CEACAMS), mesothelin, EPH Receptor A2 (EPHA2), tumor-associated calcium signal transducer 2 (Trop-2), somatostatin receptor type 2 (SSTR2), carbonic anhydrase IX (CAIX), delta-like 3 (DLL3), fibroblast activation protein alpha (FAP-alpha), B7-H4, tumor Endothelial Marker 8 (TEM8), NaPi2b, claudin 18.2, tumor-associated glycoprotein 72 (TAG72), or CD70. In some embodiments, the targeting moiety binds an oncofetal protein, a tumor surface marker, a receptor protein, a ligand protein, a cell adhesion protein, or an immunomodulation protein. In some embodiments, the cyclic peptide is cyclized by a peptide bond, alkyl bond, alkenyl bond, ester bond, thioester bond, ether bond, thioether bond, phosphate ether bond, azo bond, C—N—C bond, C═N—C bond, C═N—O bond, amide bond, lactam bridge, carbamoyl bond, urea bond, thiourea bond, amine bond, or thioamide bond. In some embodiments, the cyclic peptide is cyclized by a thioether bond. In some embodiments, the cyclic peptide is cyclized via oxime cyclization or a cyclization between cysteine and haloacyl. In some embodiments, the cyclic peptide comprises chloroacetyl group at the N-terminus, having Cys at the C-terminus, and is cyclized via a thioether bond therebetween. In some embodiments, the metal chelator comprises DOTA, DOTP, DOTMA, DOTAM, DOTAGA, DTPA, NTA, EDTA, DO3A, DO2A, NOC, NOTA, TETA, DiAmSar, CB-Cyclam, CB-TE2A, DOTA-4AMP, H₄pypa, H₄octox, H₄octapa, p-NO₂-Bn-neunpa, or NOTP. In some embodiments, the metal chelator is DOTA. In some embodiments, the conjugate comprises a linker that covalently attaches the cyclic peptide with the metal chelator. In some embodiments, the cyclic peptide is directly linked to the metal chelator through a covalent bond without a linker.

In one aspect, disclosed herein is a conjugate comprising: a cyclic binding peptide that binds a cell-surface protein, wherein the cyclic binding peptide is cyclized by a non-disulfide bond and consists of 5-40 amino acid residues, and a metal chelator configured to bind with a radionuclide, wherein the cell-surface protein is a transmembrane glycoprotein. In some embodiments, the transmembrane glycoprotein is prostate-specific membrane antigen (PSMA), tumor-associated calcium signal transducer 2 (Trop-2), somatostatin receptor type 2 (SSTR2), or carbonic anhydrase IX (CAIX). In some embodiments, the metal chelator comprises DOTA, DOTP, DOTMA, DOTAM, DOTAGA, DTPA, NTA, EDTA, DO3A, DO2A, NOC, NOTA, TETA, DiAmSar, CB-Cyclam, CB-TE2A, DOTA-4AMP, H₄pypa, H₄octox, H₄octapa, p-NO₂-Bn-neunpa, or NOTP. In some embodiments, the metal chelator is DOTA. In some embodiments, the conjugate comprises a linker that covalently attaches the cyclic peptide with the metal chelator.

In one aspect, disclosed herein is a conjugate comprising: a binding peptide that binds a cell-surface protein, a metal chelator configured to bind with a radionuclide, and a linker that covalently attaches the binding peptide to the metal chelator, wherein the cell-surface protein is an oncofetal antigen, a tight junction protein, an adhesion protein, a transporter receptor, or a tyrosine kinase receptor (TKR). In some embodiments, the binding peptide is a cyclic peptide. In some embodiments, the conjugate further comprises an alpha particle-emitting radionuclide bound to the metal chelator. In some embodiments, the cell-surface protein is an oncofetal antigen. In some embodiments, the oncofetal antigen is a carcinoembryonic antigen (CEA) or an alpha-fetoprotein (AFP). In some embodiments, the oncofetal antigen is carcinoembryonic antigen-related cell adhesion molecule 5 (CEACAMS). In some embodiments, the oncofetal antigen is tumor-associated calcium signal transducer 2 (Trop-2). In some embodiments, the cell-surface protein is an eph-related tight junction protein. In some embodiments, the tight junction protein is a claudin (such as claudin 2, 3, 4, 7, 8, 12, 15, or 18), an occludin, an oligodendrocyte-specific protein (OSP), a peripheral myelin protein (PMP), or a junctional adhesion molecule. In some embodiments, the cell-surface protein is an adhesion protein. In some embodiments, the adhesion protein is nectin cell adhesion molecule 4 (Nectin-4). In some embodiments, the adhesion protein is selected from integrins, cadherins, selectins, and immunoglobulin-like Cell Adhesion Molecules. In some embodiments, the cell-surface protein is a tyrosine kinase receptor (TKR). In some embodiments, the tyrosine kinase receptor is an eph-related tyrosine kinase receptor. In some embodiments, the eph-related tyrosine kinase receptor is an ephrinA or an ephrinB receptor. In some embodiments, the Eph receptor is EPH Receptor A2 (EPHA2). In some embodiments, the tyrosine kinase receptor is an epidermal growth factor receptor (EGFR), a fibroblast growth factor receptor (FGFR), a vascular endothelial growth factor receptor (VEGFR), an ErbB receptor, a RET receptor, or a discoidin domain receptor (DDR). In some embodiments, the ErbB receptor is a Her2 receptor. In some embodiments, the cell-surface protein is a transport receptor. In some embodiments, the transport receptor is Folate receptor 1 (FOLR1).

In some embodiments, a binding peptide described herein consists of 5 to 40 amino acid residues. In some embodiments, the binding peptide consists of 5 to 20 amino acid residues. In some embodiments, the binding peptide consists of 5 to 14 amino acid residues. In some embodiments, the binding peptide consists of 7 to 14 amino acid residues. In some embodiments, the binding peptide consists of 10 to 14 amino acid residues. In some embodiments, the binding peptide consists of 5, 6, 7, 8, 9, 10, 11, or 12 amino acid residues. In some embodiments, the binding peptide is a monocyclic peptide. In some embodiments, the cyclic peptide is cyclized by a peptide bond, alkyl bond, alkenyl bond, ester bond, thioester bond, ether bond, thioether bond, phosphate ether bond, azo bond, C—N—C bond, C═N—C bond, C═N—O bond, amide bond, lactam bridge, carbamoyl bond, urea bond, thiourea bond, amine bond, or thioamide bond. In some embodiments, the cyclic peptide is cyclized by a thioether bond. In some embodiments, the cyclic peptide is cyclized via oxime cyclization or a cyclization between cysteine and haloacyl. In some embodiments, the cyclic peptide comprises chloroacetyl group at the N-terminus, having Cys at the C-terminus, and is cyclized via a thioether bond therebetween. In some embodiments, the metal chelator comprises DOTA, DOTP, DOTMA, DOTAM, DOTAGA, DTPA, NTA, EDTA, DO3A, DO2A, NOC, NOTA, TETA, DiAmSar, CB-Cyclam, CB-TE2A, DOTA-4AMP, H₄pypa, H₄octox, H₄octapa, p-NO₂-Bn-neunpa, or NOTP. In some embodiments, the metal chelator is DOTA. In some embodiments, the conjugate comprises a linker that covalently attaches the cyclic peptide with the metal chelator. In some embodiments, the linker comprises one or more lysine residues. In some embodiments, the linker is a lysine residue. In some embodiments, the linker comprises one or more of substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. In some embodiments, the linker is attached to the binding peptide or the monocyclic peptide via the N terminus of the peptide. In some embodiments, the linker is attached to the binding peptide or the monocyclic peptide via the C terminus of the peptide. In some embodiments, the linker is attached to the binding peptide or the monocyclic peptide via a non-terminal amino acid of the peptide. In some embodiments, the cyclic peptide is directly linked to the metal chelator through a covalent bond without a linker.

In one aspect, disclosed herein is a conjugate comprising a targeting moiety that comprises a cyclic peptide having a structure of Formula (III) and a metal chelator configured to bind with a radionuclide,

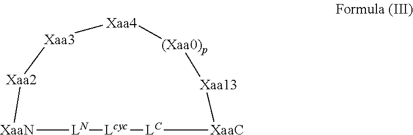

(attachment point to metal chelator not shown)
wherein
each of the XaaN, Xaa2, Xaa3, Xaa4, Xaa0, Xaa13, and XaaC is independently an amino acid residue;
p is 0, 1, 2, 3, 4, 5, 6, 7, or 8;
wherein each of the amino acid residues in the cyclic peptide is joined by a peptide bond, provided that XaaN and XaaC is connected through -L^{N}-L^{cyc}-L^{c}-;
L^{N} and L^{C} are each independently optionally substituted C₁-C₆alkylene, optionally substituted C₁-C₆heteroalkylene, or a bond, wherein the alkylene or heteroalkylene is optionally substituted;
L^{cyc} is a ring closing group comprising a structure selected from —C(═O)—CH₂—S—, —S—, —CH═CH—, —NH—,-succinimide-S—, —C(═O)—CH₂—NH—, and —C(═O)—CH₂—O—; and
wherein the cyclic peptide is monocyclic and does not contain any di-sulfide bond. In some embodiments, L^{N} and L^{C} are each independently optionally substituted C₁-C₆alkylene, optionally substituted C₁-C₆heteroalkylene, or a bond, wherein the C₁-C₆alkylene and C₁-C₆heteroalkylene are each optionally substituted with one or more R¹⁰,
each R¹⁰ is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, —Si(R^{a})₃, —P(═O)(R^{b})₂, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl, wherein the alkyl, alkenyl, alkynyl, cycloalkyl, heterocycloalkyl, aryl, and heteroaryl is optionally and independently substituted with one or more R^{10a};
   or two R¹⁰ on the same atom are taken together to form an oxo;
   or two R¹⁰ on the same atoms are taken together to form a cycloalkyl or heterocycloalkyl; each optionally substituted with one or more R^{10a};
   or two R¹⁰ on different atoms are taken together to form a cycloalkyl, heterocycloalkyl, aryl, or heteroaryl, each optionally substituted with one or more R^{10a};
   each R^{10a} is independently halogen, —CN, —NO₂, —OH, —OR', —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
   each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
   or two R^{a} are taken together with the atom to which they are attached to form a heterocycloalkyl optionally substituted with one or more R;
   each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
   or two R^{b} are taken together with the atom to which they are attached to form a heterocycloalkyl optionally substituted with one or more R;
   R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
   or R^{c} and R^{d} are taken together with the atom to which they are attached to form a heterocycloalkyl optionally substituted with one or more R; and
   each R is independently halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆hydroxyalkyl, C₁-C₆aminoalkyl, and C₁-C₆heteroalkyl;
   or two R on the same atom form an oxo.

In some embodiments, L^{N} and L^{C} are each a bond. In some embodiments, the conjugate comprises an alpha-particle emitting radionuclide bound to the metal chelator. In some embodiments, the alpha particle-emitting radionuclide is actinium-225. In some embodiments, the ring closing group is —C(═O)—CH₂—S—, —C(═O)—CH₂-NH-, or —C(═O)—CH₂-O-. In some embodiments, the ring closing group is —C(═O)—CH₂-S-. In some embodiments, each of the amino acid is independently selected from a natural amino acid or an amino acid of Tables 4A-4G. In some embodiments, the monocyclic peptide has a structure of

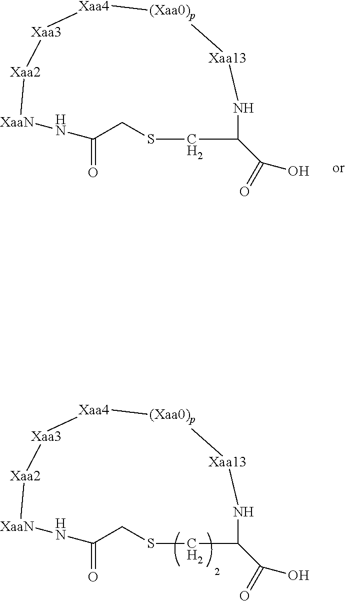

(attachment point to the chelator not shown). In some embodiments, the metal chelator comprises DOTA, DOTP, DOTMA, DOTAM, DOTAGA, DTPA, NTA, EDTA, DO3A, DO2A, NOC, NOTA, TETA, DiAmSar, CB-Cyclam, CB-TE2A, DOTA-4AMP, H₄pypa, H₄octox, H₄octapa, p-NO₂-Bn-neunpa, or NOTP. In some embodiments, the metal chelator is DOTA. In some embodiments, the conjugate comprises a linker that covalently attaches the cyclic peptide with the metal chelator. In some embodiments, the linker comprises one or more lysine residues. In some embodiments, the linker is a lysine residue. In some embodiments, the linker comprises one or more of substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. In some embodiments, the linker is attached to the binding peptide or the monocyclic peptide via the N terminus of the peptide. In some embodiments, the linker is attached to the binding peptide or the monocyclic peptide via the C terminus of the peptide. In some embodiments, the linker is attached to the binding peptide or the monocyclic peptide via a non-terminal amino acid of the peptide. In some embodiments, the cyclic peptide is directly linked to the metal chelator through a covalent bond without a linker.

In one aspect, disclosed herein is a conjugate prepared by one or more of the following steps:

a) synthesizing the peptide sequence by solid phase peptide synthesis; b) cyclizing the peptide by forming an intramolecular non-peptide bond; c) coupling the metal chelator to the peptide; and d) optionally labeling the conjugate with a radionuclide. In some embodiments, synthesizing the peptide comprises synthesizing the peptide sequence in a protected form and performing a de-protecting reaction. In some embodiments, cyclizing the peptide comprises forming a non-peptide bond between the N-terminus and the C-terminus of the peptide. In some embodiments, cyclizing the peptide comprises forming a non-peptide bond between the N-terminus and a cysteine or homocysteine of the peptide. In some embodiments, cyclizing the peptide comprises forming a ring closing group selected from —C(═O)—CH₂—S—, —S—, —CH═CH—, —NH—, -succinimide-S—, —C(═O)—CH₂—NH—, and —C(═O)—CH₂—O—. In some embodiments, the peptide has a net charge of −3, −2, −1, 0, or +1. In some embodiments, the metal chelator comprises DOTA, DOTP, DOTMA, DOTAM, DOTAGA, DTPA, NTA, EDTA, DO3A, DO2A, NOC, NOTA, TETA, DiAmSar, CB-Cyclam, CB-TE2A, DOTA-4AMP, H₄pypa, H₄octox, H₄octapa, p-NO₂-Bn-neunpa, or NOTP. In some embodiments, the metal chelator is DOTA. In some embodiments, the conjugate comprises a linker that covalently attaches the cyclic peptide with the metal chelator. In some embodiments, the linker comprises one or more lysine residues. In some embodiments, the linker is a lysine residue. In some embodiments, the linker comprises one or more of substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. In some embodiments, the linker is attached to the binding peptide or the monocyclic peptide via the N terminus of the peptide. In some embodiments, the linker is attached to the binding peptide or the monocyclic peptide via the C terminus of the peptide. In some embodiments, the linker is attached to the binding peptide or the monocyclic peptide via a non-terminal amino acid of the peptide. In some embodiments, the cyclic peptide is directly linked to the metal chelator through a covalent bond without a linker.

In some embodiments, the conjugate disclosed herein comprises two or more metal chelators. In some embodiments, the conjugate comprises two radionuclides bound to the metal chelators.

In some embodiments, the conjugate disclosed herein has an elimination half-life in rats of about 1 to 120 hours. In some embodiments, the conjugate disclosed herein has an elimination half-life in rats of about 2 to 8 hours.

In some embodiments, the conjugate disclosed herein has a residence time of about 4 to 7 days in a tumor when administered to a subject having the tumor.

In some embodiments, 2% to 99% of the conjugate or the peptide disclosed herein binds to Human Serum Albumin (HSA) in vitro as determined by HSA-HPLC method. In some embodiments, about 40% to about 80% of the conjugate or the peptide binds to HSA in vitro as determined by HSA-HPLC method.

In one aspect, disclosed herein is a pharmaceutical composition comprising a conjugate described herein and a pharmaceutically acceptable excipient or carrier.

In one aspect, disclosed herein is a method of treating cancer in a subject in need thereof comprising administering to the subject a herein-described conjugate or a herein-described pharmaceutical composition.

### INCORPORATION BY REFERENCE

All publications, patents, and patent applications mentioned in this specification are herein incorporated by reference for the specific purposes identified herein.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawing (also “figure” and “FIG.” herein), of which:

FIGS. 1A to 1D illustrate the structures of exemplary conjugates of the present disclosure. FIG. 1A illustrates a conjugate comprising a monocyclic peptide of 11 amino acid residues, a linker, and a metal chelator. FIG. 1B illustrates a conjugate comprising a monocyclic peptide of 12 amino acid residues, a linker, a metal chelator, and 225-Ac bound to the metal chelator. FIG. 1C illustrates a conjugate comprising a monocyclic peptide of 12 amino acid residues that is attached directly to a metal chelator. FIG. 1D illustrates a conjugate comprising a linker with three motifs, a metal chelator, and two peptides.

FIG. 2 illustrates the structures of representative metal chelators.

FIG. 3 illustrates the structures of representative metal chelators.

FIG. 4 illustrates the structures of representative metal chelators.

FIG. 5 illustrates the structures of representative metal chelators.

FIG. 6 illustrates the structures of representative metal chelators.

FIG. 7 illustrates the structures of representative metal chelators.

FIG. 8 illustrates the structures of representative metal chelators.

FIG. 9 illustrates the structures of representative metal chelators.

FIG. 10 illustrates the structures of representative metal chelators.

FIG. 11 illustrates the structures of representative metal chelators.

FIG. 12 illustrates the structures of representative metal chelators.

FIG. 13 illustrates the structures of representative metal chelators.

FIG. 14 illustrates the structures of representative metal chelators.

FIG. 15 illustrates the structures of representative metal chelators.

FIG. 16 illustrates the structures of representative metal chelators.

FIG. 17A illustrates the structure of an exemplary conjugate of the present disclosure comprising a cyclic peptide of 12 amino acids, including a C-terminus cysteine residue; FIG. 17B illustrates the structure of an exemplary conjugate of the present disclosure comprising a cyclic peptide of 9 amino acids, including a C-terminus cysteine residue.

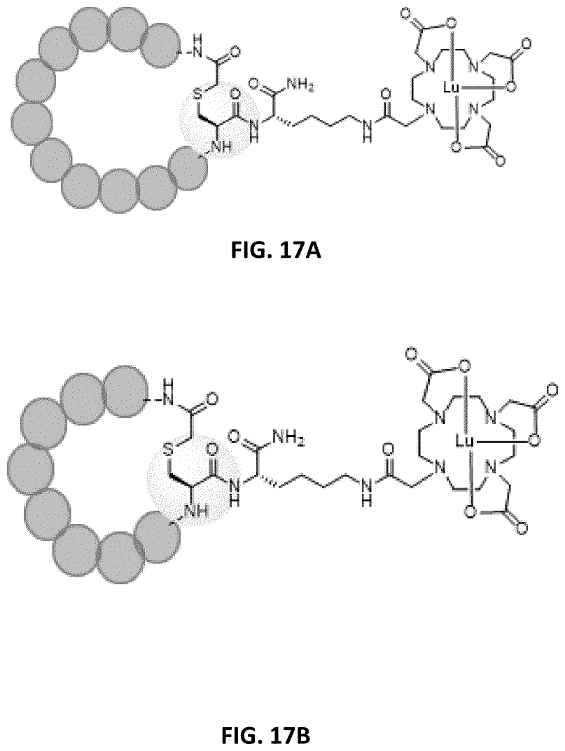

### DETAILED DESCRIPTION

The following description and examples illustrate embodiments of the present disclosure in detail. It is to be understood that this present disclosure is not limited to the particular embodiments described herein and as such can vary. Those of skill in the art will recognize that there are numerous variations and modifications of this present disclosure, which are encompassed within its scope.

Although various features of the present disclosure may be described in the context of a single embodiment, the features may also be provided separately or in any suitable combination. Conversely, although the present disclosure may be described herein in the context of separate embodiments for clarity, the present disclosure may also be implemented in a single embodiment.

The section headings used herein are for organizational purposes only and are not to be construed as limiting the subject matter described.

All terms are intended to be understood as they would be understood by a person skilled in the art. Unless defined otherwise, all technical and scientific terms used herein have the same meaning as commonly understood by one of ordinary skill in the art to which the disclosure pertains.

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only, and is not intended to be limiting.

### I. Definitions

As used in the specification and appended claims, unless specified to the contrary, the following terms have the meaning indicated below.

As used herein and in the appended claims, the singular forms “a,” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 15%, up to 10%, up to 5%, or up to 1% of a given value. Alternatively, particularly with respect to biological systems or processes, the term can mean within an order of magnitude, within 5-fold, or within 2-fold, of a value.

The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of”the described features.

“Amino” refers to the —NH₂ radical.

“Cyano” refers to the —CN radical.

“Nitro” refers to the —NO₂ radical.

“Oxo” refers to the ═O radical.

“Imino” refers to the ═N—H radical.

“Oximo” refers to the ═N—OH radical.

“Hydrazino” refers to the ═N—NH₂ radical.

“Hydroxy” or “hydroxyl” refers to the —OH radical.

“Hydroxyamino” refers to the —NH—OH radical.

“Acyl” refers to a substituted or unsubstituted alkylcarbonyl, substituted or unsubstituted alkenylcarbonyl, substituted or unsubstituted alkynylcarbonyl, substituted or unsubstituted cycloalkylcarbonyl, substituted or unsubstituted heterocycloalkylcarbonyl, substituted or unsubstituted arylcarbonyl, substituted or unsubstituted heteroarylcarbonyl, amide, or ester, wherein the carbonyl atom of the carbonyl group is the point of attachment. Unless stated otherwise specifically in the specification, an alkylcarbonyl group, alkenylcarbonyl group, alkynylcarbonyl group, cycloalkylcarbonyl group, amide group, or ester group is optionally substituted, for example, with oxo, halogen, amino, nitrile, nitro, hydroxyl, haloalkyl, alkoxy, aryl, cycloalkyl, heterocycloalkyl, heteroaryl, and the like.

“Alkyl” refers to an optionally substituted straight-chain, or optionally substituted branched-chain saturated hydrocarbon monoradical. An alkyl group can have from one to about twenty carbon atoms, from one to about ten carbon atoms, or from one to six carbon atoms. Examples include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3 -dimethyl-l-butyl, 2-ethyl-1-butyl, n-butyl, isobutyl, sec-butyl, t-butyl, n-pentyl, isopentyl, neopentyl, tert-amyl, and hexyl, and longer alkyl groups, such as heptyl, octyl, and the like. Whenever it appears herein, a numerical range such as “C₁-C₆ alkyl” means that the alkyl group consists of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁-C₁₀ alkyl, a C₁-C₉ alkyl, a C₁-C₈ alkyl, a C₁-C₇ alkyl, a C₁-C₆ alkyl, a C₁-C₅ alkyl, a C₁-C₄ alkyl, a C₁-C₃ alkyl, a C₁-C₂ alkyl, or a C₁ alkyl. Unless stated otherwise specifically in the specification, an alkyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, the alkyl is optionally substituted with oxo, halogen, —CN, —CF₃, —OH, —OMe, —NH₂, —NO₂, or —C≡CH. In some embodiments, the alkyl is optionally substituted with oxo, halogen, —CN, —CF₃, —OH, or —OMe. In some embodiments, the alkyl is optionally substituted with halogen.

“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkylene is optionally substituted with oxo, halogen, —CN, —CF₃, —OH, —OMe, —NH₂, or —NO₂. In some embodiments, an alkylene is optionally substituted with oxo, halogen, —CN, —CF₃, —OH, or —OMe. In some embodiments, the alkylene is optionally substituted with halogen. In some embodiments, the alkylene is —CH₂—, —CH₂CH₂—, —CH₂CH₂CH₂—, or —CH₂CH(CH₃)CH₂—. In some embodiments, the alkylene is —CH₂—. In some embodiments, the alkylene is —CH₂CH₂—. In some embodiments, the alkylene is —CH₂CH₂CH₂—.

“Alkenyl” refers to an optionally substituted straight-chain, or optionally substituted branched-chain hydrocarbon monoradical having one or more carbon-carbon double-bonds. In some embodiments, an alkenyl group has from two to about ten carbon atoms, or two to about six carbon atoms. The group may be in either the cis or trans configuration about the double bond(s), and should be understood to include both isomers. Examples include, but are not limited to, ethenyl (—CH═CH₂), 1-propenyl (—CH₂CH═CH₂), isopropenyl [—C(CH₃)═CH₂], butenyl, 1,3-butadienyl, and the like. Whenever it appears herein, a numerical range such as “C₂-C₆ alkenyl” means that the alkenyl group may consist of 2 carbon atoms, 3 carbon atoms, 4 carbon atoms, 5 carbon atoms, or 6 carbon atoms, although the present definition also covers the occurrence of the term “alkenyl” where no numerical range is designated. In some embodiments, the alkenyl is a C2-C₁₀ alkenyl, a C₂-C₉ alkenyl, a C₂-C₈ alkenyl, a C₂-C₇ alkenyl, a C₂-C₆ alkenyl, a C₂-C₅ alkenyl, a C₂-C₄ alkenyl, a C₂-C₃ alkenyl, or a C₂ alkenyl. Unless stated otherwise specifically in the specification, an alkenyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkenyl is optionally substituted with oxo, halogen, —CN, —CF₃, —OH, —OMe, —NH₂, or —NO₂. In some embodiments, an alkenyl is optionally substituted with oxo, halogen, —CN, —CF₃, —OH, or —OMe. In some embodiments, the alkenyl is optionally substituted with halogen.

The term “alkenylene” or “alkenylene chain” refers to an optionally substituted straight or branched divalent hydrocarbon chain in which at least one carbon-carbon double bond is present linking the rest of the molecule to a radical group. In some embodiments, the alkenylene is —CH═CH—, —CH₂CH═CH—, or —CH═CHCH₂—. In some embodiments, the alkenylene is —CH═CH—. In some embodiments, the alkenylene is —CH₂CH═CH—. In some embodiments, the alkenylene is —CH═CHCH₂—.

“Alkynyl” refers to an optionally substituted straight-chain or optionally substituted branched-chain hydrocarbon monoradical having one or more carbon-carbon triple-bonds. In some embodiments, an alkynyl group has from two to about ten carbon atoms, more preferably from two to about six carbon atoms. Examples include, but are not limited to, ethynyl, 2-propynyl, 2-butynyl, 1,3-butadiynyl, and the like. Whenever it appears herein, a numerical range such as “C₂-C₆ alkynyl” means that the alkynyl group may consist of 2 carbon atoms, 3 carbon atoms, 4 carbon atoms, 5 carbon atoms, or 6 carbon atoms, although the present definition also covers the occurrence of the term “alkynyl” where no numerical range is designated. In some embodiments, the alkynyl is a C₂-C₁₀ alkynyl, a C₂-C₉ alkynyl, a C₂-C₈ alkynyl, a C₂-C₇ alkynyl, a C₂-C₆ alkynyl, a C₂-0₅ alkynyl, a C₂-C₄ alkynyl, a C₂-C₃ alkynyl, or a C₂ alkynyl. Unless stated otherwise specifically in the specification, an alkynyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkynyl is optionally substituted with oxo, halogen, —CN, —CF₃, —OH, —OMe, —NH₂, or —NO₂. In some embodiments, an alkynyl is optionally substituted with oxo, halogen, —CN, —CF₃, —OH, or —OMe. In some embodiments, the alkynyl is optionally substituted with halogen. The term “alkynylene” refers to an optionally substituted straight-chain or optionally substituted branched-chain divalent hydrocarbon having one or more carbon-carbon triple-bonds.

“Alkylamino” refers to a radical of the formula —N(Rₐ)₂ where Rₐ is an alkyl radical as defined, or two Rₐ, taken together with the nitrogen atom, can form a substituted or unsubstituted C₂-C₇ heterocyloalkyl ring. Unless stated otherwise specifically in the specification, an alkylamino group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkylamino is optionally substituted with oxo, halogen, —CN, —CF₃, —OH, —OMe, —NH₂, or —NO₂. In some embodiments, an alkylamino is optionally substituted with oxo, halogen, —CN, —CF₃, —OH, or —OMe. In some embodiments, the alkylamino is optionally substituted with halogen.

“Alkoxy” refers to a radical of the formula —ORₐ where Rₐ is an alkyl radical as defined. Unless stated otherwise specifically in the specification, an alkoxy group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkoxy is optionally substituted with oxo, halogen, —CN, —CF₃, —OH, —OMe, —NH₂, or —NO₂. In some embodiments, an alkoxy is optionally substituted with oxo, halogen, —CN, —CF₃, —OH, or —OMe. In some embodiments, the alkoxy is optionally substituted with halogen.

“Aminoalkyl” refers to an alkyl radical, as defined above, that is substituted by one or more amines. In some embodiments, the alkyl is substituted with one amine. In some embodiments, the alkyl is substituted with one, two, or three amines. Hydroxyalkyl include, for example, aminomethyl, aminoethyl, aminopropyl, aminobutyl, or aminopentyl. In some embodiments, the hydroxyalkyl is aminomethyl.

The term “aryl” refers to a radical comprising at least one aromatic ring wherein each of the atoms forming the ring is a carbon atom. Aryl groups can be optionally substituted. Examples of aryl groups include, but are not limited to phenyl, and naphthyl. In some embodiments, the aryl is phenyl. Depending on the structure, an aryl group can be a monoradical or a diradical (i.e., an arylene group). Unless stated otherwise specifically in the specification, the term “aryl” or the prefix “ar-”(such as in “aralkyl”) is meant to include aryl radicals that are optionally substituted. In some embodiments, an aryl group comprises a partially reduced cycloalkyl group defined herein (e.g., 1,2-dihydronaphthalene). In some embodiments, an aryl group comprises a fully reduced cycloalkyl group defined herein (e.g., 1,2,3,4-tetrahydronaphthalene). When aryl comprises a cycloalkyl group, the aryl is bonded to the rest of the molecule through an aromatic ring carbon atom. An aryl radical can be a monocyclic or polycyclic (e.g., bicyclic, tricyclic, or tetracyclic) ring system, which may include fused, spiro or bridged ring systems. Unless stated otherwise specifically in the specification, an aryl may be optionally substituted, for example, with halogen, amino, alkylamino, aminoalkyl, nitrile, nitro, hydroxyl, alkyl, alkenyl, alkynyl, haloalkyl, heteroalkyl, alkoxy, aryl, cycloalkyl, heterocycloalkyl, heteroaryl, —S(O)₂NH—C₁-C₆alkyl, and the like. In some embodiments, an aryl is optionally substituted with halogen, methyl, ethyl, —CN, —CF₃, —OH, —OMe, —NH₂, —NO₂, —S(O)₂NH₂, —S(O)₂NHCH₃, —S(O)₂NHCH₂CH₃, —S(O)₂NHCH(CH₃)₂, —S(O)₂N(CH₃)2, or —S(O)₂NHC(CH₃)₃. In some embodiments, an aryl is optionally substituted with halogen, methyl, ethyl, —CN, —CF₃, —OH, or —OMe. In some embodiments, the aryl is optionally substituted with halogen. In some embodiments, the aryl is substituted with alkyl, alkenyl, alkynyl, haloalkyl, or heteroalkyl, wherein each alkyl, alkenyl, alkynyl, haloalkyl, heteroalkyl is independently unsubstituted, or substituted with halogen, methyl, ethyl, —CN, —CF₃, —OH, —OMe, —NH₂, or —NO_{2.}

The term “cycloalkyl” refers to a monocyclic or polycyclic non-aromatic radical, wherein each of the atoms forming the ring (i.e. skeletal atoms) is a carbon atom. In some embodiments, cycloalkyls are saturated or partially unsaturated. In some embodiments, cycloalkyls are spirocyclic or bridged compounds. In some embodiments, cycloalkyls are fused with an aromatic ring (in which case the cycloalkyl is bonded through a non-aromatic ring carbon atom). Cycloalkyl groups include groups having from 3 to 10 ring atoms. Representative cycloalkyls include, but are not limited to, cycloalkyls having from three to ten carbon atoms, from three to eight carbon atoms, from three to six carbon atoms, or from three to five carbon atoms. Monocyclic cycloalkyl radicals include, for example, cyclopropyl, cyclobutyl, cyclopentyl, cyclohexyl, cycloheptyl, and cyclooctyl. In some embodiments, the monocyclic cycloalkyl is cyclopentyl. In some embodiments, the monocyclic cycloalkyl is cyclopentenyl or cyclohexenyl. In some embodiments, the monocyclic cycloalkyl is cyclopentenyl. Polycyclic radicals include, for example, adamantyl, 1,2-dihydronaphthalenyl, 1,4-dihydronaphthalenyl, tetrainyl, decalinyl, 3,4-dihydronaphthalenyl-1(2H)-one, spiro[2.2]pentyl, norbornyl and bicycle[1.1.1]pentyl. Unless otherwise stated specifically in the specification, a cycloalkyl group may be optionally substituted. Representative cycloalkyls include, but are not limited to, cycloalkyls having from three to fifteen carbon atoms (C₃-C₁₅ cycloalkyl), from three to ten carbon atoms (C₃-C₁₀ cycloalkyl), from three to eight carbon atoms (C₃-C₈ cycloalkyl), from three to six carbon atoms (C₃-C₆ cycloalkyl), from three to five carbon atoms (C₃-C₅ cycloalkyl), or three to four carbon atoms (C₃-C₄ cycloalkyl). In some embodiments, the cycloalkyl is a 3- to 6-membered cycloalkyl. In some embodiments, the cycloalkyl is a 5- to 6-membered cycloalkyl. Monocyclic cycloalkyls include, for example, cyclopropyl, cyclobutyl, cyclopentyl, cyclohexyl, cycloheptyl, and cyclooctyl. Polycyclic cycloalkyls or carbocycles include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example, cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a cycloalkyl is optionally substituted with oxo, halogen, methyl, ethyl, —CN, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Halo” or “halogen” refers to bromo, chloro, fluoro, or iodo. In some embodiments, halogen is fluoro or chloro. In some embodiments, halogen is fluoro.

“Haloalkyl” refers to an alkyl radical, as defined above, that is substituted by one or more halogens. In some embodiments, the alkyl is substituted with one, two, or three halogens. In some embodiments, the alkyl is substituted with one, two, three, four, five, or six halogens. Haloalkyl can include, for example, iodoalkyl, bromoalkyl, chloroalkyl, and fluoroalkyl. For example, “fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, or combinations thereof wherein the heteroalkyl is attached to the rest of the molecule at a carbon atom of the heteroalkyl. Examples of such heteroalkyl are, for example, —CH₂—O—CH₂—, —CH₂—N(alkyl)-CH₂—, —CH₂—N(aryl)-CH₂—, —OCH₂CH₂O—, —OCH₂CH₂OCH₂CH₂O—, or —OCH₂CH₂OCH₂CH₂OCH₂CH₂O—. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

The term “heterocycloalkyl” refers to a cycloalkyl group that includes at least one hetero ring atom, e.g., a heteroatom selected from nitrogen, oxygen, and sulfur. Unless stated otherwise specifically in the specification, the heterocycloalkyl radical may be a monocyclic, or bicyclic ring system, which may include fused (when fused with an aryl or a heteroaryl ring, the heterocycloalkyl is bonded through a non-aromatic ring atom) or bridged ring systems. The nitrogen, carbon or sulfur atoms in the heterocyclyl radical may be optionally oxidized. The nitrogen atom may be optionally quaternized. The heterocycloalkyl radical is partially or fully saturated. Examples of heterocycloalkyl radicals include, but are not limited to, dioxolanyl, thienyl[1,3]dithianyl, tetrahydroquinolyl, tetrahydroisoquinolyl, decahydroquinolyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl. The term heterocycloalkyl also includes all ring forms of carbohydrates, including but not limited to monosaccharides, disaccharides and oligosaccharides. Unless otherwise noted, heterocycloalkyls have from 2 to 12 carbons in the ring. In some embodiments, heterocycloalkyls have from 2 to 10 carbons in the ring. In some embodiments, heterocycloalkyls have from 2 to 10 carbons in the ring and 1 or 2 N atoms. In some embodiments, heterocycloalkyls have from 2 to 10 carbons in the ring and 3 or 4 N atoms. In some embodiments, heterocycloalkyls have from 2 to 12 carbons, 0-2 N atoms, 0-2 O atoms, 0-2 P atoms, and 0-1 S atoms in the ring. In some embodiments, heterocycloalkyls have from 2 to 12 carbons, 1-3 N atoms, 0-1 O atoms, and 0-1 S atoms in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heterocycloalkyl is optionally substituted with oxo, halogen, methyl, ethyl, —CN, —CF₃, —OH, —OMe, —NH₂, or —NO₂. In some embodiments, a heterocycloalkyl is optionally substituted with oxo, halogen, methyl, ethyl, —CN, —CF₃, —OH, or —OMe. In some embodiments, the heterocycloalkyl is optionally substituted with halogen.

“Heteroaryl” refers to a ring system radical comprising carbon atom(s) and one or more ring heteroatoms selected from the group consisting of nitrogen, oxygen, phosphorous, and sulfur, and at least one aromatic ring. In some embodiments, heteroaryl is monocyclic, bicyclic or polycyclic. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, furazanyl, indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Illustrative examples of bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, heteroaryl is pyridinyl, pyrazinyl, pyrimidinyl, thiazolyl, thienyl, thiadiazolyl or furyl. In some embodiments, a heteroaryl contains 0-6 N atoms in the ring. In some embodiments, a heteroaryl contains 1-4 N atoms in the ring. In some embodiments, a heteroaryl contains 4-6 N atoms in the ring. In some embodiments, a heteroaryl contains 0-4 N atoms, 0-1 O atoms, 0-1 P atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉ heteroaryl. In some embodiments, monocyclic heteroaryl is a C₁-C₅ heteroaryl. In some embodiments, monocyclic heteroaryl is a 5-membered or 6-membered heteroaryl. In some embodiments, a bicyclic heteroaryl is a C₆-C₉ heteroaryl. In some embodiments, a heteroaryl group comprises a partially reduced cycloalkyl or heterocycloalkyl group defined herein (e.g., 7,8-dihydroquinoline). In some embodiments, a heteroaryl group comprises a fully reduced cycloalkyl or heterocycloalkyl group defined herein (e.g., 5,6,7,8-tetrahydroquinoline). When heteroaryl comprises a cycloalkyl or heterocycloalkyl group, the heteroaryl is bonded to the rest of the molecule through a heteroaromatic ring carbon or hetero atom. A heteroaryl radical can be a monocyclic or polycyclic (e.g., bicyclic, tricyclic, or tetracyclic) ring system, which may include fused, spiro or bridged ring systems. Unless stated otherwise specifically in the specification, a heteroaryl is optionally substituted, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroaryl is optionally substituted with halogen, methyl, ethyl, —CN, —CF₃, —OH, —OMe, —NH₂, or —NO₂. In some embodiments, a heteroaryl is optionally substituted with halogen, methyl, ethyl, —CN, —CF₃, —OH, or —OMe. In some embodiments, the heteroaryl is optionally substituted with halogen.

The term “moiety” refers to a specific segment or functional group of a molecule. Chemical moieties are often recognized chemical entities embedded in or appended to a molecule.

The terms “treat,” “prevent,” “ameliorate,” and “inhibit,” as well as words stemming therefrom, as used herein, do not necessarily imply 100% or complete treatment, prevention, amelioration, or inhibition. Rather, there are varying degrees of treatment, prevention, amelioration, and inhibition of which one of ordinary skill in the art recognizes as having a potential benefit or therapeutic effect. In this respect, the disclosed methods can provide any amount of any level of treatment, prevention, amelioration, or inhibition of the disorder in a mammal. For example, a disorder, including symptoms or conditions thereof, may be reduced by, for example, about 100%, about 90%, about 80%, about 70%, about 60%, about 50%, about 40%, about 30%, about 20%, or about 10%. Furthermore, the treatment, prevention, amelioration, or inhibition provided by the methods disclosed herein can include treatment, prevention, amelioration, or inhibition of one or more conditions or symptoms of the disorder, e.g., cancer or an inflammatory disease. As used herein, “treating” includes the concepts of “alleviating”, which refers to lessening the frequency of occurrence or recurrence, or the severity, of any symptoms or other ill effects related to a disorder and/or the associated side effects. The term “treating” also encompasses the concept of “managing” which refers to reducing the severity of a particular disease or disorder in a patient or delaying its recurrence, e.g., lengthening the period of remission in a patient who had suffered from the disease.

In certain embodiments, the term “prevent” or “preventing” as related to a disease or disorder can refer to a compound that in a statistical sample, reduces the occurrences of the disorder or condition in the treated sample relative to an untreated control sample, or delays the onset or reduces the severity of one or more symptoms of the disorder or condition relative to the untreated control sample.

The term “therapeutically effective amount” as used herein to refer to an amount effective at the dosage and duration necessary to achieve the desired therapeutic result. A therapeutically effective amount of the composition may vary depending on factors such as the individual's condition, age, sex, and weight, and the ability of the protein to elicit the desired response of the individual. A therapeutically effective amount can also be an amount that exceeds any toxic or deleterious effect of the composition that would have a beneficial effect on the treatment.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be un-substituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc.).

As used herein, the term “substituent” means positional variables on the atoms of a core molecule that are substituted at a designated atom position, replacing one or more hydrogens on the designated atom, provided that the designated atom's normal valency is not exceeded, and that the substitution results in a stable compound. Combinations of substituents and/or variables are permissible only if such combinations result in stable compounds. A person of ordinary skill in the art should note that any carbon as well as heteroatom with valences that appear to be unsatisfied as described or shown herein is assumed to have a sufficient number of hydrogen atom(s) to satisfy the valences described or shown. In certain instances one or more substituents having a double bond (e.g., “oxo” or “═O”) as the point of attachment may be described, shown or listed herein within a substituent group, wherein the structure may only show a single bond as the point of attachment to the core structure. A person of ordinary skill in the art would understand that, while only a single bond is shown, a double bond is intended for those substituents.

The term “optionally substituted” or “substituted” means that the referenced group is optionally substituted with one or more additional group(s) individually and independently selected from D, halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from D, halogen, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁-C₄alkyl, —S(═O)C₁-C₄alkyl, and —S(═O)₂C₁-C₄alkyl. In some embodiments, optional substituents are independently selected from D, halogen, —CN, —NH₂, —OH, —NH(CH₃), —N(CH₃)₂, —NH(cyclopropyl), —CH₃, —CH₂CH₃, —CF₃, —OCH₃, and —OCF₃. In some embodiments, substituted groups are substituted with one or two of the preceding groups. In some embodiments, an optional substituent on an aliphatic carbon atom (acyclic or cyclic) includes oxo (═O). When indicating the number of substituents, the term “one or more” means from one substituent to the highest possible number of substitutions, i.e. replacement of one hydrogen up to replacement of all hydrogens by substituents.

The term “unsubstituted” means that the specified group bears no substituents.

Certain compounds described herein may exist in tautomeric forms, and all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

The term “peptide” as used herein refers to a compound that includes two or more amino acids. A peptide described herein can comprise one or more unnatural amino acids. The term “peptide” also encompasses peptide mimetics. In the present disclosure, the term “amino acid” is used in its broadest meaning and it embraces not only natural amino acids but also derivatives thereof and artificial amino acids. For example, the term “amino acid” encompasses unnatural amino acids.

As used herein, the term “unnatural amino acid” refers to an amino acid other than the 20 amino acids that occur naturally in protein.

The term “protein” as used herein refers to a polypeptide (i.e., a string of at least 3 amino acids linked to one another by peptide bonds). Proteins can include moieties other than amino acids (e.g., may be glycoproteins, proteoglycans, etc.) and/or can be otherwise processed or modified. A protein can be a complete polypeptide as produced by and/or active in a cell (with or without a signal sequence). In some embodiments, a protein is or comprises a characteristic portion such as a polypeptide as produced by and/or active in a cell. A protein can include more than one polypeptide chain. For example, polypeptide chains can be linked by one or more disulfide bonds or associated by other means.

The term “peptide mimetic” or “mimetic” refers to biologically active compounds that mimic the biological activity of a peptide or a protein but are no longer entirely peptidic in chemical nature, e.g., they can contain non-peptide bonds (that are, bonds other than amide bonds between amino acids). As used herein, the term peptide mimetic is used in a broader sense to include molecules that are no longer completely peptidic in nature, such as pseudo-peptides, semi-peptides and peptoids. Whether completely or partially non-peptide, peptide mimetics described herein can provide a spatial arrangement of reactive chemical moieties that closely resemble the three-dimensional arrangement of active groups in the subject amino acid sequence or subject molecule on which the peptide mimetic is based. As a result of this similar active-site geometry, the peptide mimetic can have effects on biological systems that are similar to the biological activity of the subject entity.

In some embodiments, the peptide mimetics are substantially similar in both three-dimensional shape and biological activity to the subject amino acid sequence or subject molecule on which the peptide mimetic is based. Examples of methods of structurally modifying a peptide to create a peptide mimetic include the inversion of backbone chiral centers leading to D-amino acid residue structures that may, particularly at the N-terminus, lead to enhanced stability for proteolytical degradation without adversely affecting activity. An example is described in the paper “Tritiated D-ala1-Peptide T Binding”, Smith C. S. et al., Drug Development Res., 15, pp. 371-379 (1988). A second method is altering cyclic structure for stability, such as N to C interchain imides and lactams (Ede et al. in Smith and Rivier (Eds.) “Peptides: Chemistry and Biology”, Escom, Leiden (1991), pp. 268-270). An example of this is provided in conformationally restricted thymopentin-like compounds, such as those disclosed in US4457489. A third method is to substitute peptide bonds in the subject entity by pseudopeptide bonds that confer resistance to proteolysis.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein, C₁-Cₓ (or C₁₋ₓ) includes C₁-C₂, C₁-C₃ . . . C₁-Cₓ. By way of example only, a group designated as “C₁-C₄” indicates that there are one to four carbon atoms in the moiety, i.e. groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl. Also, by way of example, C₀-C₂ alkylene includes a direct bond, —CH₂—, and —CH₂CH₂— linkages.

The term “cyclized” or “cyclization” as used herein means that two amino acids apart from each other by at least one amino acid bind directly or bind indirectly to each other in one peptide to form a cyclic structure in the molecule. In some cases, the two amino acids bind via a linker or the like.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a companion animal such as a dog or a cat. In one aspect, the mammal is a human.

The term “therapeutically effective amount” as used herein to refer to an amount effective at the dosage to achieve the desired therapeutic result. A therapeutically effective amount of a composition may vary depending on factors such as the individual's condition (e.g., age, sex, and weight), the radiopharmaceutical conjugate, and the method of administration (e.g., oral or parenteral).

### II. Radiopharmaceutical Conjugates

Provided herein are radiopharmaceutical conjugates and pharmaceutical compositions comprising the conjugates. The conjugates and compositions can be useful for treating cancer. The conjugates and compositions can also be useful in imaging and disease diagnosis.

In one aspect, described herein is a conjugate that comprises a peptide and a metal chelator that is configured to bind with a radionuclide. The peptide can be cyclic or acyclic, and it can be monocyclic, bicyclic or polycyclic. In one aspect, described herein is a conjugate that comprises a cyclic peptide and a metal chelator that is configured to bind with a radionuclide. In some embodiments, the peptide (such as cyclic peptide) is configured to bind to a target. A conjugate described herein can further comprises a linker that covalently attaches the peptide to the metal chelator. In some embodiments, the conjugate comprises a radionuclide such as ²²⁵Ac bound to the metal chelator.

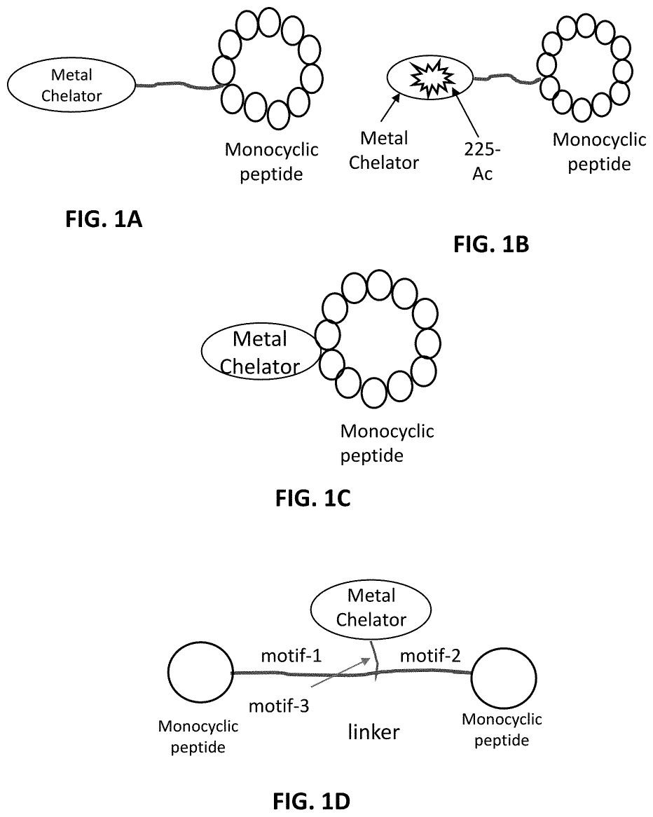

In some embodiments, described herein is a conjugate comprising: (a) a targeting moiety that comprises a monocyclic peptide and (b) a metal chelator configured to bind with a radionuclide. In some embodiments, described herein is a conjugate comprising: (a) a monocyclic peptide and (b) a metal chelator configured to bind with a radionuclide. In some embodiments, described herein is a conjugate comprising: (a) a targeting moiety that comprises a monocyclic peptide; and (b) a metal chelator configured to bind with a radionuclide. In some embodiments, described herein is a conjugate comprising: (a) a monocyclic peptide and (b) a metal chelator configured to bind with a radionuclide. In some embodiments, the monocyclic peptide is cyclized by a non-disulfide bond. In some embodiments, the monocyclic peptide does not comprise a disulfide bond. In some embodiments, the monocyclic peptide comprises 7 to 40 amino acid residues. In some embodiments, described herein is a conjugate comprising: (a) a cyclic binding peptide that binds a cell-surface protein or a protein complex, and (b) a metal chelator configured to bind with a radionuclide. In some embodiments, the cyclic binding peptide is cyclized by a non-disulfide bond and consists of 7-40 amino acid residues. In some embodiments, described herein is a conjugate comprising: (a) a binding peptide that binds a cell-surface protein; (a) a metal chelator configured to bind with a radionuclide; and (c) a linker that covalently attaches the binding peptide to the metal chelator. In some embodiments, the cell-surface protein is an oncofetal antigen, a tight junction protein, an adhesion protein, a transporter receptor, or a tyrosine kinase receptor (TKR). A conjugate described herein can further comprises a linker that covalently attaches the cyclic peptide to the metal chelator. In some embodiments, the conjugate comprises a radionuclide such as ²²⁵Ac bound to the metal chelator. Exemplary configurations of the conjugates described herein are illustrated in FIGS. 1A-1D.

In some embodiments, a herein-described conjugate comprises two or more peptides (i.e., a first peptide, a second peptide, etc.). For example, the conjugate can comprise two different peptides, wherein both of the peptides are configured to bind to the same target, either at the same binding site or at different binding sites. For another example, the conjugate can comprise two different peptides, wherein the two peptides are configured to bind to different targets. For yet another example, the conjugate can comprise two identical peptides.

In some embodiments, a conjugate described herein is designed to have a prescribed elimination profile. The elimination profile can be designed by adjusting the sequence and length of the peptide, the property of the linker, the type of radionuclide, etc. In some embodiments, the conjugate has an elimination half-life of about 30 minutes to 120 hours. In some embodiments, the conjugate has an elimination half-life of about 1 to 120 hours. In some embodiments, the conjugate has an elimination half-life of at least 15 minutes, 30 minutes, 1 hour, 2 hours, 3 hours, 4 hours, 5 hours, 7 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours. In some embodiments, the conjugate has an elimination half-life of at most 120 hour, 80 hours, 70 hours, 60 hours, 50 hours, 40 hours, 30 hours, 24 hours, 12 hours, 10 hours, or 5 hours. In some embodiments, the conjugate has an elimination half-life of about 2 to 24 hours. In some embodiments, the conjugate has an elimination half-life of about 3 to 9 hours. In some embodiments, the conjugate has an elimination half-life of about 2 to 12 hours. In some embodiments, the conjugate has an elimination half-life of about 2 to 8 hours. In some embodiments, the conjugate has an elimination half-life of about 2 to 5 hours. In some embodiments, the conjugate has an elimination half-life of about 3 to 4 hours. In some embodiments, the elimination half-life is determined in rats. In some embodiments, the elimination half-life is determined in humans.

A herein described conjugate can have an elimination half-life in a tumor and non-tumor tissue of the subject. The elimination half-life in a tumor can be the same as or different from (either longer or shorter than) the elimination half-life in a non-tumor issue. In some embodiments, the elimination half-life of the conjugate in a tumor is about 3 hours to 14 days, about 2 to 10 days, about 7 to 10 days, or about 4 to 7 days. In some embodiments, the elimination half-life of the conjugate in a tumor is more than 14 days. In some embodiments, the elimination half-life of the conjugate in a non-tumor tissue is about 1 hour to 14 days, about 12 hours to 2 days, about 1 day to 3 days, about 2 to 10 days, about 7 to 10 days, or about 4 to 7 days. In some embodiments, the elimination half-life of the conjugate in a tumor is at least 1.1, 1.2, 1.3, 1.4, 1.5, 2.0, 2.5, 3.0, 4.0, or 5.0 fold of the elimination half-life of the conjugate in a non-tumor tissue of the subject.

As used herein, the “elimination half-life” can refer to the time it takes from the maximum concentration after administration to half maximum concentration. In some embodiments, the elimination half-life is determined after intravenous administration. In some embodiments, the elimination half-life is measured as biological half-life, which is the half-life of the cold pharmaceutical in the living system. In some embodiments, the elimination half-life is measured as effective half-life, which is the half-life of a radiopharmaceutical in a living system taking into account the half-life of the radionuclide.

A conjugate described herein can have a described time-integrated activity coefficient (i.e., a) in a tumor or non-tumor tissues of a subject. As used herein, a represents the cumulative number of nuclear transformations occurring in a source tissue over a dose-integration period per unit administered activity. The a value of a conjugate can be tuned by modifications of the peptide in the conjugate, e.g., modifying the amino acid sequences and length of the peptide. The a value can be determined using a method known in the art. In some embodiments, the a value of the conjugate in a tumor is from about 6 hours to 14 days. In some embodiments, the a value in a tumor is about 2 to 10 days. In some embodiments, the a value in a tumor is about 4 to 7 days. In some embodiments, the a value in a tumor is about 7 to 10 days. In some embodiments, the a value in a tumor is from about 1 day, 2 days, 3 days, or 4 days to about 5 days, 6 days, 7 days, 8 days, 9 days, 10 days, 11 days, or 12 days. In some embodiments, the a value in a tumor is about 3 days, 4 days, 5 days, 6 days, 7 days, 8 days, 9 days, 10 days, 11 days, or 12 days. In some embodiments, the a value of the conjugate in a non-tumor tissue is from about 6 hours to 14 days. In some embodiments, the a value in a non-tumor tissue is about 2 to 10 days. In some embodiments, the a value in a non-tumor tissue is about 4 to 7 days. In some embodiments, the a value in a non-tumor tissue is about 7 to 10 days. In some embodiments, the a value in a non-tumor tissue is from about 1 day, 2 days, 3 days, or 4 days to about 5 days, 6 days, 7 days, 8 days, 9 days, 10 days, 11 days, or 12 days. In some embodiments, the a value in a non-tumor tissue is about 3 days, 4 days, 5 days, 6 days, 7 days, 8 days, 9 days, 10 days, 11 days, or 12 days. The a value of the conjugate in a tumor can be the same as the a value of the conjugate in a non-tumor tissue of the subject. The a value of the conjugate in a tumor can be longer or shorter than the a value of the conjugate in a non-tumor tissue of the subject. In some embodiments, the a value of the conjugate in a tumor is at least 1.1, 1.2, 1.3, 1.4, 1.5, 2.0, 2.5, 3.0, 4.0, or 5.0 fold of the a value of the conjugate in a non-tumor tissue of the subject.

A conjugate described herein can have an a value in an organ of a subject. In some embodiments, the conjugate has an a value in a kidney of the subject of at most 24 hours. In some embodiments, the a value of the conjugate in a kidney of the subject is at most 18 hours, 15 hours, 12 hours, 10 hours, 8 hours, 6 hours, or 5 hours. In some embodiments, the a value of the conjugate in a kidney of the subject is about 30 minutes to about 24 hours. In some embodiments, the a value of the conjugate in a kidney of the subject is about 2 to 24 hours. In some embodiments, the a value of the conjugate in a kidney of the subject is more than 24 hours. In some embodiments, the a value of the conjugate in a liver of the subject is at most 24 hours. In some embodiments, the a value of the conjugate in a liver of the subject is at most 18 hours, 15 hours, 12 hours, 10 hours, 8 hours, 6 hours, or 5 hours. In some embodiments, the a value of the conjugate in a liver of the subject is about 30 minutes to about 24 hours. In some embodiments, the a value of the conjugate in a liver of the subject is about 2 to 24 hours. In some embodiments, the a value of the conjugate in a liver of the subject is more than 24 hours.

In some cases, the elimination profile of the conjugate can be adjusted by a reversible binding between the conjugate and a plasma protein such as albumin. A suitable affinity between the conjugate and the plasma protein can utilize the plasma protein as a reservoir for the conjugates, attaching and preserving the conjugates at high concentration and releasing the conjugates at a lower concentration, thereby improving elimination profile. In some embodiments, a dissociation constant (Kd) between the conjugate and human serum albumin is at most 500 μM, as determined at room temperature in human serum condition. In some embodiments, the Kd is from about 0.1 nM to about 1000 μM. In some embodiments, the Kd is at most 100 μM. In some embodiments, the Kd is at most 15 μM. In some embodiments, the Kd is from about 1 nM to about 10 μM. In some embodiments, the Kd is from about 10 nM to about 10 μM. In some embodiments, the Kd is from about 50 nM to about 1 μM. In some embodiments, the Kd is from about 100 nM to about 10 μM.

### Peptide Ligand

In one aspect, a conjugate described herein comprises a peptide (e.g., a binding peptide). In some embodiments, the conjugate comprises two or more peptides, which can be the same or different. The peptide can be linear or cyclic. In some embodiments, the peptide is monocyclic. The peptide can comprise any suitable number of amino acid residues. In some embodiments, the peptide comprises from 5 to 50, 6 to 40, 7 to 30, 8 to 25, 12 to 25, or 9 to 20 amino acid residues. In some embodiments, the peptide comprises from 7 to 14 amino acid residues. In some embodiments, the peptide comprises from 9 to 16 amino acid residues. In some embodiments, the peptide comprises from 11 to 14 amino acid residues. In some embodiments, the peptide comprises from 10 to 15 amino acid residues. In some embodiments, the peptide comprises from 10 to 20 amino acid residues. In some embodiments, the peptide comprises from 12 to 15 amino acid residues. In some embodiments, the peptide comprises from 13 to 14 amino acid residues. In some embodiments, the peptide comprises 9 amino acid residues. In some embodiments, the peptide comprises 10 amino acid residues. In some embodiments, the peptide comprises 11 amino acid residues. In some embodiments, the peptide comprises 12 amino acid residues. In some embodiments, the peptide comprises 13 amino acid residues. In some embodiments, the peptide comprises 14 amino acid residues. In some embodiments, the peptide comprises 15 amino acid residues. In some embodiments, the peptide comprises 16 amino acid residues. In some embodiments, the peptide comprises 9 amino acid residues. In some embodiments, the peptide consists of 10 amino acid residues. In some embodiments, the peptide consists of 11 amino acid residues. In some embodiments, the peptide consists of 12 amino acid residues. In some embodiments, the peptide consists of 13 amino acid residues. In some embodiments, the peptide consists of 14 amino acid residues. In some embodiments, the peptide consists of 15 amino acid residues. In some embodiments, the peptide consists of 16 amino acid residues. In some embodiments, the conjugate comprises a monocyclic peptide of 10, 11, 12, 13, 14, or 15 amino acid residues. A peptide described herein can be a binding peptide that binds to a target. In some embodiments, the binding peptide consists of 6 to 40 amino acid residues. In some embodiments, the binding peptide consists of 9 to 20 amino acid residues. In some embodiments, the binding peptide consists of 10 to 15 amino acid residues. In some embodiments, the binding peptide consists of 6 to 12 amino acid residues. In some embodiments, the binding peptide consists of 6 to 10 amino acid residues. In some embodiments, the binding peptide consists of 6 to 8 amino acid residues. In some embodiments, the binding peptide is monocyclic.

The molecular weight of the described peptide can vary. In some embodiments, the peptide has a molecular weight of about 0.1 to about 25 kDa. In some embodiments, the peptide has a molecular weight of about 0.2 to about 20 kDa, about 0.5 to about 15 kDa, about 0.75 to about 10 kDa, about 0.5 to about 10 kDa, about 0.5 to about 5 kDa, about 0.5 to about 2.5 kDa, about 0.5 to about 2 kDa, about 0.5 to about 1.5 kDa, about 0.5 to about 1 kDa, about 1 to about 10 kDa, about 1 to about 5 kDa, about 1 to about 2.5 kDa, about 1 to about 2 kDa, about 1 to about 1.5 kDa, about 1 to about 1.25 kDa, or about 0.5 to about 1.25 kDa. In some embodiments, the peptide has a molecular weight of about 0.5 to 5 kDa. In some embodiments, the peptide has a molecular weight of about 0.5 to 2 kDa. In some embodiments, the peptide has a molecular weight of about 0.75 to 1.75 kDa. In some embodiments, the peptide has a molecular weight of about 1 to 1.5 kDa. In some embodiments, the peptide is monocyclic.

A peptide described herein can be cyclized (i.e., macrocyclized). Cyclization can be achieved less ideally via a single disulfide bond, or more ideally via a peptide bond, alkyl bond, alkenyl bond, ester bond, thioester bond, ether bond, thioether bond, phosphate ether bond, azo bond, C—S—C bond, C—N—C bond, C—N—C bond, C═N—O bond, amide bond, lactam bridge, carbamoyl bond, urea bond, thiourea bond, amine bond, thioamide bond, or the like, but not limited to them. In some embodiments, the peptide is a cyclic peptide that is cyclized by a peptide bond, alkyl bond, alkenyl bond, ester bond, thioester bond, ether bond, thioether bond, phosphate ether bond, azo bond, C—N—C bond, C═N—C bond, C═N—O bond, amide bond, lactam bridge, carbamoyl bond, urea bond, thiourea bond, amine bond, or thioamide bond. In some embodiments, the cyclic peptide is cyclized by a thioether bond. In some embodiments, the cyclic peptide is cyclized via an oxime cyclization reaction. A cyclization of a peptide sometimes stabilizes the peptide structure and thereby enhance affinity for a target. The cyclization can occur between the N- and C-terminus, or it can occur between a terminal amino acid and a non-terminal amino acid. In some embodiments, the cyclization occurs between two non-terminal amino acids. In some embodiments, the peptide is cyclized via oxime cyclization. In some embodiments, the peptide is cyclized between cysteine and haloacyl. In some embodiments, the peptide comprises a haloacetyl group (e.g., chloroacetyl or bromoacetyl) at the N-terminus. In some embodiments, the peptide comprises a haloacetyl group (e.g., chloroacetyl or bromoacetyl) at the C-terminus. In some embodiments, the peptide comprises a Cys at the C-terminus. In some embodiments, the peptide comprises a Cys at the N-terminus. In some embodiments, the cyclization occurs via a thioether bond between Cys and a haloacetyl group. In some embodiments, the cyclization occurs between the N-terminus and the C-terminus of the peptide.

As amino acids for macrocyclization, for example, an amino acid having the following functional group A and an amino acid having a corresponding functional group B can be used (see Table 1). Either the functional group A or the functional group B may be placed on the N-terminal side. The amino acid having the functional group A and the amino acid having the functional group B can each be an N-terminal amino acid or C-terminal amino acid or a non-terminal amino acid. In some embodiments, an amino acid having the functional group A is placed at the N-terminus. In some embodiments, an amino acid having the functional group A is placed at the C-terminus. In some embodiments, an amino acid having the functional group A is placed at a non-terminal amino acid. In some embodiments, an amino acid having the functional group B is placed at the N-terminus. In some embodiments, an amino acid having the functional group B is placed at the C-terminus. In some embodiments, an amino acid having the functional group B is placed at a non-terminal amino acid.

TABLE 1
Functional groups for cyclization

| | Functional group A | Functional group B |
|---|---|---|
| (I) | 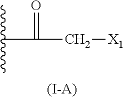 | 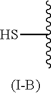 [SMILES: *S ; "(I-B)"] |
| | X₁ is a halogen such as Cl, Br or I | |
| (II) | 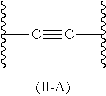 [SMILES: *C#C*] | 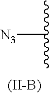 [SMILES: *N=[N+]=[N-] ; "(II-B)"] |
| (III) | 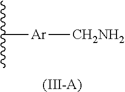 | 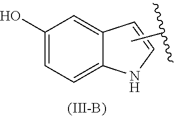 |
| | Ar is substituted or | |
| | unsubstituted aryl or heteroaryl | |
| (IV) | 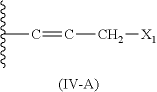 | 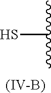 [SMILES: *S ; "(IV-B)"] |
| | X₁ is a halogen such as Cl, Br or I | |
| (V) | 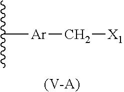 | 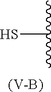 [SMILES: *S ; "(V-B)"] |
| | X₁ is a halogen such as Cl, Br or I; | |
| | Ar is substituted or | |
| | unsubstituted aryl or heteroaryl | |

In some embodiments, as the amino acid (I-A), for example, a chloroacetylated amino acid can be used. Examples of the chloroacetylated amino acids include N-chloroacetyl-L-alanine, N-chloroacetyl-L-phenylalanine, N-chloroacetyl-L-tyrosine, N-chloroacetyl-L-tryptophan, N-3-(2-chloroacetamido)benzoyl-L-phenylalanine, N-3-(2-chloroacetamido)benzoyl-L-tyrosine, N-3-(2-chloroacetamido)benzoyl-L-tryptophan, β-N-chloroacetyl-L-diaminopropanoic acid, γ-N-chloroacetyl-L-diaminobutyric acid, σ-N-chloroacetyl-L-ornithine, ε-N-chloroacetyl-L-lysine, N-3-chloromethylbenzoyl-L-tyrosine, and N-3-chloromethylbenzoyl-L-tryptophane and D-amino acid derivatives corresponding thereto (for example, N-Chloroacetyl-D-alanine, N-Chloroacetyl-D-phenylalanine, N-Chloroacetyl-D-tyrosine, and N-Chloroacetyl-D-tryptophan).

Examples of the amino acid (I-B) include, but are not limited to, cysteine, homocysteine, mercaptonorvaline, mercaptonorleucine, 2-amino-7-mercaptoheptanoic acid, 2-amino-8-mercaptooctanoic acid, and amino acids obtained by protecting the SH group of these amino acids and then eliminating the protecting group, and D-amino acid derivatives corresponding thereto.

The cyclization method can be carried out, for example, according to the method described in Kawakami, T. et al., Nature Chemical Biology 5, 888-890 (2009); Yamagishi, Y. et al., ChemBioChem 10, 1469-1472 (2009); Sako, Y. et al., Journal of American Chemical Society 130, 7932-7934 (2008); or WO2008/117833.

In some embodiments, for example, the amino acid (II-A) is selected from propargylglycine, homopropargylglycine, 2-amino-6-heptynoic acid, 2-amino-7-octynoic acid, and 2-amino-8-nonynoic acid can be used. In addition, 4-pentynoylated or 5-hexynoylated amino acids can also be used. Examples of the 4-pentynoylated amino acids include N-(4-pentenoyl)-L-alanine, N-(4-pentenoyl)-L-phenylalanine, N-(4-pentenoyl)-L-tyrosine, N-(4-pentenoyl)-L-tryptophan, N-3-(4-pentynoylamido)benzoyl-L-phenylalanine, N-3-(4-pentynoylamido)benzoyl-L-tyrosine, N-3-(4-pentynoylamido)benzoyl-L-tryptophan, β-N-(4-pentenoyl)-L-diaminopropanoic acid, γ-N-(4-pentenoyl)-L-diaminobutyric acid, σ-N-(4-pentenoyl)-L-ornithine, and ε-N-(4-pentenoyl)-L-lysine, and D-amino acid derivatives corresponding thereto.

In some embodiments, for example, the amino acid (II-B) is selected from azidoalanine, 2-amino-4-azidobutanoic acid, azidoptonorvaline, azidonorleucine, 2-amino-7-azidoheptanoic acid, and 2-amino-8-azidooctanoic acid can be used. In addition, azidoacetylated or 3-azidopentanoylated amino acids can also be used. Examples of the azidoacetylated amino acids include N-azidoacetyl-L-alanine, N-azidoacetyl-L-phenylalanine, N-azidoacetyl-L-tyrosine, N-azidoacetyl-L-tryptophan, N-3-(4-pentynoylamido)benzoyl-L-phenylalanine, N-3-(4-pentynoylamido)benzoyl-L-tyrosine, N-3-(4-pentynoylamido)benzoyl-L-tryptophan, β-N-azidoacetyl-L-diaminopropanoic acid, γ-N-azidoacetyl-L-diaminobutyric acid, α-N-azidoacetyl-L-ornithine, and ε-N-azidoacetyl-L-lysine, and D-amino acid derivatives corresponding thereto.

The cyclization method can be performed, for example, according to the method described in Sako, Y. et al., Journal of American Chemical Society 130, 7932-7934 (2008) or WO2008/117833.

Examples of amino acid (III-A) include, but are not limited to, N-(4-aminomethyl-benzoyl)-phenylalanine (AMBF) and 4-3-aminomethyltyrosine.

Examples of the amino acid (III-B) include, but are not limited to, 5-hydroxytryptophan (WoH). The cyclization method can be performed, for example, according to the method described in Yamagishi, Y. et al., ChemBioChem 10, 1469-1472 (2009) or WO2008/117833.

Examples of the amino acid (IV-A) include, but are not limited to, 2-amino-6-chloro-hexynoic acid, 2-amino-7-chloro-heptynoic acid, and 2-amino-8-chloro-octynoic acid.

Examples of the amino acid (IV-B) include, but are not limited to, cysteine, homocysteine, mercaptonorvaline, mercaptonorleucine, 2-amino-7-mercaptoheptanoic acid, and 2-amino-8-mercaptooctanoic acid, amino acids obtained by protecting the SH group of these amino acids and then eliminating the protecting group, and D-amino acid derivatives corresponding thereto. The cyclization method can be performed, for example, according to the method described in WO2012/074129.

Examples of the amino acid (V-A) include, but are not limited to, N-3-chloromethylbenzoyl-L-phenylalanine, N-3 -chloromethylbenzoyl-L-tyrosine, and N-3 -chloromethylbenzoyl-L-tryptophane.

Examples of the amino acid (V-B) include, but are not limited to, cysteine, homocysteine, mercaptonorvaline, mercaptonorleucine, 2-amino-7-mercaptoheptanoic acid, and 2-amino-8-mercaptooctanoic acid, and amino acids obtained by protecting the SH group of these amino acids and then eliminating the protecting group, and D-amino acid derivatives corresponding thereto.

The amino acids I-A to V-A and I-B to V-B can be introduced into the peptide in a known manner by chemical synthesis or translation and synthesis described herein. In some embodiments, the cyclization reaction comprises forming a thioether bond using an amino acid comprising a sulfanyl group, e.g., cysteine, homocysteine, mercaptonorvaline, mercaptovaline, mercaptonorleucine, 2-amino-7-mercaptoheptanoic acid, and 2-amino-8-mercaptooctanoic acid.

A peptide described herein can comprise one or more negatively charged amino acids and/or one or more positively charged amino acids. Positively charged amino acids include, for example, lysine, arginine, histidine, and amino acids that contain additional amine groups. Positively charged amino acids can comprise a heteroaryl substitution such as pyridine, imidazole, pyrazole, or triazole that has one or more ring nitrogen atoms. Negatively charged amino acids include, for example, amino acids that contain an additional carboxylic acid group such as glutamic acid or the like.

For example, a peptide described herein can comprise a macrocyclic peptide represented by Formula (I):

(SEQ ID NO: 12)
Xaa1-Xaa2-Xaa3-Xaa4-Xaa5-Xaa6-Xaa7-Xaa8-Xaa9-
Xaa10-Xaa11-Xaa12-Xaa13-Xaa14
Formula (I)

wherein

Xaa1 is F;

Xaa2 is I or T;

Xaa3 is D;

Xaa4 is D;

Xaa5 is Y;

Xaa6 is G;

Xaa7 is I;

Xaa8 is T;

Xaa9 is D;

Xaa10 is Y;

Xaa11 is D,

Xaa12is AorE;

Xaa13 is A, D, V, or G; and

Xaa14 is C; wherein each of the Xaa1 to Xaa14 is independently an amino acid residue designated above or a derivative thereof.

In one aspect, provided herein a cyclic peptide having a structure of Formula (II),

XaaN-Xaa2-Xaa3-Xaa4-Xaa5-Xaa6-Xaa7-Xaa8-Xaa9-Xaa10-Xaa11-Xaa12-Xaa13-XaaC Formula (II)
wherein each of the XaaN, Xaa2, Xaa3, Xaa4, Xaa5, and Xaa6 is independently an amino acid residue, and
each of the Xaa7, Xaa8, Xaa9, Xaa10, Xaa11, Xaa12, Xaa13, and XaaC is independently absent or an amino acid residue.

In some embodiments, provided herein a cyclic peptide having a structure of Formula (II),

XaaN-Xaa2-Xaa3-Xaa4-Xaa5-Xaa6-Xaa7-Xaa8-Xaa9-Xaa10-Xaa11-Xaa12-Xaa13-XaaC Formula (II)
wherein
each of the XaaN, Xaa2, Xaa3, Xaa4, Xaa5, and Xaa6 is independently an amino acid residue;
each of the Xaa7, Xaa8, Xaa9, Xaa10, Xaa11, Xaa12, Xaa13, and XaaC is independently absent or an amino acid residue; and

wherein the cyclic peptide comprises a ring closing group selected from:
—(CH₂)ₘ—C(═O)—CH₂—S—(CH₂)ₙ—, —C(═O)—CH₂—S—CH₂—CH₂—, —(CH₂)ₘ—NH—CO—(CH₂)ₙ—, —(CH₂)ₘ—CO—NH—(CH₂)ₙ—, —(CH₂)ₘ—S—(CH₂)ₙ—, —(CH₂)ₘ—CH═CH—(CH₂)ₙ—, —(CH₂)ₘ—NH—(CH₂)ₙ—, —(CH₂)ₘ—S—CH₂-benzene-CH₂—S—(CH₂)ₙ—, —(CH₂)ₘ-triazine-(CH₂)ₙ—, —(CH₂)ₘ-succinimide-S—(CH₂)ₙ—, —C(═O)—CH₂—NH—CH₂—, and —C(═O)—CH₂—O—CH₂—, where m and n are each independently 0 or an integer from 1 to 6.

In some embodiments, m is 0. In some embodiments, m is 1. In some embodiments, m is 2. In some embodiments, m is 3. In some embodiments, m is 4. In some embodiments, m is 5. In some embodiments, m is 6. In some embodiments, n is 0. In some embodiments, n is 1. In some embodiments, n is 2. In some embodiments, n is 3. In some embodiments, n is 4. In some embodiments, n is 5. In some embodiments, n is 6.

In some embodiments, the ring closing group is formed by the cyclization of the cyclic peptide. The ring closing group can be formed by forming a bond between XaaN and XaaC. The ring closing group can be formed by forming a bond between the N- and C-terminus of the peptide. In some embodiments, a cyclic peptide of Formula (II) comprises a ring closing group of —C(═O)—CH₂—S—. In some embodiments, the ring closing group —C(═O)—CH₂—S— is formed between XaaN and a cysteine, wherein XaaN is chloroacetylated. In some embodiments, the cysteine is located at the C-terminus of the cyclic peptide. In some embodiments, the cysteine is located within 3 amino acids from the C-terminus of the cyclic peptide.

In one aspect, provided herein a cyclic peptide having a structure of Formula (III),

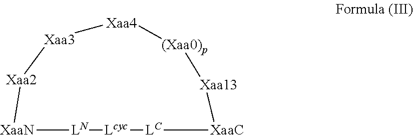

wherein
each of the XaaN, Xaa2, Xaa3, Xaa4, Xaa0, Xaa13, and XaaC is independently an amino acid residue;
p is 0, 1, 2, 3, 4, 5, 6, 7, or 8;
wherein each of the amino acid residues in the cyclic peptide is joined by a peptide bond, provided that XaaN and XaaC is connected through -L^{N}-L^{cyc}-L^{c}-;
L^{N} and L^{C} are each independently optionally substituted C₁-C₆alkylene, optionally substituted C₁-C₆heteroalkylene, or a bond;
L^{cyc} is a ring closing group; and
wherein the cyclic peptide is monocyclic.

In some embodiments of Formula (III), -L^{N}-L^{cyc}-L^{c}- is formed by reacting the functional groups of XaaN and XaaC. For example, when XaaN is functionalized with —C(═O)—CH₂Cl and XaaC is cysteine,

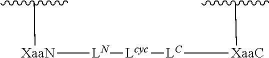

can be

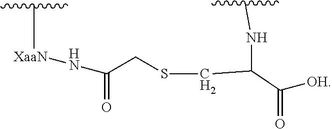

As described in Formula (III), only the cyclic portion of the peptide sequence is included. In some embodiments, a conjugate comprising a peptide of Formula (III) may further comprise amino acid residues at the N and/or C terminus of the peptide, which is not part of the cyclic structure. In some embodiments, a conjugate comprising a peptide of Formula (III) may further comprise one or more amino acid residues attached to XaaN, which is not part of the cyclic structure. In some embodiments, a conjugate comprising a peptide of Formula (III) may further comprise one or more amino acid residues attached to XaaC, which is not part of the cyclic structure.

In some embodiments of Formula (III), -(Xaa0)p- is -Xaa5-Xaa6-Xaa7-Xaa8-Xaa9-Xaa10-Xaa11-Xaa12-, wherein each of the Xaa5, Xaa6, Xaa7, Xaa8, Xaa9, Xaa10, Xaa11, and Xaa12 is independently absent or an amino acid residue.

In some embodiments of Formula (III), L^{N} is a bond. In some embodiments of Formula (III), L^{N} is optionally substituted C₁-C₆alkylene, wherein the alkylene is optionally substituted with one or more substituents selected from halogen, —OH, amino, C₁-C₆alkyl, C₁-C₆haloalkyl, C₁-C₆hydroxyalkyl, C₁-C₆aminoalkyl, and C₁-C₆heteroalkyl. In some embodiments of Formula (III), L^{N} is unsubstituted C₁-C₆alkylene. In some embodiments of Formula (III), L^{N} is unsubstituted C₁-C₆₃alkylene. In some embodiments of Formula (III), L^{N} is unsubstituted C₁-C₄alkylene. In some embodiments of Formula (III), L^{N} is —CH₂—, —CH₂—CH₂—, —CH₂—CH₂—CH₂—, or —CH₂—CH₂—CH₂—CH₂—.

In some embodiments of Formula (III), L^{C} is a bond. In some embodiments of Formula (III), L^{C} is optionally substituted C₁-C₆alkylene, wherein the alkylene is optionally substituted with one or more substituents selected from halogen, —OH, amino, C₁-C₆alkyl, C₁-C₆haloalkyl, C₁-C₆hydroxyalkyl, C₁-C₆aminoalkyl, and C₁-C₆heteroalkyl. In some embodiments of Formula (III), L^{C} is unsubstituted C₁-C₆alkylene. In some embodiments of a cyclic peptide of formula (III), L^{C} is C₁-C₃alkylene. In some embodiments of Formula (III), L^{C} is unsubstituted C₁-C₄alkylene. In some embodiments of Formula (III), L^{C} is —CH₂—, —CH₂—CH₂—, —CH₂—CH₂—CH₂—, or —CH₂—CH₂—CH₂—CH₂—.

In some embodiments of Formula (III), L^{N} and L^{C} are each independently optionally substituted C₁-C₆alkylene, optionally substituted C₁-C₆heteroalkylene, or a bond, wherein the C₁-C₆alkylene and C₁-C₆heteroalkylene are each optionally substituted with one or more R¹⁰,
each R¹⁰ is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, —Si(R^{a})₃, —P(═O)(R^{b})₂, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl, wherein the alkyl, alkenyl, alkynyl, cycloalkyl, heterocycloalkyl, aryl, and heteroaryl is optionally and independently substituted with one or more R^{10a};
or two R¹⁰ on the same atom are taken together to form an oxo;
or two R¹⁰ on the same atoms are taken together to form a cycloalkyl or heterocycloalkyl; each optionally substituted with one or more R^{10a};
or two R¹⁰ on different atoms are taken together to form a cycloalkyl, heterocycloalkyl, aryl, or heteroaryl, each optionally substituted with one or more R^{10a};
each R^{10a} is independently halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
or two R^{a} are taken together with the atom to which they are attached to form a heterocycloalkyl optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
or two R^{b} are taken together with the atom to which they are attached to form a heterocycloalkyl optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R is independently halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆hydroxyalkyl, C₁-C₆aminoalkyl, and C₁-C₆heteroalkyl;
or two R on the same atom form an oxo.

In some embodiments, XaaN of Formula (II) or Formula (III) is a natural amino acid or unnatural amino acid. In some embodiments, XaaN is a natural amino acid. In some embodiments, XaaN is an unnatural amino acid. In some embodiments, XaaN is an unnatural amino acid selected from Tables 4A-4G. In some embodiments, XaaN is an unnatural amino acid selected from Table 4G. In some embodiments, XaaN is an aliphatic amino acid, e.g., alanine, glycine, isoleucine, leucine, proline, valine, or a derivative thereof. In some embodiments, XaaN is an aromatic amino acid, e.g., phenylalanine, tryptophan, tyrosine, or a derivative thereof. In some embodiments, XaaN is an acidic amino acid, e.g., aspartic acid, glutamic acid, or a derivative thereof. In some embodiments, XaaN is a basic amino acid, e.g., arginine, histidine, lysine, or a derivative thereof. In some embodiments, XaaN is hydroxylic amino acid, e.g., serine, threonine, or a derivative thereof. In some embodiments, XaaN is a sulphur-containing amino acid, e.g., cysteine, methionine, or a derivative thereof. In some embodiments, XaaN is an amidic (containing amide group) amino acid, e.g., asparagine, glutamine, or a derivative thereof. In some embodiments, XaaN does not carry any charges. In some embodiments, XaaN carries a −1 charge, e.g., aspartic acid, glutamic acid, or a derivative thereof. In some embodiments, XaaN carries a +1 charge, e.g., arginine, histidine, lysine, or a derivative thereof. In some embodiments, XaaN is lysine.

In some embodiments, Xaa2 of Formula (II) or Formula (III) is a natural amino acid or unnatural amino acid. In some embodiments, Xaa2 is a natural amino acid. In some embodiments, Xaa2 is an unnatural amino acid. In some embodiments, Xaa2 is an unnatural amino acid selected from Tables 4A-4G. In some embodiments, Xaa2 is an unnatural amino acid selected from Table 4G. In some embodiments, Xaa2 is an aliphatic amino acid, e.g., alanine, glycine, isoleucine, leucine, proline, valine, or a derivative thereof. In some embodiments, Xaa2 is an aromatic amino acid, e.g., phenylalanine, tryptophan, tyrosine, or a derivative thereof. In some embodiments, Xaa2 is an acidic amino acid, e.g., aspartic acid, glutamic acid, or a derivative thereof. In some embodiments, Xaa2 is a basic amino acid, e.g., arginine, histidine, lysine, or a derivative thereof. In some embodiments, Xaa2 is hydroxylic amino acid, e.g., serine, threonine, or a derivative thereof. In some embodiments, Xaa2 is a sulphur-containing amino acid, e.g., cysteine, methionine, or a derivative thereof. In some embodiments, Xaa2 is an amidic (containing amide group) amino acid, e.g., asparagine, glutamine, or a derivative thereof. In some embodiments, Xaa2 does not carry any charges. In some embodiments, Xaa2 carries a −1 charge, e.g., aspartic acid, glutamic acid, or a derivative thereof. In some embodiments, Xaa2 carries a +1 charge, e.g., arginine, histidine, lysine, or a derivative thereof.

In some embodiments, Xaa3 of Formula (II) or Formula (III) is a natural amino acid or unnatural amino acid. In some embodiments, Xaa3 is a natural amino acid. In some embodiments, Xaa3 is an unnatural amino acid. In some embodiments, Xaa3 is an unnatural amino acid selected from Tables 4A-4G. In some embodiments, Xaa3 is an unnatural amino acid selected from Table 4G. In some embodiments, Xaa3 is an aliphatic amino acid, e.g., alanine, glycine, isoleucine, leucine, proline, valine, or a derivative thereof. In some embodiments, Xaa3 is an aromatic amino acid, e.g., phenylalanine, tryptophan, tyrosine, or a derivative thereof. In some embodiments, Xaa3 is an acidic amino acid, e.g., aspartic acid, glutamic acid, or a derivative thereof. In some embodiments, Xaa3 is a basic amino acid, e.g., arginine, histidine, lysine, or a derivative thereof. In some embodiments, Xaa3 is hydroxylic amino acid, e.g., serine, threonine, or a derivative thereof. In some embodiments, Xaa3 is a sulphur-containing amino acid, e.g., cysteine, methionine, or a derivative thereof. In some embodiments, Xaa3 is an amidic (containing amide group) amino acid, e.g., asparagine, glutamine, or a derivative thereof. In some embodiments, Xaa3 does not carry any charges. In some embodiments, Xaa3 carries a −1 charge, e.g., aspartic acid, glutamic acid, or a derivative thereof. In some embodiments, Xaa3 carries a +1 charge, e.g., arginine, histidine, lysine, or a derivative thereof.

In some embodiments, Xaa4 of Formula (II) or Formula (III) is a natural amino acid or unnatural amino acid. In some embodiments, Xaa4 is a natural amino acid. In some embodiments, Xaa4 is an unnatural amino acid. In some embodiments, Xaa4 is an unnatural amino acid selected from Tables 4A-4G. In some embodiments, Xaa4 is an unnatural amino acid selected from Table 4G. In some embodiments, Xaa4 is an aliphatic amino acid, e.g., alanine, glycine, isoleucine, leucine, proline, valine, or a derivative thereof. In some embodiments, Xaa4 is an aromatic amino acid, e.g., phenylalanine, tryptophan, tyrosine, or a derivative thereof. In some embodiments, Xaa4 is an acidic amino acid, e.g., aspartic acid, glutamic acid, or a derivative thereof. In some embodiments, Xaa4 is a basic amino acid, e.g., arginine, histidine, lysine, or a derivative thereof. In some embodiments, Xaa4 is hydroxylic amino acid, e.g., serine, threonine, or a derivative thereof. In some embodiments, Xaa4 is a sulphur-containing amino acid, e.g., cysteine, methionine, or a derivative thereof. In some embodiments, Xaa4 is an amidic (containing amide group) amino acid, e.g., asparagine, glutamine, or a derivative thereof. In some embodiments, Xaa4 does not carry any charges. In some embodiments, Xaa4 carries a −1 charge, e.g., aspartic acid, glutamic acid, or a derivative thereof. In some embodiments, Xaa4 carries a +1 charge, e.g., arginine, histidine, lysine, or a derivative thereof.

In some embodiments, Xaa5 of Formula (II) or Formula (III) is a natural amino acid or unnatural amino acid. In some embodiments, Xaa5 is a natural amino acid. In some embodiments, Xaa5 is an unnatural amino acid. In some embodiments, Xaa5 is an unnatural amino acid selected from Tables 4A-4G. In some embodiments, Xaa5 is an unnatural amino acid selected from Table 4G. In some embodiments, Xaa5 is an aliphatic amino acid, e.g., alanine, glycine, isoleucine, leucine, proline, valine, or a derivative thereof. In some embodiments, Xaa5 is an aromatic amino acid, e.g., phenylalanine, tryptophan, tyrosine, or a derivative thereof. In some embodiments, Xaa5 is an acidic amino acid, e.g., aspartic acid, glutamic acid, or a derivative thereof. In some embodiments, Xaa5 is a basic amino acid, e.g., arginine, histidine, lysine, or a derivative thereof. In some embodiments, Xaa5 is hydroxylic amino acid, e.g., serine, threonine, or a derivative thereof. In some embodiments, Xaa5 is a sulphur-containing amino acid, e.g., cysteine, methionine, or a derivative thereof. In some embodiments, Xaa5 is an amidic (containing amide group) amino acid, e.g., asparagine, glutamine, or a derivative thereof. In some embodiments, Xaa5 does not carry any charges. In some embodiments, Xaa5 carries a −1 charge, e.g., aspartic acid, glutamic acid, or a derivative thereof. In some embodiments, Xaa5 carries a +1 charge, e.g., arginine, histidine, lysine, or a derivative thereof. In some embodiments, Xaa5 is absent.

In some embodiments, Xaa6 of Formula (II) or Formula (III) is a natural amino acid or unnatural amino acid. In some embodiments, Xaa6 is a natural amino acid. In some embodiments, Xaa6 is an unnatural amino acid. In some embodiments, Xaa6 is an unnatural amino acid selected from Tables 4A-4G. In some embodiments, Xaa6 is an unnatural amino acid selected from Table 4G. In some embodiments, Xaa6 is an aliphatic amino acid, e.g., alanine, glycine, isoleucine, leucine, proline, valine, or a derivative thereof. In some embodiments, Xaa6 is an aromatic amino acid, e.g., phenylalanine, tryptophan, tyrosine, or a derivative thereof. In some embodiments, Xaa6 is an acidic amino acid, e.g., aspartic acid, glutamic acid, or a derivative thereof. In some embodiments, Xaa6 is a basic amino acid, e.g., arginine, histidine, lysine, or a derivative thereof. In some embodiments, Xaa6 is hydroxylic amino acid, e.g., serine, threonine, or a derivative thereof. In some embodiments, Xaa6 is a sulphur-containing amino acid, e.g., cysteine, methionine, or a derivative thereof. In some embodiments, Xaa6 is an amidic (containing amide group) amino acid, e.g., asparagine, glutamine, or a derivative thereof. In some embodiments, Xaa6 does not carry any charges. In some embodiments, Xaa6 carries a −1 charge, e.g., aspartic acid, glutamic acid, or a derivative thereof. In some embodiments, Xaa6 carries a +1 charge, e.g., arginine, histidine, lysine, or a derivative thereof. In some embodiments, Xaa6 is absent.

In some embodiments, Xaa7 of Formula (II) or Formula (III) is a natural amino acid or unnatural amino acid. In some embodiments, Xaa7 is a natural amino acid. In some embodiments, Xaa7 is an unnatural amino acid. In some embodiments, Xaa7 is an unnatural amino acid selected from Tables 4A-4G. In some embodiments, Xaa7 is an unnatural amino acid selected from Table 4G. In some embodiments, Xaa7 is an aliphatic amino acid, e.g., alanine, glycine, isoleucine, leucine, proline, valine, or a derivative thereof. In some embodiments, Xaa7 is an aromatic amino acid, e.g., phenylalanine, tryptophan, tyrosine, or a derivative thereof. In some embodiments, Xaa7 is an acidic amino acid, e.g., aspartic acid, glutamic acid, or a derivative thereof. In some embodiments, Xaa7 is a basic amino acid, e.g., arginine, histidine, lysine, or a derivative thereof. In some embodiments, Xaa7 is hydroxylic amino acid, e.g., serine, threonine, or a derivative thereof. In some embodiments, Xaa7 is a sulphur-containing amino acid, e.g., cysteine, methionine, or a derivative thereof. In some embodiments, Xaa7 is an amidic (containing amide group) amino acid, e.g., asparagine, glutamine, or a derivative thereof. In some embodiments, Xaa7 does not carry any charges. In some embodiments, Xaa7 carries a −1 charge, e.g., aspartic acid, glutamic acid, or a derivative thereof. In some embodiments, Xaa7 carries a +1 charge, e.g., arginine, histidine, lysine, or a derivative thereof. In some embodiments, Xaa7 is absent.

In some embodiments, Xaa8 of Formula (II) or Formula (III) is a natural amino acid or unnatural amino acid. In some embodiments, Xaa8 is a natural amino acid. In some embodiments, Xaa8 is an unnatural amino acid. In some embodiments, Xaa8 is an unnatural amino acid selected from Tables 4A-4G. In some embodiments, Xaa8 is an unnatural amino acid selected from Table 4G. In some embodiments, Xaa8 is an aliphatic amino acid, e.g., alanine, glycine, isoleucine, leucine, proline, valine, or a derivative thereof. In some embodiments, Xaa8 is an aromatic amino acid, e.g., phenylalanine, tryptophan, tyrosine, or a derivative thereof. In some embodiments, Xaa8 is an acidic amino acid, e.g., aspartic acid, glutamic acid, or a derivative thereof. In some embodiments, Xaa8 is a basic amino acid, e.g., arginine, histidine, lysine, or a derivative thereof. In some embodiments, Xaa8 is hydroxylic amino acid, e.g., serine, threonine, or a derivative thereof. In some embodiments, Xaa8 is a sulphur-containing amino acid, e.g., cysteine, methionine, or a derivative thereof. In some embodiments, Xaa8 is an amidic (containing amide group) amino acid, e.g., asparagine, glutamine, or a derivative thereof. In some embodiments, Xaa8 does not carry any charges. In some embodiments, Xaa8 carries a −1 charge, e.g., aspartic acid, glutamic acid, or a derivative thereof. In some embodiments, Xaa8 carries a +1 charge, e.g., arginine, histidine, lysine, or a derivative thereof. In some embodiments, Xaa8 is absent.

In some embodiments, Xaa9 of Formula (II) or Formula (III) is a natural amino acid or unnatural amino acid. In some embodiments, Xaa9 is a natural amino acid. In some embodiments, Xaa9 is an unnatural amino acid. In some embodiments, Xaa9 is an unnatural amino acid selected from Tables 4A-4G. In some embodiments, Xaa9 is an unnatural amino acid selected from Table 4G. In some embodiments, Xaa9 is an aliphatic amino acid, e.g., alanine, glycine, isoleucine, leucine, proline, valine, or a derivative thereof. In some embodiments, Xaa9 is an aromatic amino acid, e.g., phenylalanine, tryptophan, tyrosine, or a derivative thereof. In some embodiments, Xaa9 is an acidic amino acid, e.g., aspartic acid, glutamic acid, or a derivative thereof. In some embodiments, Xaa9 is a basic amino acid, e.g., arginine, histidine, lysine, or a derivative thereof. In some embodiments, Xaa9 is hydroxylic amino acid, e.g., serine, threonine, or a derivative thereof. In some embodiments, Xaa9 is a sulphur-containing amino acid, e.g., cysteine, methionine, or a derivative thereof. In some embodiments, Xaa9 is an amidic (containing amide group) amino acid, e.g., asparagine, glutamine, or a derivative thereof. In some embodiments, Xaa9 does not carry any charges. In some embodiments, Xaa9 carries a −1 charge, e.g., aspartic acid, glutamic acid, or a derivative thereof. In some embodiments, Xaa9 carries a +1 charge, e.g., arginine, histidine, lysine, or a derivative thereof. In some embodiments, Xaa9 is absent.

In some embodiments, Xaa10 of Formula (II) or Formula (III) is a natural amino acid or unnatural amino acid. In some embodiments, Xaa10 is a natural amino acid. In some embodiments, Xaa10 is an unnatural amino acid. In some embodiments, Xaa10 is an unnatural amino acid selected from Tables 4A-4G. In some embodiments, Xaa10 is an unnatural amino acid selected from Table 4G. In some embodiments, Xaa10 is an aliphatic amino acid, e.g., alanine, glycine, isoleucine, leucine, proline, valine, or a derivative thereof. In some embodiments, Xaa10 is an aromatic amino acid, e.g., phenylalanine, tryptophan, tyrosine, or a derivative thereof. In some embodiments, Xaa10 is an acidic amino acid, e.g., aspartic acid, glutamic acid, or a derivative thereof. In some embodiments, Xaa10 is a basic amino acid, e.g., arginine, histidine, lysine, or a derivative thereof. In some embodiments, Xaa10 is hydroxylic amino acid, e.g., serine, threonine, or a derivative thereof. In some embodiments, Xaa10 is a sulphur-containing amino acid, e.g., cysteine, methionine, or a derivative thereof. In some embodiments, Xaa10 is an amidic (containing amide group) amino acid, e.g., asparagine, glutamine, or a derivative thereof. In some embodiments, Xaa10 does not carry any charges. In some embodiments, Xaa10 carries a −1 charge, e.g., aspartic acid, glutamic acid, or a derivative thereof. In some embodiments, Xaa10 carries a +1 charge, e.g., arginine, histidine, lysine, or a derivative thereof. In some embodiments, Xaa10 is absent.

In some embodiments, Xaa11 of Formula (II) or Formula (III) is a natural amino acid or unnatural amino acid. In some embodiments, Xaa11 is a natural amino acid. In some embodiments, Xaa11 is an unnatural amino acid. In some embodiments, Xaa11 is an unnatural amino acid selected from Tables 4A-4G. In some embodiments, Xaa11 is an unnatural amino acid selected from Table 4G. In some embodiments, Xaa11 is an aliphatic amino acid, e.g., alanine, glycine, isoleucine, leucine, proline, valine, or a derivative thereof. In some embodiments, Xaa11 is an aromatic amino acid, e.g., phenylalanine, tryptophan, tyrosine, or a derivative thereof. In some embodiments, Xaa11 is an acidic amino acid, e.g., aspartic acid, glutamic acid, or a derivative thereof. In some embodiments, Xaa11 is a basic amino acid, e.g., arginine, histidine, lysine, or a derivative thereof. In some embodiments, Xaa11 is hydroxylic amino acid, e.g., serine, threonine, or a derivative thereof. In some embodiments, Xaa11 is a sulphur-containing amino acid, e.g., cysteine, methionine, or a derivative thereof. In some embodiments, Xaa11 is an amidic (containing amide group) amino acid, e.g., asparagine, glutamine, or a derivative thereof. In some embodiments, Xaa11 does not carry any charges. In some embodiments, Xaa11 carries a −1 charge, e.g., aspartic acid, glutamic acid, or a derivative thereof. In some embodiments, Xaa11 carries a +1 charge, e.g., arginine, histidine, lysine, or a derivative thereof. In some embodiments, Xaa11 is absent.

In some embodiments, Xaa12 of Formula (II) or Formula (III) is a natural amino acid or unnatural amino acid. In some embodiments, Xaa12 is a natural amino acid. In some embodiments, Xaa12 is an unnatural amino acid. In some embodiments, Xaa12 is an unnatural amino acid selected from Table 4G. In some embodiments, XaaC is an unnatural amino acid selected from Tables 4A-4G. In some embodiments, Xaa12 is an aliphatic amino acid, e.g., alanine, glycine, isoleucine, leucine, proline, valine, or a derivative thereof. In some embodiments, Xaa12 is an aromatic amino acid, e.g., phenylalanine, tryptophan, tyrosine, or a derivative thereof. In some embodiments, Xaa12 is an acidic amino acid, e.g., aspartic acid, glutamic acid, or a derivative thereof. In some embodiments, Xaa12 is a basic amino acid, e.g., arginine, histidine, lysine, or a derivative thereof. In some embodiments, Xaa12 is hydroxylic amino acid, e.g., serine, threonine, or a derivative thereof. In some embodiments, Xaa12 is a sulphur-containing amino acid, e.g., cysteine, methionine, or a derivative thereof. In some embodiments, Xaa12 is an amidic (containing amide group) amino acid, e.g., asparagine, glutamine, or a derivative thereof. In some embodiments, Xaa12 does not carry any charges. In some embodiments, Xaa12 carries a −1 charge, e.g., aspartic acid, glutamic acid, or a derivative thereof. In some embodiments, Xaa12 carries a +1 charge, e.g., arginine, histidine, lysine, or a derivative thereof. In some embodiments, Xaa12 is absent.

In some embodiments, Xaa13 of Formula (II) or Formula (III) is a natural amino acid or unnatural amino acid. In some embodiments, Xaa13 is a natural amino acid. In some embodiments, Xaa13 is an unnatural amino acid. In some embodiments, Xaa13 is an unnatural amino acid selected from Tables 4A-4G. In some embodiments, Xaa13 is an unnatural amino acid selected from Table 4G. In some embodiments, Xaa13 is an aliphatic amino acid, e.g., alanine, glycine, isoleucine, leucine, proline, valine, or a derivative thereof. In some embodiments, Xaa13 is an aromatic amino acid, e.g., phenylalanine, tryptophan, tyrosine, or a derivative thereof. In some embodiments, Xaa13 is an acidic amino acid, e.g., aspartic acid, glutamic acid, or a derivative thereof. In some embodiments, Xaa13 is a basic amino acid, e.g., arginine, histidine, lysine, or a derivative thereof. In some embodiments, Xaa13 is hydroxylic amino acid, e.g., serine, threonine, or a derivative thereof. In some embodiments, Xaa13 is a sulphur-containing amino acid, e.g., cysteine, methionine, or a derivative thereof. In some embodiments, Xaa13 is an amidic (containing amide group) amino acid, e.g., asparagine, glutamine, or a derivative thereof. In some embodiments, Xaa13 does not carry any charges. In some embodiments, Xaa13 carries a −1 charge, e.g., aspartic acid, glutamic acid, or a derivative thereof. In some embodiments, Xaa13 carries a +1 charge, e.g., arginine, histidine, lysine, or a derivative thereof. In some embodiments of Formula (II), Xaa13 is absent.

In some embodiments, XaaC of Formula (II) or Formula (III) is a natural amino acid or unnatural amino acid. In some embodiments, XaaC is a natural amino acid. In some embodiments, XaaC is an unnatural amino acid. In some embodiments, XaaC is an unnatural amino acid selected from Tables 4A-4G. In some embodiments, XaaC is an unnatural amino acid selected from Table 4G. In some embodiments, XaaC is an aliphatic amino acid, e.g., alanine, glycine, isoleucine, leucine, proline, valine, or a derivative thereof. In some embodiments, XaaC is an aromatic amino acid, e.g., phenylalanine, tryptophan, tyrosine, or a derivative thereof. In some embodiments, XaaC is an acidic amino acid, e.g., aspartic acid, glutamic acid, or a derivative thereof. In some embodiments, XaaC is a basic amino acid, e.g., arginine, histidine, lysine, or a derivative thereof. In some embodiments, XaaC is hydroxylic amino acid, e.g., serine, threonine, or a derivative thereof. In some embodiments, XaaC is a sulphur-containing amino acid, e.g., cysteine, methionine, or a derivative thereof. In some embodiments, XaaC is an amidic (containing amide group) amino acid, e.g., asparagine, glutamine, or a derivative thereof. In some embodiments, XaaC does not carry any charges. In some embodiments, XaaC carries a −1 charge, e.g., aspartic acid, glutamic acid, or a derivative thereof. In some embodiments, XaaC carries a +1 charge, e.g., arginine, histidine, lysine, or a derivative thereof. In some embodiments, XaaC is Cys. In some embodiments, XaaC is a homocysteine. In some embodiments of Formula (II), XaaC is absent.

In some embodiments, each of Xaa0 of Formula (III) is independently a natural amino acid or unnatural amino acid. In some embodiments, Xaa0 is a natural amino acid. In some embodiments, Xaa0 is an unnatural amino acid. In some embodiments, Xaa0 is an unnatural amino acid selected from Tables 4A-4G. In some embodiments, Xaa0 is an unnatural amino acid selected from Table 4G. In some embodiments, Xaa0 is an aliphatic amino acid, e.g., alanine, glycine, isoleucine, leucine, proline, valine, or a derivative thereof. In some embodiments, Xaa0 is an aromatic amino acid, e.g., phenylalanine, tryptophan, tyrosine, or a derivative thereof. In some embodiments, Xaa0 is an acidic amino acid, e.g., aspartic acid, glutamic acid, or a derivative thereof. In some embodiments, Xaa0 is a basic amino acid, e.g., arginine, histidine, lysine, or a derivative thereof. In some embodiments, Xaa0 is hydroxylic amino acid, e.g., serine, threonine, or a derivative thereof. In some embodiments, Xaa0 is a sulphur-containing amino acid, e.g., cysteine, methionine, or a derivative thereof. In some embodiments, Xaa0 is an amidic (containing amide group) amino acid, e.g., asparagine, glutamine, or a derivative thereof. In some embodiments, Xaa0 does not carry any charges. In some embodiments, Xaa0 carries a −1 charge, e.g., aspartic acid, glutamic acid, or a derivative thereof. In some embodiments, Xaa0 carries a +1 charge, e.g., arginine, histidine, lysine, or a derivative thereof.

In some embodiments of Formula (III), p is 0, 1, 2, 3, 4, 5, 6, 7, or 8. In some embodiments, p is 0. In some embodiments, p is 1. In some embodiments, p is 2. In some embodiments, p is 3. In some embodiments, p is 4. In some embodiments, p is 5. In some embodiments, p is 6. In some embodiments, p is 7. In some embodiments, p is 8.

In some embodiments, a cyclic peptide of Formula (I), Formula (II) or Formula (III) has a net charge of −3 to +1. In some embodiments, the cyclic peptide has a net charge of −3. In some embodiments, the cyclic peptide has a net charge of −2. In some embodiments, the cyclic peptide has a net charge of −1. In some embodiments, the cyclic peptide has a net charge of 0. In some embodiments, the cyclic peptide has a net charge of +1. In some embodiments, a cyclic peptide of Formula (I), Formula (II) or Formula (III) has a net charge of at most −4. In some embodiments, the cyclic peptide has a net charge of −4. In some embodiments, a cyclic peptide of Formula (I), Formula (II) or Formula (III) has a net charge of at least +2. In some embodiments, the cyclic peptide has a net charge of +2. In some embodiments, the cyclic peptide has a net charge of +3. The net charge can be determined by aggregating the charge of each of the Xaal to Xaa14 amino acids (or each of the amino acid in the peptide). For example, aspartic acid (D) and glutamic acid (E) each has a charge of −1, lysine (K), arginine (R) and histidine (H) each has a charge of +1, and the rest of the canonical amino acids each has a charge of 0.

In some embodiments, a cyclic peptide of formula (III) has a net charge of −3 to +1. In some embodiments, the cyclic peptide has a net charge of −3. In some embodiments, the cyclic peptide has a net charge of −2. In some embodiments, the cyclic peptide has a net charge of −1. In some embodiments, the cyclic peptide has a net charge of 0. In some embodiments, the cyclic peptide has a net charge of +1. The net charge can be determined by aggregating the charge of each of the amino acids of the cyclic peptide.

In some embodiments, a cyclic peptide described herein (e.g., a cyclic peptide of Formula (I), Formula (II) or Formula (III)) is configured to bind to a plasma protein with a prescribed affinity, for example, measured as Plasma Protein Albumin Binding (PPB) percentage. The % bound can be determined by HSA-HPLC method (measurement of drug protein binding by immobilized human serum albumin-HPLC). PPB can be determined in vitro by HPLC (e.g., Example B3) or by other suitable means known in the art. In some embodiments, 1% to 99% of the cyclic peptide binds to Human Serum Albumin (HSA) in vitro as determined by HPLC, according to the conditions described in Example B3. In some embodiments, about 2% to about 99%, about 5% to about 99%, about 10% to about 99%, about 20% to about 99%, about 30% to about 99%, about 40% to about 99%, about 50% to about 99%, about 60% to about 99%, about 70% to about 99%, or about 80% to about 99% of the cyclic peptide binds to HSA in vitro as determined by HPLC. In some embodiments, about 10% to about 95% of the cyclic peptide binds to HSA in vitro (i.e., PPB of about 10% to about 95%). In some embodiments, about 20% to about 90% of the cyclic peptide binds to HSA in vitro. In some embodiments, about 20% to about 60% of the cyclic peptide binds to HSA in vitro. In some embodiments, about 40% to about 95% of the cyclic peptide binds to HSA in vitro. In some embodiments, about 40% to about 80% of the cyclic peptide binds to HSA in vitro. In some embodiments, about 40% to about 60% of the cyclic peptide binds to HSA in vitro. In some embodiments, about 60% to about 99% of the cyclic peptide binds to HSA in vitro. In some embodiments, about 60% to about 95% of the cyclic peptide binds to HSA in vitro. In some embodiments, about 60% to about 80% of the cyclic peptide binds to HSA in vitro. In some embodiments, about 60% to about 70% of the cyclic peptide binds to HSA in vitro. In some embodiments, about 40% to about 50% of the cyclic peptide binds to HSA in vitro. In some embodiments, about 50% to about 60% of the cyclic peptide binds to HSA in vitro. In some embodiments, about 70% to about 80% of the cyclic peptide binds to HSA in vitro. In some embodiments, about 80% to about 99% of the cyclic peptide binds to HSA in vitro. In some embodiments, about 80% to about 85% of the cyclic peptide binds to HSA in vitro.

In some embodiments, a conjugate described herein (e.g., a conjugate comprising a cyclic peptide of Formula (I), Formula (II) or Formula (III)) is configured to bind to a plasma protein with a prescribed affinity, for example, measured as Plasma Protein Albumin Binding (PPB) percentage. PPB can be determined in vitro by HPLC (e.g., Example B3) or by other suitable means known in the art. In some embodiments, 1% to 99% of the conjugate binds to Human Serum Albumin (HSA) in vitro as determined by HPLC, according to the conditions described in Example B3. In some embodiments, about 2% to about 99%, about 5% to about 99%, about 10% to about 99%, about 20% to about 99%, about 30% to about 99%, about 40% to about 99%, about 50% to about 99%, about 60% to about 99%, about 70% to about 99%, or about 80% to about 99% of the conjugate binds to HSA in vitro as determined by HPLC. In some embodiments, about 10% to about 95% of the conjugate binds to HSA in vitro (i.e., PPB of about 10% to about 95%). In some embodiments, about 20% to about 90% of the conjugate binds to HSA in vitro. In some embodiments, about 20% to about 60% of the conjugate binds to HSA in vitro. In some embodiments, about 40% to about 95% of the conjugate binds to HSA in vitro. In some embodiments, about 40% to about 80% of the conjugate binds to HSA in vitro. In some embodiments, about 40% to about 60% of the conjugate binds to HSA in vitro. In some embodiments, about 60% to about 99% of the conjugate binds to HSA in vitro. In some embodiments, about 60% to about 95% of the conjugate binds to HSA in vitro. In some embodiments, about 60% to about 80% of the conjugate binds to HSA in vitro. In some embodiments, about 60% to about 70% of the conjugate binds to HSA in vitro. In some embodiments, about 40% to about 50% of the conjugate binds to HSA in vitro. In some embodiments, about 50% to about 60% of the conjugate binds to HSA in vitro. In some embodiments, about 70% to about 80% of the conjugate binds to HSA in vitro. In some embodiments, about 80% to about 99% of the conjugate binds to HSA in vitro. In some embodiments, about 80% to about 85% of the conjugate binds to HSA in vitro.

In some embodiments, a cyclic peptide of Formula (I), Formula (II) or Formula (III) does not contain any S-S bond.

In some embodiments of a cyclic peptide of formula (III), L^{cyc} is formed by reacting a functional group A with the corresponding functional group B of Table 1. The functional group A can be a part of the amino acid XaaN, or XaaN can be functionalized with the functional group A. The functional group B can be a part of the amino acid XaaC, or XaaC can be functionalized with the functional group B. In some embodiments, L^{cyc} is a group formed by reacting functional group (I-A) with functional group (I-B) of Table 1. In some embodiments, L^{cyc} is a group formed by reacting functional group (II-A) with functional group (II-B) of Table 1. In some embodiments, L^{cyc} is a group formed by reacting functional group (III-A) with functional group (III-B) of Table 1. In some embodiments, L^{cyc} is a group formed by reacting functional group (IV-A) with functional group (IV-B) of Table 1. In some embodiments, L^{cyc} is a group formed by reacting functional group (V-A) with functional group (V-B) of Table 1.

In some embodiments of a cyclic peptide of formula (III), L^{cyc} is —C(═O)—CH₂—S—. In some embodiments of a cyclic peptide of formula (III), XaaC is cysteine, XaaN is chloroacetylated, L^{N} and L^{C} are absent, and L^{cyc} is formed by reacting —C(═O)—CH₂Cl on the XaaN with the —SH group on the cysteine.

In some embodiments of a cyclic peptide of formula (III), XaaC is cysteine, homocysteine, lysine, homolysine, ornithine, diaminobutric acid, serine, homoserine, threonine, or homothreonine, XaaN is chloroacetylated, and L^{cyc} is —C(═O)—CH₂—S—, —C(═O)—CH₂—NH—, or —C(═O)—CH₂—O— formed by reacting —C(═O)—CH₂Cl on the XaaN with the functional group of XaaC (such as —SH, —NH₂, —OH, —COOH). In some embodiments, L^{N} and L^{C} are absent (i.e., a bond).

In some embodiments of a cyclic peptide of formula (III), XaaC is aspartic acid, glutamic acid, or homoglutamic acid, XaaN comprises or is functionalized with —NH₂, and L^{cyc} is an amide formed by reacting —NH₂ on the XaaN with the functional group —COOH of XaaC.

In some embodiments of a cyclic peptide of formula (III), XaaC is lysine, homolysine, ornithine, or diaminobutric acid, XaaN comprises or is functionalized with —COOH, and L^{cyc} is an amide formed by reacting —COOH on the XaaN with the functional group —NH₂ of XaaC.

In some embodiments of a cyclic peptide of formula (III), XaaC is cysteine or homocysteine, XaaN comprises or is functionalized with halogen such as —Br, and L^{cyc} is —S— formed by reacting —Br on the XaaN with the functional group —SH of XaaC.

In some embodiments of a cyclic peptide of formula (III), XaaC is allyl-glycine or homoallyl-glycine, XaaN comprises or is functionalized with —CH═CH₂, and L^{cyc} is —CH═CH₂— formed by reacting —CH═CH₂ on the XaaN with the functional group —CH═CH₂ of XaaC.

In some embodiments of a cyclic peptide of formula (III), XaaC is aspartate-4-semialdehyde or glutamate-5-semialdehyde, XaaN comprises or is functionalized with —NH₂, and L^{cyc} is —NH— formed by reacting —NH₂ on the XaaN with the functional group —C(═O) of XaaC.

In some embodiments of a cyclic peptide of formula (III), XaaC is cysteine or homocysteine conjugated with 1,2- or 1,3- or 1,4-bis-(bromomethyl)benzene, XaaN comprises or is functionalized with —SH, and L^{cyc} is —S—CH₂-benzene-CH₂—S— formed by reacting —SH on the XaaN with the functional group of XaaC.

In some embodiments of a cyclic peptide of formula (III), XaaC is functionalized with azide, XaaN is functionalized with an alkyne, and L^{cyc} is a triazole formed by a click reaction between the azide and the alkyne. In some embodiments of a cyclic peptide of formula (III), XaaC is functionalized with an alkyne, XaaN is functionalized with an azide, and L^{cyc} is a triazole formed by a click reaction between the azide and the alkyne.

In some embodiments of a cyclic peptide of formula (III), XaaC is functionalized with azide, XaaN is functionalized with an alkene, and L^{cyc} is a triazoline formed by a cycloaddition reaction between the azide and the alkene. In some embodiments of a cyclic peptide of formula (III), XaaC is functionalized with an alkene (e.g., XaaC being alkynyl-glycine, homoalkynyl-glycine), XaaN is functionalized with an azide, and L^{cyc} is a triazoline formed by a cycloaddition reaction between the azide and the alkene.

In some embodiments of a cyclic peptide of formula (III), XaaC is cysteine or homocysteine, XaaN comprises or is functionalized with maleimide, and L^{cyc} is -succinimide-S— formed by reacting -maleimide on the XaaN with —SH group —CH═CH₂ of XaaC.

In some embodiments of a cyclic peptide of formula (III), Lis a structure of Table 2A, wherein n is 0 and m is 0. In some embodiments of a cyclic peptide of formula (II), the ring closing group is a structure of Table 2A.

For example, a cyclic peptide of formula (III) can have a structure of

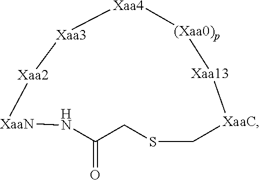

wherein XaaC is cysteine.

In some embodiments, a cyclic peptide of formula (III) can have a structure of

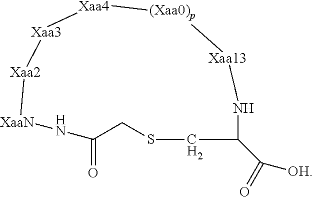

In some embodiments, a cyclic peptide of formula (III) can have a structure of

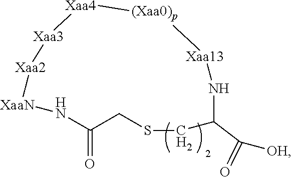

In some embodiments, a conjugate comprising a cyclic peptide of formula (III) has a structure of

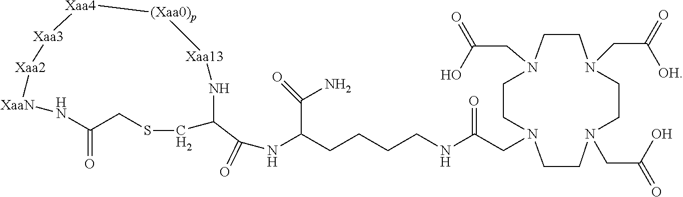

In some embodiments of a cyclic peptide of formula (III), L^{N}-L^{cyc}-L^{C} is —C(═O)—CH₂—S—, —C(═O)—CH₂—S—CH₂—, —C(═O)—CH₂—S—CH₂—CH₂—, —(CH₂)ₘ—NH—CO—(CH₂)ₙ—, —(CH₂)ₘ—CO—NH—(CH₂)ₙ—, —(CH₂)ₘ—S—(CH₂)ₙ—, —(CH₂)ₘ—CH═CH—(CH₂)ₙ—, —(CH₂)ₘ—NH—(CH₂)ₙ—, —(CH₂)ₘ—S—CH₂-benzene-CH₂-S-(CH₂)ₙ—, —(CH₂)ₘ-triazine-(CH₂)ₙ—, —(CH₂)ₘ-succinimide-S—(CH₂)ₙ—, —C(═O)—CH₂—NH—CH₂—, or —C(═O)—CH₂—O—CH₂—, where m and n are each independently 0 or an integer from 1 to 6. In some embodiments of a cyclic peptide of formula (III), L^{cyc} is a group of Table 2A.

In some embodiments of a cyclic peptide of formula (III), L^{cyc} is formed by reacting the functional group of XaaN with XaaC or a functional group of XaaC, as shown in Table 2B.

In some embodiments, a conjugate comprising a cyclic peptide of formula (III) has a structure of

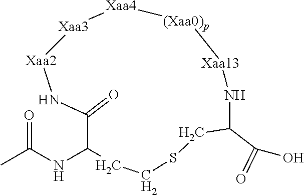

where the side chain of XaaN is conjugated to the side chain of XaaC.

TABLE 2A
Ring Closing Groups
(m and n are independently 0 or an integer from 1 to 6.)

—C(═O)—CH₂—S—
—C(═O)—CH₂—S—CH₂—
—C(═O)—CH₂—S—CH₂—CH₂—
—(CH₂)ₘ—NH—CO—(CH₂)ₙ—
—(CH₂)ₘ—CO—NH—(CH₂)ₙ—
—(CH₂)ₘ—S—(CH₂)ₙ—
—(CH₂)ₘ—CH═CH—(CH₂)ₙ—
—(CH₂)ₘ—NH—(CH₂)ₙ—
—(CH₂)ₘ—S—CH₂-benzene-CH₂—S—(CH₂)ₙ—
—(CH₂)ₘ-triazole-(CH₂)ₙ—
—(CH₂)ₘ-succinimide-S—(CH₂)ₙ—
—C(═O)—CH₂—NH—CH₂—
—C(═O)—CH₂—O—CH₂—

In some embodiments of Formula (III), L^{cyc} is a structure of Table 2A, wherein m is 0 and n is 0. In some embodiments of Formula (III), L^{cyc} comprises a structure of Table 2A, wherein m is 0 and n is 0.

In some embodiments, m is 0. In some embodiments, m is 1. In some embodiments, m is 2. In some embodiments, m is 3. In some embodiments, m is 4. In some embodiments, m is 5. In some embodiments, m is 6. In some embodiments, n is 0. In some embodiments, n is 1. In some embodiments, n is 2. In some embodiments, n is 3. In some embodiments, n is 4. In some embodiments, n is 5. In some embodiments, n is 6.

TABLE 2B
Formation of Ring Closing Groups

| Functional group | |
|---|---|
| of XaaN | XaaC (or functional group of XaaC) |
| —C(═O)—CH₂Cl | Cysteine, homocysteine, lysine, homolysine, |
| | ornithine, diaminobutric acid, serine, homoserine, |
| | threonine, homothreonine |
| —(CH₂)ₙ—NH₂ | Aspartic acid, glutamic acid, homoglutamic acid |
| —(CH₂)ₙ—CO₂H | Lysine, homolysine, ornithine, diaminobutric |
| | acid, |
| —(CH₂)ₙ—Br | Cysteine, homocysteine |
| —(CH₂)ₙ—CH═CH₂ | Allyl-glycine, homoallyl-glycine |
| —(CH₂)ₙ—NH₂ | Aspartate-4-semialdehyde, glutamate-5- |
| | semialdehyde |
| —(CH₂)ₙ—SH | Cysteine, homocysteine conjugated with 1,2- or |
| | 1,3- or 1,4-bis-(bromomethyl)benzene |
| —(CH₂)ₙ-alkyne | XaaC is an amino acid with a side chain with |
| | azide |
| —(CH₂)ₙ—N₃ | Alkynyl-glycine, homoalkynyl-glycine |
| —(CH₂)ₙ-maleimide | Cysteine, homocysteine |

In some embodiments, a cyclic peptide of formula (III) can represented by a compound in Table 3. In some embodiments, the conjugate further comprises a metal chelator and optionally a linker. In some embodiments, the conjugate further comprises a radionuclide such as Ac-225 or Lutetium-177.

TABLE 3
Exemplary cyclic peptides

| | Ring | | | | |
|---|---|---|---|---|---|
| | Closing | N- | | | |
| | group | terminus | | SEQ | |
| Compound | (upon | (before | | ID | |
| No. | cyclization) | cyclization) | Peptide sequence | NO: | Target |
| P-01 | -C(═O)- | -C(═O)- | AHYTAhpMeGWMeFRVRWYMeFC | 1 | Nectin4 |
| | CH₂-S- | CH₂Cl | | | |
| P-02 | -C(═O)- | -C(═O)- | MeFSWMeGYNYMeNal1GWWC | 2 | Nectin4 |
| | CH₂-S- | CH₂Cl | | | |
| P-10 | -C(═O)- | -C(═O)- | ARLMeFDYDTMeYMeYWYC | 3 | FOLR1 |
| | CH₂-S- | CH₂Cl | | | |
| P-11 | -C(═O)- | -C(═O)- | DRChgMeFDF3CDTMeYMeYWYC | 4 | FOLR1 |
| | CH₂-S- | CH₂Cl | | | |
| P-12 | -C(═O)- | -C(═O)- | DRChgMeFDYDTMeYMeYWYC | 5 | FOLR1 |
| | CH₂-S- | CH₂Cl | | | |
| P-20 | -C(═O)- | -C(═O)- | AIMeFYDDMeYYDMeYC | 6 | PSMA |
| | CH₂-S- | CH₂Cl | | | |
| P-21 | -C(═O)- | -C(═O)- | WmBphDVDSDCbaWmBphDC | 7 | PSMA |
| | CH₂-S- | CH₂Cl | | | |
| P-22 | -C(═O)- | -C(═O)- | MeFDDSVWDHphYC | 8 | PSMA |
| | CH₂-S- | CH₂Cl | | | |
| P-30 | -C(═O)- | -C(═O)- | dF3PyIL3PyMeAMeASDMeYFIIC | 13 | EphA2 |
| | CH₂-S- | CH₂Cl | | | |
| P-31 | -C(═O)- | -C(═O)- | AVF3CYRGMeFTGW6CVMeGAhpC | 9 | EphA2 |
| | CH₂-S- | CH₂Cl | | | |
| P-40 | -C(═O)- | -C(═O)- | dADBphIYPABphSSESMeFC | 14 | Trop-2 |
| | CH₂-S- | CH₂Cl | | | |

As described in Table 3, only the cyclic portion of the peptide sequence is included. In some embodiments, a conjugate comprising any one of peptide of Table 3 may further comprise amino acid residues at the N and/or C terminus of the peptide, which is not part of the cyclic structure.

As described in Table 3, abbreviations have the following meanings:

Low case d means D-amino acids, e.g., dF refers to d-phenylalanine;

Me refers to a methyl group, e.g., MeG represents N-Methyl-Glycine;

Ahp refers to 2-aminoheptanoic acid;

Nal1 refers to 1-naphthylalanine;

Chg refers to cyclohexylglycine;

F3C refers to 3-chlorophenylalanine;

mBph refers to 3-phenylphenylalanine;

Cba refers to cyclobutylalanine;

Hph refers to homophenylalanine; and

W6C refers to 6-chlorotryptaphan.

A peptide described herein can be a peptide mimetic. For example, the peptide can comprise non-peptide bonds and it can comprise one or more unnatural amino acids. Unless stated otherwise, each of the amino acid in a peptide described herein (except the natural amino acid glycine) can independently be in its D or L form. Both D and L forms are encompassed by the present disclosure.

In the present disclosure, the term amino acid embraces derivatives of amino acids. The derivatives include, for example, amino acids obtained by modifying a natural amino acid constituting a protein produced by cellular DNA-encoded biological matter. Examples of such non-natural amino acids include hydroxyproline and hydroxylysine, which are amino acids having a hydroxyl group introduced therein, and diaminopropionic acid, which is an amino acid having an amino group introduced therein.

A peptide described herein can comprise an N-substituted amino acid. In some embodiments, the N-substituted amino acid is a derivative of tryptophan, phenylalanine, tyrosine, arginine, histidine, isoleucine, leucine, lysine, or valine. In some embodiments, the N-substitution is an N-alkyl, such as N-methyl and N-ethyl. In some embodiments, the N-substitution is an N-aryl, such as N-phenyl or N-biphenyl. In some embodiments, the N-substitution is an N-heteroaryl such as N-pyridyl. In some embodiments, the N-substituted amino acid is at the N-terminus of the peptide. In some embodiments, the N-substituted amino acid is a non-terminal amino acid.

In some embodiments, peptides described herein comprise one or more amino acids in Tables 4A to 4G.

TABLE 4A
Amino Acids at N or C-terminus

| | |
|---|---|
| N-Chloroacetyl-L-alanine | Acetyl-L-alanine |
| N-Chloroacetyl-L-phenylalanine | Acetyl-L-phenylalanine |
| N-Chloroacetyl-L-phenylalanine | Acetyl-L-tyrosine |
| N-Chloroacetyl-L-tyrosine | Acetyl-L-tryptophan |
| N-Chloroacetyl-L-tryptophan | Acetyl-D-alanine |
| N-Chloroacetyl-D-alanine | Acetyl-D-phenylalanine |
| N-Chloroacetyl-D-phenylalanine | Acetyl-D-tyrosine |
| N-Chloroacetyl-D-tyrosine | Acetyl-D-tryptophan |
| N-Chloroacetyl-D-tryptophan | N-3-chloromethylbenzoyl-L-tyrosine |
| | N-3-chloromethylbenzoyl-L-tryptophan |

TABLE 4B
Amino Acids That Crosslink With A Peptide

Nγ-(2-chloroacetyl)-α,γ-diaminobutylic acid
Nγ-(2-chloroacetyl)-α,γ-diaminopropanoic acid

TABLE 4C
D-amino Acids

D-Serine
D-Phenylalanine
D-Tyrosine
D-Tryptophan

TABLE 4D
N-alkylamino Acids

N-alkyl-Glycine
N-alkyl-Alanine
N-alkyl-Phenylalanine
N-alkyll-Tyrosine
N-alkyl-Serine
N-alkyll-Histidine
N-alkyl-Tryptophan

Exemplary alkyl groups for Table 1D include methyl, ethyl, and propyl groups.

TABLE 4E
Peptoid Blocks

N-ethyl-Glycine
N-n-propyl-Glycine
N-n-butyl-Glycine
N-n-pentyl-Glycine
N-n-hexyl-Glycine
N-n-heptyl-Glycine
N-n-octyl-Glycine
N-isopentyl-Glycine
N-(2-phenylethyl)-Glycine
N-(3-phenylpropyl)-Glycine
N-[2-(p-hydroxyphenyl)ethyl]-Glycine

TABLE 4F
Exemplary Unnatural Amino Acids

p-biphenylalanine
p-trifluoromethylphenylalanine
p-azidophenylalanine
p-biotinyl-aminophenylalanine
e-N-Biotinyl-lysine
e-N-Acetyl-lysine
L-Citrulline
L-5-Hydroxytryptphan
L-1,2,3,4,-Tetrahydroisoquinoline-3-carboxylic acid
Aminoisobutyric acid
N-methyl-aminoisobutyric acid
N-methyl-Phenylglycine

TABLE 4G
Amino Acids that can be incorporated into a cyclic
peptide described herein

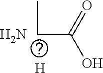

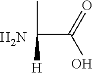

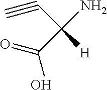

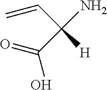

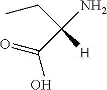

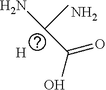

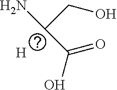

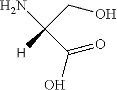

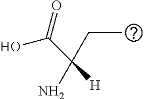

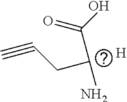

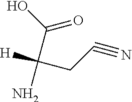

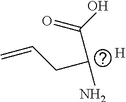

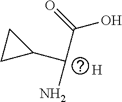

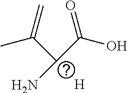

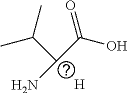

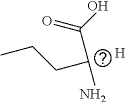

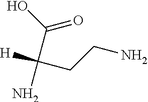

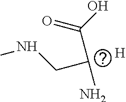

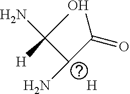

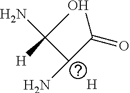

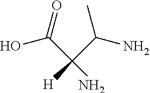

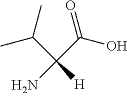

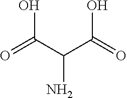

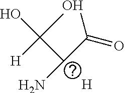

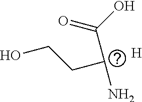

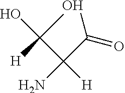

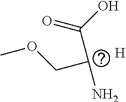

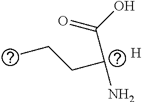

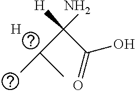

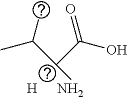

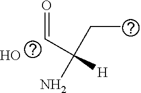

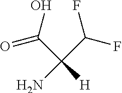

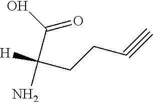

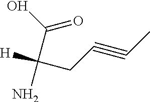

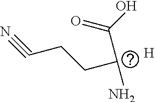

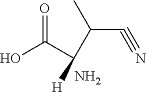

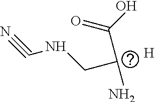

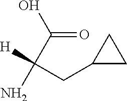

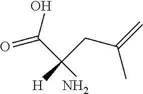

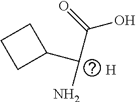

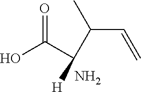

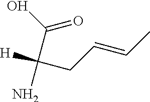

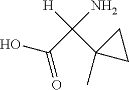

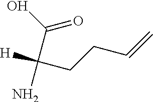

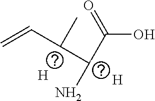

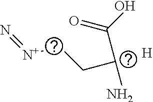

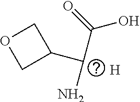

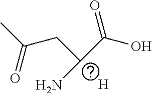

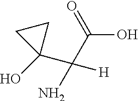

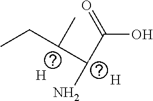

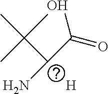

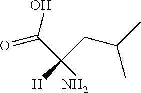

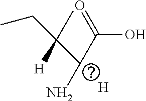

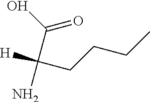

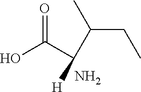

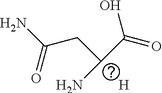

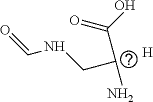

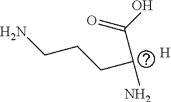

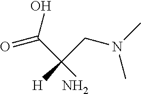

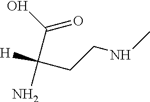

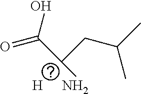

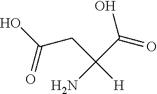

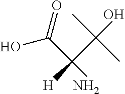

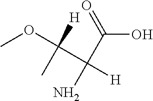

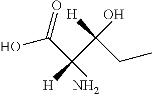

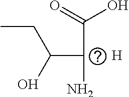

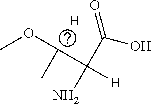

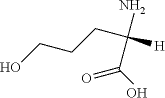

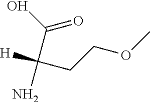

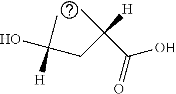

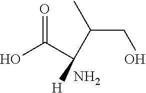

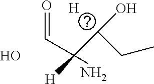

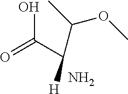

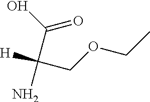

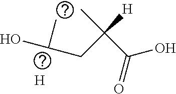

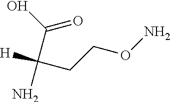

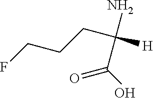

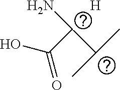

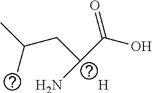

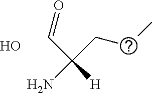

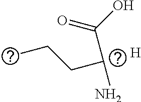

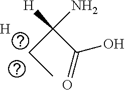

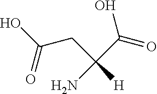

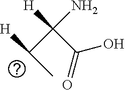

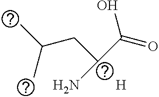

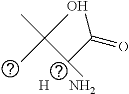

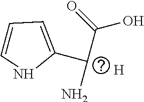

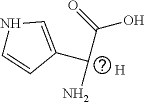

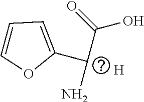

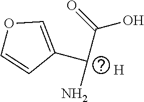

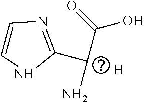

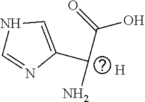

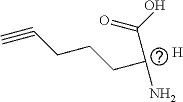

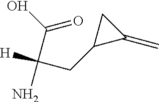

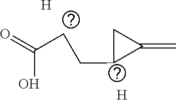

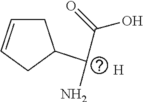

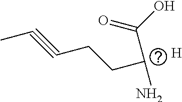

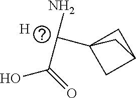

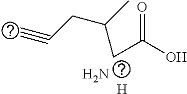

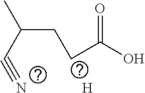

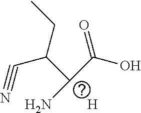

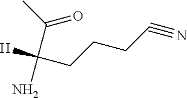

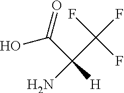

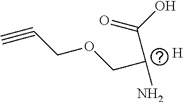

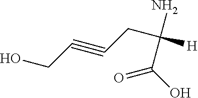

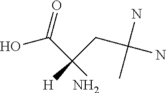

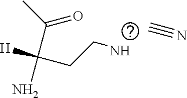

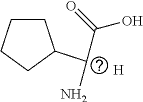

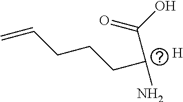

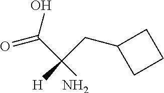

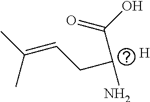

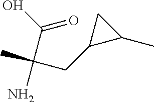

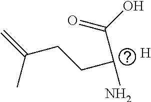

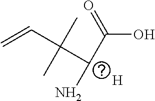

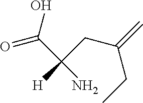

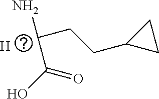

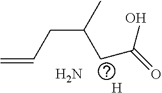

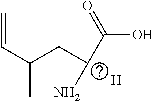

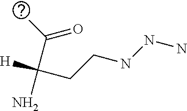

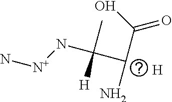

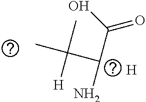

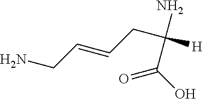

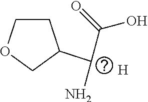

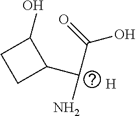

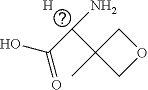

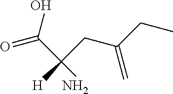

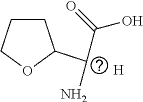

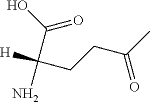

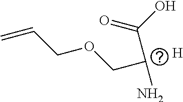

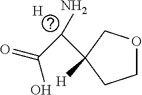

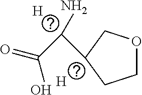

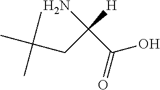

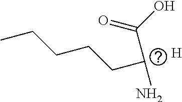

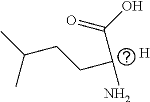

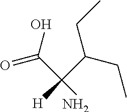

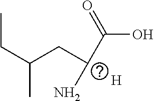

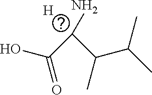

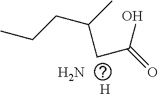

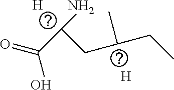

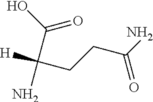

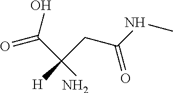

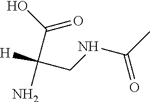

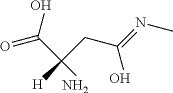

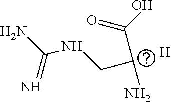

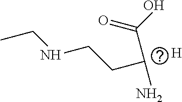

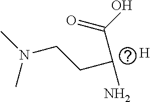

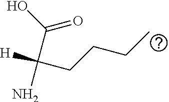

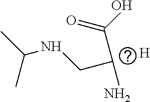

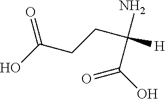

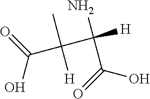

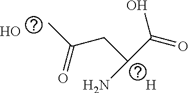

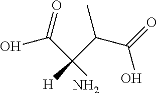

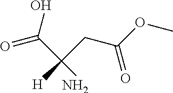

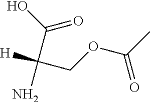

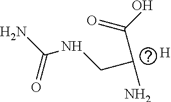

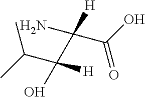

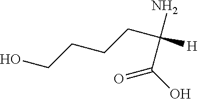

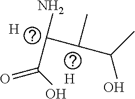

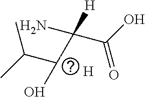

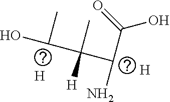

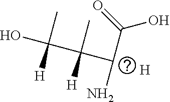

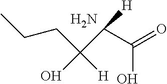

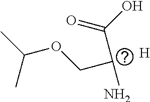

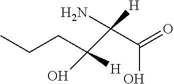

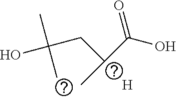

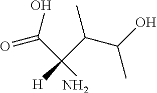

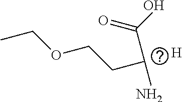

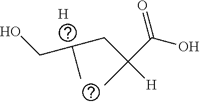

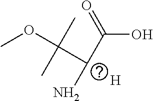

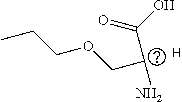

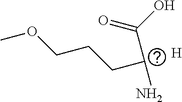

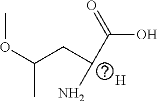

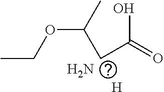

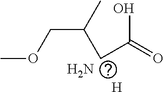

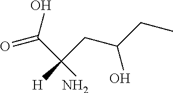

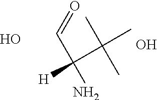

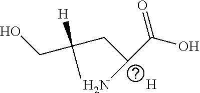

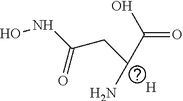

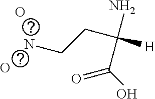

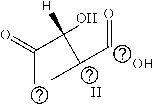

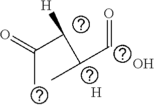

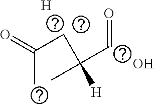

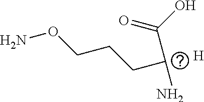

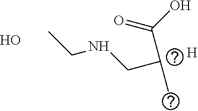

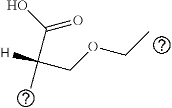

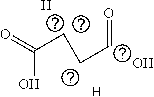

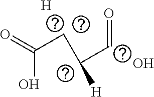

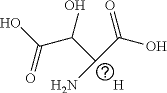

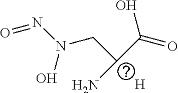

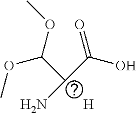

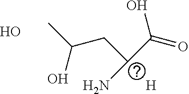

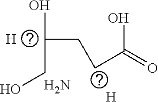

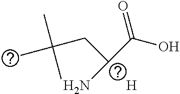

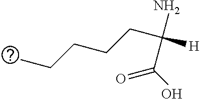

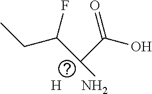

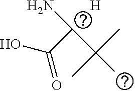

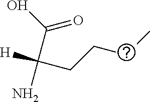

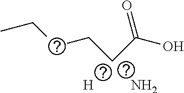

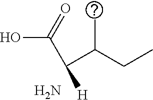

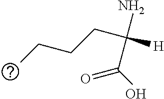

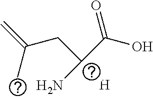

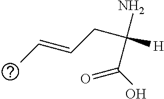

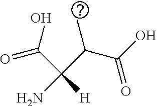

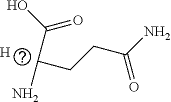

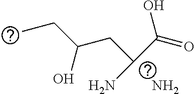

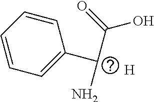

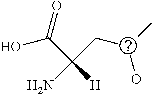

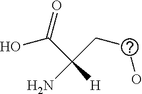

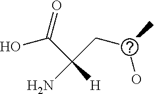

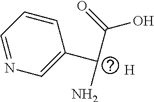

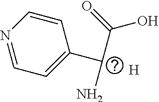

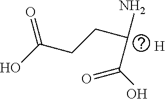

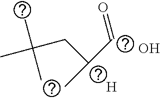

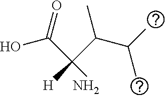

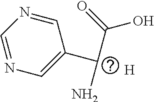

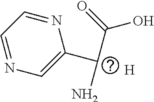

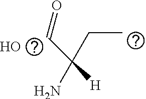

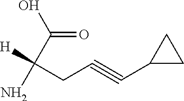 [SMILES: N[C@@H](CC#CC1CC1)C(=O)O]

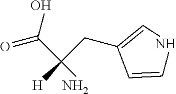 [SMILES: N[C@@H](Cc1cc[nH]c1)C(=O)O]

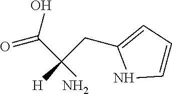 [SMILES: N[C@@H](Cc1ccc[nH]1)C(=O)O]

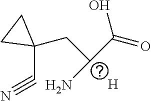

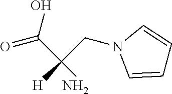 [SMILES: N[C@@H](Cn1cccc1)C(=O)O]

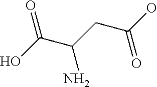

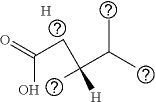

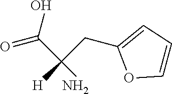 [SMILES: N[C@@H](Cc1ccco1)C(=O)O]

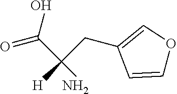 [SMILES: N[C@@H](Cc1ccoc1)C(=O)O]

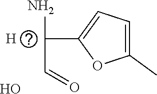

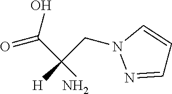 [SMILES: N[C@@H](Cn1cccn1)C(=O)O]

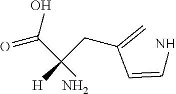

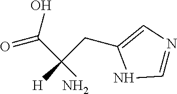 [SMILES: N[C@@H](Cc1cnc[nH]1)C(=O)O]

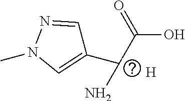

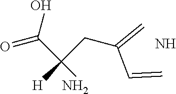

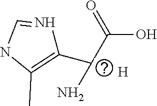

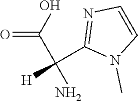

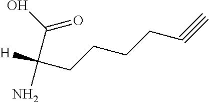

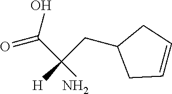 [SMILES: N[C@@H](CC1CC=CC1)C(=O)O]

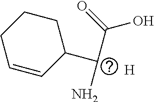

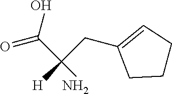 [SMILES: N[C@@H](CC1=CCCC1)C(=O)O]

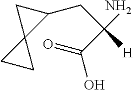 [SMILES: N[C@@H](CC1CC12CC2)C(=O)O]

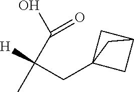

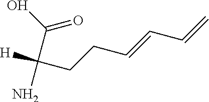

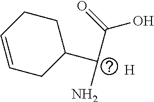

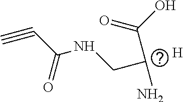

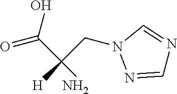 [SMILES: N[C@@H](Cn1cncn1)C(=O)O]

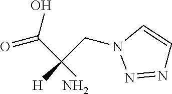 [SMILES: N[C@@H](Cn1ccnn1)C(=O)O]

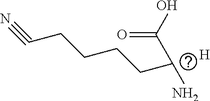

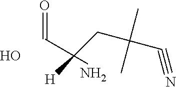

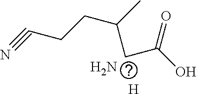

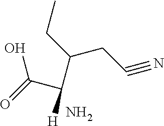

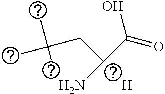

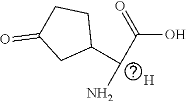

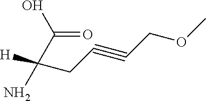

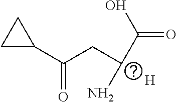

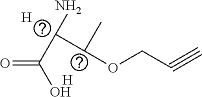

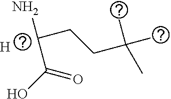

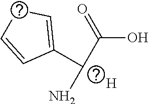

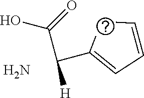

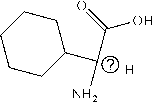

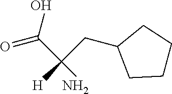 [SMILES: N[C@@H](CC1CCCC1)C(=O)O]

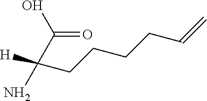

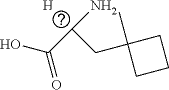

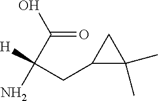

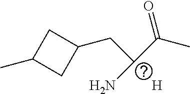

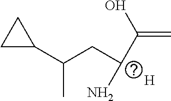

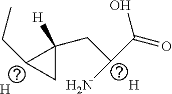

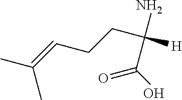

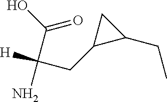

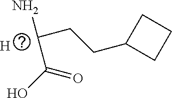

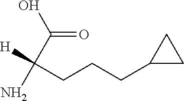 [SMILES: N[C@@H](CCCC1CC1)C(=O)O]

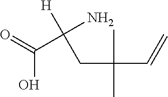

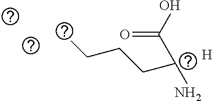

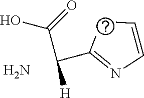

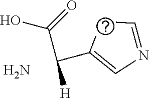

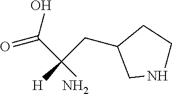 [SMILES: N[C@@H](CC1CCNC1)C(=O)O]

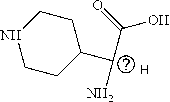

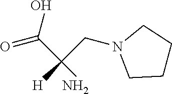 [SMILES: N[C@@H](CN1CCCC1)C(=O)O]

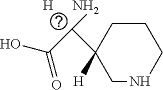

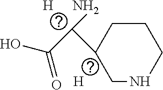

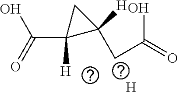

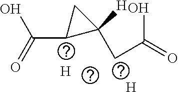

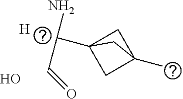

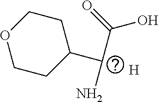

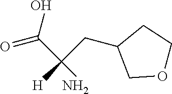 [SMILES: N[C@@H](CC1CCOC1)C(=O)O]

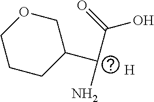

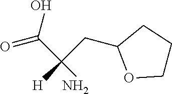 [SMILES: N[C@@H](CC1CCCO1)C(=O)O]

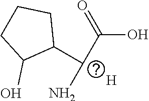

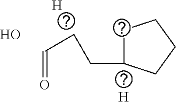

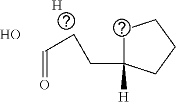

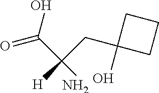 [SMILES: N[C@@H](CC1(O)CCC1)C(=O)O]

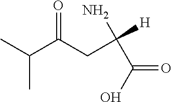

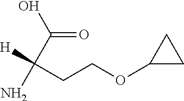 [SMILES: N[C@@H](CCOC1CC1)C(=O)O]

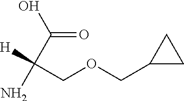 [SMILES: N[C@@H](COCC1CC1)C(=O)O]

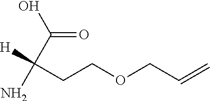

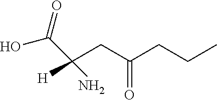

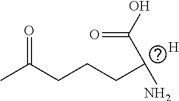

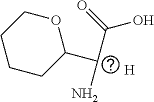

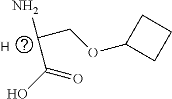

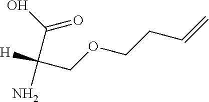

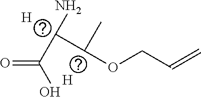

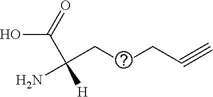

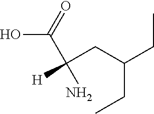

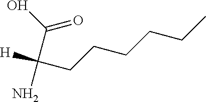

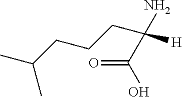

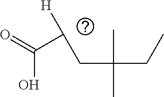

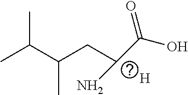

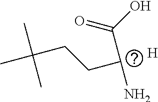

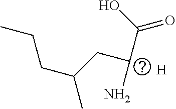

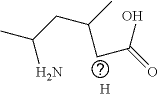

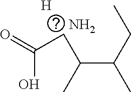

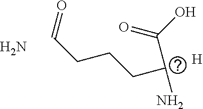

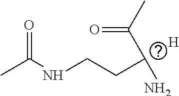

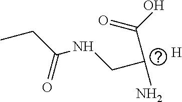

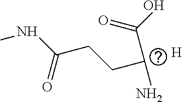

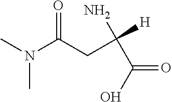

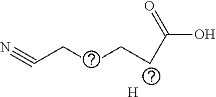

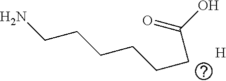

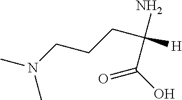

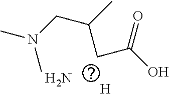

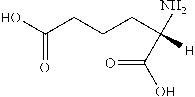 [SMILES: N[C@@H](CCCC(=O)O)C(=O)O]

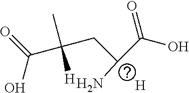

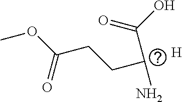

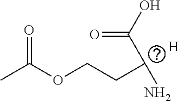

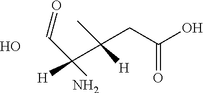

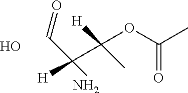

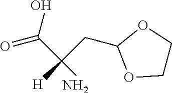 [SMILES: N[C@@H](CC1OCCO1)C(=O)O]

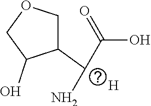

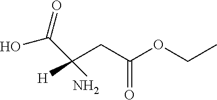

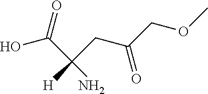

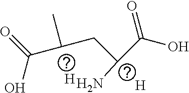

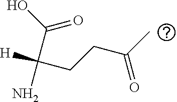

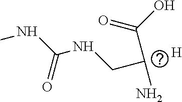

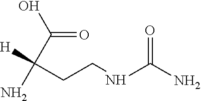 [SMILES: NC(=O)NCC[C@H](N)C(=O)O]

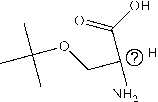

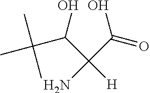

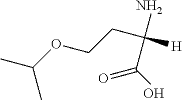

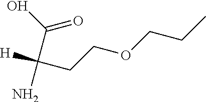

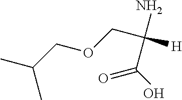

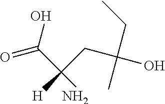

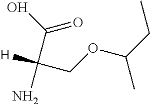

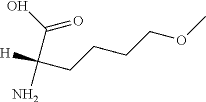

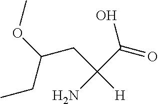

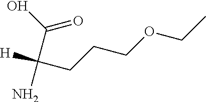

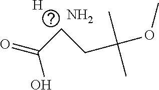

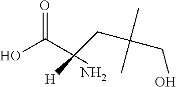

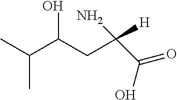

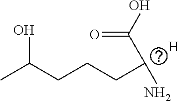

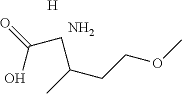

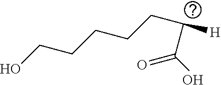

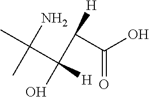

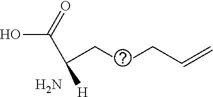

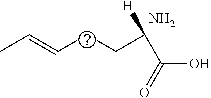

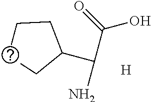

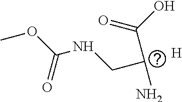

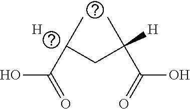

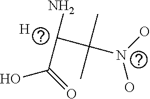

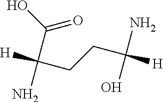

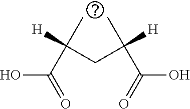

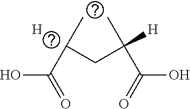

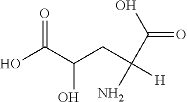 [SMILES: NC(CC(O)C(=O)O)C(=O)O]

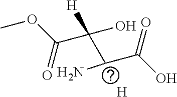

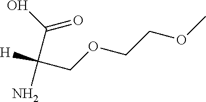

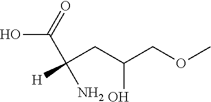

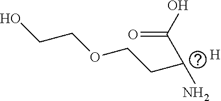

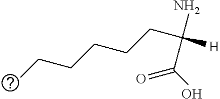

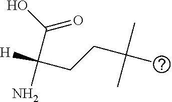

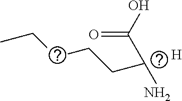

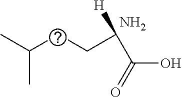

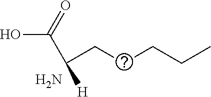

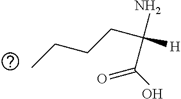

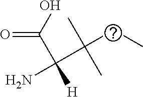

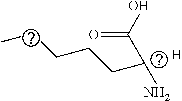

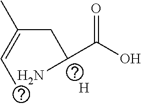

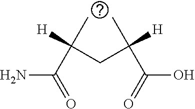

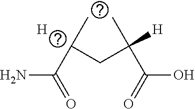

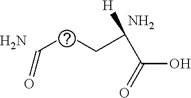

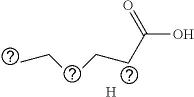

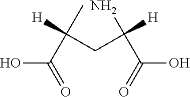

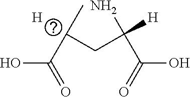

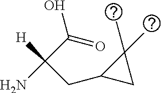

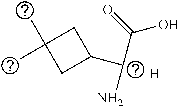

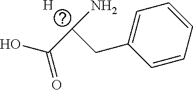

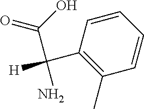

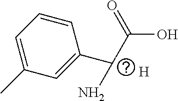

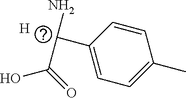

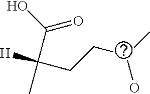

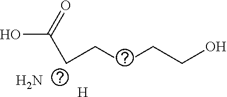

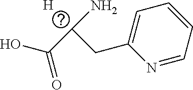

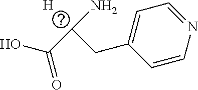

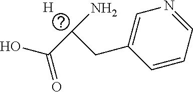

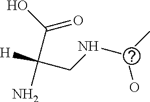

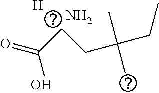

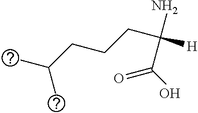

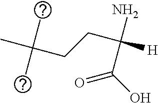

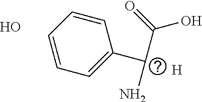

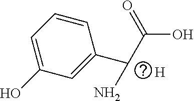

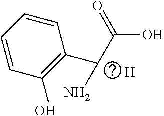

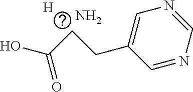

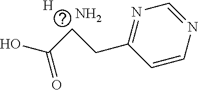

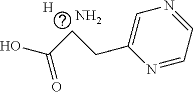

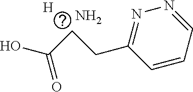

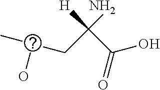

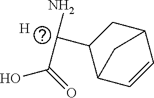

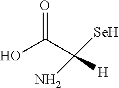

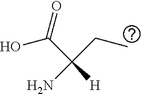

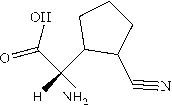 [SMILES: N#CC1CCCC1[C@H](N)C(=O)O]

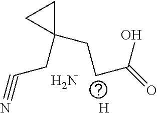

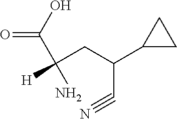 [SMILES: N#CC(C[C@H](N)C(=O)O)C1CC1]

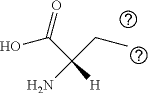

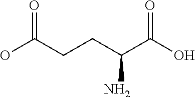

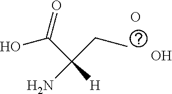

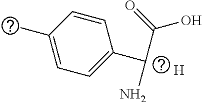

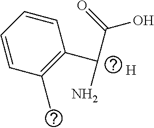

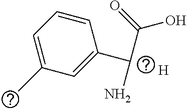

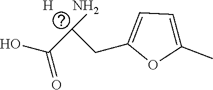

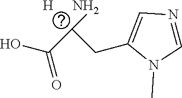

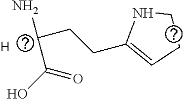

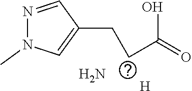

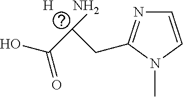

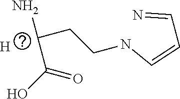

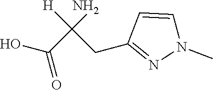

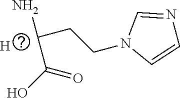

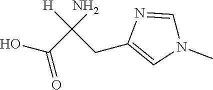

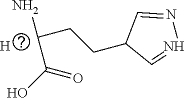

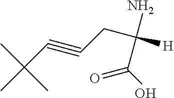

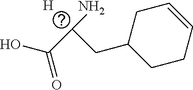

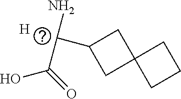

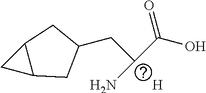

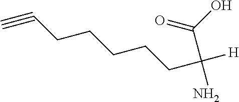

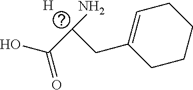

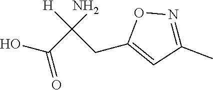

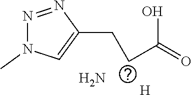

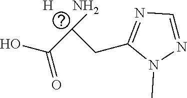

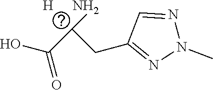

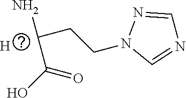

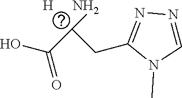

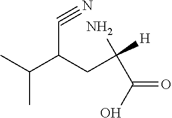

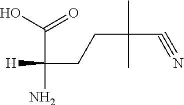

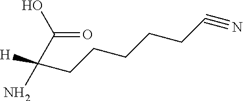 [SMILES: N#CCCCCC[C@H](N)C(=O)O]

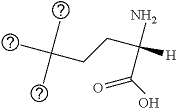

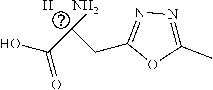

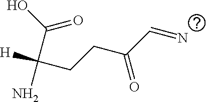 [SMILES: N=CC(=O)CC[C@H](N)C(=O)O]

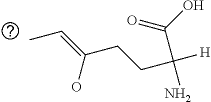

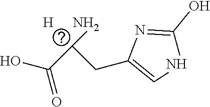

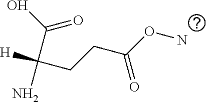 [SMILES: NOC(=O)CC[C@H](N)C(=O)O]

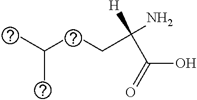

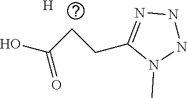

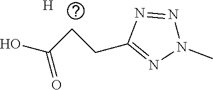

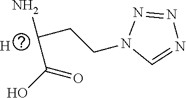

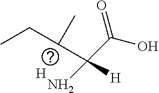

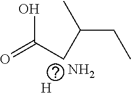

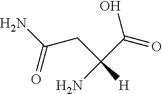

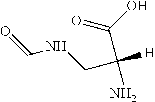

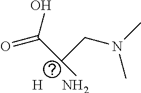

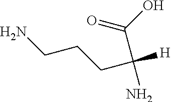

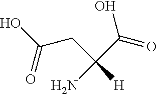

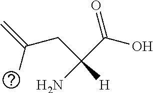

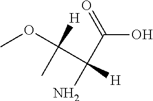

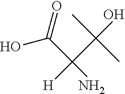

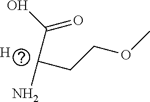

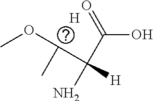

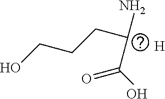

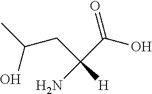

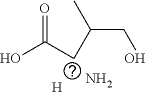

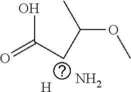

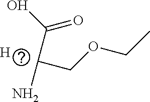

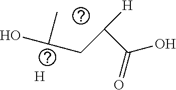

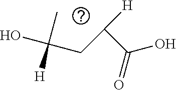

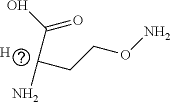

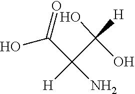

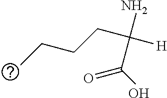

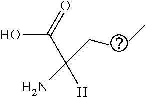

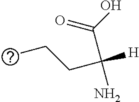

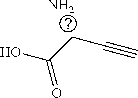

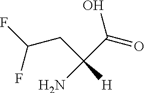

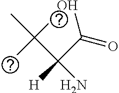

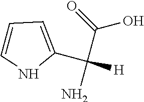

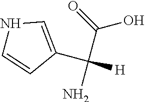

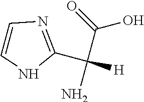

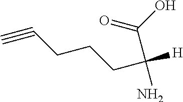

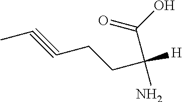

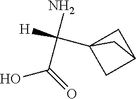

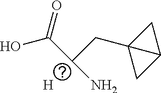

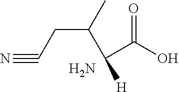

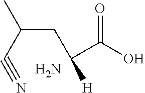

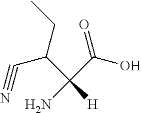

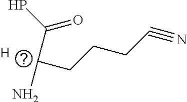

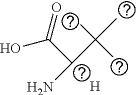

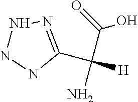

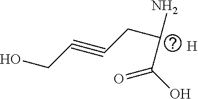

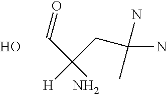

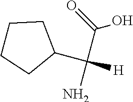

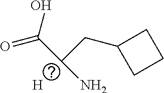

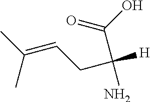

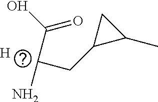

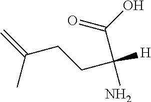

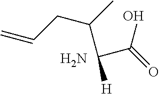

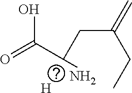

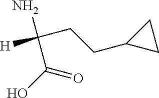

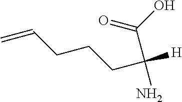

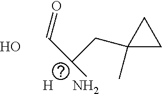

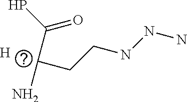

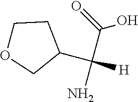

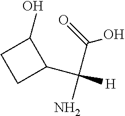

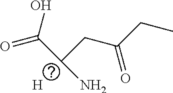

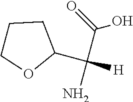

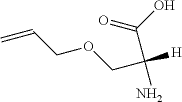

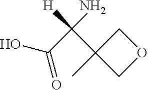

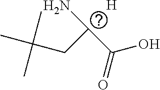

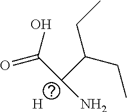

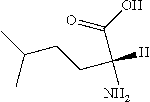

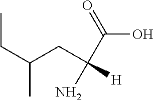

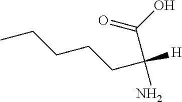

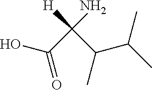

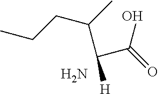

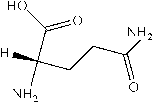

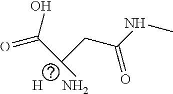

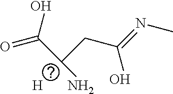

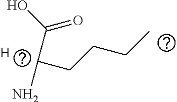

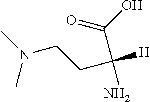

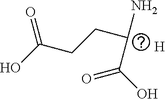

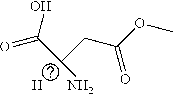

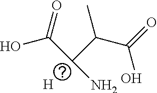

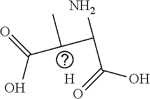

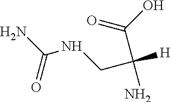

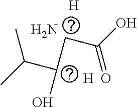

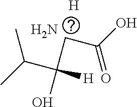

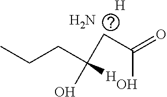

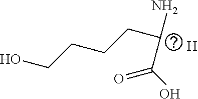

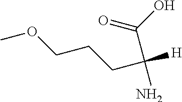

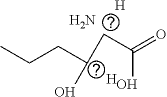

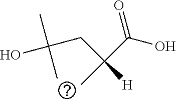

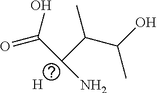

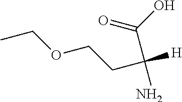

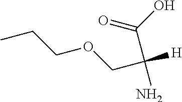

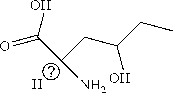

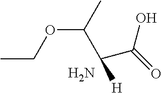

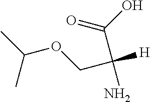

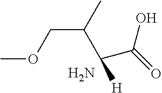

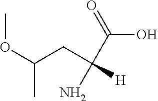

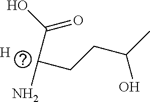

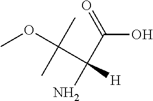

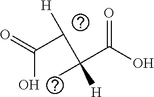

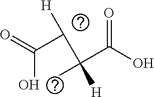

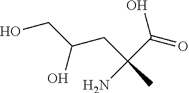

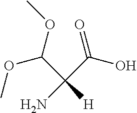

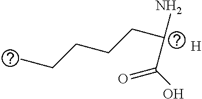

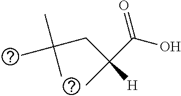

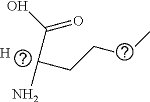

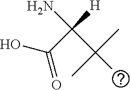

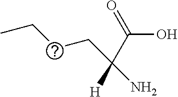

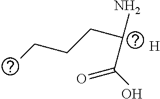

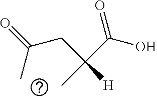

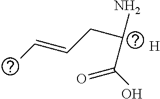

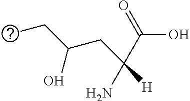

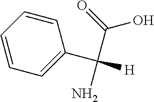 [SMILES: N[C@@H](C(=O)O)c1ccccc1]

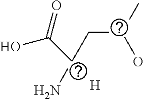

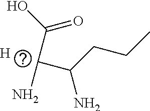

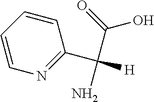 [SMILES: N[C@@H](C(=O)O)c1ccccn1]

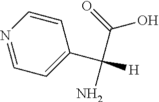 [SMILES: N[C@@H](C(=O)O)c1ccncc1]

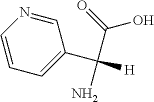 [SMILES: N[C@@H](C(=O)O)c1cccnc1]

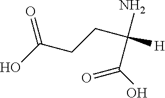

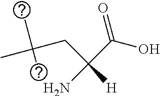

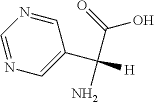 [SMILES: N[C@@H](C(=O)O)c1cncnc1]

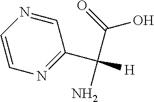 [SMILES: N[C@@H](C(=O)O)c1cnccn1]

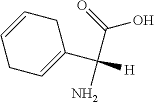 [SMILES: N[C@@H](C(=O)O)C1=CCC=CC1]

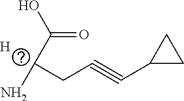

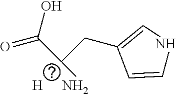

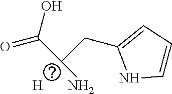

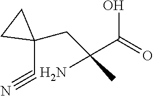

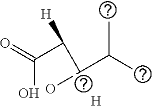

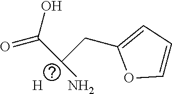

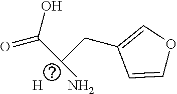

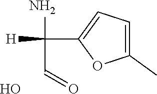

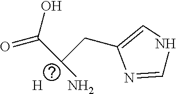

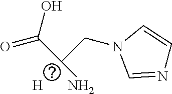

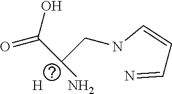

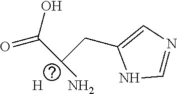

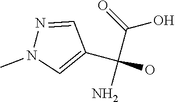

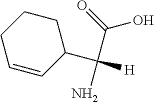 [SMILES: N[C@@H](C(=O)O)C1C=CCCC1]

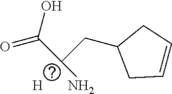

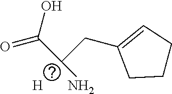

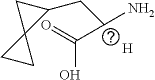

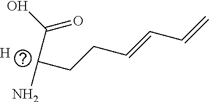

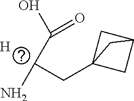

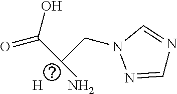

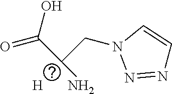

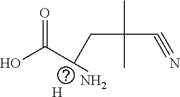

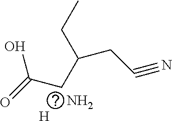

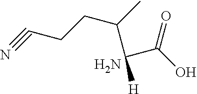

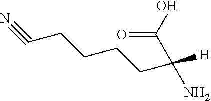 [SMILES: N#CCCCC[C@@H](N)C(=O)O]

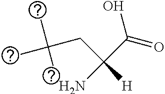

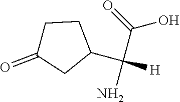 [SMILES: N[C@@H](C(=O)O)C1CCC(=O)C1]

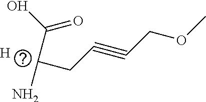

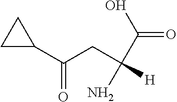 [SMILES: N[C@H](CC(=O)C1CC1)C(=O)O]

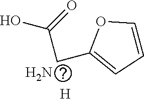

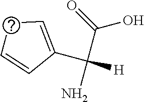

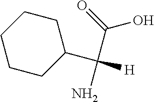 [SMILES: N[C@@H](C(=O)O)C1CCCCC1]

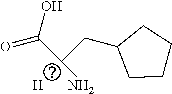

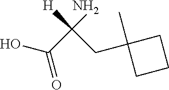

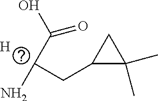

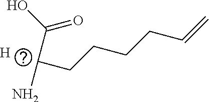

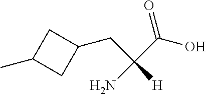

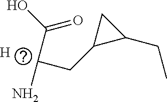

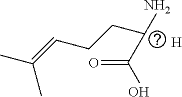

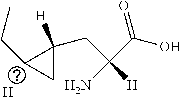

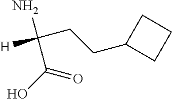 [SMILES: N[C@H](CCC1CCC1)C(=O)O]

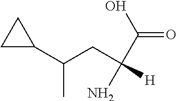

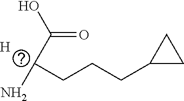

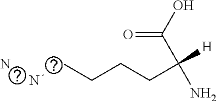 [SMILES: NNCCCC[C@@H](N)C(=O)O]

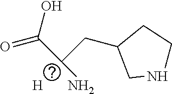

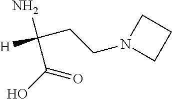 [SMILES: N[C@H](CCN1CCC1)C(=O)O]

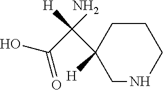 [SMILES: N[C@@H](C(=O)O)[C@@H]1CCCNC1]

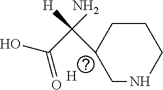

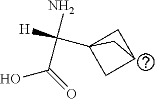

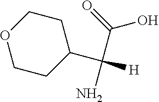 [SMILES: N[C@@H](C(=O)O)C1CCOCC1]

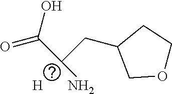

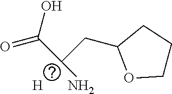

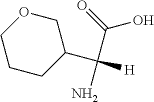 [SMILES: N[C@@H](C(=O)O)C1CCCOC1]

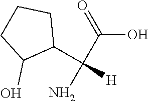 [SMILES: N[C@@H](C(=O)O)C1CCCC1O]

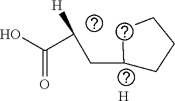

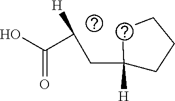

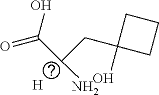

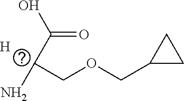

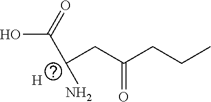

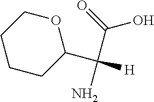 [SMILES: N[C@@H](C(=O)O)C1CCCCO1]

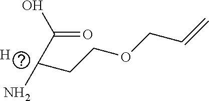

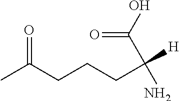

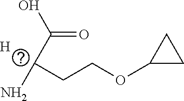

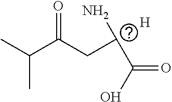

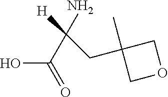

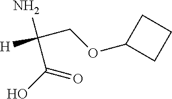 [SMILES: N[C@H](COC1CCC1)C(=O)O]

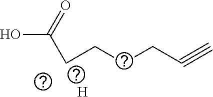

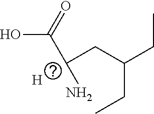

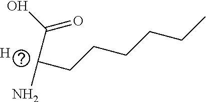

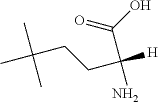

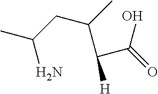

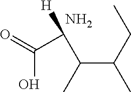

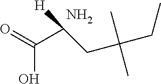

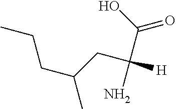

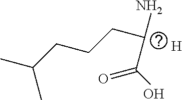

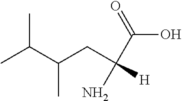

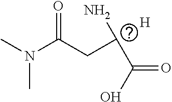

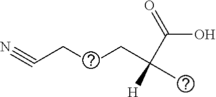

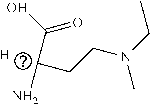

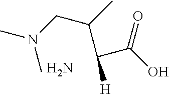

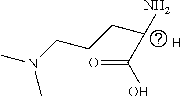

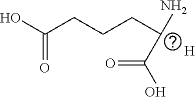

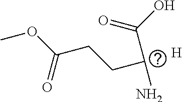

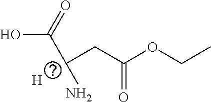

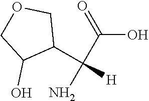 [SMILES: N[C@@H](C(=O)O)C1COCC1O]

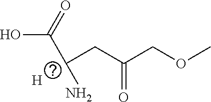

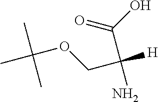

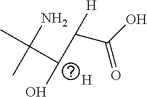

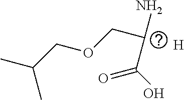

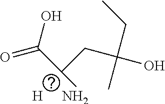

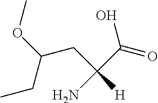

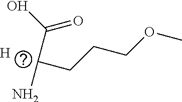

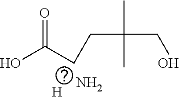

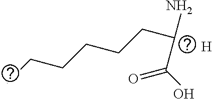

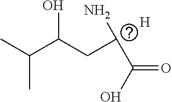

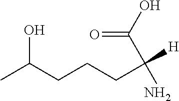

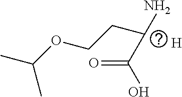

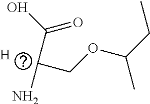

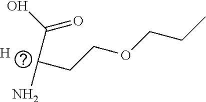

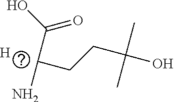

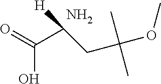

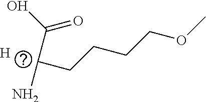

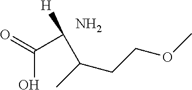

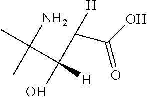

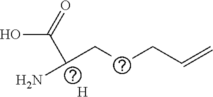

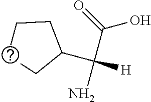

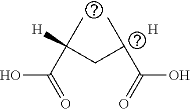

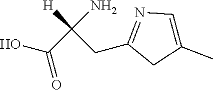

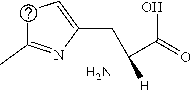

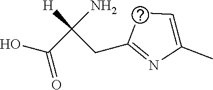

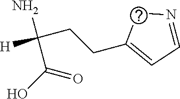 [SMILES: N[C@H](CCC1=CC=NC1)C(=O)O]

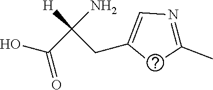

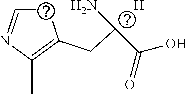

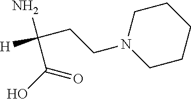 [SMILES: N[C@H](CCN1CCCCC1)C(=O)O]

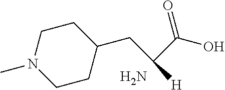

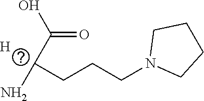

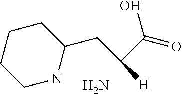

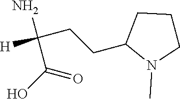

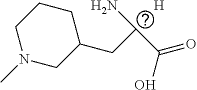

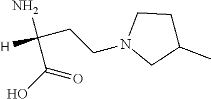

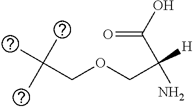

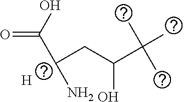

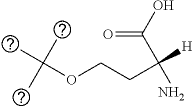

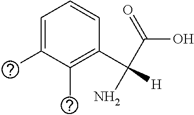

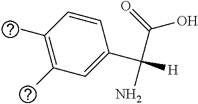

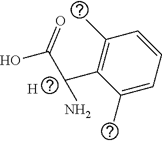

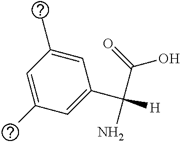

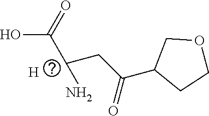

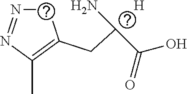

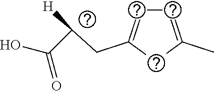

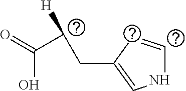

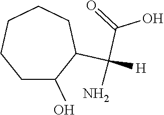 [SMILES: N[C@@H](C(=O)O)C1CCCCCC1O]

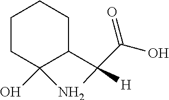

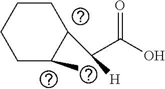

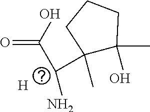

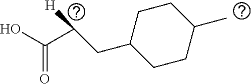

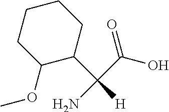

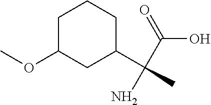

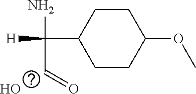

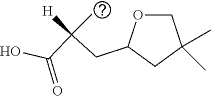

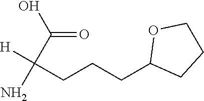 [SMILES: NC(CCCC1CCCO1)C(=O)O]

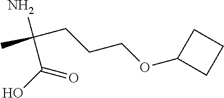

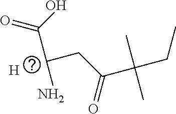

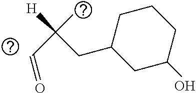

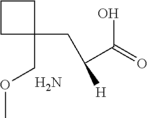

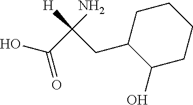 [SMILES: N[C@H](CC1CCCCC1O)C(=O)O]

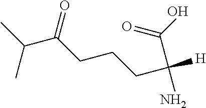

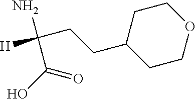 [SMILES: N[C@H](CCC1CCOCC1)C(=O)O]

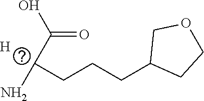

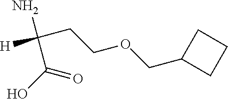 [SMILES: N[C@H](CCOCC1CCC1)C(=O)O]

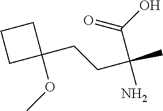

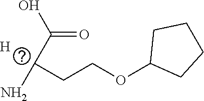

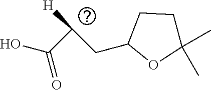

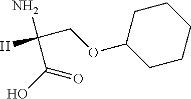 [SMILES: N[C@H](COC1CCCCC1)C(=O)O]

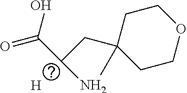

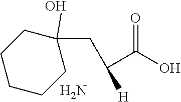

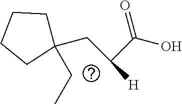

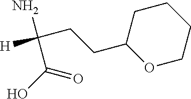 [SMILES: N[C@H](CCC1CCCCO1)C(=O)O]

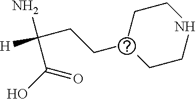 [SMILES: N[C@H](CCC1CCNCC1)C(=O)O]

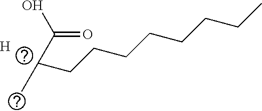

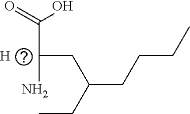

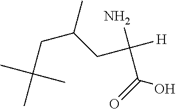

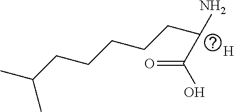

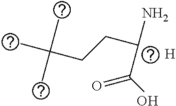

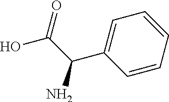 [SMILES: N[C@@H](C(=O)O)c1ccccc1]

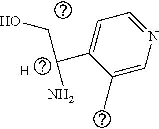

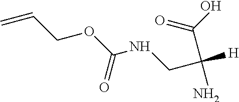

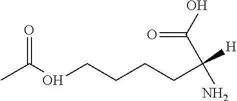

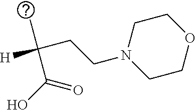

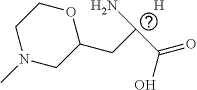

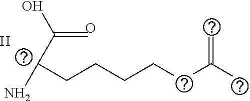

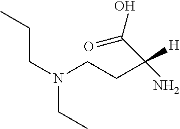

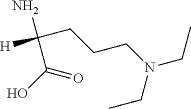

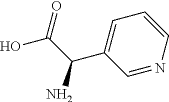 [SMILES: N[C@@H](C(=O)O)c1cccnc1]

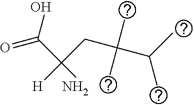

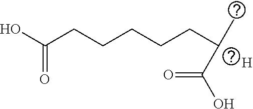

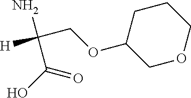 [SMILES: N[C@H](COC1CCCOC1)C(=O)O]

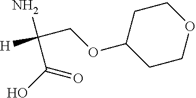 [SMILES: N[C@H](COC1CCOCC1)C(=O)O]

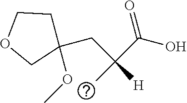

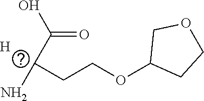

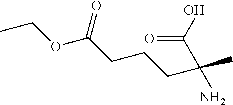

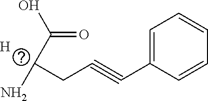

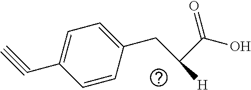

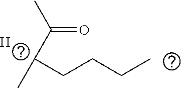

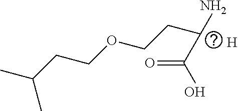

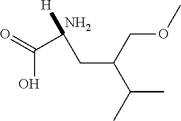

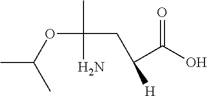

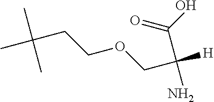

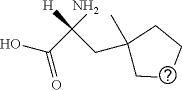

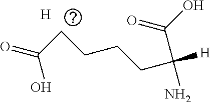

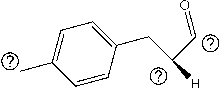

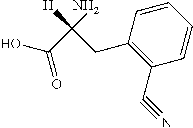 [SMILES: N#Cc1ccccc1C[C@@H](N)C(=O)O]

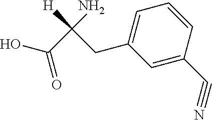 [SMILES: N#Cc1cccc(C[C@@H](N)C(=O)O)c1]

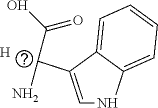

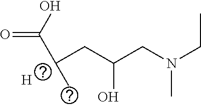

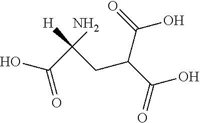 [SMILES: N[C@H](CC(C(=O)O)C(=O)O)C(=O)O]

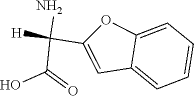 [SMILES: N[C@@H](C(=O)O)c1cc2ccccc2o1]

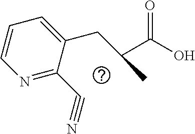

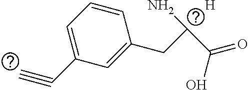

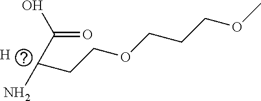

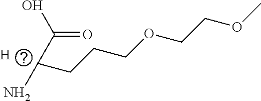

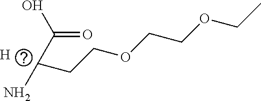

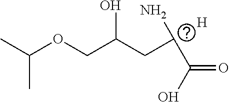

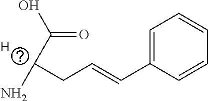

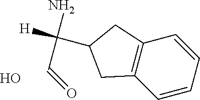

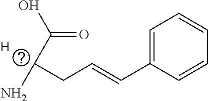

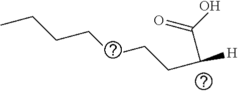

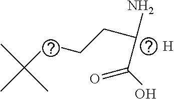

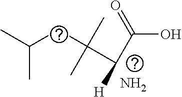

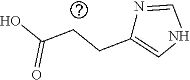

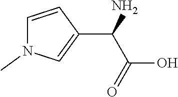

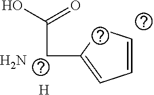

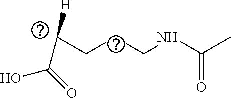

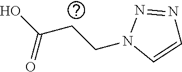

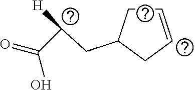

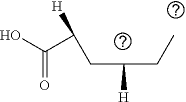

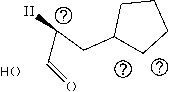

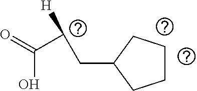

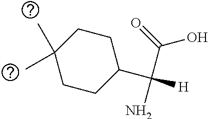

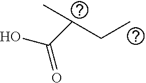

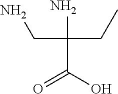

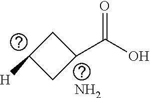

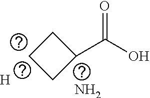

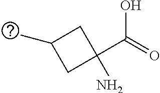

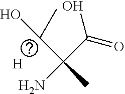

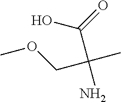

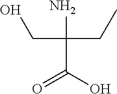

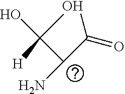

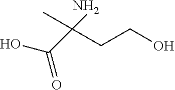

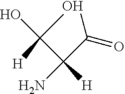

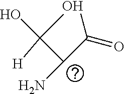

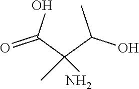

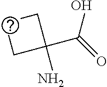

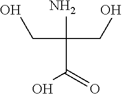

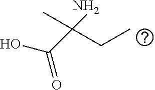

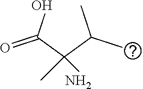

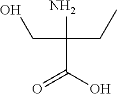

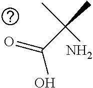

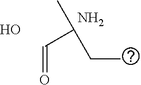

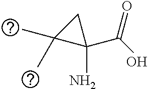

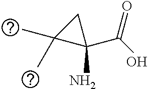

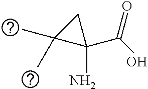

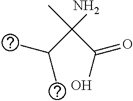

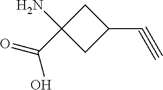

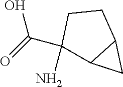

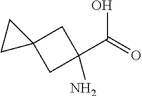

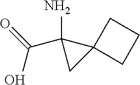

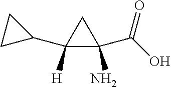

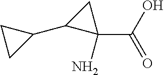

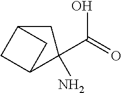

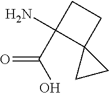

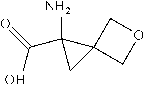

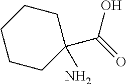

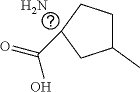

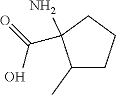

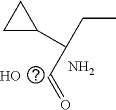

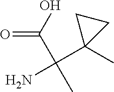

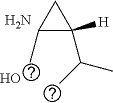

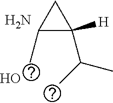

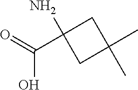

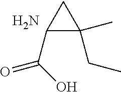

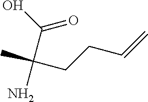

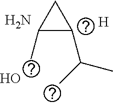

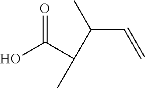

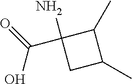

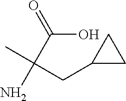

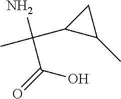

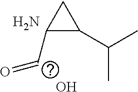

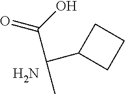

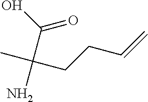

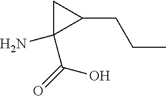

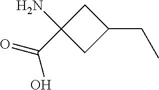

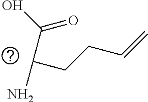

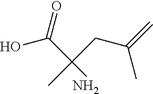

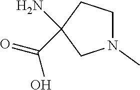

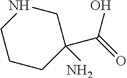

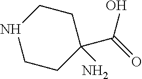

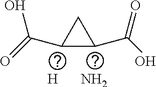

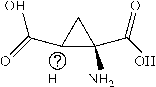

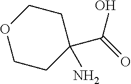

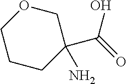

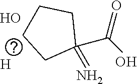

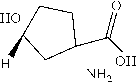

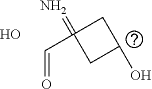

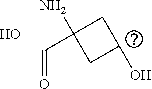

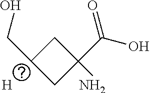

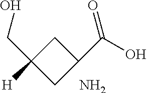

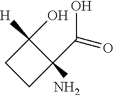

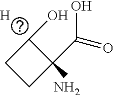

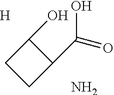

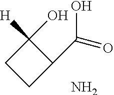

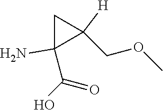

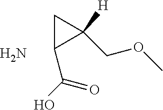

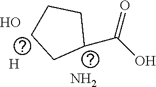

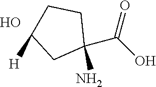

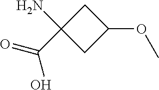

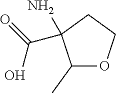

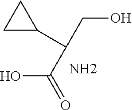

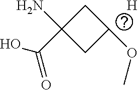

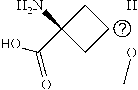

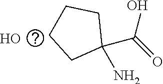

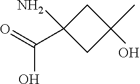

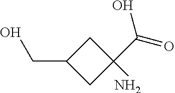

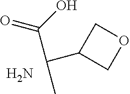

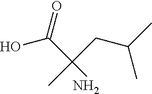

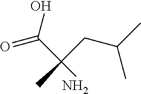

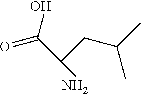

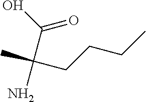

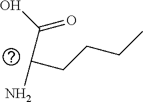

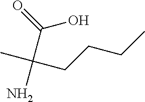

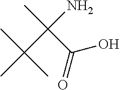

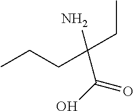

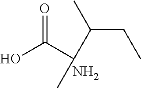

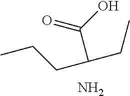

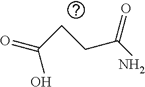

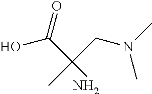

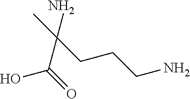

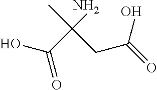

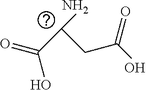

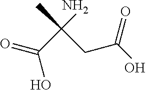

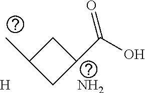

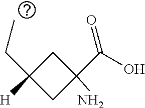

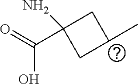

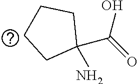

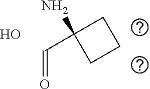

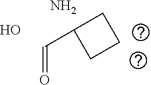

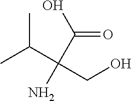

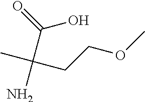

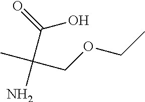

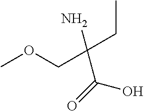

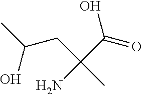

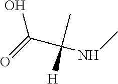

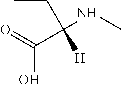

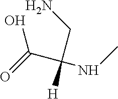

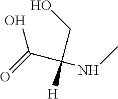

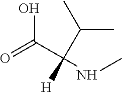

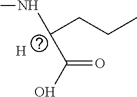

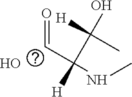

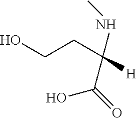

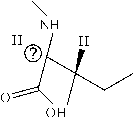

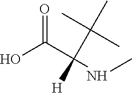

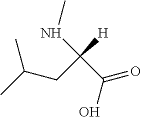

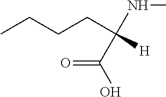

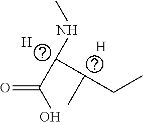

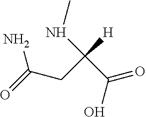

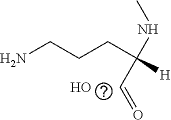

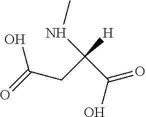

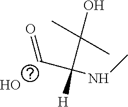

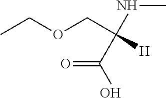

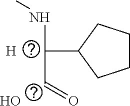

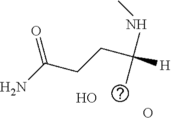

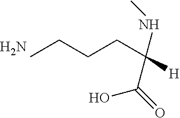

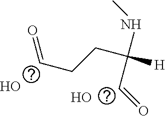

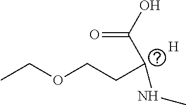

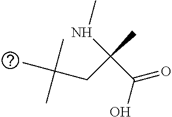

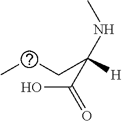

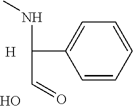

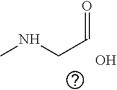

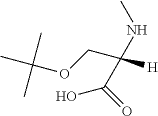

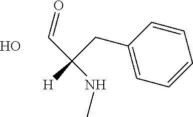

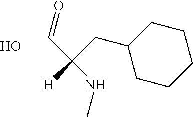

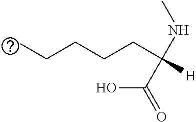

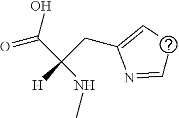

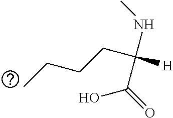

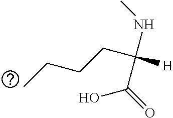

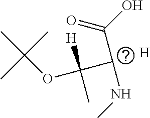

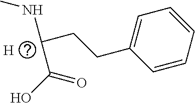

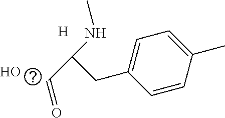

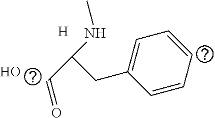

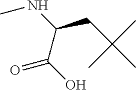

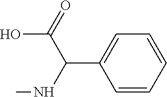

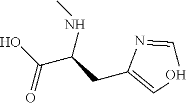

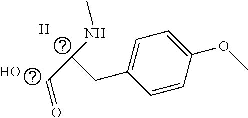

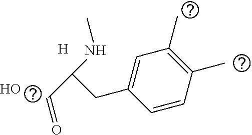

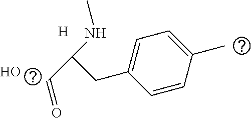

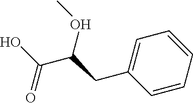

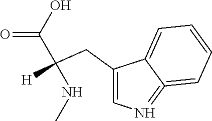

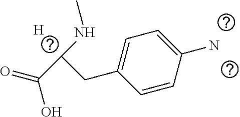

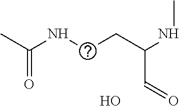

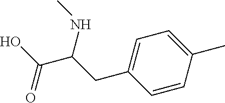

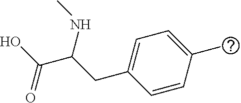

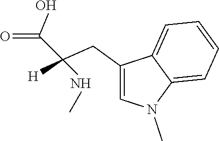 [SMILES: CN[C@@H](Cc1cn(C)c2ccccc12)C(=O)O]

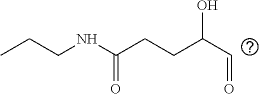

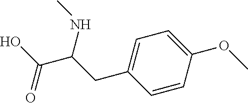

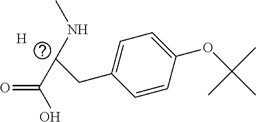

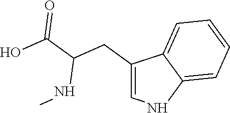 [SMILES: CNC(Cc1c[nH]c2ccccc12)C(=O)O]

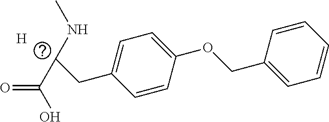

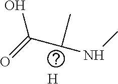

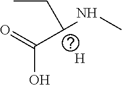

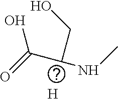

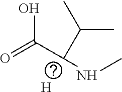

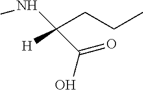

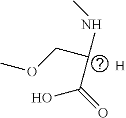

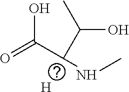

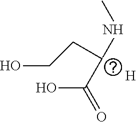

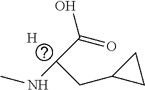

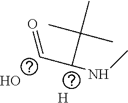

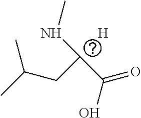

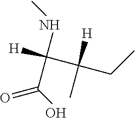

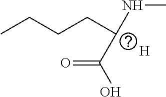

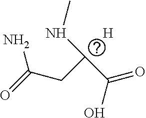

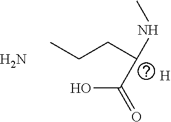

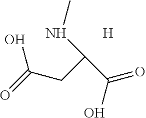

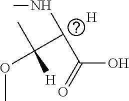

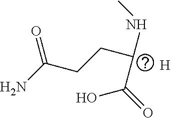

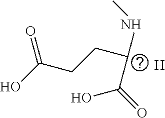

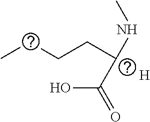

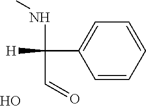

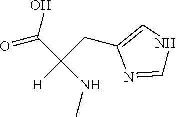

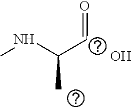

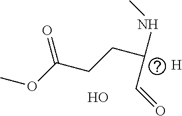

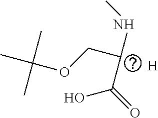

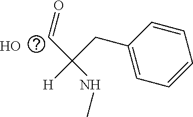

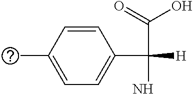

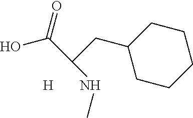

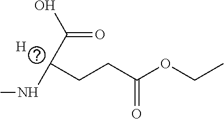

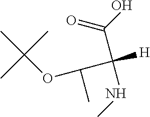

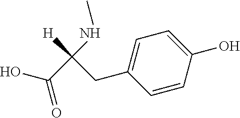

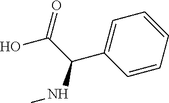

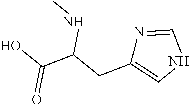

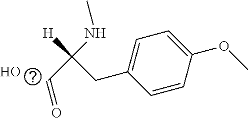

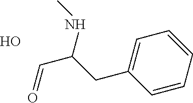

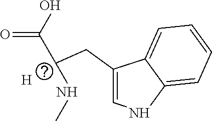

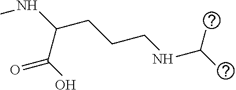

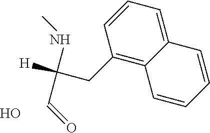

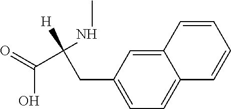 [SMILES: CN[C@H](Cc1ccc2ccccc2c1)C(=O)O]

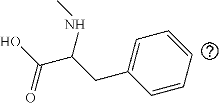

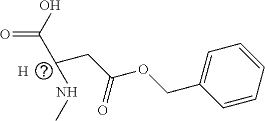

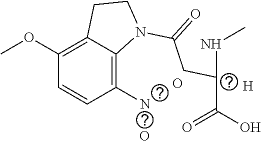

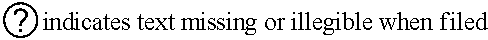

Examples of the amino acids include natural protein L-amino acids, unnatural amino acids, and chemically synthesized compounds having properties known in the art as characteristics of an amino acid. Examples of the unnatural amino acids include, but not limited to, a,a-disubstituted amino acids (such as a-methylalanine), N-alkyl-a-amino acids, D-amino acids, (3-amino acids, and a-hydroxy acids, each having a backbone structure different from that of natural amino acids; amino acids (such as norleucine and homohistidine) having a side-chain structure different from that of natural amino acids; amino acids (such as “homo” amino acids, homophenylalanine, and homohistidine) having extra methylene in the side chain thereof; and amino acids (such as cysteic acid) obtained by substituting a carboxylic acid functional amino group in the side chain thereof by a sulfonic acid group.

The peptides described herein can comprise one or more unnatural amino acids. Unnatural amino acids include, but are not limited to, (1) amino acids corresponding to an amino acid residue on a polypeptide subjected to modification after expression (ex. phosphorylated tyrosine, acetylated lysine, or farnesylated cysteine), (2) amino acids that cannot be used in expression on a ribosome but occur naturally, and (3) artificial amino acids that do not occur naturally (unnatural amino acids). Non-limiting examples of unnatural amino acids include: p-acetyl-L-phenylalanine, p-iodo-L-phenylalanine, p-methoxyphenylalanine, O-methyl-L-tyrosine, p-propargyloxyphenylalanine, p-propargyl-phenylalanine, L-3-(2-naphthyl)alanine, 3-methyl-phenylalanine, O-4-allyl-L-tyrosine, 4-propyl-L-tyrosine, tri-O-acetyl-GlcNAcp-serine, L-Dopa, fluorinated phenylalanine, isopropyl-L-phenylalanine, p-azido-L-phenylalanine, p-acyl-L-phenylalanine, p-benzoyl-L-phenylalanine, Boronophenylalanine, O-propargyltyrosine, L-phosphoserine, phosphonoserine, phosphonotyrosine, p-bromophenylalanine, selenocysteine, p-amino-L-phenylalanine, isopropyl-L-phenylalanine, and azido-lysine (AzK). In some embodiments, the unnatural amino acid is an unnatural analogue of a tyrosine amino acid; an unnatural analogue of a glutamine amino acid; an unnatural analogue of a phenylalanine amino acid; an unnatural analogue of an alanine amino acid; an unnatural analogue of a serine amino acid; an unnatural analogue of a threonine amino acid; an alkyl, aryl, acyl, azido, cyano, halo, hydrazine, hydrazide, hydroxyl, alkenyl, alkynl, ether, thiol, sulfonyl, seleno, ester, thioacid, borate, boronate, phospho, phosphono, phosphine, heterocyclic, enone, imine, aldehyde, hydroxylamine, keto, or amino substituted amino acid; or a combination thereof. In some embodiments, the unnatural amino acid is an amino acid with a photoactivatable cross-linker; a spin-labeled amino acid; a fluorescent amino acid; a metal binding amino acid; a metal-containing amino acid; a radioactive amino acid; a photocaged and/or photoisomerizable amino acid; a biotin or biotin-analogue containing amino acid; a keto containing amino acid; an amino acid comprising polyethylene glycol or polyether; a heavy atom substituted amino acid; a chemically cleavable or photocleavable amino acid; an amino acid with an elongated side chain; an amino acid containing a toxic group; a sugar substituted amino acid; a carbon-linked sugar-containing amino acid; a redox-active amino acid; an a-hydroxy containing acid; an amino thio acid; an α, α-disubstituted amino acid; a β-amino acid; a cyclic amino acid other than proline or histidine, or an aromatic amino acid other than phenylalanine, tyrosine or tryptophan.

In some embodiments, the unnatural amino acids incorporated into the peptides include one or more of: 1) a ketone functional group (as found in para or meta acetyl-phenylalanine) that can be specifically reacted with hydrazines, hydroxylamines and their derivatives (Addition of the keto functional group to the genetic code of Escherichia coli. Wang L, Zhang Z, Brock A, Schultz P G. Proc Natl Acad Sci USA. 2003 Jan. 7; 100(1):56-61; Bioorg Med Chem Lett. 2006 Oct. 15; 16(20):5356-9. Genetic introduction of a diketone-containing amino acid into proteins. Zeng H, Xie J, Schultz P G), 2) azides (as found in p-azido-phenylalanine) that can be reacted with alkynes via copper catalyzed “click chemistry” or strain promoted (3+2) cycloadditions to form the corresponding triazoles (Addition of p-azido-L-phenylalanine to the genetic code of Escherichia coli. Chin J W, Santoro S W, Martin A B, King D S, Wang L, Schultz P G. J Am Chem Soc. 2002 Aug. 7; 124(31):9026-7; Adding amino acids with novel reactivity to the genetic code of Saccharomyces cerevisiae. Deiters A, Cropp T A, Mukherji M, Chin J W, Anderson J C, Schultz P G. J Am Chem Soc. 2003 Oct. 1; 125(39):11782-3), or azides that can be reacted with aryl phosphines, via a Staudinger ligation (Selective Staudinger modification of proteins containing p-azidophenylalanine. Tsao M L, Tian F, Schultz P G. Chembiochem. 2005 December; 6(12):2147-9), to form the corresponding amides, 3) alkynes that can be reacted with azides to form the corresponding triazole (In vivo incorporation of an alkyne into proteins in Escherichia coli. Deiters A, Schultz P G. Bioorg Med Chem Lett. 2005 Mar. 1; 15(5):1521-4), 4) boronic acids (boronates) than can be specifically reacted with compounds containing more than one appropriately spaced hydroxyl group or undergo palladium mediated coupling with halogenated compounds (Angew Chem Int Ed Engl. 2008; 47(43):8220-3. A genetically encoded boronate-containing amino acid., Brustad E, Bushey M L, Lee J W, Groff D, Liu W, Schultz P G), and 5) metal chelating amino acids, including those bearing bipyridyls, that can specifically co-ordinate a metal ion (Angew Chem Int Ed Engl. 2007; 46(48):9239-42. A genetically encoded bidentate, metal-binding amino acid. Xie J, Liu W, Schultz P G).

The peptide of the present disclosure embraces various derivatives thereof. Examples of the derivatives include derivatives having an amide, ester, or carboxyl group as the C-terminus and/or N-terminus thereof. Additional examples of the derivatives of the peptide include those obtained by modification such as phosphorylation, methylation, acetylation, adenylylation, ADP-ribosylation, or glycosylation and fused protein obtained by fusion with another peptide or protein. These derivatives can be prepared by those skilled in the art in a known manner or a method based thereon.

In some embodiments, the peptide described herein comprises a basic amino acid. Examples of the basic amino acid include arginine, lysine, citrulline, ornithine, creatine, histidine, diaminobutanoic acid, and diaminopropionic acid.

In some embodiments, provided herein is a peptide having 90% or more sequence identity to any of sequences disclosed herein. In some embodiments, the sequence identity is at least 95% or 99%.

Percent sequence identity can be calculated using computer programs or direct sequence comparison. Preferred computer program methods to determine identity between two sequences include, but are not limited to, the GCG program package, FASTA, BLASTP, and TBLASTN (see, e.g., D. W. Mount, 2001, Bioinformatics: Sequence and Genome Analysis, Cold Spring Harbor Laboratory Press, Cold Spring Harbor, N.Y.). The BLASTP and TBLASTN programs are publicly available from NCBI and other sources. The Smith Waterman algorithm can also be used to determine percent identity. Exemplary parameters for amino acid sequence comparison include the following: 1) algorithm from Needleman and Wunsch (J. Mol. Biol., 48:443-453 (1970)); 2) BLOSSUM62 comparison matrix from Hentikoff and Hentikoff (Proc. Nat. Acad. Sci. USA., 89:10915-10919 (1992)) 3) gap penalty=12; and 4) gap length penalty=4. A program useful with these parameters can be publicly available as the “gap” program (Genetics Computer Group, Madison, Wis.). The aforementioned parameters are the default parameters for polypeptide comparisons (with no penalty for end gaps). Alternatively, polypeptide sequence identity can be calculated using the following equation: % identity−(the number of identical residues)/(alignment length in amino acid residues)*100. For this calculation, alignment length includes internal gaps but does not include terminal gaps.

In some embodiments, the peptide is bicyclic or polycyclic. In some embodiments, a conjugate described herein comprises a bicyclic peptide. Exemplary bicyclic peptides include the bicyclic targeting peptides of BT5528, BT1718, and BT8009. Exemplary bicyclic peptides are described in US20180200378, U.S. Pat. No. 10,441,663, U.S. Pat. No. 8,680,022B2, US20180280525, and US20200215199, each of which is hereby incorporated by reference in its entirety.

In some embodiments, the peptide is a lasso peptide. Lasso peptides can be synthetic or naturally produced by bacteria, and they possess a distinctive threaded lariat fold that offers a 3D array of functionality for engaging biological targets. This lasso structure can enable beneficial properties such as affinity, stability and potent biological activities. Suitable lasso structure can be designed by algorithms. Exemplary lasso peptides are provided in Hegemann, J. D., et al., Lasso Peptides: An Intriguing Class of Bacterial Natural Products, Acc. Chem. Res., 2015, 48, 1909-1919; Tietz, J. I., et al., A new genome-mining tool redefines the lasso peptide biosynthetic landscape, Nature Chem Bio, 2017, 13, 470-478; DiCaprio, A. J., et al., Enzymatic Reconstitution and Biosynthetic Investigation of the Lasso Peptide Fusilassin, J. Am. Chem. Soc., 2019, 141, 290-297; Al Toma, R. S., et al., Site-Directed and Global Incorporation of Orthogonal and Isostructural Noncanonical Amino Acids into the Ribosomal Lasso Peptide Capistruin, ChemBioChem, 2015, 16, 503-509.

Further exemplary peptides include BMS-753493, Somatostatins, Octreotide, Octreotate, Lanreotide, Pasireotide, JR-11, L-779,976, BIM-23120, Satoreotide, depreotide, 18F-KYNDRLPLYISNP (SEQ ID NO: 10), CaIX-P1, and FAP-2286.

The peptide of the present disclosure embraces salts thereof. As the salts of the peptide, salts with physiologically acceptable base or acid are used. Examples include addition salts with an inorganic acid (such as hydrochloric acid, hydrobromic acid, hydroiodic acid, sulfuric acid, or phosphoric acid), addition salts with an organic acid (such as p-toluenesulfonic acid, methanesulfonic acid, oxalic acid, p-bromophenylsulfonic acid, carboxylic acid, succinic acid, citric acid, benzoic acid, or acetic acid), inorganic bases (such as ammonium hydroxide, alkali or alkaline earth metal hydroxide, carbonate, or bicarbonate), and an amino acid.

The peptide of the present disclosure can be prepared by a known peptide preparation method, for example, chemical synthesis method such as liquid-phase method, solid-phase method, or hybrid method using a liquid-phase method and a solid-phase method in combination; or gene recombination method.

In solid-phase method, an esterification reaction can be performed, for example, between the hydroxyl group of a hydroxyl-containing resin and the carboxyl group of a first amino acid (usually, C-terminal amino acid of an intended peptide) having an a-amino group protected with a protecting group. As the esterifying catalyst, a dehydration condensation agent such as 1-mesitylenesulfonyl-3-nitro-1,2,4-triazole (MSNT), dicyclohexylcarbodiimide (DCC), and diisopropylcarbodiimide (DIPCDI) may be used. Next, the protecting group of the a-amino group of the first amino acid is eliminated and at the same time, a second amino acid having all the functional groups protected except the main chain carboxyl group is added to activate the carboxyl group and bind the first and second amino acids to each other. Then, the a-amino group of the second amino acid is deprotected, a third amino acid having all the functional groups protected except the main chain carboxyl group is added, and the carboxyl group is activated to bind the second and third amino acids to each other. The above-described reactions are repeated to synthesize a peptide having an intended length. Then, all the functional groups are deprotected. Examples of the resin for solid-phase synthesis include Merrifield resin, MBHA resin, CI-Trt resin, SASRIN resin, Wang resin, Rink amide resin, HMFS resin, Amino-PEGA resin (Merck), and HMPA-PEGA resin (Merck). These resins may be provided for use after washed with a solvent (dimethylformamide (DMF), 2-propanol, methylene chloride, or the like). A peptide chain can be cleaved from the resin by treating it with an acid such as TFA or hydrogen fluoride (HF).

Examples of the protecting group of the a-amino group include a benzyloxycarbonyl (Cbz or Z) group, a tert-butoxycarbonyl (Boc) group, a fluorenylmethoxycarbonyl (Fmoc) group, a benzyl group, an allyl group, and an allyloxycarbonyl (Alloc) group. The Cbz group can be deprotected using hydrofluoric acid, hydrogenation, or the like; the Boc group can be deprotected using trifluoroacetic acid (TFA); and the Fmoc group can be deprotected by the treatment with piperidine. For protection of the a-carboxyl group, a methyl ester, an ethyl ester, a benzyl ester, a tert-butyl ester, a cyclohexyl ester, or the like can be used. As other functional groups of an amino acid, the hydroxyl group of serine or threonine can be protected with a benzyl group or a tert-butyl group and the hydroxyl group of tyrosine can be protected with a 2-bromobenzyloxycarbonyl group or a tert-butyl group. The amino group of a lysine side chain or the carboxyl group of glutamic acid or aspartic acid can be protected in a manner similar to the a-amino group or a-carboxyl group.

The carboxyl group can be activated with a condensation agent. Examples of the condensation agent include di cyclohexylcarbodiimide (DCC), diisopropylcarbodiimide (DIPCDI), 1-ethyl-3-(3-dimethylaminopropyl)carbodiimide (EDC or WSC), (1H-benzotriazol-1-yloxy)tris(dimethylamino)phosphonium hexafluorophosphate (BOP), and 1-[bis(dimethylamino)methyl]-1H-benzotriazolium-3-oxide hexafluorophosphate (HBTU).

Peptide preparation based on the recombinant method (translation and synthesis system) can be performed using a nucleic acid encoding the peptide of the present disclosure. The nucleic acid encoding the peptide can be either DNA or RNA. The nucleic acid encoding the peptide can be prepared in a known method. For example, it can be synthesized using an automated synthesizer. The DNA thus obtained may have therein a restriction enzyme recognition site for inserting it into a vector or may have therein a base sequence that encodes an amino acid sequence for cleavage of the resulting peptide chain by an enzyme. The peptide obtained may be converted from a free peptide to a salt thereof or from a salt thereof to a free peptide by a known method or a method based thereon.

In order to suppress decomposition by a host-derived protease, a chimera protein expression method that expresses the intended peptide as a chimera peptide with another peptide can be used. In this case, as the nucleic acid, a nucleic acid encoding the intended peptide and a peptide that binds thereto is used. Then, an expression vector is prepared using the nucleic acid encoding the peptide of the present invention. The nucleic acid can be inserted into downstream of a promoter of an expression vector as it is, or after digestion with a restriction enzyme or addition of a linker. Examples of the vector include Escherichia coli-derived plasm ids (such as pBR322, pBR325, pUC12, pUC13, pUC18, pUC19, pUC118, and pBluescript II), Bacillus subtilis-derived plasmids (such as pUB110, pTP5, pC1912, pTP4, pE194, and pC194), yeast-derived plasmids (such as pSH19, pSH15, YEp, YRp, Ylp, and YAC), bacteriophages (such as e phage and M13 phage), viruses (retrovirus, vaccinia virus, adenovirus, adeno-associated virus (AAV), cauliflower mosaic virus, tobacco mosaic virus, and baculovirus), and cosmids. The promoter can be selected as needed, depending on the type of the host. When the host is an animal cell, for example, a SV40 (simian virus 40)-derived promoter or a CMV (cytomegalovirus)-derived promoter can be used. When the host is Escherichia coli, a trp promoter, a T7 promoter, a lac promoter, or the like can be used. The expression vector may incorporate therein a nucleic acid encoding a DNA replication origin (ori), a selection marker (antibiotic resistance, nutrition requirement, or the like), an enhancer, a splicing signal, a polyadenylation signal, a tag (FLAG, HA, GST, GFP, or the like), or the like.

Next, an appropriate host cell is then transformed using the above-described vector. The host can be selected as needed based on the relation with a vector and for example, Escherichia coli, Bacillus subtilis, Bacillus bacteria), yeasts, insects or inset cells, and animal cells can be used. Examples of the animal cells include HEK293T cells, CHO cells, COS cells, myeloma cells, HeLa cells, and Vero cells. Transformation can be performed in a known manner such as lipofection, calcium phosphate method, electroporation, microinjection, or particle gun technology, depending on the type of hosts. By culturing the transformant in a conventional manner, an intended peptide is expressed. The peptide from the cultured product of the transformant can be purified in the following manner. Cultured cells collected and then suspended in an appropriate buffer are destructed by ultrasonic treatment, freezing and thawing method, or the like and the resulting destructed product centrifuged or filtered to obtain a crude extract. When the peptide is secreted in the culture fluid, a supernatant is collected. Purification of the crude extract or culture supernatant can also be performed by a known method or a method based thereon (for example, salting-out, dialysis, ultrafiltration, gel filtration, SDS-PAGE, ion exchange chromatography, affinity chromatography, or reverse-phase high-performance liquid chromatography).

The system for translation and synthesis may be a cell-free translation system. The cell-free translation system may include, for example, a ribosome protein, aminoacyl tRNA synthetase (ARS), ribosome RNA, an amino acid, rRNA, GTP, ATP, a translation initiation factor (IF), an elongation factor (EF), a release factor (RF), a ribosome regeneration factor (RRF), and other factors necessary for translation. An Escherichia coli extract or wheat bran extract may be added in order to increase the expression efficiency. Further, a rabbit erythrocyte extract or insect cell extract may be added. Continuous energy supply to a system containing the above by dialysis can enable production of several hundred μg to several mg/mL of a protein. The system may contain RNA polymerase for carrying out transcription from DNA at the same time. As a commercially available cell-free translation system, an Escherichia-coli derived system such as “RTS-100™” of Roche Diagnostics Corporation or PURESYSTEM™ of PGI Corporation or a system using wheat germ extract such as that of ZOEGENE Corporation or Cell-free Science may be used. By using the cell-free translation system, a high-purity peptide can be obtained without purifying the expression product.

In the cell-free translation system, an artificial aminoacyl tRNA obtained by linking (acylating) a desired amino acid or hydroxy acid to tRNA can be used instead of an aminoacyl tRNA synthesized by a native aminoacyl tRNA synthetase. Such an aminoacyl tRNA can be synthesized using an artificial ribozyme. Examples of such a ribozyme include flexizymes (H. Murakami, A. Ohta, H. Ashigai, H. Suga (2006) Nature Methods 3, 357-359 “The flexizyme system: a highly flexible tRNA aminoacylation tool for the synthesis of nonnatural peptides”; WO2007/066627; and the like). Flexizyme is also known as, as well as flexizyme (Fx) in original form, dinitrobenzyl flexizyme (dFx), enhanced flexizyme (eFx), or aminoflexizyme (aFx), each obtained by modifying the original one. By using a tRNA having a desired amino acid or hydroxy acid linked thereto and prepared using flexizyme, a desired codon can be translated while associating the codon with the desired amino acid or hydroxy acid. As the desired amino acid, a non-canonical amino acid may be used. For example, a non-natural amino acid necessary for the above-described cyclization can be introduced into the peptide by this method. For example, an in vitro system can be employed wherein nonsense mutations are suppressed using chemically aminoacylated suppressor tRNAs. Transcription and translation of plasmids containing nonsense mutations can be carried out in a cell-free system comprising e.g., an E. coli S30 extract and commercially available enzymes and other reagents. Peptides can be purified by chromatography. As another example, translation can be carried out in Xenopus oocytes by microinjection of mutated mRNA and chemically aminoacylated suppressor tRNAs. For yet another example, E coli cells can be cultured in the absence of a natural amino acid that is to be replaced (e.g., phenylalanin.e) and in the presence of the desired non-naturally occurring amino acid(s) (e.g., 2-azaphenylalanine, 3-azaphenylalanine, azaphenylalanine, or 4-fluorophenylalanine). The non-naturally occurring amino add can be incorporated into the peptide in place of its natural counterpart. Naturally occurring amino acid residues can also be converted to non-naturally occurring species by in vitro chemical modification. Chemical modification can be combined with site-directed mutagenesis to further expand the range of substitutions.

Peptides described herein can also be produced by chemical synthesis (e.g., by the methods described in Solid Phase Peptide Synthesis, 2nd Edition, The Pierce Chemical Co., Rockford, Ill. (1984)). Modifications to the protein can also be produced by chemical synthesis.

### Target

In some embodiments, a peptide described herein or a conjugate comprising the peptide binds a structure on a cell, e.g., a cell-surface protein. The structure can be a protein or peptide or glycan that is overexpressed or selectively expressed on a diseased cell such as a cancer cell. In some embodiments, the structure can be an isoform or splice variant of the protein or peptide. In some embodiments, the structure can be a protein or peptide that is modified post-translationally. In some embodiments, the structure can be a protein or peptide from a mutated sequence of the gene from which it derives. In some embodiments, the structure can be a fragment or cleaved form of the protein or peptide. The structure can be a glycan that is expressed on a diseased cell such as a cancer cell. The glycan can be an O-glycan, an N-glycan, GSL-glycan, glycolipid, or the like. In some embodiments, the peptide binds to a glycan that comprises a cancer glycan epitope. In some embodiments, the peptide binds to a cancer cell. In some embodiments the peptide binds to a non-cancer cell in the tumor environment. In some embodiments, the peptide binds an oncofetal antigen, a tight junction protein, an adhesion protein, an eph-related tyrosine kinase receptor (TKR), and/or a transporter protein. In some embodiments, the cell-surface protein is an oncofetal antigen. In some embodiments, the oncofetal antigen is a carcinoembryonic antigen (CEA) or an alpha-fetoprotein (AFP). In some embodiments, the oncofetal antigen is carcinoembryonic antigen-related cell adhesion molecule 5 (CEACAM5). In some embodiments, the oncofetal antigen is tumor-associated calcium signal transducer 2 (Trop-2). In some embodiments, the cell-surface protein is an eph-related tight junction protein. In some embodiments, the tight junction protein is a claudin (such as claudin 2, 3, 4, 7, 8, 12, 15, or 18), an occludin, an oligodendrocyte-specific protein (OSP), a peripheral myelin protein (PMP), or a junctional adhesion molecule. In some embodiments, the cell-surface protein is an adhesion protein. In some embodiments, the adhesion protein is nectin cell adhesion molecule 4 (Nectin-4), carcinoembryonic antigen-related cell adhesion molecule 5 (CEACAM5), mesothelin, or an ephrin receptor. In some embodiments, the adhesion protein is selected from integrins, cadherins, selectins, and immunoglobulin-like Cell Adhesion Molecules. In some embodiments, the cell-surface protein is a tyrosine kinase receptor (TKR). In some embodiments, the tyrosine kinase receptor is an eph-related tyrosine kinase receptor. In some embodiments, the eph-related tyrosine kinase receptor is an ephrinA or an ephrinB receptor. In some embodiments, the Eph receptor is EPH Receptor A2 (EPHA2). In some embodiments, the tyrosine kinase receptor is an epidermal growth factor receptor (EGFR), a fibroblast growth factor receptor (FGFR), a vascular endothelial growth factor receptor (VEGFR), an ErbB receptor, a RET receptor, or a discoidin domain receptor (DDR). In some embodiments, the ErbB receptor is a Her2 receptor. In some embodiments, the cell-surface protein is a transport receptor. In some embodiments, the transport receptor is Folate receptor 1 (FOLR1) or NaPi2b.

In some embodiments, a peptide described herein or a conjugate comprising the peptide binds a transmembrane glycoprotein. In some embodiments, the transmembrane glycoprotein is prostate-specific membrane antigen (PSMA), tumor-associated calcium signal transducer 2 (Trop-2), somatostatin receptor type 2 (SSTR2), or carbonic anhydrase IX (CAIX). In some embodiments, the peptide binds to prostate-specific membrane antigen (PSMA). In some embodiments, the peptide binds to tumor-associated calcium signal transducer 2 (Trop-2). In some embodiments, the peptide binds to folate receptor 1 (FOLR1). In some embodiments, the peptide binds to nectin cell adhesion molecule 4 (Nectin-4). In some embodiments, the peptide binds to carcinoembryonic antigen-related cell adhesion molecule 5 (CEACAM5). In some embodiments, the peptide binds to EPH Receptor A2 (EPHA2). In some embodiments, the peptide binds to mesothelin, EPH Receptor A2 (EPHA2), tumor-associated calcium signal transducer 2 (Trop-2), somatostatin receptor type 2 (SSTR2), carbonic anhydrase IX (CAIX), delta-like 3 (DLL3), fibroblast activation protein alpha (FAP-alpha), B7-H₄, tumor Endothelial Marker 8 (TEM8), NaPi2b, claudin 18.2, tumor-associated glycoprotein 72 (TAG72), or CD70.

A peptide described herein or a conjugate comprising the peptide can bind to an oncofetal antigen. In some embodiments, the peptide binds to an oncofetal antigen encoded by a gene selected from: CEACAM1 (CEA), CEACAM5, CEACAM6, 5T4 (TPBG), ALPPL2, DLK1, PLAC1, SPA17, and AKAP4. In some embodiments, the peptide binds to an oncofetal antigen that is Carcinoembryonic antigen-related cell adhesion molecule 1, Carcinoembryonic antigen-related cell adhesion molecule 5, Carcinoembryonic antigen-related cell adhesion molecule 6, Trophoblast glycoprotein, Alkaline phosphatase (germ cell type), Protein delta homolog 1, Placenta-specific protein 1, Sperm surface protein Sp17, or A-kinase anchor protein 4.

A peptide described herein or a conjugate comprising the peptide can bind to a tumor surface marker. In some embodiments, the peptide binds to a tumor surface marker encoded by a gene selected from: ANKRD30A (NY-BR-1), C4.4A, CSPG4, CTA 16.88, GD2, GPC3, GPNMB, KAAG1, LAMP-1, PSCA, PSMA, TAG-72, TROP2, and CD248 (TEM1). In some embodiments, the peptide binds to a tumor surface marker selected from: Ankyrin repeat domain-containing protein 30A, homologue of the urokinase receptor, Chondroitin sulfate proteoglycan 4, tumor-associated antigen 16.88, ganglioside GD2, Glypican-3, Transmembrane glycoprotein NMB, Kidney-associated antigen 1, Lysosome-associated membrane glycoprotein 1, Prostate stem cell antigen, Glutamate carboxypeptidase 2, tumor-associated glycoprotein 72, Tumor-associated calcium signal transducer 2, and Endosialin.

A peptide described herein or a conjugate comprising the peptide can bind to a receptor or ligand. In some embodiments, the peptide binds to a receptor or ligand encoded by a gene selected from: AMHR2, AXL, c-MET, CD123 (IL3Ra), CD142 (TF), KIT, MET, TDGF1 (CRIPTO), CS1 (SLAMF7), DLL3, DLL4, DRS (TRAILR2), EDNRB, EFNA4, EGFR, EGFRv111, EPHA2, HER2 (ERBB2), ETBR, FGFR2, FGFR2b, FGFR3, FLT3, FOLR1, GCC (GUCY2C), GFRA1, GRPR, HER3 (ERBB3), IGF1R, IL13RA2, IL1RAP, IL3RA, KDR (VEGFR2), LIF, NOTCH3, NTSR1, PRLR, PTK7, ROR1, ROR2, SORT1, TENB2 (TMEFF2), TFR1, and TMEFF1. In some embodiments, the peptide binds to a receptor or ligand selected from: Anti-Muellerian hormone type-2 receptor, Tyrosine-protein kinase receptor UFO, Hepatocyte growth factor receptor, Interleukin-3 receptor subunit alpha, Tissue Factor, Mast/stem cell growth factor receptor, Hepatocyte growth factor receptor, Teratocarcinoma-derived growth factor 1, SLAM family member 7, Delta-like protein 3, Delta-like protein 4, TNF-related apoptosis-inducing ligand receptor 2, Endothelin receptor type B, Ephrin-A4, Epithelial growth factor receptor, Epithelial growth factor receptor variant III, Ephrin type-A receptor 2, Receptor tyrosine-protein kinase erbB-2, Endothelin B receptor, Fibroblast growth factor receptor 2, Fibroblast growth factor receptor 2, subtype b, Fibroblast growth factor receptor 3, Receptor-type tyrosine-protein kinase FLT3, Folate receptor alpha, Guanylyl cyclase C, GDNF family receptor alpha-1, Gastrin-releasing peptide receptor, GRP-R, Receptor tyrosine-protein kinase erbB-3, Insulin-like growth factor 1 receptor, Interleukin-13 receptor subunit alpha-2, Interleukin-1 receptor accessory protein, Interleukin-3 receptor subunit alpha, Vascular endothelial growth factor receptor 2, Leukemia inhibitory factor, Neurogenic locus notch homolog protein 3, Neurotensin receptor type 1, Prolactin receptor, Inactive tyrosine-protein kinase 7, Inactive tyrosine-protein kinase transmembrane receptor ROR1, Tyrosine-protein kinase transmembrane receptor ROR2, Sortilin, Tomoregulin-2, Transferrin receptor protein 1, and Tomoregulin-1.

A peptide described herein or a conjugate comprising the peptide can bind to a cell adhesion and matrix protein. In some embodiments, the peptide binds to a cell adhesion and matrix protein encoded by a gene selected from: ADAM9, ALCAM, CDH₆, CD105 (ENG), CD138 (SDC1), CD44, CD44v6, CD52, CD56 (NCAM-1), Claudin 18.2, EPCAM (TROP1), ITGAV, see below , L1CAM, LRRC15, LYPD3, MSLN (mesothelin), MUC1, MUC16, MUC5AC, NCAM, Nectin-4, NRXN1, CDH₃ (P-cadherin), TEM8, and TNC. In some embodiments, the peptide binds to a cell adhesion and matrix protein selected from: Disintegrin and metalloproteinase domain-containing protein 9, Activated leukocyte cell adhesion molecule, Cadherin-6, Endoglin, Syndecan-1, CD44 antigen, CD44 antigen variant 6, CAMPATH-1 antigen , Neural cell adhesion molecule 1, Claudin-18 splice 2, Epithelial cell adhesion molecule, Integrin alpha-V, Integrins, Neural cell adhesion molecule L1, Leucine-rich repeat-containing protein 15, Ly6/PLAUR domain-containing protein 3 , Mesothelin, Mucin-1, Mucin-16, Mucin-5AC, Neural cell adhesion molecule, Nectin-4, Neurexin-1, Cadherin-3, Anthrax toxin receptor 1, and Tenascin.

A peptide described herein or a conjugate comprising the peptide can bind to an immunomodulation protein. In some embodiments, the peptide binds to an immunomodulation protein encoded by a gene selected from: B7H4 (VTCN), BCMA (TNFRSF17), CD19, CD205 (LY75), CD206 (MRC1), CD22 (Siglec2), CD25 (IL2RA), CD274 (PD-L1), CD27L, CD3, CD30 (TNFRSF8), CD32a, CD33 (SIGLEC3), CD37 (TSPAN26), CD46 (MCP), CD70, CD74, CD79b, FCRL5, ICOS, LILRB4, LY6E, CD20 (MS4A1), NKG2D, TIM-1 (HAVCR1) , CD137 (4-1BB, TNFRSF9), ULBP1, ULBP2, and CD276 (B7-H3). In some embodiments, the peptide binds to an immunomodulation protein selected from: V-set domain-containing T-cell activation inhibitor 1, Tumor necrosis factor receptor superfamily member 17, B-lymphocyte antigen CD19, Lymphocyte antigen 75, Macrophage mannose receptor 1, B-cell receptor CD22, Interleukin-2 receptor subunit alpha, Programmed cell death 1 ligand 1, Tumor necrosis factor receptor superfamily member 7, T-cell surface glycoprotein CD3 , Tumor necrosis factor receptor superfamily member 8, Low affinity immunoglobulin gamma Fc region receptor Iia, Myeloid cell surface antigen CD33, Leukocyte antigen CD37, Membrane cofactor protein, CD70 antigen, HLA class II histocompatibility antigen gamma chain, B-cell antigen receptor complex-associated protein beta chain, Fc receptor-like protein 5, Inducible T-cell costimulator, Leukocyte immunoglobulin-like receptor subfamily B member 4, Lymphocyte antigen 6E, B-lymphocyte antigen CD20 , NKG2-D type II integral membrane protein, T-cell immunoglobulin and mucin domain 1 , Tumor necrosis factor receptor superfamily member 9, UL16-binding protein 1, UL16-binding protein 2, and CD276 antigen.

A peptide described herein or a conjugate comprising the peptide can bind to a transporter protein. In some embodiments, the peptide binds to a transporter protein encoded by a gene selected from: ASCT2 (SLC1A5), LIV1 (SLC39A6), NaPi2b, and SLC44A4. In some embodiments, the peptide binds to a transporter protein selected from: Neutral amino acid transporter B, Zinc transporter ZIP6, Sodium-dependent phosphate transport protein 2B, and Choline transporter-like protein 4.

A peptide described herein or a conjugate comprising the peptide can bind to a membrane enzyme. In some embodiments, the peptide binds to a membrane enzyme encoded by a gene selected from: ASPH, CA6, CA9 (CAIX) , CD174 (FUT1), CD38, CDS, ENPP3, FAP, and MMP14 (MT1-MMP). In some embodiments, the peptide binds to a membrane enzyme selected from: Aspartyl/asparaginyl beta-hydroxylase, Carbonic anhydrase 6, Carbonic anhydrase 9, Galactoside alpha-(1,2)-fucosyltransferase 1, ADP-ribosyl cyclase/cyclic ADP-ribose hydrolase 1, Phosphatidate cytidylyltransferase 1, Ectonucleotide pyrophosphatase/phosphodiesterase family member 3, Prolyl endopeptidase FAP, and Matrix metalloproteinase-14.

A peptide described herein or a conjugate comprising the peptide can bind to a multispan protein. In some embodiments, the peptide binds to a multispan protein encoded by a gene selected from: SSTR2, STEAP1, SEZ6, and SLITRK6. In some embodiments, the peptide binds to a multispan protein selected from: Somatostatin receptor type 2, Metalloreductase STEAP1, Seizure protein 6 homolog, and SLIT and NTRK-like protein 6.

### Linker

A conjugate described herein can comprise one or more linkers. In some embodiments, the linker covalently attaches the peptide with the metal chelator. In some embodiments, the peptide attaches directly to the metal chelator without a linker.

A linker can comprise one or more amino acid residues. In some embodiments, the linker comprises 1 to 3, 1 to 5, 1 to 10, 5 to 10, or 5 to 20 amino acid residues. In some embodiments, the linker comprises 1, 2, 3, 4, 5, 6, 7, 8, 9, or 10 amino acid residues. In some embodiments, the linker comprises 1 to 5 amino acid residues. For example, the linker can comprise one or more lysine (K) residues such as K, KK, or KKK sequences. In some embodiments, the linker comprises a lysine or a derivative thereof. In some embodiments, the linker comprises a lysine. In some embodiments, one or more amino acids of the linker are unnatural amino acids.

A herein-described linker can attach to the N-terminus of the peptide, the C-terminus of the peptide, or a non-terminal amino acid of the peptide, or it can attach to the peptide through a combination of the above. In some embodiments, the linker is attached to the peptide via its N-terminus. In some embodiments, the linker is attached to the peptide via its C-terminus. In some embodiments, the linker is attached to the peptide via a non-terminal amino acid. The linker can be bonded to the peptide, the metal chelator, or both, for example, through a chemically reactive group. Exemplary chemically reactive groups include, but are not limited to, a free amino, imino, hydroxyl, thiol or carboxyl group (e.g., to the N- or C-terminus, to the epsilon amino group of one or more lysine residues, the free carboxylic acid group of one or more glutamic acid or aspartic acid residues, or to the sulfhydryl group of one or more cysteinyl residues). The site to which the linker is bound to the peptide can be a natural or unnatural amino acid of the peptide and/or it can be introduced into the peptide, e.g., by DNA recombinant technology (e.g., by introducing a cysteine or protease cleavage site in the amino acid sequence) or by protein biochemistry (e.g., reduction, pH adjustment or proteolysis). Exemplary methods for attaching the linker includes carbodiimide reaction, reactions using bifunctional agents such as dialdehydes or imidoesters, Schiff base reaction, Suzuki-Miyaura cross-coupling reactions, Isothiocyanates as coupling agents, and click chemistry.

The linker can have a prescribed length thereby linking the metal chelator (and optionally radionuclide) and the peptide while allowing an appropriate distance therebetween. In some embodiments, the linker has 1 to 100 atoms, 1 to 60 atoms, 1 to 30 atoms, 1 to 15 atoms, 1 to 10 atoms, 1 to 5, or 2 to 20 atoms in length. In some embodiments, the linker has 1 to 10 atoms in length.

The linker can comprise flexible and/or rigid regions. Exemplary flexible linker regions include those comprising Gly and Ser residues (“GS” linker), glycine residues, alkylene chain, PEG chain, etc. Exemplary rigid linker regions include those comprising alpha helix-forming sequences (e.g., EAAAK (SEQ ID NO: 11)), proline-rich sequences, and regions rich in double and/or triple bonds.

The linker can be cleavable, e.g., under physiological conditions, e.g., under intracellular conditions, such that cleavage of the linker releases the chelator and radionuclide in the intracellular environment. The linker can be, e.g., a peptidyl linker that is cleaved by an intracellular peptidase or protease enzyme, including, but not limited to, a lysosomal or endosomal protease. In some embodiments, the peptidyl linker is at least two amino acids long or at least three amino acids long. Cleaving agents can include cathepsins B and D and plasmin. In other embodiments, the linker is not cleavable. In some embodiments, the linker is pH-sensitive, i.e., sensitive to hydrolysis at certain pH values. For example, the pH-sensitive linker can be hydrolyzable under acidic conditions. For example, a linker can be an acid-labile linker that is hydrolyzable in the lysosome (e.g., a hydrazone, semicarbazone, thiosemicarbazone, cis-aconitic amide, orthoester, acetal, ketal, or the like). Such linkers can be relatively stable under neutral pH conditions, such as those in the blood, but are unstable at below pH 5.5 or 5.0, the approximate pH of the lysosome. In some embodiments, the hydrolyzable linker is a thioether linker.

In some embodiments, the linker comprises one or more of substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. In some embodiments, the linker comprises substituted or unsubstituted C₁-C₃₀ alkylene. In some embodiments, the linker comprises polyethylene glycol such as (—CH₂—CH₂—O—)₁₋₁₀. In some embodiments, the linker comprises a structure selected from:

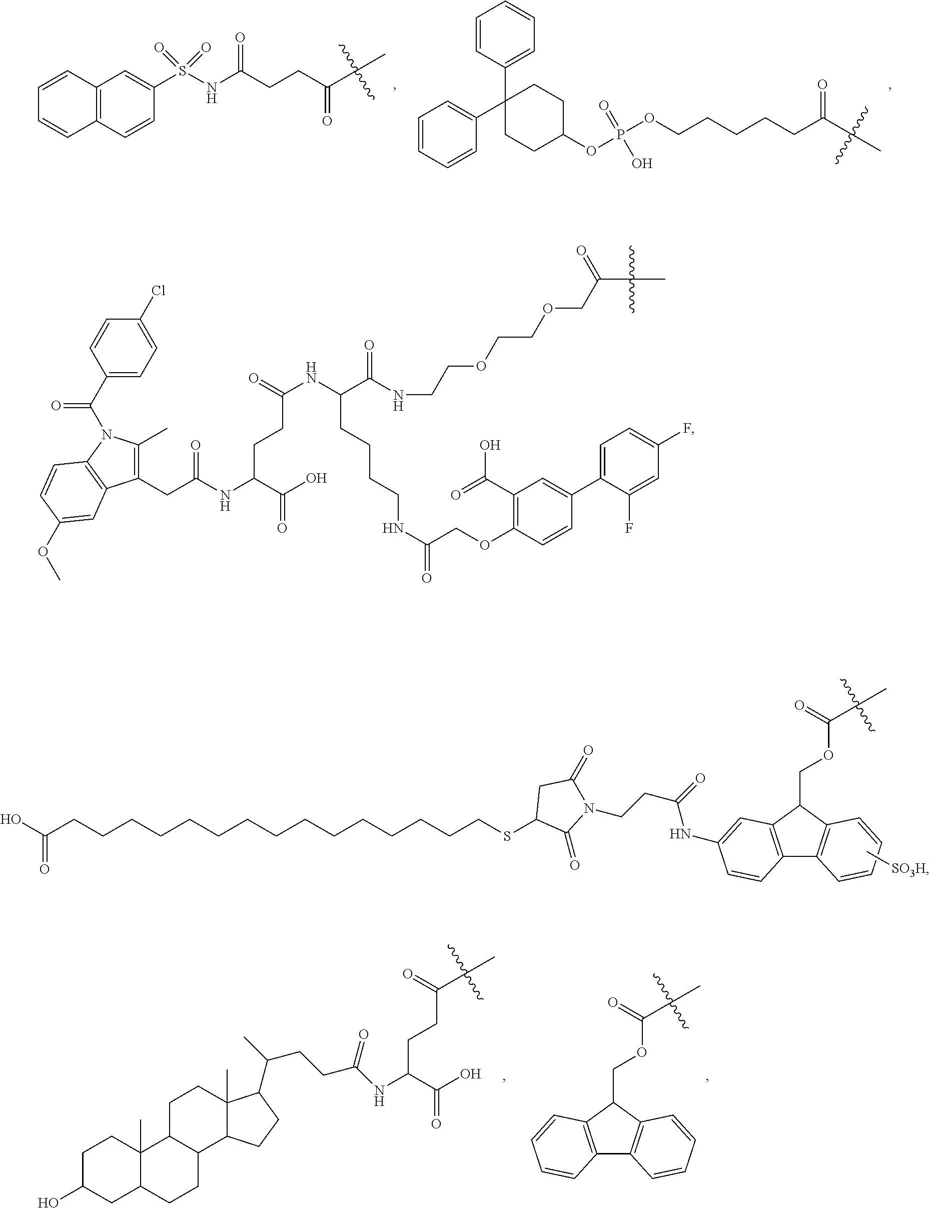

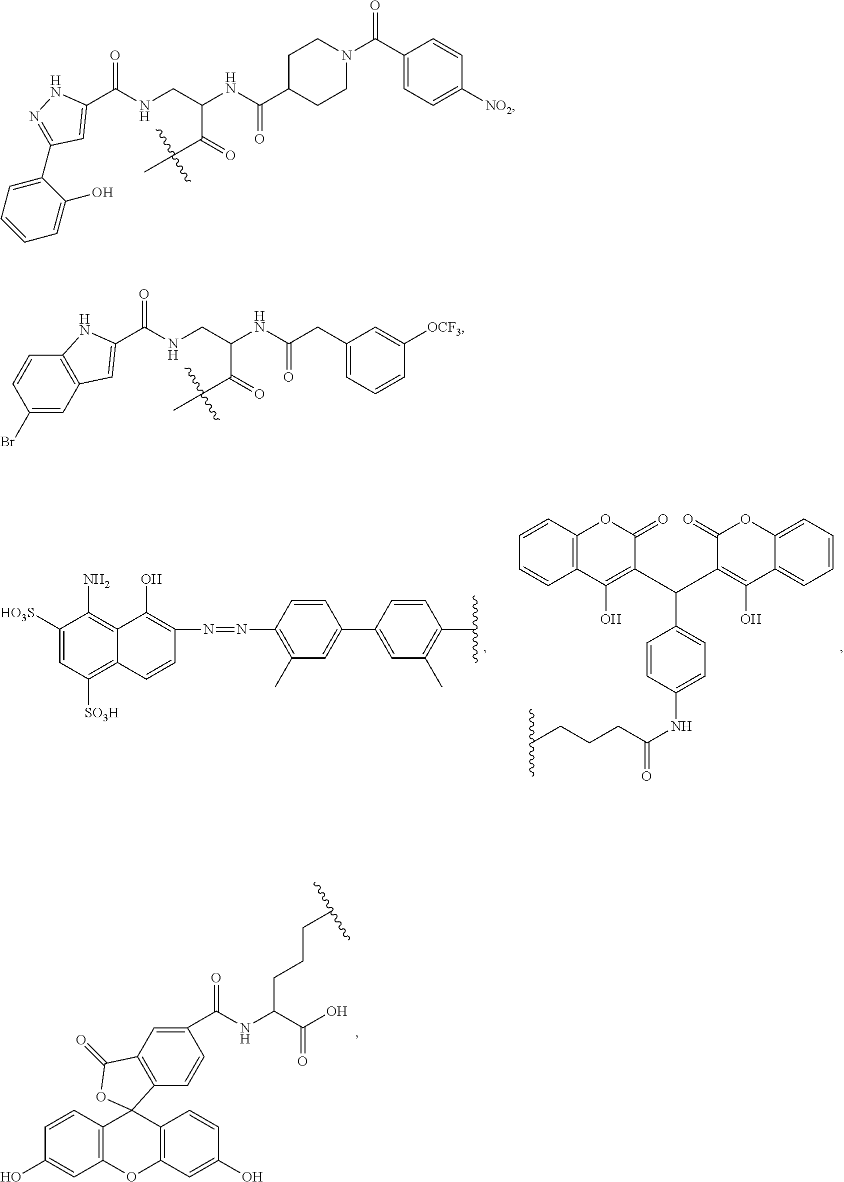 [SMILES: *C(=O)C(CNC(=O)c1cc(-c2ccccc2O)n[nH]1)NC(=O)C1CCN(C(=O)c2ccc([N+](=O)[O-])cc2)CC1 ; *C(=O)C(CNC(=O)c1cc2cc(Br)ccc2[nH]1)NC(=O)Cc1cccc(OC(F)(F)F)c1 ; *c1ccc(-c2ccc(N=Nc3ccc4c(S(=O)(=O)O)cc(S(=O)(=O)O)c(N)c4c3O)c(C)c2)cc1C ; *CCCC(=O)Nc1ccc(C(c2c(O)c3ccccc3oc2=O)c2c(O)c3ccccc3oc2=O)cc1 ; *CCCC(NC(=O)c1ccc2c(c1)C(=O)OC21c2ccc(O)cc2Oc2cc(O)ccc21)C(=O)O]

and structures derived from any one thereof.

In some embodiments, the linker comprises a click chemistry residue. In some embodiments, the linker is attached to the peptide, to the metal chelator, or both via click chemistry, thereby forming a click chemistry residue. For example, the peptide can comprise an azide group (at N- or C-terminus or at a non-terminal amino acid) that reacts with an alkyne moiety of the linker. For another example, the peptide can comprise an alkyne group (at N- or C-terminus or at a non-terminal amino acid) that reacts with an azide of the linker. The metal chelator and the linker can be attached similarly. In some embodiments, the linker comprises an azide moiety, an alkyne moiety, or both. In some embodiments, the linker comprises a triazole.

In some embodiments, the click chemistry residue is

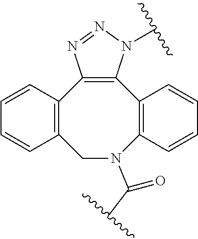 [SMILES: *C(=O)N1Cc2ccccc2-c2nnn(*)c2-c2ccccc21]

(DBCO-azide residue),

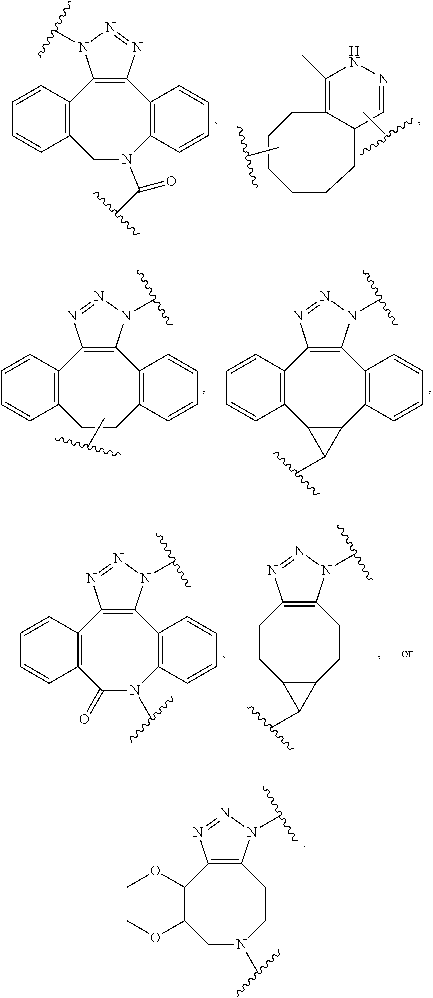

In some embodiments, the click chemistry residue is a DIBO-azide residue, BARAC-azide residue, DBCO-azide residue, DIFO-azide residue, COMBO-azide residue, BCN-azide residue, or DIMAC-azide residue. In some embodiments, the linker comprises a residue of nitrone dipole cycloaddition. In some embodiments, the linker comprises a residue of tetrazine ligation. In some embodiments, the linker comprises a residue of quadricyclane ligation. Exemplary groups of click chemistry residue are shown in Hein at al., “Click Chemistry, A Powerful Tool for Pharmaceutical Sciences,” Pharmaceutical Research volume 25, pages2216-2230 (2008); Thirumurugan et al, “Click Chemistry for Drug Development and Diverse Chemical—Biology Applications,” Chem. Rev. 2013, 113, 7, 4905-4979; US20160107999A1; U.S. Pat. No. 10,266,502B2; and US20190204330A1, each of which is incorporated by reference in its entirety.

In some embodiments, a linker described herein comprises two or more motifs. In some embodiments, one or more of the motifs are connected via click chemistry such that they can be clicked in/out of the linker. Each of the motifs in a linker can have independent functions. For example, a linker can comprise a motif that functions to adjust plasma half-life and/or a motif that functions as a spacer between the peptide and metal chelator.

In some embodiments, the linker has a structure of

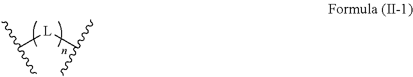

wherein each L is independently —O—, —NR^{L}—, —N(R^{L})₂⁺—, —OP(═O)(OR^{L})O—, —S—, —S(═O)—, —S(═O)₂—, ═CH—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}C(═S)NR^{L}—, —CR^{L}═N—, —N═CR^{L}, —NR^{L}S(═O)₂—, —S(═O)₂NR^{L}—, —C(═O)NR^{L}S(═O)₂—, —S(═O)₂NR^{L}C(═O)—, substituted or unsubstituted C₃-C₁₅ cycloalkyl, substituted or unsubstituted C₁-C₁₂ heterocycloalkyl, substituted or unsubstituted aryl, substituted or unsubstituted heteroaryl, substituted or unsubstituted C₁-C₃₀ alkylene, substituted or unsubstituted C₂-C₃₀ alkenylene, substituted or unsubstituted C₂-C₃₀ alkynylene, substituted or unsubstituted C₁-C₃₀ heteroalkylene, —(C₁-C₃₀ alkylene)-O—, —O—(C₁-C₃₀ alkylene)-, —(C₁-C₃₀ alkylene)-NR^{L}—, —NR^{L}-(C₁-C₃₀ alkylene)-, —(C₁-C₃₀ alkylene)-N(R^{L})₂⁺—, —N(R^{L})₂⁺—(C₁-C₃₀ alkylene)-, or a click chemistry residue; and

each R^{L} is independently hydrogen, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₈ cycloalkyl, substituted or unsubstituted C₂-C₇ heterocycloalkyl, substituted or unsubstituted aryl, or substituted or unsubstituted heteroaryl; and

n is 1,2,3,4, 5,6,7, 8,9, 10, 11, 12, 13, 14, or 15.

In some embodiments, the linker has a structure of

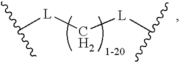

wherein each L is independently —O—, —NR^{L}—, —N(R^{L})₂⁺—, —OP(═O)(OR^{L})O—, —S—, —S(═O)—, —S(═O)₂—, —CH═CH—, ═CH—, —C≡C—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}S(═O)₂—, —S(═O)₂NR^{L}—, —C(═O)NR^{L}S(═O)₂—, or —S(═O)₂NR^{L}C(═O)—.

In some embodiments, the linker comprises substituted or unsubstituted C₁-C₃₀ alkylene, C₁-C₁₂ alkylene, C₁-C₈ alkylene, C₁-C₆ alkylene, or C₂-C₆ alkylene. In some embodiments, the linker comprises C₂-C₆ alkylene. In some embodiments, the linker comprises C₄-C₆ alkylene.

In some embodiments, the linker has a structure of

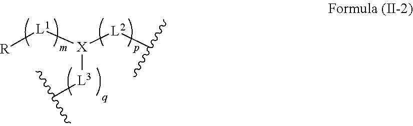

wherein each L¹, L², and L³ is independently —O—, —NR^{L}—, —N(R^{L})₂⁺—, —OP(═O)(OR^{L})O—, —S—, —S(═O)—, —S(═O)₂—, ═CH—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}C(═S)NR^{L}—, —CR^{L}═N—, —N═CR^{L}, —NR^{L}S(═O)₂—, —S(═O)₂NR^{L}—, —C(═O)NR^{L}S(═O)₂—, —S(═O)₂NR^{L}C(═O)—, substituted or unsubstituted C₃-C₁₅ cycloalkyl, substituted or unsubstituted C₁-C₁₂ heterocycloalkyl, substituted or unsubstituted aryl, substituted or unsubstituted heteroaryl, substituted or unsubstituted C₁-C₃₀ alkylene, substituted or unsubstituted C₂-C₃₀ alkenylene, substituted or unsubstituted C₂-C₃₀ alkynylene, substituted or unsubstituted C₁-C₃₀ heteroalkylene, substituted or unsubstituted C₁-C₁₅ arylene, —(C₁-C₃₀ alkylene)-O—, —O—(C₁-C₃₀ alkylene)-, —(C₁-C₃₀ alkylene)-NR^{L}—, —NR^{L}—(C₁-C₃₀ alkylene)-, —(C₁-C₃₀ alkylene)-N(R^{L})₂⁺—, —N(R^{L})₂⁺-(C₁-C₃₀ alkylene)-, or a click chemistry residue; and

R is hydrogen, azide, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted cycloalkynyl, substituted or unsubstituted C₃-C₃₀ cycloalkyl, substituted or unsubstituted C₂-C₃₀ heterocycloalkyl, substituted or unsubstituted aryl, or substituted or unsubstituted heteroaryl;

R^{L} is hydrogen, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₃₀ cycloalkyl, substituted or unsubstituted C₂-C₃₀ heterocycloalkyl, substituted or unsubstituted aryl, or substituted or unsubstituted heteroaryl;

X is N or CR^{L}; and
each of m, p, and q is independently 0, 1, 2, 3, 4, 5, 6, 7, 8, 9, 10, 11, 12, 13, 14, or 15.

In some embodiments, the linker has a structure of

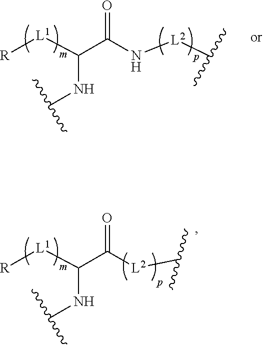

wherein

wherein each L¹ and L² is independently —O—, —NR^{L}—, —N(R^{L})₂⁺—, —OP(═O)(OR^{L})O—, —S—, —S(═O)—, —S(═O)₂—, —CH═CH—, ═CH—, —C≡C—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}S(═O)₂—, —S(═O)₂NR^{L}—, —C(═O)NR^{L}S(═O)₂—, —S(═O)₂NR^{L}C(═O)—, substituted or unsubstituted C₁-C₂₀ alkylene, or —(CHR^{L}—CHR^{L}—O)₁₋₁₀—;

R^{L} is hydrogen, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₈ cycloalkyl, or substituted or unsubstituted C₂-C₇ heterocycloalkyl;

R is hydrogen, azide, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted cycloalkynyl, substituted or unsubstituted C₃-C₈ cycloalkyl, substituted or unsubstituted C₂-C₇ heterocycloalkyl, substituted or unsubstituted aryl, or substituted or unsubstituted heteroaryl;

m is 1 to 10; and p is 0-3.

In some embodiments, L² is —O—, —NR^{L}—, —N(R^{L})₂⁺—, —OP(═O)(OR^{L})O—, —S—, —S(═O)—, —S(═O)₂—, —C(═O)—, —C(═O)O—, —OC(═O)—, —OC(═O)O—, —NR^{L}C(═O)—, —OC(═O)NR^{L}—, —NR^{L}C(═O)O—, —NR^{L}C(═O)NR^{L}—, —NR^{L}S(═O)₂—, —S(═O)₂NR^{L}—, —C(═O)NR^{L}S(═O)₂—, —S(═O)₂NR^{L}C(═O)—, substituted or unsubstituted C₁-C₆ alkylene, or —(CH₂—CH₂-O)₁₋₆—;

L¹ is —O—, —NR^{L}—, —N(R^{L})₂⁺—, —OP(═O)(OR^{L})O—, —S—, —S(═O)—, —S(═O)₂—, —CH═CH—, ═CH—, —C≡C—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}S(═O)₂—, —S(═O)₂NR^{L}—, —C(═O)NR^{L}S(═O)₂—, —S(═O)₂NR^{L}C(═O)—, substituted or unsubstituted alkylene, or —(CH₂—CH₂—O)₁₋₆—;

R^{L} is hydrogen or substituted or unsubstituted C₁-C₄ alkyl;

R is hydrogen, substituted or unsubstituted C₁-C₄ alkyl, substituted or unsubstituted C₃-C₃₀ cycloalkyl, substituted or unsubstituted C₂-C₃₀ heterocycloalkyl, substituted or unsubstituted aryl, or substituted or unsubstituted heteroaryl;

m is 1 to 10; and p is 0-3.

In some embodiments, the linker has a structure of

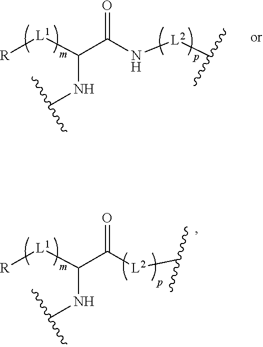

wherein

L² is —O—, —NR^{L}—, —N(R^{L})₂⁺—, —OP(═O)(OR^{L})O—, —S—, —S(═O)—, —S(═O)₂—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}S(═O)₂—, —S(═O)₂NR^{L}—, —C(═O)NR^{L}S(═O)₂—, —S(═O)₂NR^{L}C(═O)—, substituted or unsubstituted C₁-C₆ alkylene, or —(CH₂—CH₂—O)₁₋₆—;

L¹ is —O—, —NR^{L}—, —N(R^{L})₂⁺—, —OP(═O)(OR^{L})O—, —S—, —S(═O)—, —S(═O)₂—, —CH═CH—, ═CH—, —C≡C—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}S(═O)₂—, —S(═O)₂NR^{L}—, —C(═O)NR^{L}S(═O)₂—, —S(═O)₂NR^{L}C(═O)—, substituted or unsubstituted C₁-C₂₀ alkylene, or —(CH₂—CH₂—O)₁₋₆—;

R^{L} is hydrogen or substituted or unsubstituted C₁-C₄ alkyl;

R is hydrogen, substituted or unsubstituted C₁-C₄ alkyl, substituted or unsubstituted C₃-C₃₀ cycloalkyl, substituted or unsubstituted C₂-C₃₀ heterocycloalkyl, substituted or unsubstituted C₆-C₁₀ aryl, or substituted or unsubstituted C₅-C₉ heteroaryl;

m is 1 to 4; and p is 0-3.

In some embodiments, R is hydrogen, substituted or unsubstituted C₆-C₁₀ aryl, substituted or unsubstituted C₅-C₉ heteroaryl, or a sterol.

In some embodiments, at least one L¹ is unsubstituted C₃-C₂₀ alkylene.

In some embodiments, the linker comprises one or more of a substituted or unsubstituted C₆-C₁₀ aryl, substituted or unsubstituted C₅-C₉ heteroaryl, a sterol, sulfonamide, phosphate ester, polyethylene glycol, or C₃-C₂₀ alkylene, or amino acid residues.

In some embodiments, the linker is

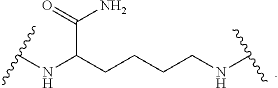 [SMILES: *NCCCCC(N*)C(N)=O]

In some embodiments, the linker is or comprises lysine. In some embodiments, the linker comprises C₁-C₁₂ alkylene. In some embodiments, the linker comprises C₃-C₉ alkylene. In some embodiments, the linker comprises C₂-C₈ alkylene. In some embodiments, the linker comprises 1 to 10 repeating ethylene glycol units. In some embodiments, the linker comprises 2 to 4 repeating ethylene glycol units. In some embodiments, the linker comprises 5 to 8 repeating ethylene glycol units. In some embodiments, the linker comprises NH₂—(CH₂)n-COOH, wherein n is 1 to 12. In some embodiments, the linker comprises NH₂—(CH₂)₂—COOH. In some embodiments, the linker comprises NH₂—(CH₂)₃—COOH. In some embodiments, the linker comprises NH₂—(CH₂)₄—COOH. In some embodiments, the linker comprises NH₂—(CH₂)₅—COOH. In some embodiments, the linker comprises NH₂—(CH₂)₆—COOH. In some embodiments, the linker comprises NH₂—(CH₂)₇—COOH. In some embodiments, the linker comprises NH₂—(CH₂)₈—COOH. In some embodiments, the linker comprises NH₂—(CH₂)₁₀—COOH. In some embodiments, the linker is absent.

In some embodiments, the linker is configured to reversibly bind to a plasma protein such as albumin. In some embodiments, a dissociation constant (Kd) between the linker and human serum albumin is at most 15 μM, as determined at room temperature in human serum condition. In some embodiments, the Kd is from about 0.1 nM to about 10 μM. In some embodiments, the Kd is from about 10 nM to about 10 μM. In some embodiments, the Kd is from about 50 nM to about 1 μM. In some embodiments, the Kd is from about 100 nM to about 10 μM.

### Metal Chelator

In one aspect, described herein are conjugates that comprise a metal chelator that is configured to bind with a radionuclide. The metal chelator can refer to a moiety of the conjugate that is configured to bind with a radionuclide. In some embodiments, a conjugate described herein comprises two or more independent metal chelators, e.g., 2, 3, 4, 5, or more metal chelators. In some embodiments, a conjugate described herein comprises two metal chelators, which can be the same or different. The metal chelator can be attached to the linker or the peptide through any suitable group/atom of the chelator.

In some embodiments, the metal chelator is capable of binding a radioactive atom. The binding can be direct, e.g., the metal chelator can make hydrogen bonds or electrostatic interactions with the radioactive atom. The binding can also be indirect, e.g., the metal chelator binds to a molecule that comprises a radioactive atom. In some embodiments, the metal chelator comprises, or is, a macrocycle. In some embodiments, the metal chelator comprises, or is, 2,2′,2″,2′″-(1,4,7,10-Tetraazacyclododecane-1,4,7,10-tetrayl)tetraacetic acid (DOTA) or 1,4,7-triazacyclononane-1,4,7-triacetic acid (NOTA). In some embodiments, the metal chelator comprises a macrocycle, e.g., a macrocycle comprising an O and/or a N, DOTA, NOTA, one or more amines, one or more ethers, one or more carboxylic acids, EDTA, DTPA, TETA, DO3A, PCTA, or desferrioxamine.

In some embodiments, the metal chelator comprises a plurality of amines. In some embodiments, the metal chelator includes 4 or more N, 4 or more carboxylic acid groups, or a combination thereof. In some embodiments, the metal chelator does not comprise S. In some embodiments, the metal chelator comprises a ring. In some embodiments, the ring comprises an O and/or an N. In some embodiments, the metal chelator is a ring that includes 3 or more N, 3 or more carboxylic acid groups, or a combination thereof. In some embodiments, the metal chelator is poly polydentate.

In some embodiments, a metal chelator described herein comprises a cyclic chelating agent. Exemplary cyclic chelating agents include, but are not limited to, AAZTA, BAT, BAT-TM, Crown, Cyclen, DO2A, CB-DO2A, DO3A, H3HP-DO3A, Oxo-DO3A, p-NH₂-Bn-Oxo-DO3A, DOTA, DOTA-3py, DOTA-PA, DOTA-GA, DOTA-4AMP, DOTA-2py, DOTA-1py, p-SCN-Bn-DOTA, CHX-A″-EDTA, MeO-DOTA-NCS EDTA, DOTAMAP, DOTAGA, DOTAGA-anhydride, DOTMA, DOTASA, DOTAM, DOTP, CB-Cyclam, TE2A, CB-TE2A, CB-TE2P, DM-TE2A, MM-TE2A, NOTA, NOTP, HEHA, HEHA-NCS, p-SCN-Bn-HEHA, DTPA, CHX-A″-DTPA, p-NH₂-Bn-CHX-A″-DTPA, p-SCN-DTPA, p-SCN-Bz-Mx-DTPA, 1B4M-DTPA, p-SCN-Bn1B-DTPA, p-SCN-Bn-1B4M-DTPA, p-SCN-Bn-CHX-A″-DTPA, PEPA, p-SCN-Bn-PEPA, TETPA, DOTPA, DOTMP, DOTPM, t-Bu-calix[4]arene-tetracarboxylic acid, macropa, macropa-NCS, macropid, H₃L¹, H₃L⁴, H₂azapa, H₅decapa, bispa², H₄pypa, H₄octapa, H₄CHXoctapa, p-SCN-Bn-H₄octapa, p-SCN-Bn-H₄octapa, TTHA, p-NO₂-Bn-neunpa, H₄octox, H₂macropa, H₂bispa², H₄phospa, H₆phospa, p-SCN-Bn-H₆phospa, TETA, p-NO₂-Bn-TETA, TRAP, TPA, HBED, SHBED, HBED-CC, (HBED-CC)TFP, DMSA, DMPS, DHLA, lipoic acid, TGA, BAL, Bis-thioseminarabazones, p-SCN-NOTA, nNOTA, NODAGA, CB-TE1A1P, 3P-C-NETA-NCS, 3p-C-DEPA, 3P-C-DEPA-NCS, TCMC, PCTA, NODIA-Me, TACN, pycup1A1B, pycup2A, THP, DEDPA, H₂DEDPA, p-SCN-Bn-H₂DEDPA, p-SCN-Bn-TCMC, motexafin, NTA, NOC, 3p-C-NETA, p-NH₂-Bn-TE3A, SarAr, DiAmSar, SarAr-NCS, AmBaSar, BaBaSar, TACN-TM, CP256, C-NE3TA, C-NE3TA-NCS, NODASA, NETA-monoamide, C-NETA, NOPO, BPCA, p-SCN-Bn-DFO, DFO-ChX-Mal, DFO, DFO-IAC, DFO-BAC, DiP-LICAM, EC, SBAD, BAPEN, TACHPYR, NEC-SP, L^{py}, L1, L2, L3, and EuK-106. In some embodiments, the metal chelator is DOTA, TRITA, TETA, DOTA-MA, DO3A-HP, DOTMA, DOTA-pNB, DOTP, DOTMP, DOTEP, DOTMPE, F-DOTPME, DOTPP, DOTBzP, DOTA-monoamide, p-NCS-DOTA, p-NCS-PADOTA, BAT, DO3TMP-Monoamide, p-NCS-TRITA, NOTA, or CHX-A″-DTPA. In some embodiments, a metal chelator described herein comprises an acyclic chelating agent. Exemplary acyclic chelating agents include, but are not limited to, DTA, CyEDTA, EDTMP, DTPMP, DTPA, CyDTPA, Cy2DTPA, DTPA-MA, DTPA-BA, and BOPA. In some embodiments, a metal chelator described herein comprises DOTA, DOTP, DOTMA, DOTAM, DTPA, NTA, EDTA, DO3A, DO2A, NOC, NOTA, TETA, TACN, DiAmSar, CB-Cyclam, CB-TE2A, DOTA-4AMP, or NOTP. In some embodiments, a metal chelator described herein comprises H₄pypa, H₄octox, H₄octapa, p-NO₂-Bn-neunpa, p-SCN-Bn-H₄neunpa, TTHA, ^{t}Bu₄pypa-C7-NHS, H₄neunpa, H₂macropa, HP-DO3A, BT-DO3A, DO3A-Nprop, DO3AP, DO2A2P, DOA3P, DOTP, DOTPMB, DOTAMAE, DOTAMAP, DO3AM^{Bu}, DOTMA, TCE-DOTA, DEPA, PCTA, p-NO₂-Bn-PCTA, p-NO₂-Bn-DOTA, symPC2APA, symPCA2PA, asymPC2APA, asymPCA2PA, TRAP, AAZTA, DATAm, THP, HEHA, or HBED.

In some embodiments, the metal chelator is DO3A. In some embodiments, the metal chelator is PEPA. In some embodiments, the metal chelator is EDTA. In some embodiments, the metal chelator is CHX-A″-DTPA. In some embodiments, the metal chelator is HEHA. In some embodiments, the metal chelator is DOTMP. In some embodiments, the metal chelator is t-Bu-calix[4]arene-tetracarboxylic acid. In some embodiments, the metal chelator is macropa. In some embodiments, the metal chelator is macropa-NCS. In some embodiments, the metal chelator is H₄pypa. In some embodiments, the metal chelator is H₄octapa. In some embodiments, the metal chelator is H₄CHXoctapa. In some embodiments, the metal chelator is DOTP. In some embodiments, the metal chelator is crown.

In some embodiments, the metal chelator is DOTA. In some embodiments, the metal chelator is a chiral derivative of DOTA. Exemplary chiral DOTA chelators are described in Dai et al., Nature Communications (2018) 9:857. In some embodiments, the metal chelator is 2,2′,2″,2′″-((2S,5 S,8 S,11 S)-2,5,8,11-tetramethyl-1,4,7,10-tetraazacyclododecane-1,4,7,10-tetrayl)tetraacetic acid. In some embodiments, the metal chelator has a structure of

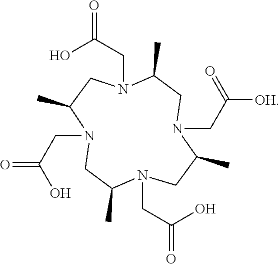 [SMILES: C[C@H]1CN(CC(=O)O)[C@@H](C)CN(CC(=O)O)[C@@H](C)CN(CC(=O)O)[C@@H](C)CN1CC(=O)O]

In some embodiments, the metal chelator is 2,2′,2″,2′″-((2S,5S,8S,11S)-2,5,8,11-tetraethyl-1,4,7,10-tetraazacyclododecane-1,4,7,10-tetrayl)tetraacetic acid. In some embodiments, the metal chelator has a structure of

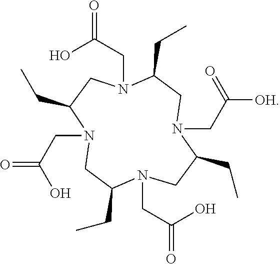 [SMILES: CC[C@H]1CN(CC(=O)O)[C@@H](CC)CN(CC(=O)O)[C@@H](CC)CN(CC(=O)O)[C@@H](CC)CN1CC(=O)O]

In some embodiments, the metal chelator has a structure of

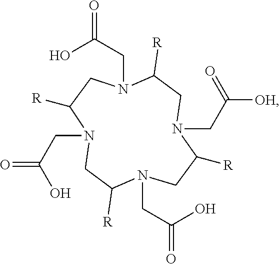

wherein each R is independently selected from hydrogen, alkyl, haloalkyl, hydroxyalkyl, aminoalkyl, heteroalkyl, alkenyl, alkynyl, cycloalkyl, heterocycloalkyl, aryl, heteroaryl, alkylcycloalkyl, alkylheterocycloalkyl, alkylaryl, alkylheteroaryl, or an amino acid side chain. In some embodiments, the metal chelator has a structure of

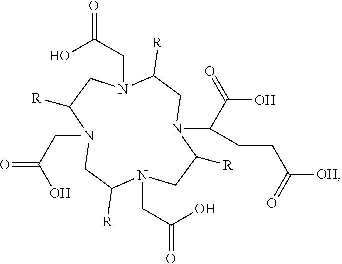

wherein each R is independently selected from hydrogen, alkyl, haloalkyl, hydroxyalkyl, aminoalkyl, heteroalkyl, alkenyl, alkynyl, cycloalkyl, heterocycloalkyl, aryl, heteroaryl, alkylcycloalkyl, alkylheterocycloalkyl, alkylaryl, alkylheteroaryl, or an amino acid side chain.

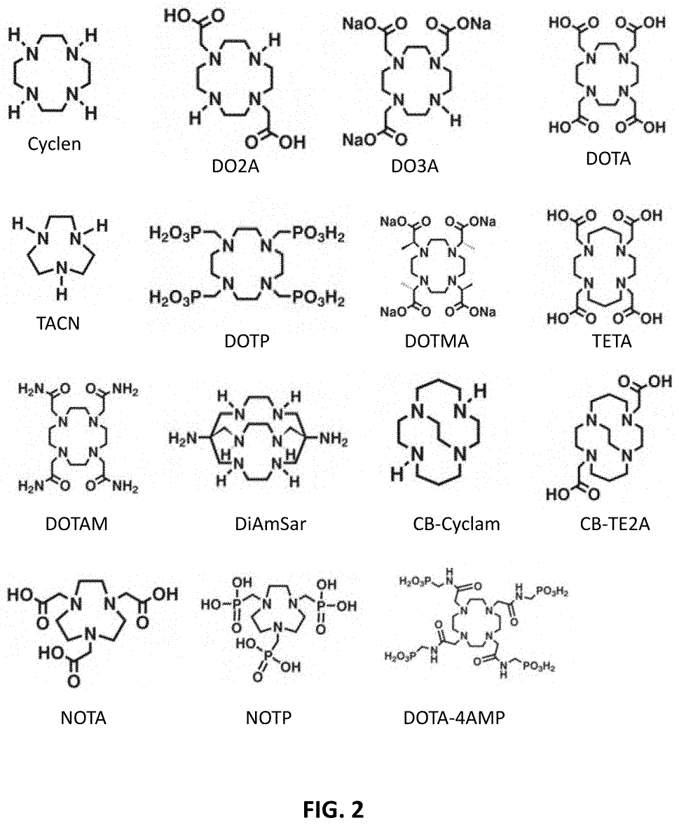

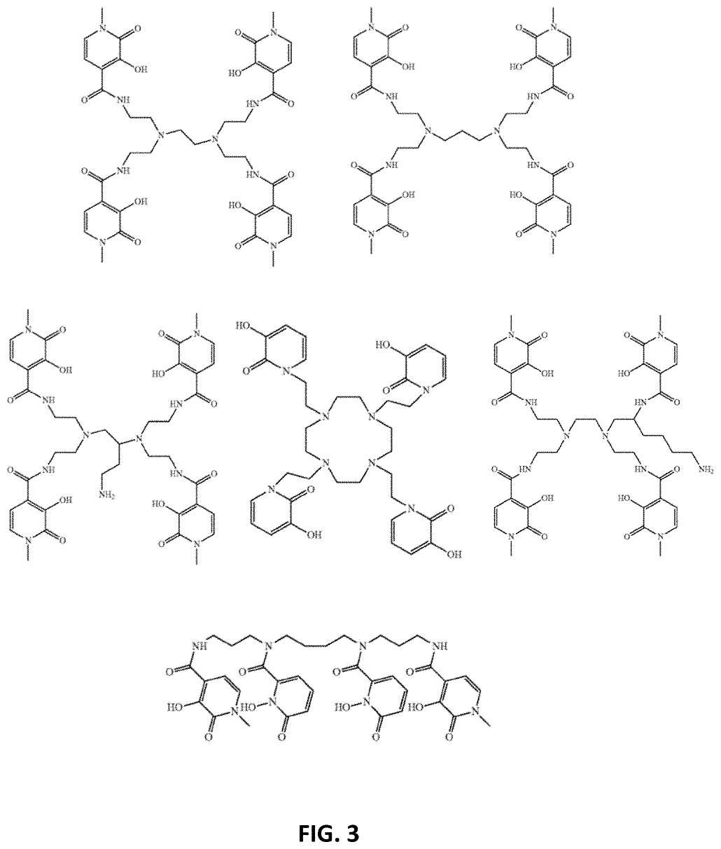

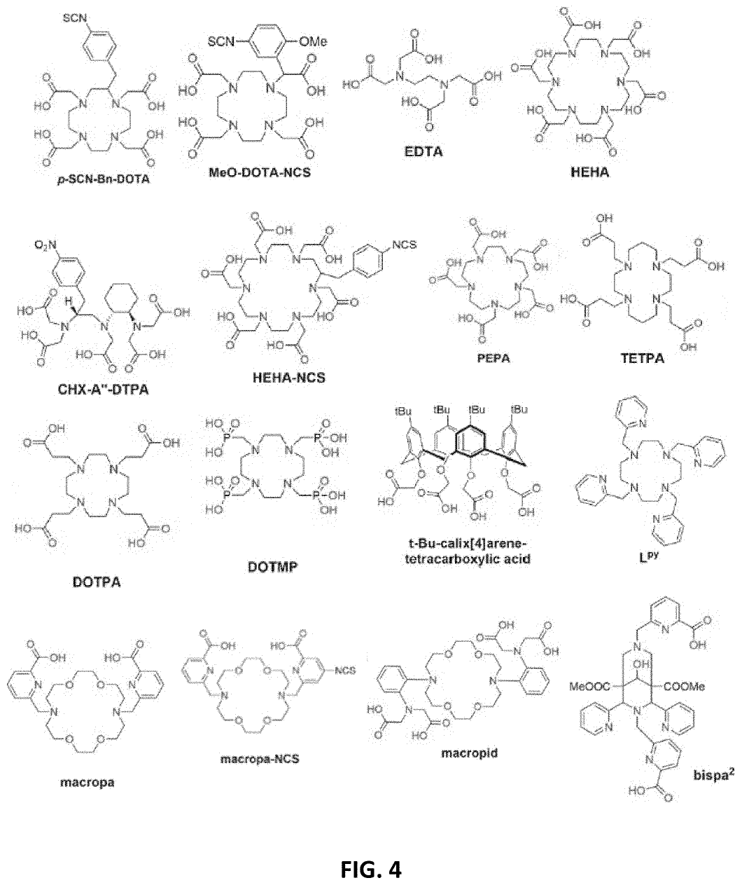

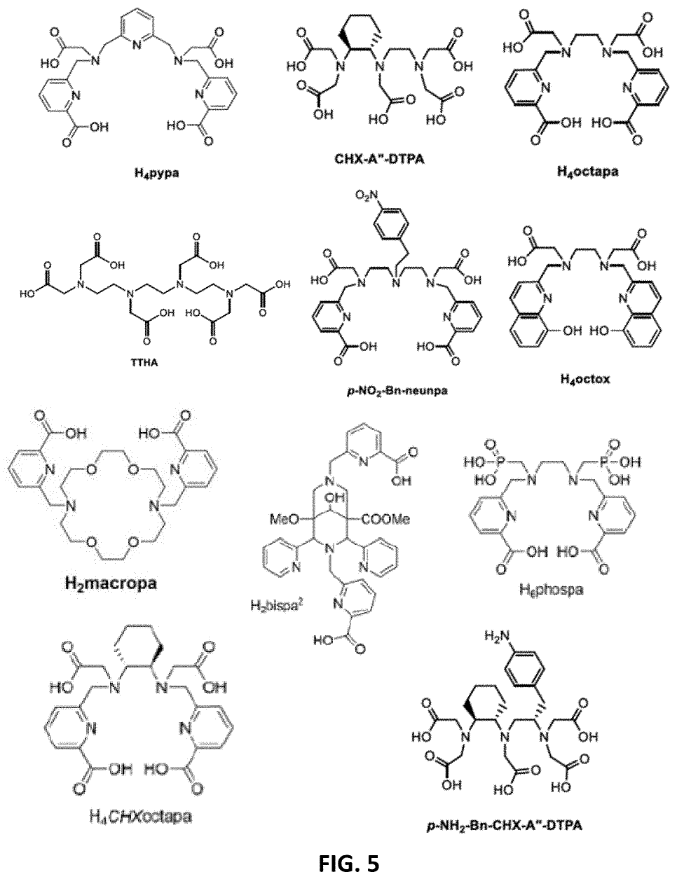

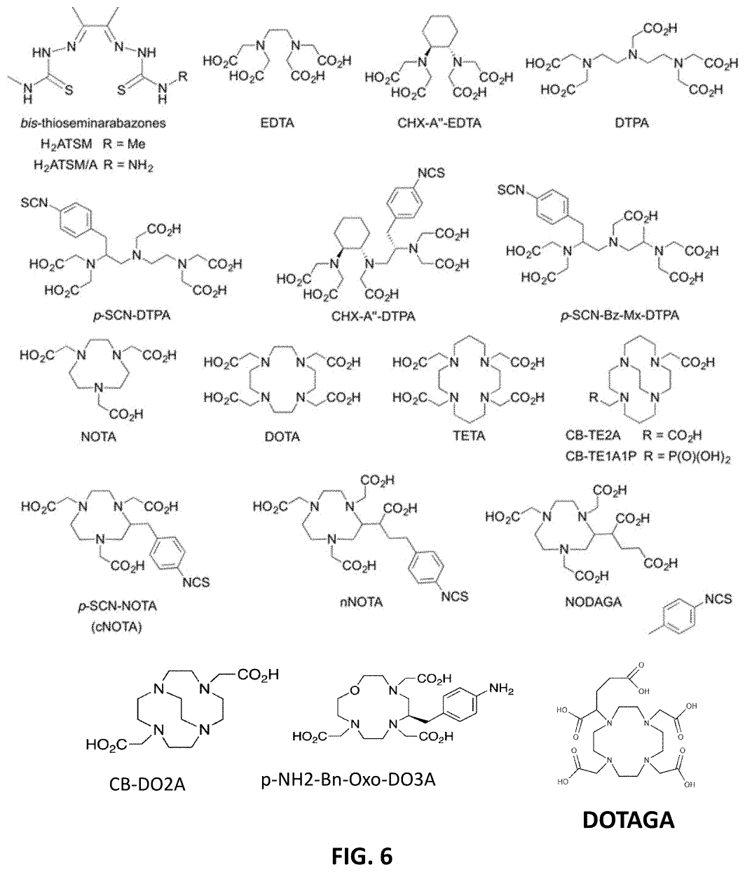

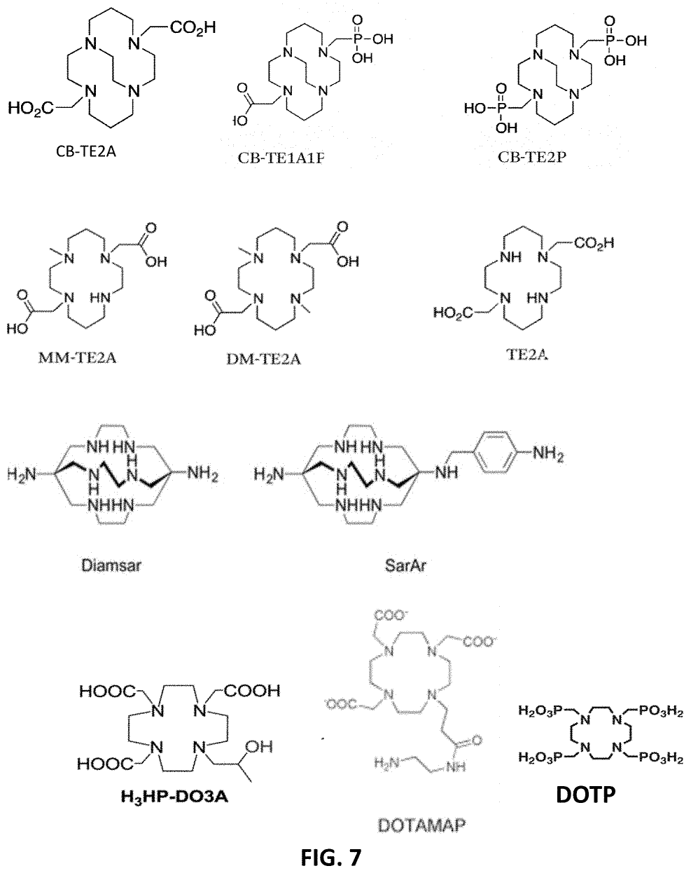

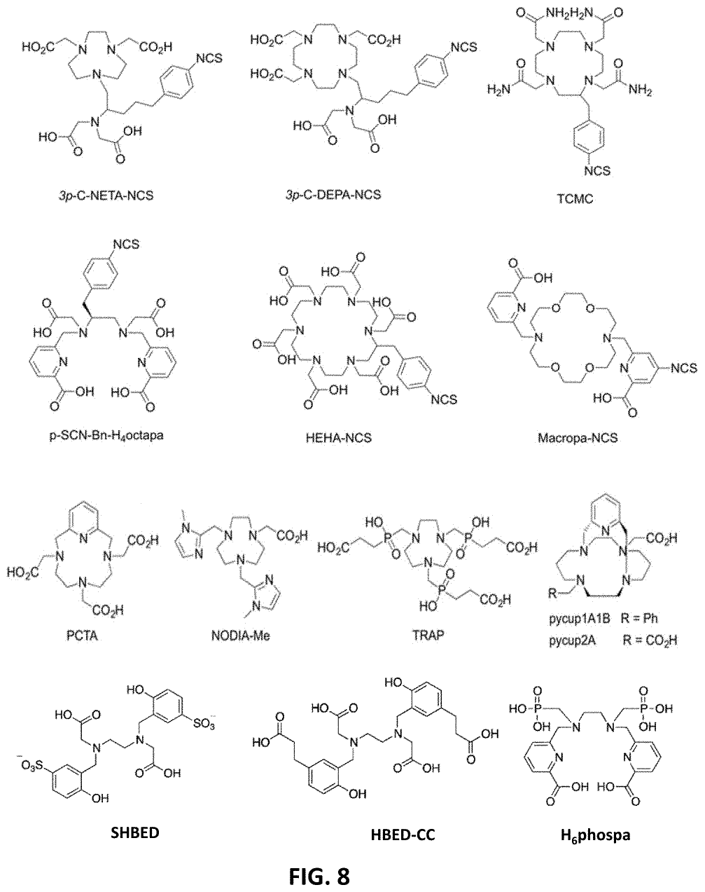

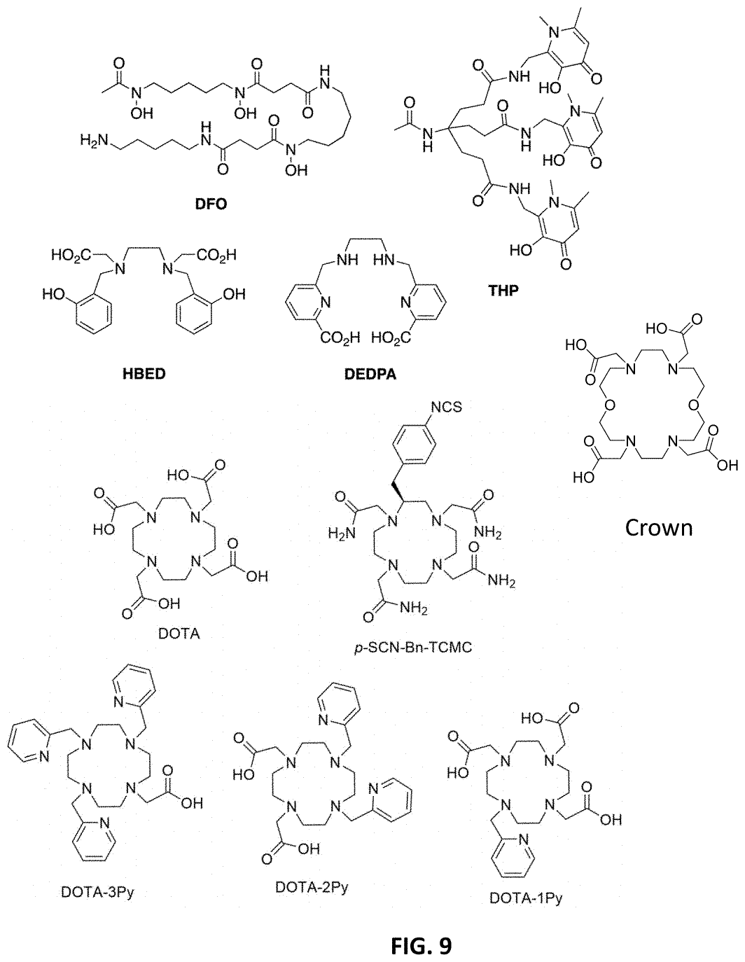

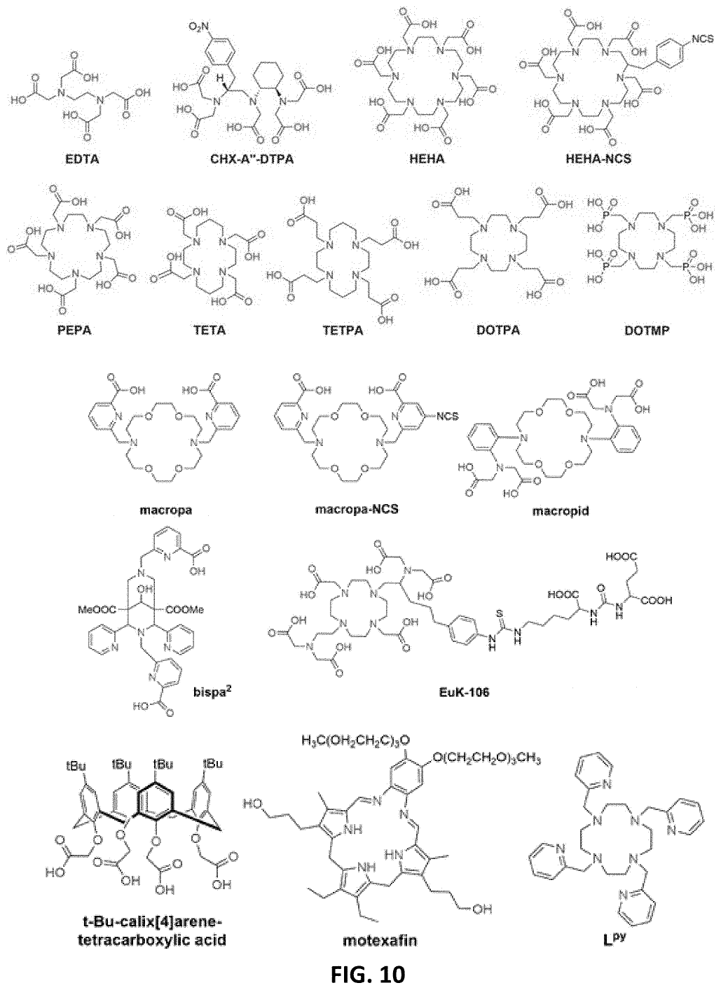

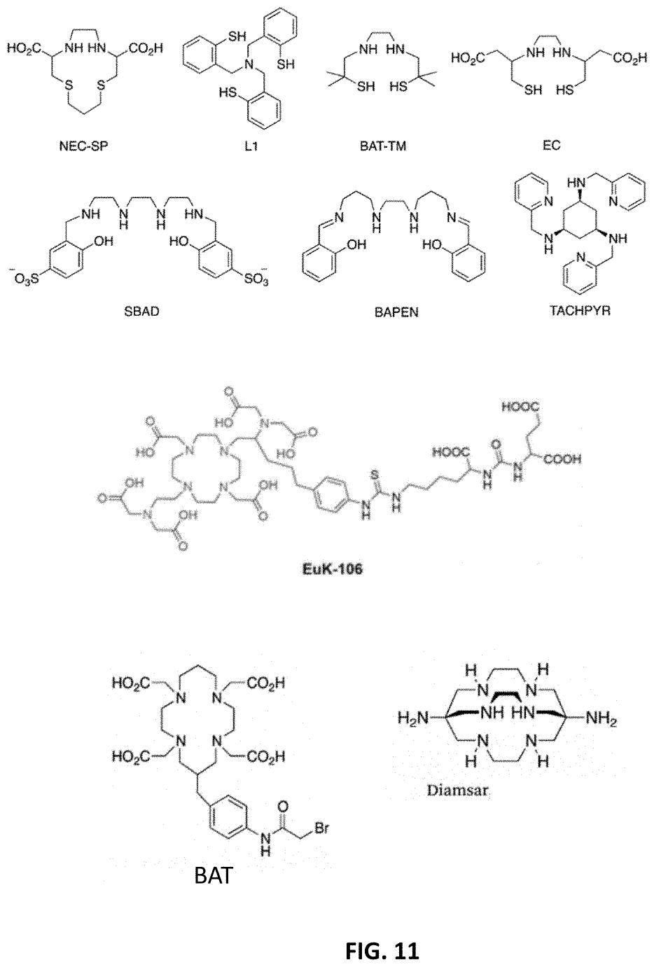

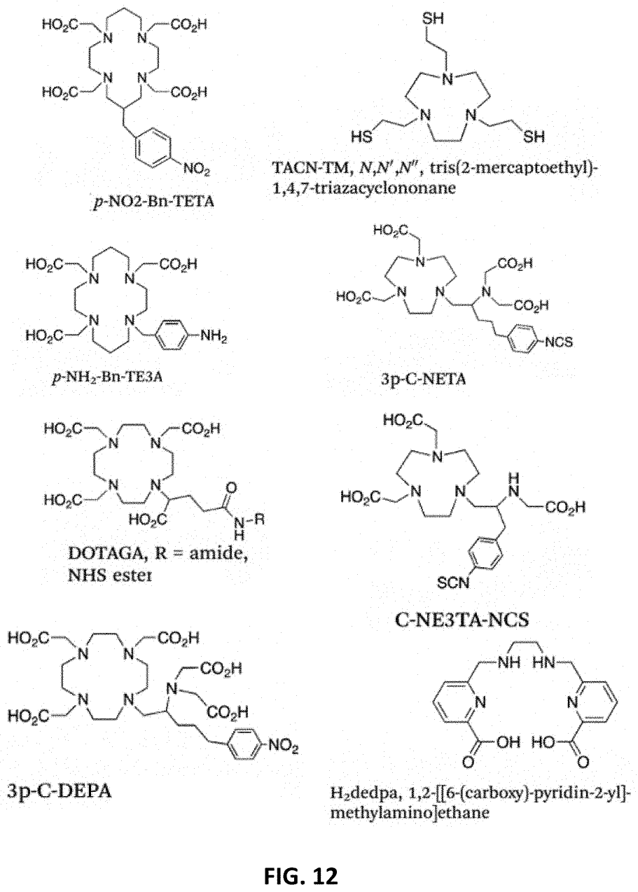

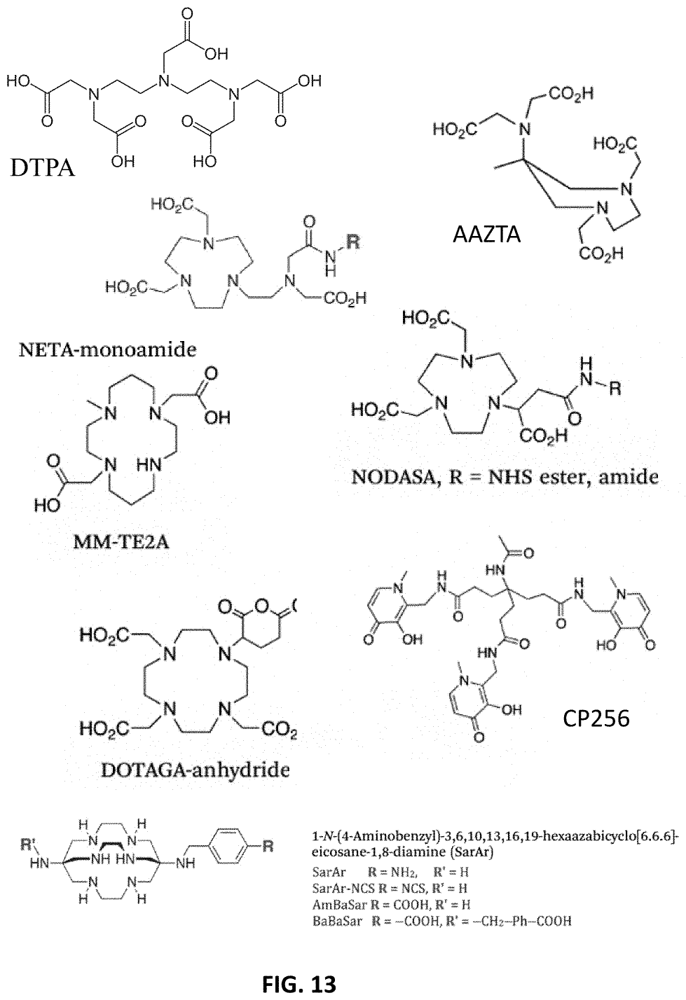

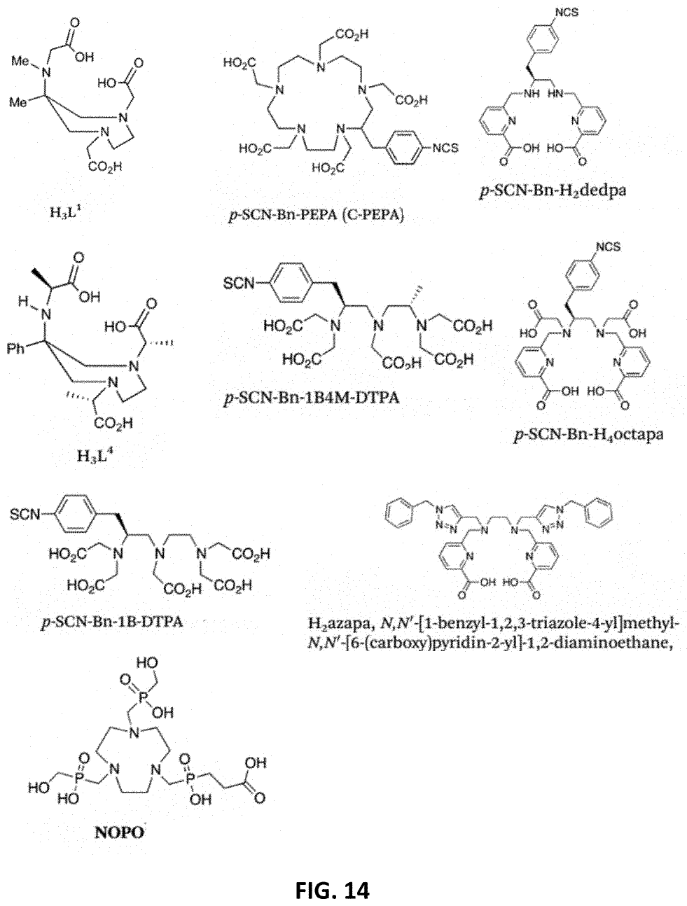

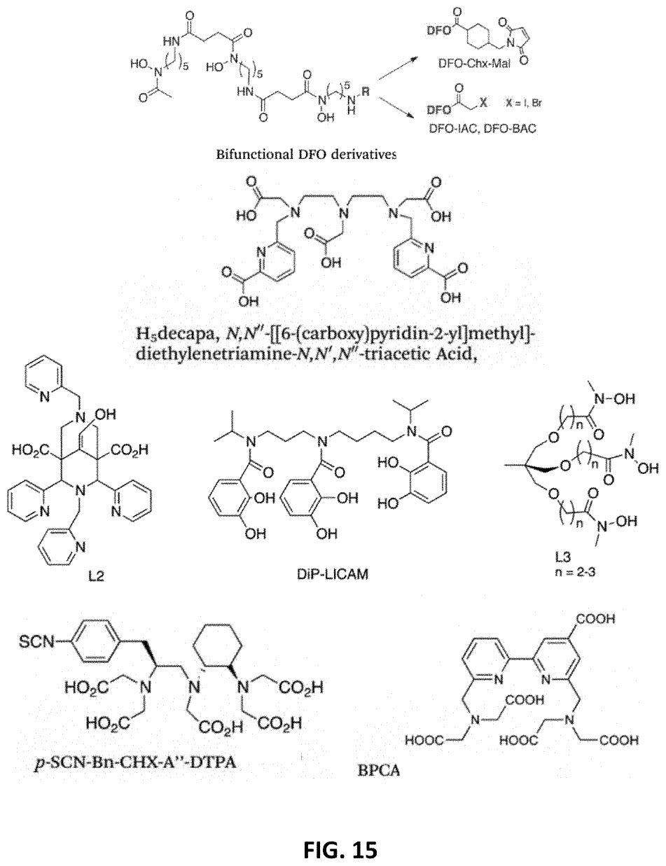

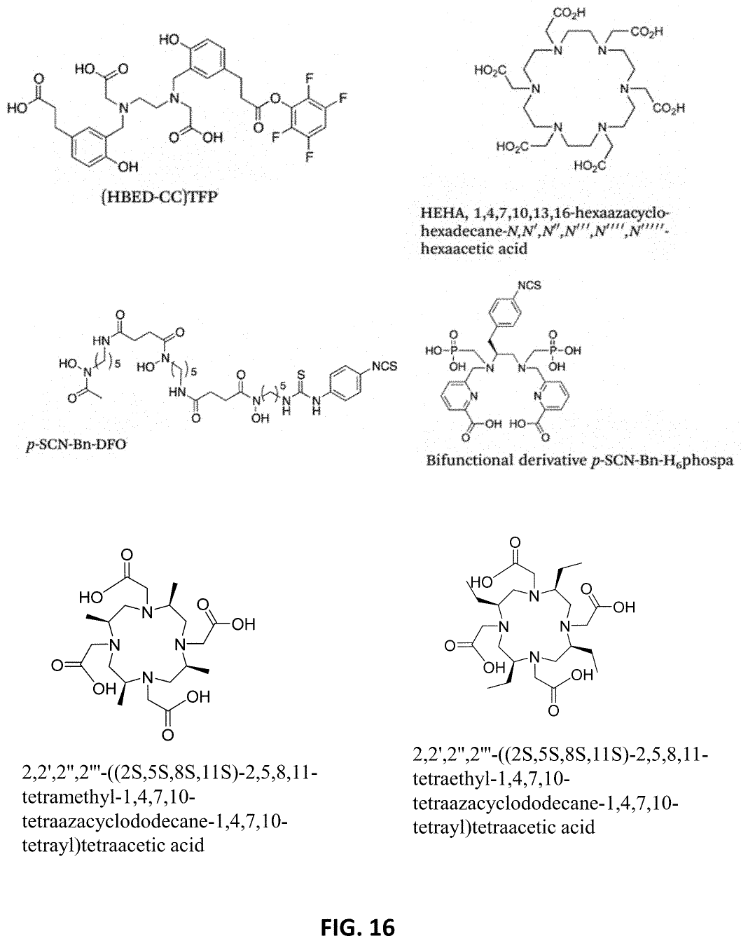

In some embodiments, the conjugate comprises DOTA. In some embodiments, the conjugate comprises a DOTA derivative such as p-SCN-Bn-DOTA and MeO-DOTA-NCS. In some embodiments, the conjugate comprises two independent metal chelators, and at least one or both are DOTA. The structures of some exemplary metal chelators are illustrated in FIGS. 2-16 (without showing the attachment points). Exemplary metal chelators are further described in WO2012/174136; US20130183235A1; US20120219495A1; Ramogidaand et al., EJNMMI radiopharm. chem. 4, 21 (2019); Thiele et al., Cancer Biotherapy and Radiopharmaceuticals 2018; Li et al., Bioconjugate Chem. 2019, 30, 5, 1539-1553; and Baranyai et al., Eur. J. Inorg. Chem. 36-56 (2020), each of which is incorporated by reference in its entirety.

### Radionuclide

In one aspect, described herein are conjugates that comprise a radionuclide. Generally, the type of radionuclide used in a therapeutic radiopharmaceutical can be tailored to the specific type of cancer, the type of targeting moiety (e.g., binding peptide), etc. Radionuclides that undergo a-decay produce particles composed of two neutrons and two protons, and radionuclides that undergo β-decay emit energetic electrons from their nuclei. Some radionuclides can also emit Auger. In some embodiments, the conjugate comprises an alpha particle-emitting radionuclide. Alpha radiation can cause direct, irreparable double-strand DNA breaks compared with gamma and beta radiation, which can cause single-stranded breaks via indirect DNA damage. The range of these particles in tissue and the half-life of the radionuclide can also be considered in designing the radiopharmaceutical conjugate. Tables 5A and 5B below illustrate some properties of exemplary radionuclides.

TABLE 5A
Exemplary radionuclides

| Nuclide | Emission | Half-life (days) |
|---|---|---|
| Actinium-225 (Ac-225) | α | about 10.0 |
| Lutetium (Lu-177) | β | about 6.7 |
| Radium-223 | α | about 11.4 |
| Radium-224 | α | about 3.63 |
| Astatine-211 | α | about 0.3 |
| Yttrium-90 | β | about 2.7 |
| Iodine-131 | β | about 8 |
| Samarium-153 | β | about 1.9 |
| Lead-212 | β | about 0.4 |
| Bismuth-212 | α | about 0.04 |
| Thorium-227 | α | about 18.7 |
| Terbium-149 | α | about 0.17 |

TABLE 5B
Exemplary radionuclides

| Nuclide | Half-life |
|---|---|
| Lutetium-177 (Lu-177) | about 6.7 days |
| Indium-111 (In-111) | about 2.8 days |
| Gallium-68 (Ga-68) | about 68 minutes |
| Copper-64 (Cu-64) | about 12.7 hours |
| Zirconium-89 (Zr-89) | about 78.4 hours |
| Iodine-124 (I-124) | about 4.2 days |
| Cerium-134 (Ce-134) | about 3.2 days |
| Fluorine-18 (F-18) | about 110 minutes |

In some embodiments, a conjugate described herein comprises one or more independent radionuclides. In some embodiments, the conjugate comprises two radionuclides. In some embodiments, each of the one or more radionuclides is bound to a metal chelator of the conjugate. In some embodiments, two radionuclides of a conjugate are bound to the same metal chelator. In some embodiments, two radionuclides of a conjugate are bound to two independent metal chelators. In some embodiments, each of the one or more radionuclides is an alpha particle-emitting radionuclide.

In some embodiments, a conjugate described herein comprises an alpha particle-emitting radionuclide. In some embodiments, the alpha particle-emitting radionuclide is actinium-225 (²²⁵Ac), astatine-211 (²¹¹At) radium-223 (²²³Ra), radium-224 (²²⁴Ra), bismuth-213 (²¹³Bi), Terbium-149 (¹⁴⁹Tb), or thorium-227 (²²⁷Th). In some embodiments, the alpha particle-emitting radionuclide is ²²⁵Ac. In some embodiments, the alpha particle-emitting radionuclide is ²¹³Bi. In some embodiments, the alpha particle-emitting radionuclide is ²¹²Bi. In some embodiments, the alpha particle-emitting radionuclide is ²¹²Pb. In some embodiments, the alpha particle-emitting radionuclide is ²²⁴Ra. In some embodiments, the alpha particle-emitting radionuclide is ²²³Ra. In some embodiments, the alpha particle-emitting radionuclide is ²²⁷Th. In some embodiments, the alpha particle-emitting radionuclide is ²¹¹At. In some embodiments, the alpha particle-emitting radionuclide is ¹⁴⁹Tb. In some embodiments, the radionuclide is Zirconium-89 (⁸⁹Zr). In some embodiments, the radionuclide is Zirconium-89 (⁸⁹Zr). In some embodiments, a conjugate described herein comprises a radionuclide selected from ⁶⁷Cu, ⁶⁴Cu, ⁸⁹Zr, ⁹⁰Y, ¹⁰⁹Pd, ¹¹¹Ag, ¹⁴⁹Pm, ¹⁵³Sm, ¹⁶⁶Ho, ₉₉ₘTc, ⁶⁷Ga, ⁶⁸Ga, ¹¹¹In, ⁹⁰Y, ¹⁷⁷Lu, ¹⁸⁶Re, ¹⁸⁸Re, ¹⁹⁷Au, ¹⁹⁸Au, ¹⁹⁹Au, ¹⁰⁵Rh, ¹⁶⁵Ho, ¹⁶¹Tb, ¹⁴⁹Pm, ⁴⁴Sc, ⁴⁷Sc, ⁷⁰As, ⁷¹As, ⁷²As, ⁷³As, ⁷⁴As, ⁷⁶As, ⁷⁷As, ²¹²Pb, ²¹²Bi, ²¹³Bi, ²²⁵Ac, ^{117m}Sn, ⁶⁷Ga, ²⁰¹Tl, ¹²³I, ¹³¹I, ¹⁶⁰Gd, ¹⁴⁸Nd, ⁸⁹Sr, and ²¹¹At. In some embodiments, the radionuclide is ²²⁵AC. In some embodiments, the radionuclide is a decay daughter of ²²⁵Ac such as ²²¹Fr, ²¹⁷At, ²¹³Bi, ²¹³Po, ²⁰⁹Tl, ²⁰⁹Pb, or ²⁰⁹Bi. In some embodiments, the conjugate comprises two ²²⁵Ac radionuclides. In some embodiments, the radionuclide is ¹⁷⁷Lu. In some embodiments, the conjugate comprises two ¹⁷⁷Lu radionuclides.

In some embodiments, the conjugate comprises an alpha particle-emitting radionuclide bound to the metal chelator. In some embodiments, the alpha particle-emitting radionuclide is actinium-225, astatine-211, thorium-227, or radium-223. In some embodiments, the alpha particle-emitting radionuclide is actinium-225.

In some embodiments, the conjugate comprises a beta particle-emitting radionuclide bound to the metal chelator. In some embodiments, the beta particle emitting radionuclide is zircronium-89, yttrium-90, iodine-131, samarium-153, lutetium-177, or lead-212.

In some embodiments, the conjugate comprises a gamma particle emitting radionuclide. In some embodiments, the gamma particle emitting radionuclide is indium-111.

In some embodiments, conjugates described herein do not contain any radionuclide, i.e., a cold conjugate. For example, in some cases, a radionuclide can be replaced with a surrogate (e.g., ²²⁵Ac replaced with lanthanum) for testing and experimental purposes.

### Conjugates Comprising Non-Radioactive Drugs

In one aspect, described herein is a conjugate that comprises a peptide, a non-radioactive drug, and optionally a linker. In some embodiments, the conjugate further comprises a metal chelator and optionally a radionuclide bound to the metal chelator. The peptide can be cyclic or acyclic, and it can be monocyclic, bicyclic or polycyclic. In one aspect, described herein is a conjugate that comprises a cyclic peptide and non-radioactive drug. In some embodiments, the peptide (such as cyclic peptide) is configured to bind to a target. A conjugate described herein can further comprises a linker.

The non-radioactive drug can be a toxin. In some embodiments, the toxin is selected from pseudomonas exotoxin (PE), deBouganin, Bouganin, diphtheria toxin (DT) and ricin. The non-radioactive drug can be a chemotherapy agent.

The non-radioactive drug can be a cytotoxic drug. Exemplary cytotoxic drugs include aplidin, azaribine, anastrozole, azacytidine, bleomycin, bortezomib, bryostatin-1, busulfan, calicheamycin, camptothecin, 10-hydroxycamptothecin, carmustine, celebrex, chlorambucil, cisplatin, irinotecan (CPT-11), SN-38, carboplatin, cladribine, cyclophosphamide, cytarabine, dacarbazine, docetaxel, dactinomycin, daunomycin glucuronide, daunorubicin, dexamethasone, diethylstilbestrol, doxorubicin, 2-pyrrolinodoxorubicin (2P-DOX), cyano-morpholino doxorubicin, doxorubicin glucuronide, epirubicin glucuronide, ethinyl estradiol, estramustine, etoposide, etoposide glucuronide, etoposide phosphate, floxuridine (FUdR), 3′,5′-O-dioleoyl-FudR (FUdR-dO), fludarabine, flutamide, fluorouracil, fluoxymesterone, gemcitabine, hydroxyprogesterone caproate, hydroxyurea, idarubicin, ifosfamide, L-asparaginase, leucovorin, lomustine, mechlorethamine, medroprogesterone acetate, megestrol acetate, melphalan, mercaptopurine, 6-mercaptopurine, methotrexate, mitoxantrone, mithramycin, mitomycin, mitotane, phenyl butyrate, prednisone, procarbazine, paclitaxel, pentostatin, PSI-341, semustine streptozocin, tamoxifen, taxanes, taxol, testosterone propionate, thalidomide, thioguanine, thiotepa, teniposide, topotecan, uracil mustard, velcade, vinblastine, vinorelbine, vincristine, ricin, abrin, ribonuclease, onconase, rapLR1, DNase I, Staphylococcal enterotoxin-A, pokeweed antiviral protein, gelonin, diphtheria toxin, Pseudomonas exotoxin, Pseudomona endotoxin, or combinations of these.

In some embodiments, the non-radioactive drug is selected from duocarmycin and its analogues, dolastatins, combretastatin, calicheamicin, N-acetyl-□-calicheamycin (CMC), a calicheamycin derivative, maytansine and analogues thereof, DM-I, auristatin E, auristatin EB (AEB), auristatin EFP (AEFP), monomethyl auristatin E (MMAE), monomethyl auristatin F (MMAF), tubulysin, disorazole, the epothilones, Paclitaxel, docetaxel, Topotecan, echinomycin, estramustine, cemadotine, eleutherobin, methopterin, actinomycin, daunorubicin, the daunorubicin conjugates, mitomycin C, mitomycin A, vincristine, retinoic acid, camptothecin, a camptothecin derivative, SN38, maytansine, a derivative of the maytansinoid type, DM1, DM4, TK1, amanitin, a pyrrolobenzodiazepine, a pyrrolobenzodiazepine dimer, methotrexate, ilomedine, aspirin, an IMIDs, lenalidomide, pomalidomide.

### Isomers/Stereoisomers

In some embodiments, the compounds described herein exist as geometric isomers. In some embodiments, the compounds described herein possess one or more double bonds. The compounds presented herein include cis, trans, syn, anti, entgegen (E), and zusammen (Z) isomers as well as the corresponding mixtures thereof. In some situations, the compounds described herein possess one or more chiral centers and each center exists in the R configuration or S configuration. The compounds described herein include diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers, and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent.

### Tautomers

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

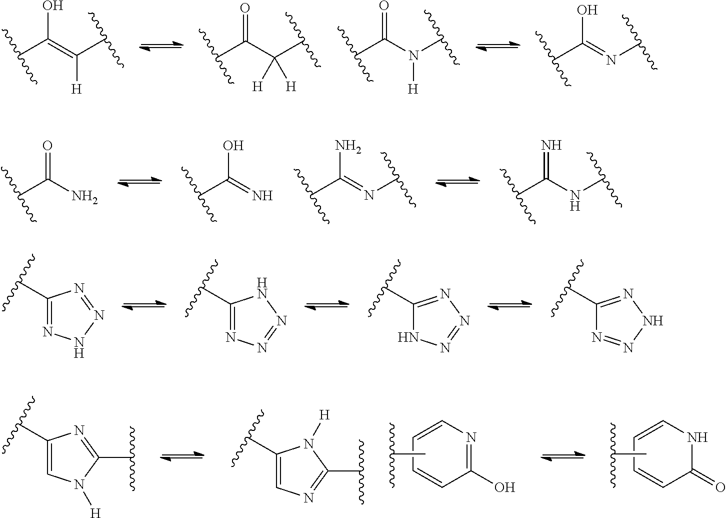

In some instances, the compounds disclosed herein exist in tautomeric forms. The structures of said compounds are illustrated in the one tautomeric form for clarity. The alternative tautomeric forms are expressly included in this disclosure.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds described herein, or a solvate, or stereoisomer thereof, include isotopes of hydrogen, carbon, nitrogen, oxygen, phosphorous, sulfur, fluorine, and chloride, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds described herein, and the pharmaceutically acceptable salts, solvates, or stereoisomers thereof which contain the aforementioned isotopes and/or other isotopes of other atoms are within the scope of this disclosure. Certain isotopically-labeled compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are notable for their ease of preparation and detectability. Further, substitution with heavy isotopes such as deuterium, i.e.,²H, produces certain therapeutic advantages resulting from greater metabolic stability, for example increased in vivo half-life or reduced dosage requirements. In some embodiments, the isotopically labeled compound or a pharmaceutically acceptable salt, solvate, or stereoisomer thereof is prepared by any suitable method.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions. As used herein, a “pharmaceutically acceptable salt” refers to any salt of a compound that is useful for therapeutic purposes of a subject.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds disclosed herein, or by separately reacting a purified compound in its free form with a suitable acid or base, and isolating the salt thus formed.

Examples of pharmaceutically acceptable salts include those salts prepared by reaction of the compounds described herein with a mineral acid, organic acid, or inorganic base, such salts including acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate, metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate, undeconate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid, metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3 -(4-hydroxybenzoyl)benzoic acid, cinnamic acid, mandelic acid, aryl sulfonic acid, methanesulfonic acid, ethanesulfonic acid, 1,2-ethanedi sulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene- 1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

In some embodiments, those compounds described herein which comprise a free acid group react with a suitable base, such as the hydroxide, carbonate, bicarbonate, or sulfate of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts, and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine, and the like. It should be understood that the compounds described herein also include the quaternization of any basic nitrogen-containing groups they contain. In some embodiments, water or oil-soluble or dispersible products are obtained by such quaternization.

### Solvates

In some embodiments, the compounds described herein exist as solvates. This disclosure provides for methods of treating diseases by administering such solvates. This disclosure further provides for methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein. Accordingly, one aspect of the present disclosure pertains to hydrates and solvates of compounds of the present disclosure and/or their pharmaceutical acceptable salts, as described herein, that can be isolated and characterized by methods known in the art, such as, thermogravimetric analysis (TGA), TGA-mass spectroscopy, TGA-Infrared spectroscopy, powder X-ray diffraction (PXRD), Karl Fisher titration, high resolution X-ray diffraction, and the like.

### Preparation of the Compounds

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, Pa.), Aldrich Chemical (Milwaukee, Wis., including Sigma Chemical and Fluka), Apin Chemicals Ltd. (Milton Park, UK), Avocado Research (Lancashire, U.K.), BDH, Inc. (Toronto, Canada), Bionet (Cornwall, U.K.), Chem Service Inc. (West Chester, Pa.), Crescent Chemical Co. (Hauppauge, N.Y.), Eastman Organic Chemicals, Eastman Kodak Company (Rochester, N.Y.), Fisher Scientific Co. (Pittsburgh, Pa.), Fisons Chemicals (Leicestershire, UK), Frontier Scientific (Logan, Utah), ICN Biomedicals, Inc. (Costa Mesa, Calif.), Key Organics (Cornwall, U.K.), Lancaster Synthesis (Windham, N.H.), Maybridge Chemical Co. Ltd. (Cornwall, U.K.), Parish Chemical Co. (Orem, Utah), Pfaltz & Bauer, Inc. (Waterbury, Conn.), Polyorganix (Houston, Tex.), Pierce Chemical Co. (Rockford, Ill.), Riedel de Haen AG (Hanover, Germany), Spectrum Quality Product, Inc. (New Brunswick, N.J.), TCI America (Portland, Oreg.), Trans World Chemicals, Inc. (Rockville, Md.), and Wako Chemicals USA, Inc. (Richmond, Va.).

Suitable reference books and treatises that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatises that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R.V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J.C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as on-line. Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

In one aspect, described herein is a method of making a conjugate that comprises a cyclic peptide, a metal chelator, optionally a linker, and optionally a radionuclide such as ¹⁷⁷Lu or 225Ac. In some embodiments, the conjugate is prepared by one or more of the following steps: (a) synthesizing the peptide sequence by solid phase peptide synthesis; (b) cyclizing the peptide by forming an intramolecular non-peptide bond; (c) coupling the metal chelator to the peptide; (d) and optionally labeling the conjugate with a radionuclide. In some embodiments, steps (a), (b), (c) and (d) are performed in the recited order. In some embodiments, synthesizing the peptide comprises synthesizing the peptide sequence in a protected form and performing a de-protecting reaction. In some embodiments, cyclizing the peptide comprises forming a non-peptide bond between the N-terminus and the C-terminus of the peptide. In some embodiments, cyclizing the peptide comprises forming a non-peptide bond between the N-terminus and a cysteine or homocysteine of the peptide. In some embodiments, cyclizing the peptide comprises forming a ring closing group selected from —C(═O)—CH₂—S—, —S—, —CH═CH—, —NH—, -maleimide-S—, —C(═O)—CH₂—NH—, and —C(═O)—CH₂—O—. In some embodiments, cyclizing the peptide comprises forming a ring closing group of Table 2A. In some embodiments, cyclizing the peptide comprises reacting a pair of functional groups or amino acids described in Table 2B. In some embodiments, solid phase peptide synthesis can be replaced with other suitable peptide synthesis methods known in the art.

### III. Pharmaceutical Compositions

The radiopharmaceutical conjugate described herein, including e.g., pharmaceutically acceptable salt or solvate thereof, can be administered per se as a pure chemical or as a component of a pharmaceutically acceptable formulation. In some embodiments, a conjugate described herein is combined with a pharmaceutically suitable or acceptable carrier selected on the basis of a chosen route of administration and standard pharmaceutical practice as described, for example, in Remington: The Science and Practice of Pharmacy (Gennaro, 21^{st} Ed. Mack Pub. Co., Easton, Pa. (2005)). Provided herein is a pharmaceutical composition comprising at least one conjugate described herein, or a stereoisomer, pharmaceutically acceptable salt, amide, ester, solvate, or N-oxide thereof, together with one or more pharmaceutically acceptable carriers. The carrier(s) (or excipient(s)) is acceptable or suitable if the carrier is compatible with the other ingredients of the composition and not deleterious to the recipient (i.e., the subject or patient) of the composition.

In one aspect, the disclosure provides a pharmaceutical composition comprising a herein described conjugate, or a pharmaceutically acceptable salt or solvate thereof, and a pharmaceutically acceptable excipient or carrier. In certain embodiments, the conjugate as described is substantially pure, in that it contains less than about 10%, less than about 5%, or less than about 1%, or less than about 0.1%, of other organic small molecules, such as unreacted intermediates or synthesis by-products that are created, for example, in one or more of the steps of a synthesis method.

Pharmaceutical compositions can include pharmaceutically acceptable carriers, diluents or excipients. Exemplary pharmaceutically acceptable carriers include solvents (aqueous or non-aqueous), solutions, emulsions, dispersion media, coatings, isotonic and absorption promoting or delaying agents, compatible with pharmaceutical administration. Such formulations can be contained in a liquid; emulsion, suspension, syrup or elixir, or solid form; tablet (coated or uncoated), capsule (hard or soft), powder, granule, crystal, or microbead. Supplementary components (e.g., preservatives, antibacterial, antiviral and antifungal agents) can also be incorporated into the compositions. Pharmaceutical compositions can be formulated to be compatible with a particular local or systemic route of administration. Thus, pharmaceutical compositions include carriers, diluents, or excipients suitable for administration by particular routes.

The compounds and pharmaceutical compositions of the current disclosure can be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. The term parenteral as used herein includes e.g., subcutaneous, intravenous, intramuscular, intrasternal, intraperitoneal, and infusion techniques. The term parenteral also includes injections, into the eye or ocular, intravitreal, intrabuccal, transdermal, intranasal, into the brain, including intracranial and intradural, into the joints, including ankles, knees, hips, shoulders, elbows, wrists, and the like, and in suppository form. In certain embodiments, the compounds and/or formulations are administered orally. In certain embodiments, the compounds and/or formulations are administered by systemic administration. In certain embodiments, the compounds and/or formulations are administered parenterally. In certain embodiments, the compounds and/or formulations are administered locally at a targeted site.

In some embodiments, conjugates, or pharmaceutically acceptable salts or solvates thereof, and pharmaceutical compositions described herein are administered via parenteral injection as liquid solution, which can include other chemical components, such as carriers, stabilizers, diluents, dispersing agents, suspending agents, thickening agents, preservatives, or excipients. Parenteral injections can be formulated for bolus injection or continuous infusion. The pharmaceutical compositions can be in a form suitable for parenteral injection as a sterile suspension, solution or emulsion in oily or aqueous vehicles, and can contain formulatory agents such as suspending, stabilizing or dispersing agents. Pharmaceutical formulations for parenteral administration include aqueous solutions of the active compounds in water soluble form. Solutions or suspensions used for parenteral, intradermal, or subcutaneous application can include: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid, gentisic acid, or sodium bisulfite; chelating agents such as ethylenediaminetetraacetic acid; buffers such as acetates, citrates or phosphates; surfactants such as polysorbate 80; and agents for the adjustment of tonicity such as sodium chloride or dextrose. pH can be adjusted with acids or bases, such as hydrochloric acid or sodium hydroxide. In some embodiments, the pharmaceutical composition comprises a reductant. The presence of a reductant can help minimize potential radiolysis. In some embodiments, the reductant is ascorbic acid, gentisic acid, sodium thiosulfate, citric acid, tartaric acid, or a combination thereof.

Pharmaceutical compositions comprising the conjugates or pharmaceutically acceptable salts or solvates thereof described herein can be prepared according to standard techniques and further comprise a pharmaceutically acceptable carrier. In some embodiments, normal saline can be employed as the pharmaceutically acceptable carrier. Other suitable carriers include, e.g., water, buffered water, 0.9% isotonic saline, 0.4% saline, 0.3% glycine, and the like, including glycoproteins for enhanced stability, such as albumin, lipoprotein, globulin, etc. These compositions can be sterilized by conventional sterilization techniques. The resulting aqueous solutions may be packaged for use or filtered under aseptic conditions and lyophilized. In some embodiments, the lyophilized preparation is combined with a sterile aqueous solution prior to administration. The compositions can contain pharmaceutically acceptable auxiliary substances as appropriate to approximate physiological conditions, such as pH adjusting and buffering agents, tonicity adjusting agents and the like, for example, sodium acetate, sodium lactate, sodium chloride, potassium chloride, calcium chloride, sodium lactate, sorbitan monolaurate, triethanolamine oleate, etc. Pharmaceutical compositions can be selected according to their physical characteristic, including, but not limited to fluid volumes, viscosities and other parameters in accordance with the particular mode of administration selected. The amount of conjugates administered can depend upon the particular targeting moiety used, the disease state being treated, the therapeutic agent being delivered, and the judgment of the clinician.

The concentration of the conjugates or pharmaceutically acceptable salts or solvates thereof described herein in the pharmaceutical formulations can vary. In some embodiments, the conjugate is present in the pharmaceutical composition from about 0.05% to about 1% by weight, about 1% to about 2% by weight, about 2% to about 5% by weight, about 5% to about 10% by weight, about 10% to about 30% by weight, about 30% to about 50% by weight, about 50% to about 75% by weight, or about 75% to about 99% by weight.

Pharmaceutical compositions are administered in a manner appropriate to the disease to be treated. An appropriate dose and a suitable duration and frequency of administration will be determined by such factors as the condition of the subject, the type and severity of the subject's disease, the particular form of the active ingredient, and the method of administration. In some embodiments, an appropriate dose and treatment regimen provides the composition(s) in an amount sufficient to provide therapeutic and/or prophylactic benefit (e.g., an improved clinical outcome), or a lessening of symptom severity. Optimal doses are generally determined using experimental models and/or clinical trials. The optimal dose depends upon the body mass, weight, or blood volume of the subject.

The amount of conjugates or pharmaceutically acceptable salts or solvates thereof and/or pharmaceutical compositions administered can be sufficient to deliver a therapeutically effective dose of the particular subject. In some embodiments, conjugate dosages can be between about 0.1 pg and about 50 mg per kilogram of body weight, 1 μg and about 50 mg per kilogram of body weight, or between about 0.1 and about 10 mg/kg of body weight. Therapeutically effective dosages can also be determined at the discretion of a physician. By way of example only, the dose of the conjugate or a pharmaceutically acceptable salt or solvate thereof described herein for methods of treating a disease as described herein is about 0.001 mg/kg to about 1 mg/kg body weight of the subject per dose. In some embodiments, the dose of conjugate or a pharmaceutically acceptable salt or solvate thereof described herein for the described methods is about 0.001 mg to about 1000 mg per dose for the subject being treated. In some embodiments, a conjugate or a pharmaceutically acceptable salt or solvate thereof described herein is administered to a subject at a dosage of from about 0.01 mg to about 500 mg, from about 0.01 mg to about 100 mg, or from about 0.01mg to about 50 mg. In some embodiments, a conjugate or a pharmaceutically acceptable salt or solvate thereof described herein is administered to a subject at a dosage of about 0.01 picomole to about 1 mole, about 0.1 picomole to about 0.1 mole, about 1 nanomole to about 0.1 mole, or about 0.01 micromole to about 0.1 millimole. In some embodiments, a conjugate or a pharmaceutically acceptable salt or solvate thereof described herein is administered to a subject at a dosage of about 0.01 Gbq to about 1000 Gbq, about 0.5 Gbq to about 100 Gbq, or about 1 Gbq to about 50 Gbq. In some embodiments, the dose is administered once a day, 1 to 3 times a week, 1 to 4 times a month, or 1 to 12 times a year.

The pharmaceutical formulations can be packaged in unit dosage form for ease of administration and uniformity of dosage. A unit dosage form can refer to physically discrete units suited as unitary dosages for the subject to be treated; each unit containing a predetermined quantity of active compound calculated to produce the desired therapeutic effect in association with the pharmaceutical carrier or excipient.

### IV. Method of Treatment

In one aspect, the disclosure provides methods of treating a disease or condition in a subject in need thereof. In some embodiments, the methods comprise administering a conjugate or a pharmaceutically acceptable salt or solvate thereof described herein, or a pharmaceutical composition comprising the same to the subject in need thereof. In some embodiments, provided herein is a method of providing a therapeutic and/or prophylactic benefit to a subject in need thereof comprising administering a compound or pharmaceutical composition described herein.

In some embodiments, the methods comprise administering to a subject a therapeutically effective amount of a conjugate or a pharmaceutically acceptable salt or solvate thereof. In some embodiments, the conjugate or pharmaceutically acceptable salt or solvate thereof is administered in a pharmaceutical composition. In some embodiments, the subject has cancer. In some embodiments, the cancer is a solid tumor or hematological cancer.

In some embodiments, provided herein are methods for killing a cell comprising contacting the cell with a conjugate or a pharmaceutically acceptable salt or solvate thereof. In some embodiments, the cell expresses a receptor described herein, e.g., PSMA. In some embodiments, the conjugate or pharmaceutically acceptable salt or solvate thereof binds to a structure on the cell. In some embodiments, the conjugate or pharmaceutically acceptable salt or solvate thereof releases a number of alpha particles by natural radioactive decay. In some embodiments, the conjugate or pharmaceutically acceptable salt or solvate thereof releases a number of beta particles, gamma rays, and/or Auger electrons by natural radioactive decay. The conjugate described herein can kill a cell by radiation. In some embodiments, the conjugate kills the cell directly by radiation. In some embodiments, the radiation creates, in the cell, oxidized bases, abasic sites, single-stranded breaks, double-stranded breaks, DNA crosslink, chromosomal rearrangement, or a combination thereof. In some embodiments, the conjugate kills the cell by inducing double-stranded DNA breaks. In some embodiments, the released alpha particles are sufficient to kill the cell. In some embodiments, the released alpha particles are sufficient to stop cell growth. In some embodiments, the conjugate kills the cell indirectly via the production of reactive oxygen species (ROS) such as free hydroxyl radicals. In some embodiments, the conjugate kills the cell indirectly by releasing tumor antigens from one or more different cells, which can have vaccine effect. In some embodiments, the conjugate kills the cell by abscopal effect. In some embodiments, the cell is a cancer cell. In some embodiments, the method comprises killing a cell with an alpha-particle emitting radionuclide.

After contacting a cell, the described conjugate can be internalized by the cell. The internalization can be mediated by cell receptors, cell membrane endocytosis, etc. In some embodiments, rapid internalization rate into cancer cells accompanied by a slow externalization rate can offer therapeutic benefit.

In one aspect, the disclosed conjugate or a pharmaceutically acceptable salt or solvate thereof is configured to treat cancer by ablating tumor cells. In some embodiments, the conjugate or a pharmaceutically acceptable salt or solvate thereof does not modulate the biology of the tumor cell and/or the surrounding stroma. In some embodiments, the conjugate or a pharmaceutically acceptable salt or solvate thereof does not modulate immune cells. In some embodiments, the ablating of tumor cells can lead to a downstream immunological cascade.

Non-limiting examples of cancers to be treated by the methods of the present disclosure can include melanoma (e.g., metastatic malignant melanoma), renal cancer (e.g., clear cell carcinoma), prostate cancer (e.g., hormone refractory prostate adenocarcinoma), pancreatic adenocarcinoma, breast cancer, colon cancer, lung cancer (e.g., non-small cell lung cancer), esophageal cancer, squamous cell carcinoma of the head and neck, liver cancer, ovarian cancer, cervical cancer, thyroid cancer, glioblastoma, glioma, leukemia, lymphoma, and other neoplastic malignancies. In some embodiments, a subject or population of subj ects to be treated with a pharmaceutical composition of the present disclosure have a solid tumor. In some embodiments, a solid tumor is a melanoma, renal cell carcinoma, lung cancer, bladder cancer, breast cancer, cervical cancer, colon cancer, gall bladder cancer, laryngeal cancer, liver cancer, thyroid cancer, stomach cancer, salivary gland cancer, prostate cancer, pancreatic cancer, or Merkel cell carcinoma. In some embodiments, a subject or population of subjects to be treated with a pharmaceutical composition of the present disclosure have a hematological cancer. In some embodiments, the subject has a hematological cancer such as Diffuse large B cell lymphoma (“DLBCL”), Hodgkin's lymphoma (“HL”), Non-Hodgkin's lymphoma (“NHL”), Follicular lymphoma (“FL”), acute myeloid leukemia (“AML”), or Multiple myeloma (“MM”). In some embodiments, a subject or population of subjects to be treated having the cancer selected from the group consisting of ovarian cancer, lung cancer and melanoma.

In some embodiments, provided herein are methods and compositions for treating a disease or condition. Exemplary disease or condition includes refractory or recurrent malignancies whose growth may be inhibited using the methods of treatment of the present disclosure. In some embodiments, the disease or condition is a cancer. In some embodiments, the cancer is breast cancer, head and neck squamous cell carcinoma, non-small cell lung cancer, hepatocellular cancer, colorectal cancer, gastric adenocarcinoma, melanoma, or advanced cancer. In some embodiments, a cancer to be treated by the methods of treatment of the present disclosure is selected from the group consisting of carcinoma, squamous carcinoma, adenocarcinoma, sarcomata, endometrial cancer, breast cancer, ovarian cancer, cervical cancer, fallopian tube cancer, primary peritoneal cancer, colon cancer, colorectal cancer, squamous cell carcinoma of the anogenital region, melanoma, renal cell carcinoma, lung cancer, non-small cell lung cancer, squamous cell carcinoma of the lung, stomach cancer, bladder cancer, gall bladder cancer, liver cancer, thyroid cancer, laryngeal cancer, salivary gland cancer, esophageal cancer, head and neck cancer, glioblastoma, glioma, squamous cell carcinoma of the head and neck, prostate cancer, pancreatic cancer, mesothelioma, sarcoma, hematological cancer, leukemia, lymphoma, neuroma, and combinations thereof. In some embodiments, a cancer to be treated by the methods of the present disclosure include, for example, carcinoma, squamous carcinoma (for example, cervical canal, eyelid, tunica conjunctiva, vagina, lung, oral cavity, skin, urinary bladder, tongue, larynx, and gullet), and adenocarcinoma (for example, prostate, small intestine, endometrium, cervical canal, large intestine, lung, pancreas, gullet, rectum, uterus, stomach, mammary gland, and ovary). In some embodiments, a cancer to be treated by the methods of the present disclosure further include sarcomata (for example, myogenic sarcoma), leukosis, neuroma, melanoma, and lymphoma. In some embodiments, a cancer to be treated by the methods of the present disclosure is breast cancer. In some embodiments, a cancer to be treated by the methods of treatment of the present disclosure is triple negative breast cancer (TNBC). In some embodiments, a cancer to be treated by the methods of treatment of the present disclosure is pancreatic cancer.

In addition to the methods of treatment described above, the compounds and compositions described herein can be used to image, and/or as part of a treatment for diseases. Conjugates for imaging applications, e.g., single-photon emission computed tomography (SPECT) and positron emission tomography (PET), can comprise a radionuclide suitable for use as imaging isotopes such as the isotopes in Table 5B. Accordingly, the conjugate can be administered as a companion diagnostic.

### Combination Therapy

In some embodiments, a conjugate described herein can be administered alone or in combination with one or more additional therapeutic agents. For example, the combination therapy can include a composition comprising a conjugate described herein co-formulated with, and/or co-administered with, one or more additional therapeutic agents, e.g., one or more anti-cancer agents, e.g., cytotoxic or cytostatic agents, immune checkpoint inhibitors, hormone treatment, vaccines, and/or immunotherapies. In some embodiments, the conjugate is administered in combination with other therapeutic treatment modalities, including surgery, cryosurgery, and/or chemotherapy. Such combination therapies may advantageously utilize lower dosages of the administered therapeutic agents, thus avoiding possible toxicities or complications associated with the various monotherapies.

When administered in combination, two (or more) different treatments can be delivered to the subject during the course of the subject's affliction with the disorder, e.g., the two or more treatments are delivered after the subject has been diagnosed with the disorder and before the disorder has been cured or eliminated. In some embodiments, the delivery of one treatment is still occurring when the delivery of the second begins, so that there is overlap. This is sometimes referred to herein as “simultaneous” or “concurrent delivery.” In some embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In some embodiments, the herein-described conjugate is used in combination with a chemotherapeutic agent, e.g., a DNA damaging chemotherapeutic agent. Non-limiting examples of DNA damaging chemotherapeutic agents include topoisomerase I inhibitors, topoisomerase II inhibitors; alkylating agents; DNA intercalators; DNA intercalators and free radical generators such as bleomycin; and nucleoside mimetics. In some embodiments, the herein-described conjugate is used in combination with a radiation sensitizer, which makes tumor cells more sensitive to radiation therapy. In some embodiments, the herein-described conjugate is used in combination with a DNA damage repair inhibitor (or DNA damage response (DDR) inhibitor).

In some embodiments, the subject is 4 to 100 years old. In some embodiments, the subject is 5 to 10, 5 to 15, 5 to 18, 5 to 25, 5 to 35, 5 to 45, 5 to 55, 5 to 65, 5 to 75, 10 to 15, 10 to 18, 10 to 25, 10 to 35, 10 to 45, 10 to 55, 10 to 65, 10 to 75, 15 to 18, 15 to 25, 15 to 35, 15 to 45, 15 to 55, 15 to 65, 15 to 75, 18 to 25, 18 to 35, 18 to 45, 18 to 55, 18 to 65, 18 to 75, 25 to 35, 25 to 45, 25 to 55, 25 to 65, 25 to 75, 35 to 45, 35 to 55, 35 to 65, 35 to 75, 45 to 55, 45 to 65, 45 to 75, 55 to 65, 55 to 75, or 65 to 75 years old. In some embodiments, the subject is at least 5, 10, 15, 18, 25, 35, 45, 55, or 65 years old. In some embodiments, the subject is at most 10, 15, 18, 25, 35, 45, 55, 65, or 75 years old.

Although the present disclosure and its advantages have been described in detail, it should be understood that various changes, substitutions and alterations can be made herein without departing from the spirit and scope of the disclosure as defined in the appended claims.

The present disclosure is further illustrated in the following Examples which are given for illustration purposes only and are not intended to limit the disclosure in any way.

### EXAMPLES

### A: Synthesis of the Compounds

### Example A1

### Peptide and Conjugate Synthesis

Linear precursors of macrocyclic peptides are prepared with an amidated C-terminus by standard Fmoc-solid phase synthesis. Its N-terminus is chloroacetylated. The peptides are cleaved from the resin by incubation with a trifluoroacetic acid (TFA) cleavage cocktail (TFA/2,2′-(ethylenedioxy)diethanethiol/triisopropyl silane/H₂O (92.5:2.5:2.5:2.5)) before filtration, concentration in vacuo and precipitation with ice-cold diethyl ether. Crude peptides are washed with diethyl ether, dried and resuspended in DMSO. The pH is raised to >8 using triethylamine to allow cyclization. The cyclized peptides are purified by HPLC.

The peptide is coupled with a metal chelator DOTA and then a radionuclide or a surrogate is chelated with DOTA, thereby producing the conjugate.

### Example A2

### Analytical Conditions

High performance liquid chromatography (HPLC) analyses are performed on an Agilent 1260 series equipped with a binary pump G7112A, micro vacuum degasser, standard autosampler ALS G7129A, thermostatted column compartment TCC G7116A, variable wavelength detector VWD G7114A, and data are analyzed by OpenLab CDS 2.2 network workstation software from Agilent Technologies. HPLC solvents consisted of H₂O containing 0.1% trifluoroacetic acid (mobile phase A) and acetonitrile containing 0.075% trifluoroacetic acid (mobile phase B). Conditions: a Phenomenex Gemini-NX C-18 (5 μm, 110 Å, 4.6×250 mm) column is used with a flow rate of 1.0 mL/min.

LCMS analysis was performed on an Agilent 1260 Infinity II HPLC and MSD (G6135B) system using the following conditions: Luna C18(2)-HST column (reverse phase, 50 mm×3 mm, 2.5 μm); Column temperature of 40° C.; Mobile phase A: water doped with 0.05% TFA, mobile phase B: acetonitrile doped with 0.0375% TFA; linear gradient from 95:5 to 5:95 mobile phase A:B—gradient was held at 95:5 (mobile phase A:B) for 1 min, followed by a linear gradient to 5:95 (A:B) over 8 min, then held at 5:95 (A:B) for 3 min; flow rate: 0.6 mL/min; UV photodiode array detection.

### Example A3

### Synthesis of C-40 and Lu-C-40

Conjugate C-40 contains a monocyclic peptide P-40 of Table 3.

Solid phase peptide synthesis (SPPS) was performed in a standard manual reaction vessel under nitrogen. Rink Amide-MBHA resin was purchased from Sunresin New Materials Co. (China) . Fmoc protected amino acids were purchased from GL Biochem (China). HBTU and HATU were purchased from Highfine Biotech Co. (China). Piperidine was purchased from Damao Chemical Reagent Factory (China). The peptides and their derivatives were purified on a Gilson GX-281 preparative HPLC system using reverse-phase C18 columns (Gemini, 5 μm, 110 Å+luna, 10 μm, 100 Å) at 30° C. HPLC solvents consisted of H₂O containing 0.075% trifluoroacetic acid (mobile phase A) and acetonitrile (mobile phase B).

### Example A3-1

### Synthesis of Peptidyl-Resin 1 (SEQ ID NO: 15)

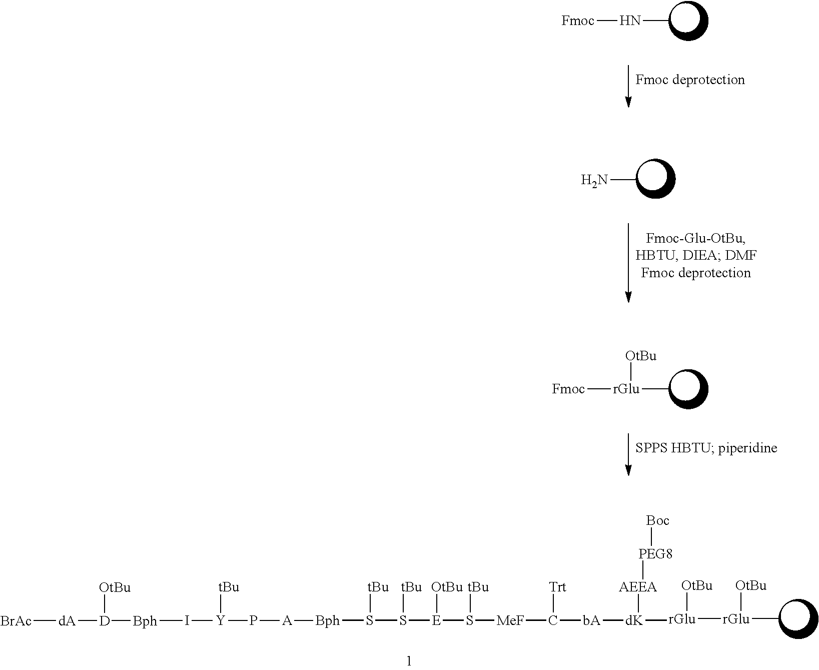

To the swollen Rink Amide-MBHA Resin (0.30 mmol, 0.32 mmol/g, 1.00 equiv) Fmoc was removed via 20 min agitation with 20% piperidine in DMF followed by filtration and washing. Then the resin was added Fmoc-Glu-OtBu (0.39 g, 0.9 mmol) HBTU (0.32g, 0.85 mmol) and DIEA (0.23g, 1.8 mmol) in dry DMF. The mixture was agitated for 30 min under nitrogen. After the reaction solution was removed through filtration, the resin was washed three times with DMF (10 mL). The Fmoc protecting group was removed via 30 min agitation with 20% piperidine in DMF followed by filtration and washing.

Subsequent amino acids were coupled using Fmoc-protected amino acid (3.00 equiv), HBTU (2.85 equiv) and DIEA (6.00 equiv) in dry DMF, shaking for 30 min. Pre-activation of any amino acid was not performed prior to coupling. Between amino acid couplings, the Fmoc protecting group was removed via 30 min agitation with 20% piperidine in DMF followed by filtration and washing. Success of Fmoc removal steps and amino acid couplings were monitored qualitatively using a ninhydrin test.

### Example A3-2

### Synthesis of Lu-C-40. Image Discloses SEQ ID NOS 15-17, 18, and 18, Respectively, in Order of Appearance

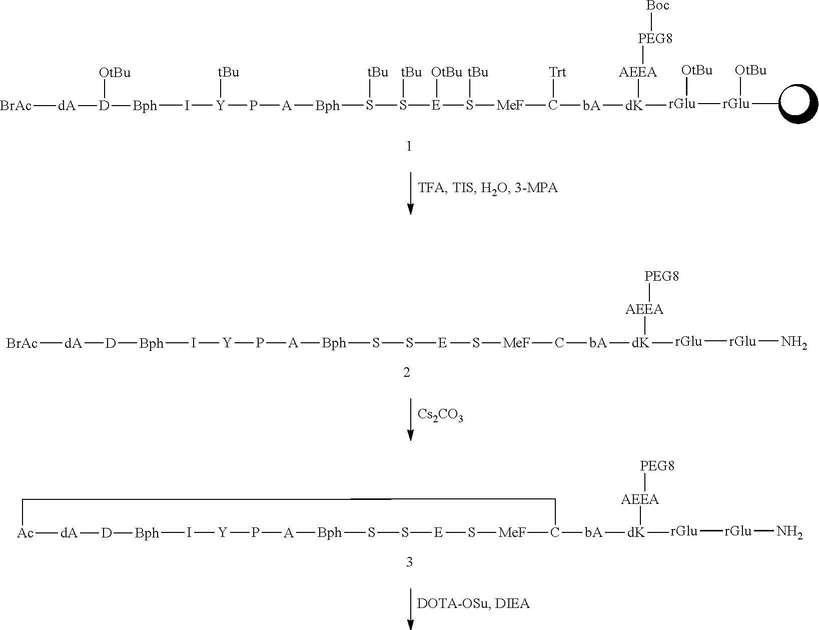

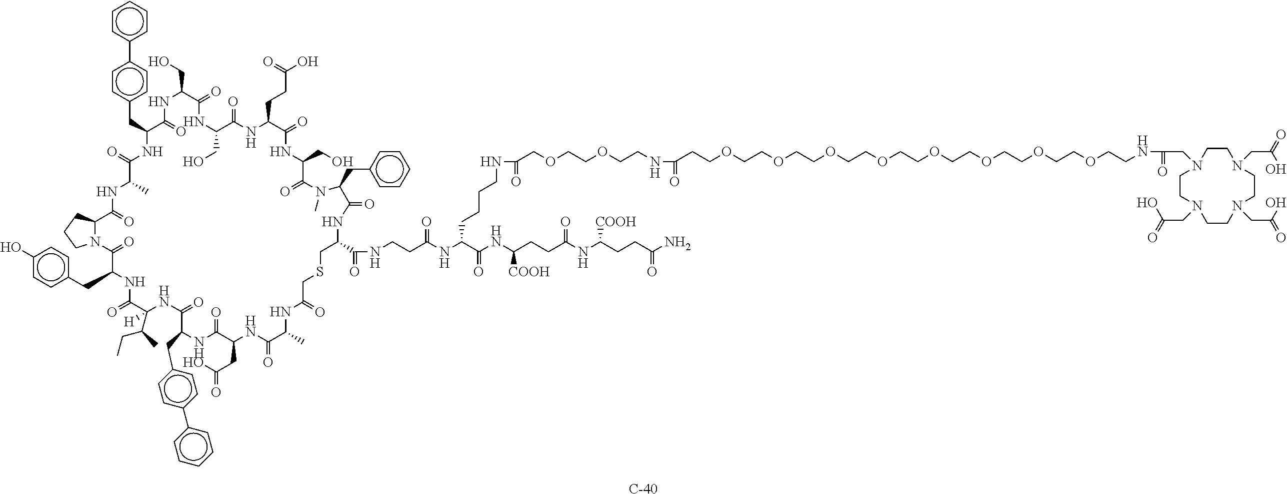

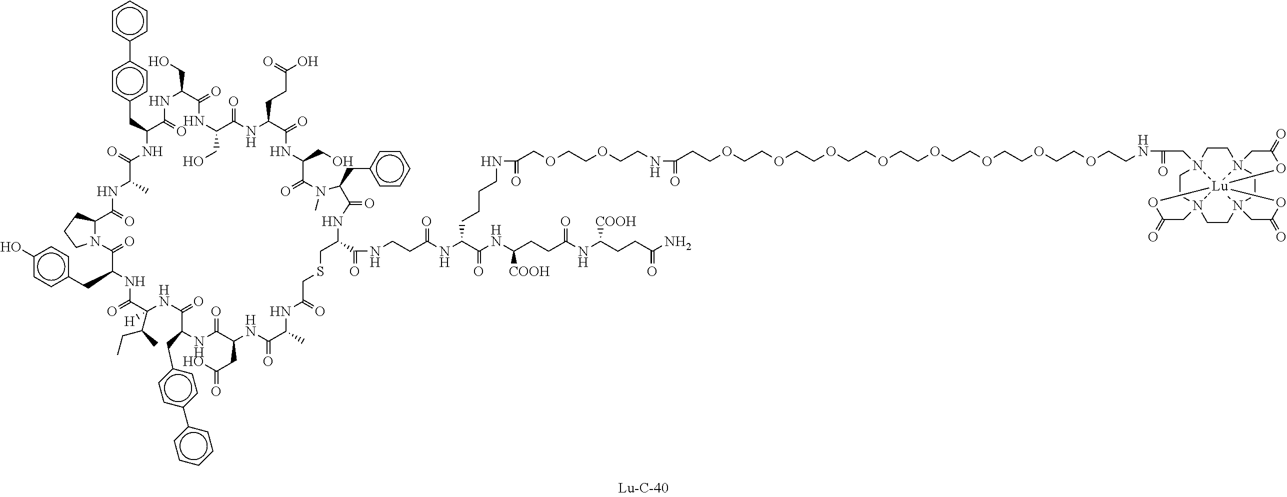

After the resin was washed three times with MeOH and dried under vacuum, a cocktail of trifluoroacetic acid/H₂O/triisopropyl silane/3-mercaptopropionic acid (90:2.5:2.5:5.0) was added. The resulting mixture was stirred for 2 h at room temperature. Cold isopropyl ether was added. The precipitated crude linear peptide-2 was collected through filtration and dried under vacuum.

Cyclization of peptide-2: To a solution of crude 2 (0.82 g) in water (250 mL) and MeCN (150 mL) were added Cs₂CO₃ (3.0 eq). The resulting mixture was stirred at room temperature for 0.5 hour. The pH of the solution was then adjusted to 5.0 using 1.0 N HCl. After lyophilization, the crude was purified by preparative HPLC to afford peptide-3 (120 mg) as a white solid.

To a solution of peptide-3 (120 mg, 1.0 eq) in DMF (6 mL) were added DOTA-OSu (1.5 eq), DIEA (3.0 eq). The resulting mixture was stirred at room temperature for 0.5 hour, the crude was purified by preparative HPLC to afford C-40 (78 mg, 95.05% purity) as a white solid.

Lu³⁺ complexation: To a solution of C-40 (60 mg, 95.05% purity, 1.0 eq) in H₂O (2 mL) and MeCN (1 mL) was added LuCl₃ (26.3 mg, 5.0 eq) and then 1M Na₂CO₃ aqueous solution was adjusted to pH=5˜6. The resulting mixture was stirred at 40° C. for 1 h. After filtration, the crude product was purified by preparative HPLC to afford Lu-C-40 (35.8 mg, 96.44% purity) as a white solid.

### Example A4

### Procedures for Cyclic Peptide Cyclization

Example A4-1

### α-Halocarbonyl-Cysteine Cyclization in Solution

In a typical procedure, a linear peptide of the general formula Cl—CH₂—C(═O)—NH-XaaN-Xaa2-Xaa3-Xaa4-(Xaa0)p-Xaa12-XaaC-CH₂—SH is dissolved in water/acetonitrile. The mixture is adjusted to pH 8 with ammonia solution and typically stirred for 16 hours. After work-up, the crude peptide of general formula cyclo[CH₂—C(═O)—NH-XaaN-Xaa2-Xaa3-Xaa4-(Xaa0)p-Xaa13-XaaC-CH₂—S] is obtained. If this peptide is fully protected, then standard treatment with a suitable TFA cleavage cocktail follows. Finally, the resulting crude is purified by HPLC (e.g. C18 column; ISCO ACCQ; 3 to 60% acetonitrile in water, using 0.1% TFA as modifier). This procedure showcases a head-to-tail cyclization; however, side chain cyclizations (staples) can be introduced with careful planning of addition and removal of protecting groups. This procedure can be applied to homologous analogs of cysteine and lysine.

Conjugates comprising cyclic peptides of Table 3 were synthesized and complexed with Lu according to Examples A1 and A3, and cyclized according to the method described in Example A4-1. LCMS characterization of select conjugates is provided in Table 6 below.

TABLE 6
LCMS data for select Lu complexed conjugates disclosed herein. Table
discloses SEQ ID NOS 19-24, respectively, in order of appearance.

| | | C18 | | |
|---|---|---|---|---|
| | | Retention | Mass | |
| Compound | Compound | Time | Expected | Mass |
| No. | Structure | (min) | [M/2 + 2H]+ | Observed |
| Lu-C-11 | 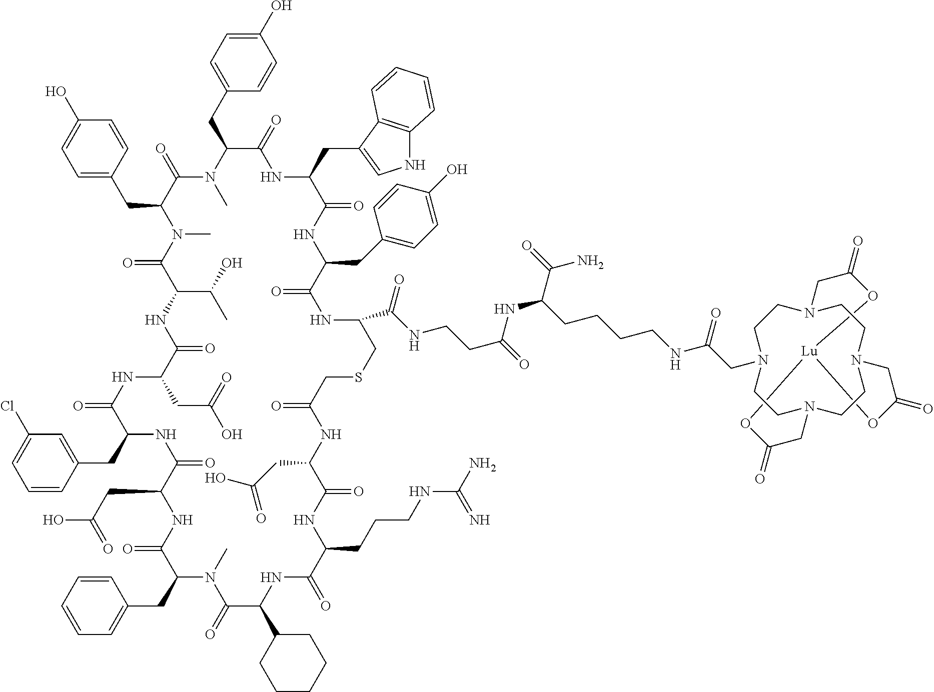 | 6.12 | 1354.0 | 1353.6 |
| Lu-C-12 | 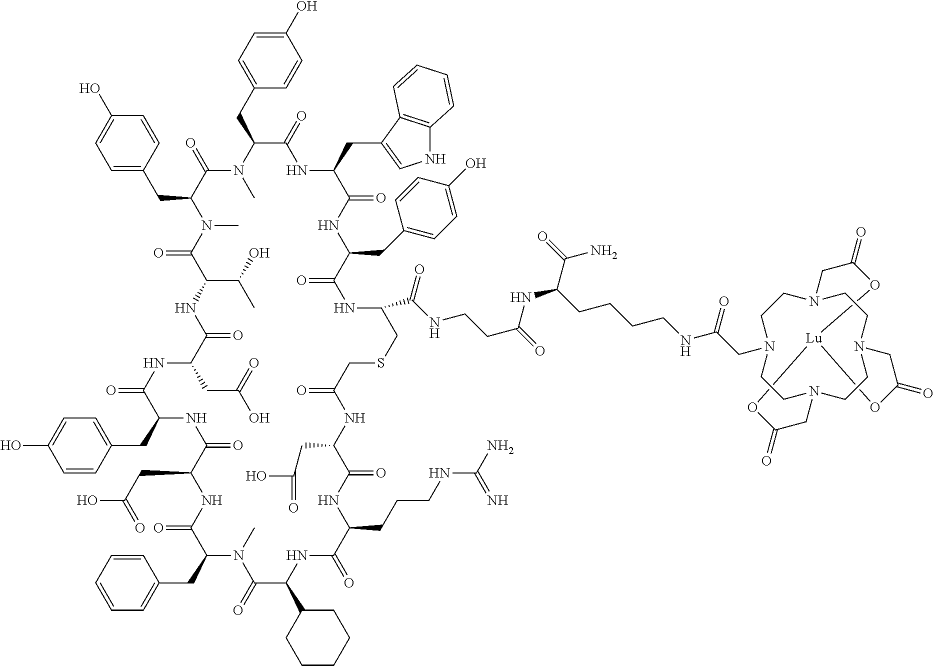 | 5.62 | 1345.0 | 1344.8 |
| Lu-C-21 | 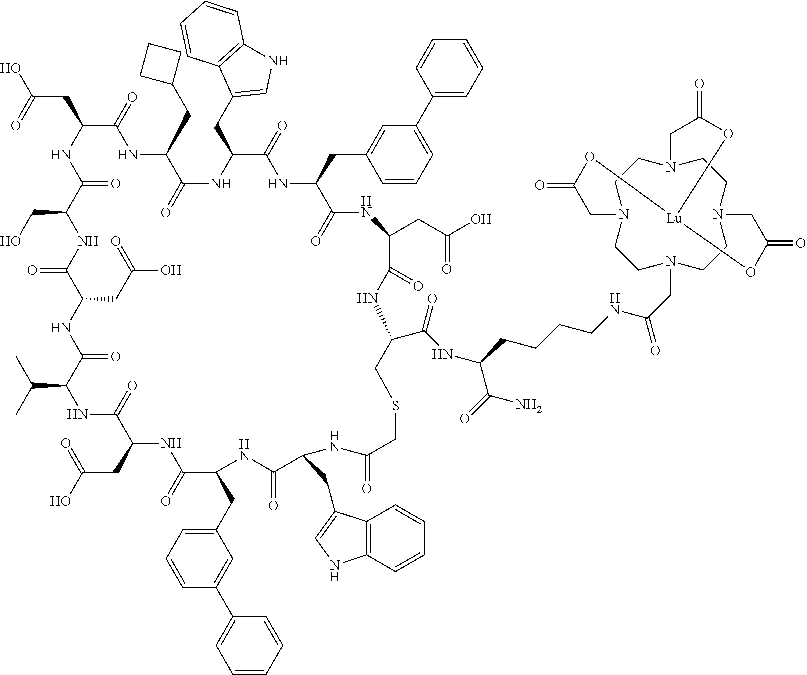 | 6.29 | 1219.9 | 1219.4 |
| Lu-C-30 | 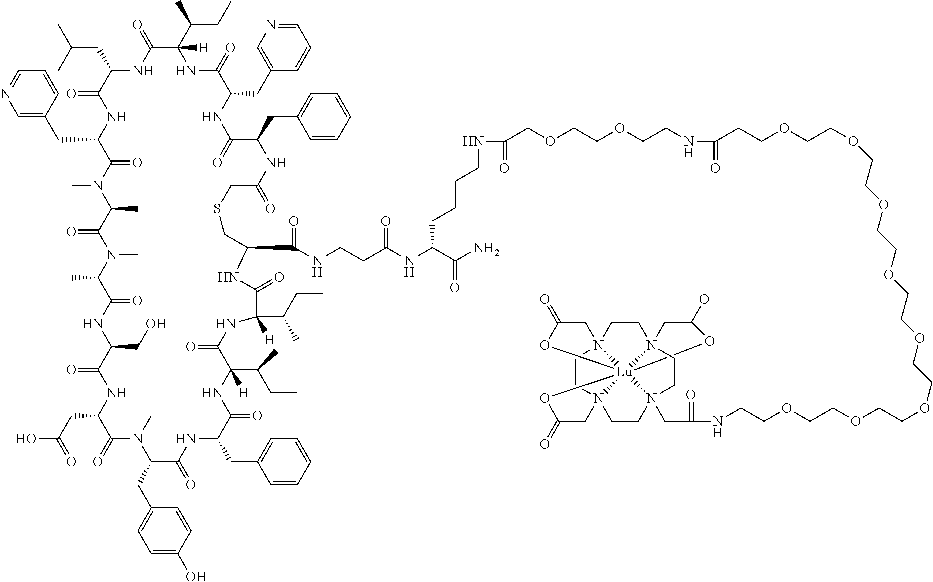 | 5.16 | 1540.7 | 1540.8 |
| Lu-C-31 | 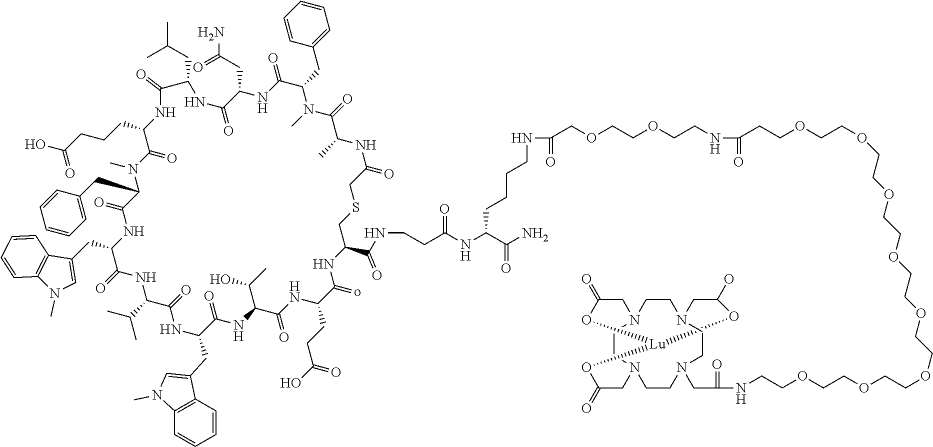 | 6.45 | 1491.2 | 1491.0 |
| Lu-C-41 | 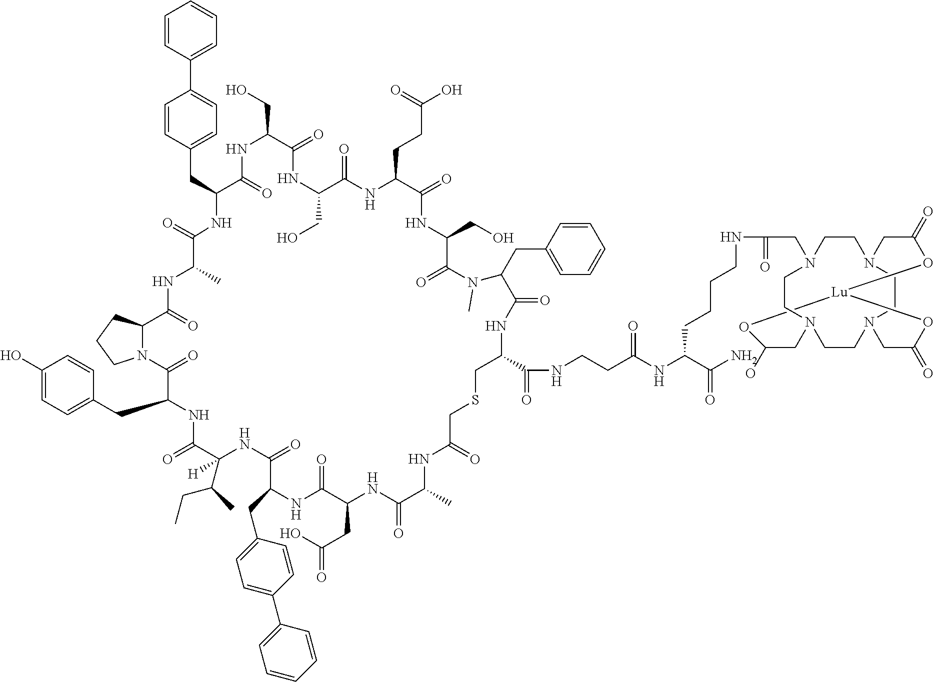 | 6.12 | 1274.5 | 1274.0 |

### Example A4-2

### α-Halocarbonyl-Homocysteine Cyclization in Solution

A typical procedure is virtually the same as the cyclization of α-halocarbonyl-cysteine, however the C-terminal cysteine is substituted for a homocysteine.

### Example A4-3

### Macrolactamization on Solid Phase

In a typical procedure, a linear peptide of the general formula H-XaaN-Xaa2-Xaa3-Xaa4-(Xaa0)p-Xaa13-XaaC-(Wang resin)-OH (100 mg, 0.25 mmol/g) is vortexed at room temperature for 4 h with a solution of TBTU (or other coupling reagent of choice; 1 eqmol) and DIEA (or other base of choice; 2 eqmol) in DMF (1 ml). After cyclization, the resin is washed with DMF (3×2 min) and DCM (2×2 min). After cleavage from the Wang resin via a suitable TFA cleavage cocktail, the resulting crude cyclic peptide product is purified by HPLC (e.g. C18 column; ISCO ACCQ; 3 to 60% acetonitrile in water, using 0.1% TFA as modifier). This procedure showcases a head-to-tail macrocyclization; however, side chain lactamizations (staples) can be introduced with careful planning of addition and removal of protecting groups. This procedure can be applied to homologous analogs of aspartic acid and lysine.
In some cases, this procedure is more suitable for cyclic peptides having 7 or more amino acids. In some cases, careful selection of cyclization reagents and conditions are needed for cyclic peptides having less than 7 amino acids.

### Example A4-4

### Macrolactamization in Solution

In a typical procedure, a linear peptide of the general formula H-XaaN-Xaa2-Xaa3-Xaa4-(Xaa0)p-Xaa13-XaaC-OH is dissolved in DMF in a concentration ranging from 0.01 mM to 20 mM, depending on the specific sequence. The side chains of the linear peptide are masked by protective groups. To the solution, is added DIPEA (or other base of choice; 2.1 eqmol) and EDCI (or other coupling reagent of choice; 1.0 eqmol). The resulting mixture is stirred at RT (or at the temperature of choice), and upon completion the DMF is concentrated by rotatory evaporation. The resulting crude material is optionally dissolved in DCM (or EtOAc), and the DCM (orEtOAc) solution is washed with water. The DCM (or EtOAc) layer is concentrated and dried under high vacuum. The resulting crude cyclic peptide product obtained with or without work-up is purified by HPLC (e.g. C18 column; ISCO ACCQ; 3 to 60% acetonitrile in water, using 0.1% TFA as modifier). This procedure showcases a head-to-tail macrocyclization; however, side chain lactamizations (staples) can be introduced with careful planning of addition and removal of protecting groups. This procedure can be applied to homologous analogs of aspartic acid and lysine.
In some cases, this procedure is more suitable for cyclic peptides having 7 or more amino acids. In some cases, careful selection of cyclization reagents and conditions are needed for cyclic peptides having less than 7 amino acids.

### Example A4-5

### Thioether Macrolactamization on Solid Phase

In a typical procedure, a linear peptide of the general formula Fmoc-[(ClCH₂CH₂)-XaaN]-Xaa2-Xaa3-Xaa4-(Xaa0)p-Xaa13- XaaC-CH₂—SH-(resin) (100 mg, 0.25 mmol/g) is vortexed at room temperature with continuous shaking in 0.1 M NaHCO3 in DMF/H2O (3:2) for 72 h to complete the cyclization. The side chains of the linear peptide are masked by protective groups. After cleavage from the resin via a suitable TFA cleavage cocktail, the resulting crude cyclic peptide product the is purified by HPLC (e.g. C18 column; ISCO ACCQ; 3 to 60% acetonitrile in water, using 0.1% TFA as modifier). This procedure showcases a head-to-tail cyclization; however, side chain cyclizations (staples) can be introduced with careful planning of addition and removal of protecting groups. This procedure can be applied to homologous analogs of cysteine and Cl-homoalanine.

### Example A4-6

### Thioether Macrolactamization in Solution

In a typical procedure, a linear peptide of the general formula Fmoc-[(ClCH₂CH₂)-XaaN]-Xaa2-Xaa3-Xaa4-(Xaa0)p-Xaa13-XaaC-CH₂—SH is vortexed at room temperature with continuous shaking in a 1:1 mixture of acetonitrile and water, containing 1 mg/mL of chloropeptide and 10 mg/mL of NaHCO₃ to complete cyclization. The side chains of the linear peptide are unmasked or masked by protective groups. When masked, the use of an additional organic cosolvent (e.g. THF, DMF, etc.) might be needed. The removal of protective groups is achieved by standard treatment with a suitable TFA cleavage cocktail, if needed. The resulting crude cyclic peptide product is purified by HPLC (e.g. C18 column; ISCO ACCQ; 3 to 95% acetonitrile in water, using 0.1% TFA as modifier). This procedure showcases a head-to-tail cyclization; however, side chain cyclizations (staples) can be introduced with careful planning of addition and removal of protecting groups. This procedure can be applied to homologous analogs of cysteine and Cl-homoalanine.

### Example A4-7

### Reductive Amination Macrocyclization in Solution

In a typical procedure, a linear glycinal peptide of the general formula H-XaaN-Xaa2-Xaa3-Xaa4-(Xaa0)p-Xaa13-XaaC-CH₂—CHO is dissolved to a concentration of 1 mM in NaOAc/AcOH buffer (0.4 M, pH=5.5, or other optimal pH), and NaBH3CN (10 eqmol) is added. The side chains of the linear peptide are unmasked or masked by protective groups. When masked, the used of an organic cosolvent (e.g. THF, DMF, etc.) might be needed. The resulting solution is stirred at room temperature to completion. When needed, the removal of protective groups is achieved by standard treatment with a suitable TFA cleavage cocktail. The reaction is purified in appropriate aliquots by HPLC (e.g. C18 column; ISCO ACCQ; 3 to 95% acetonitrile in water, using 0.1% TFA as modifier) to afford the peptide macrocycle. This procedure can be applied to homologous analogs of formylglycine.

### Example A4-8

### Strecker Macrocyclization in Solution

In a typical procedure, a linear glycinal peptide of the general formula H-XaaN-Xaa2-Xaa3-Xaa4-(Xaa0)p-Xaa13-XaaC-CH₂—CHO is dissolved in water to a concentration of 1 mM, and KCN (1.2 eqmol) is added. The side chains of the linear peptide are unmasked or masked by protective groups. When masked, the used of an organic cosolvent (e.g. THF, DMF, etc.) might be needed. The resulting solution is stirred at room temperature to completion. When needed, the removal of protective groups is achieved by standard treatment with a suitable TFA cleavage cocktail. The reaction is purified in appropriate aliquots by HPLC (e.g. C18 column; ISCO ACCQ; 3 to 60% acetonitrile in water, using 0.1% TFA as modifier) to afford the peptide macrocycle. This procedure can be applied to homologous analogs of formylglycine.

### Example A4-9

### Grubbs Metathesis Macrocyclization on Resin

In a typical procedure, a fully protected linear bis-alkene peptide of the general formula H—[(—CH═CH₂)XaaN]-Xaa2-Xaa3-Xaa4-(Xaa0)p-Xaa13-[(CH═CH₂)XaaC]-(resin) (300 mg, 0.20 mmol/g) in 22 mL of CH2Cl2 is added via syringe a solution of Grubbs or Grubbs-Hoveyda catalyst (e.g. 1,3-Bis-(2,4,6-trimethylphenyl)-2-imidazolidinylidene)dichloro(o-isopropoxyphenylmethylene)ruthenium; 0.5 eqmol) 6 mM in CH2Cl2. The suspension is heated to 40° C. and gently stirred to completion. The beads are then filtered, rinsed with CH2Cl2, DMF, and MeOH, respectively, and dried under high vacuum. After cleavage from the resin via a suitable TFA cleavage cocktail, the resulting crude cyclic peptide product the is purified by HPLC (e.g. C18 column; ISCO ACCQ; 3 to 60% acetonitrile in water, using 0.1% TFA as modifier). This procedure showcases a head-to-tail macrocyclization; however, side chain cyclizations (staples) can be introduced with careful planning of addition and removal of protecting groups.

### Example A4-10

### Grubbs Metathesis Macrocyclization in Solution

In a typical procedure, a fully protected linear bis-alkene peptide of the general formula H—[(—CH═CH₂)XaaN]-Xaa2-Xaa3-Xaa4-(Xaa0)p- Xaa13-[(CH═CH₂)XaaC]-OH is dissolved to a concentration of 0.5 mM in DCM is added via syringe to a 1.8 mM solution of Grubbs or Grubbs-Hoveyda catalyst (e.g. 1,3-Bis-(2,4,6-trimethylphenyl)-2-imidazolidinylidene)dichloro(o-isopropoxyphenylmethylene)ruthenium; 0.3 eqmol) in CH2Cl2. The solution is stirred at 45° C. for 20 h. The resulting solution is stirred at room temperature to completion. After cleavage of the protective groups via a suitable TFA cleavage cocktail, the resulting crude cyclic peptide product obtained with or without work-up is purified by HPLC (e.g. C18 column; ISCO ACCQ; 3 to 60% acetonitrile in water, using 0.1% TFA as modifier). This procedure showcases a head-to-tail macrocyclization; however, side chain cyclizations (staples) can be introduced with careful planning of addition and removal of protecting groups.

### Example A4-11

### Staudinger Macrocyclization

In a typical procedure, a crude linear C-terminus thioester peptide of the general formula H—[(—N3)XaaN]-Xaa2-Xaa3-Xaa4-(Xaa0)p- Xaa13-[XaaC-SCH₂PH⁺Ph2] is dissolved in DMF to a concentration of 7 mM or lower. Diisopropylethylamine (2 eqmol) is added and the reaction is stirred until completion, and then concentrated to a residue per vacuum techniques. The resulting crude cyclic peptide product is purified by HPLC (e.g. C18 column; ISCO ACCQ; 3 to 95% acetonitrile in water, using 0.1% TFA as modifier). This procedure showcases a head-to-tail cyclization; however, side chain to C-terminus cyclizations can be introduced with careful planning of addition and removal of protecting groups.

### Example A4-12

### CuAAC Macrocyclization

In a typical procedure, a linear azido-alkyne terminated peptide of the general formula H—[(—N3)XaaN]-Xaa2-Xaa3-Xaa4-(Xaa0)p-Xaa13-[(-C═CH)XaaC]-OH or H—[(—C═CH)XaaN]-Xaa2-Xaa3-Xaa4-(Xaa0)p-Xaa13-[(-N3)XaaC]-OH is dissolved in acetonitrile to a concentration of 0.2 mM or lower. The reaction is degassed by Ar or N2 bubbling for 15 min. Diisopropylethylamine (2 eqmol), 2,6-lutidine (2 eqmol), tris-(benzyltriazolylmethyl)amine (2 eqmol), and copper(I) iodide (2 eqmol) are added and Ar bubbling continued for five additional minutes. The reaction is then allowed to stir under inert atmosphere until completion, and then concentrated to a residue per vacuum techniques. The resulting crude cyclic peptide product is purified by HPLC (e.g. C18 column; ISCO ACCQ; 3 to 95% acetonitrile in water, using 0.1% TFA as modifier). This procedure showcases a head-to-tail cyclization; however, side chain cyclizations (staples) can be introduced with careful planning of addition and removal of protecting groups. This protocol can be applied to fully protected or unprotected peptides.

### Example A4-13

### Native Chemical Ligation Macrocyclization

In a typical procedure, a crude linear C-terminus thioester peptide of the general formula H—[(—CH₂SH)XaaN]-Xaa2-Xaa3-Xaa4-(Xaa0)p-Xaa13-XaaC-SR is dissolved in the ligation buffer (6 M guanidine-HCl, 200 mM Na₂HPO₄, pH 8.5) to a concentration of 5 mM or lower. Thiophenol is added to the ligation mixture as a 2% volume (e.g., 2 μL of thiophenol to a 100 μL ligation), emulsifying by raking the mixture. The pH is adjusted to ˜7.1 with the aid of 1M NaOH or 1M HCl solutions. The reaction is quenched by diluting the full reaction in 10 ligation reaction volumes of 50% MeCN in H₂O containing 0.05% TFA and then TCEP is added (15 eqmol) and reacted for 15 minutes. The reaction is purified in appropriate aliquots by HPLC (e.g. C18 column; ISCO ACCQ; 3 to 95% acetonitrile in water, using 0.1% TFA as modifier) to afford the peptide macrocycle. This protocol could be adapted to the application of STL (serine/threonine ligation) of a properly modified peptide by following the general protocol outlined in =Front. Chem. 2:28. doi: 10.3389/fchem.2014.00028 which is hereby incorporated by reference in its entirety.

### Example A5

### Synthesis of 177-Lutetium Chelated Conjugates

A peptide having a structure of Formula (II) or Formula (III) is synthesized according to Examples A1 and A3, and cyclized according to Example A4. The peptide is coupled with a metal chelator (e.g., DOTA or other chelator described herein), optionally through a linker attaching to the C-terminus of the cyclic peptide, thereby producing a conjugate comprising a cyclic peptide and a metal chelator.

General procedure for ¹⁷⁷Lu-labeling [¹⁷⁷Lu]LuCl₃ in HCl (50 MBq) is added to a mixture of a DOTA-cyclic peptide construct (1 nmol) in NaOAc buffer (5% EtOH, 0.25 M, pH 5.0-5.5, total volume 120 μL) in a 1.8 mL Eppendorf tube. The resulting mixture is heated at 80° C. in a thermal mixer at a shaking speed of 600 rpm for 15-30 min. If necessary, the mixture is purified using a C8 column. Radiochemical purity is determined by radio-RP-HPLC and iTLC.

### Example A6

### Synthesis of 225-Actinium Chelated Conjugates

A peptide having a structure of Formula (II) or Formula (III) is synthesized according to Examples A1 and A3, and cyclized according to Example A4. The peptide is coupled with a metal chelator (e.g., DOTA or other chelator described herein), optionally through a linker attaching to the C-terminus of the cyclic peptide, thereby producing a conjugate comprising a cyclic peptide and a metal chelator.

General procedure for ²²⁵Ac-labeling [²²⁵Ac]Ac(NO₃)₃ in 1 mM HCl (50 kBq) is added to a mixture of a DOTA-cyclic peptide construct (1 nmol) in NaOAc buffer (100 μL, 0.4 M, pH 5.5-6.5) in a 1.8 mL Eppendorf tube. The resulting mixture is heated at 80-100° C. in a thermal mixer at a shaking speed of 500 rpm for 15-30 min. Radiochemical purity is determined by iTLC.

### B: Biological Assays.

### Example B1

### In Vivo Pharmacokinetic Studies in Female CD-1 Mice

This experiment is conducted with cold conjugates, wherein radionuclide such as ²²⁵Ac is replaced by lanthanum. (La³⁺)
The pharmacokinetics of the La³⁺-labelled peptide surrogates are determined using female CD-1 (ICR) mice purchased from Vital River Laboratory Animal Co., Ltd., Beijing, China. All animal studies are conducted in accordance with the highest standards of care as outlined in the NIH Guide for Care and Use of Laboratory. Following injection of the mice (10 mg/kg, 3 mice per test compound) with aliquots of the peptides in PBS (10 mM, pH 7.4) via the tail vein, blood samples are collected into pre-chilled tubes containing Heparin-Na (3 μL, 1000 I.U./mL) at 5, 30, 60, and 240 minutes.

General sample processing procedure: An aliquot of 12 μL diluted blood sample (10× dilution factor for 30, 60, and 240 min blood samples; 20× dilution factor for 5 min blood samples), calibration standard, dilution quality control, single blank or double blank samples are added to the individual wells of a low binding 96-well plate. Each sample (except the double blank) is quenched with 120 μL IS in methanol respectively (double blank sample is quenched with 120 μL MeOH). The resulting mixtures are mixed for 10 min at 800 rpm and centrifuged at 3220 g (4000 rpm) for 15 min at 4° C. Supernatant aliquots (50 μL) are transferred to a clean low binding 96-well plate and centrifuged at 3220 g (4000 rpm) for 5 min at 4° C., then the samples are directly injected for LC-MS/MS analysis.

The analytes are detected by a multiple reaction monitoring method using a SCIEX Triple Quad 6500+ system equipped with an ACQUITY UPLC HSS T3 column (100 Å, 1.8 μm, 2.1 mm×30 mm). Mobile phase A: water/acetonitrile (95/5, v/v) with 0.1% formic acid and 2 mM ammonium formate; Mobile phase B: acetonitrile/water (95/5, v/v) with 0.1% formic acid and 2 mM ammonium formate. Or Mobile phase A: water with 0.1% formic acid; Mobile phase B: acetonitrile with 0.1% formic acid. Column temperature: 60° C.

The plasma concentration-time data is subjected to IV-noncompartmental pharmacokinetics analysis by using Phoenix WinNonlin (version 6.3, Pharsight Corp., Mountain View, Calif., USA). The linear/log trapezoidal rule is applied in obtaining the PK parameters.

Pharmacokinetics profiles of the conjugates, including clearance, AUC (0-inf), and elimination half-life will be determined.

### Example B2

### Biodistribution Studies in Mice

Mice bearing 22Rv1 xenograft receive a bolus injection of 50-100 μCi of ¹⁷⁷Lu-labelled peptides via tail vein. Mice are euthanized by lateral chest puncture under anesthesia and dissected at 2, 24 h after injection (n=3 per time point). Blood is collected by cardiac puncture immediately prior to euthanasia at each time point. Liver, heart, kidneys, lungs, muscle, spleen, salivary, tumor and blood are collected into 20 mL pre-weighed glass scintillation vials and capped, where they are placed into a Hidex automated gamma counter (Hidex AMG) to be counted and weighed. The % injected dose (ID) per gram of tissue is calculated against a standard of known activity.

### Example B3

### Determination of Conjugate Plasma Protein Binding

HSA-HPLC method (measurement of drug protein binding by immobilized human serum albumin-HPLC). A 13-minute HPLC (Thermo Vanquish Horizon with Diode Array Detector) gradient method was used to determine the HSA (Human Serum Albumin) binding of novel compounds using a chemically bonded protein stationary phase (ChiralPAK HSA HPLC column, 50×4 mm). The HSA binding values were derived from the gradient retention times that were converted to the logarithm of the equilibrium constant using data from a calibration set of molecules. The % bound to plasma values for the calibrator compounds were converted to the linear free energy values using the following equation: LogK=log[% PPB/(101−% PPB)]. The logarithmic value of the gradient retention times from the experiment were plotted against the linearized values of the percent bound to plasma. The slope and the intercept were used to convert the retention times to linear free energy values (LogK), from which the estimated % protein binding was calculated using the following equation: % Binding=[(101·10^{LogK})/(1+10^{LogK})]. Aqueous mobile phase (A) was 50 mM ammonium acetate solution, ph7.4 and the organic mobile phase (B) was 2-propanol. The flow rate was set at 0.350mL/min and injection volume was 5 uL, with samples prepared at 0.5 mg/mL concentration in 50:50 mobile phase. The initial LC conditions were conducted at room temperature, set at 0% B and ramped to 50% B over 8.5 minutes, then held at 50% B for 1.5 minutes before going back to initial conditions and re-equilibrating the column for 2.5 minutes. Chromatograms were recorded at 280 nm by a diode array UV absorption detector.

The percent binding values of Table 7 are defined as the following:
90% bound to HSA<A≤100% bound to HSA;
70% bound to HSA<B≤90% bound to HSA;
20% bound to HSA<C≤70% bound to HSA; and
0% bound to HSA<D≤20% bound to HSA.

TABLE 7
Percent HSA binding of compounds of the present disclosure

| | % Bound to |
|---|---|
| Compound | HSA at r.t. |
| P-10 | A |
| P-11 | A |
| P-12 | A |
| P-21 | A |
| P-30 | B |
| P-31 | B |
| P-40 | A |

### Example B4

### Determination of Conjugate Binding Affinity to Target Proteins

Cyclic peptides and cyclic peptide conjugates were used to determine binding to target antigens. The presence or absence of the metal chelation moiety does not impact the of cyclic peptides of the present invention and their biological target.

### Example B4-1

### Peptide Binding to FOLR1

For analysis of peptide binding, a Biacore 8K instrument was used utilizing either a SA chip or a CAP chip. FOLR1-Fc-Avi (AcroBiosystems, F01-H₈₂F9) was immobilized on the chip using streptavidin-biotin chemistry at 25° C. in PBS-P+ buffer (20 mM phosphate, 2.7 mM KCl, 137 mM NaCl, 0.05% Tween-20, pH 7.4) to a level of 500-2500 RU (dependent on the analyte molecular weight). A dilution series of peptides was prepared in this buffer with a final DMSO concentration of 1% with a top peptide concentration at 50 or 100 nM and 8 further 2-fold dilutions. The SPR analysis was run at 25° C. at a flow rate of 100 μL/min in PBS-P+ with 1% DMSO running buffer and with a 120 second association and 10,000 second dissociation using single cycle kinetics methodology. All data was analyzed using Biacore Insight Evaluation Software version 3.) Data was fitted using a Langmuir 1:1 binding model.

K_{d} values of peptides of the present disclosure are given in Table 8 below, where K_{d} 0 nM<A≤10 nM.

TABLE 8
Peptide binding to FOLR1

| Compound | K_{d} (nM) |
|---|---|
| P-10 | A |
| P-11 | A |
| P-12 | A |

### Example B4-2

### Peptide Binding to PSMA

For analysis of peptide binding, a Biacore 8K instrument was used utilizing a SA chip. His-Avi-PSMA (AcroBiosystems, PSA-H82Qb) was immobilized on the chip using streptavidin-biotin chemistry at 25° C. in HBS-P+ buffer (10 mM HEPES, 150 mM NaCl, 0.05% Tween-20, pH 7.4) to a level of 800-2200 RU (dependent on the analyte molecular weight). A dilution series of peptides was prepared in this buffer with a final DMSO concentration of 1% with a top peptide concentration between 10-100 nM and 8 further 2-fold dilutions. The SPR analysis was run at 25° C. at a flow rate of 100 μL/min in HBS-P+ with 1% DMSO running buffer and with a 120 second association and 10,000 second dissociation using single cycle kinetics methodology. All data was analyzed using Biacore Insight Evaluation Software version 3. Data was fitted using a Langmuir 1:1 binding model.

K_{d} values of compounds of the present disclosure are given in Table 9 below, where K_{d} 0 nM<A≤10 nM.

TABLE 9
Peptide binding to PSMA

| Compound | K_{d} (nM) |
|---|---|
| P-21 | A |

### Example B4-3

### Conjugate Binding to Trop-2

For analysis of peptide binding, a GatorPrime instrument was used utilizing SMAP probes. Biotinylated peptides of 1 μM were immobilized on the sensor using streptavidin-biotin chemistry at 25° C. in HBS-P+ buffer (10 mM HEPES, 150 mM NaCl, 0.05% Tween-20, pH 7.4). A dilution series of His-tagged Trop-2 (AcroBiosystems, TR2-H5223) was prepared in this buffer with a top peptide concentration at 100 nM and 6 further 3-fold dilutions. The BLI analysis was run at 30° C. with a 120 second association and 120 second dissociation. All data was analyzed using GatorLaunch software. Data was fitted using a Langmuir 1:1 binding model.

K_{d} values of compounds of the present disclosure are given in Table 10 below, where K_{d} 0 nM<A≤10 nM; 10 nM<B≤1000 nM.

TABLE 10
Conjugate binding to Trop-2

| Compound | K_{d} (nM) |
|---|---|
| C-40 | B |

### Example B5

### Determination of Conjugate Binding Affinity to Cells Expressing Target Proteins

### Example B5-1

### Conjugate Binding to FOLR1 Expressing Cells

Dilute peptides:
(1) Compounds were reconstituted to 10 mM stock in DMSO aliquots and stored at −80.
(2) Compound dilution plates were made: first diluted dose was made and further serial dilution as needed in DMSO.
(3) Added 3 ul of diluted compounds to 97 ul of testing buffer (i.e. hPlasma/PBS) (33.3× dilution)
(4) Added 10 ul of 3. to 90 ul of cells in 96 well plates (another 10× dilution)
(5) Example: if first does to be tested is 1 uM=>take 1 ul of 10 mM stock to 29 ul of DMSO=>if Making 5× serial dilutions, take 2 ul from 1st dose to 8 ul of DMSO and continue this action to the last dose=>take 3 ul of all diluted compounds to 97 ul hPlasma/PBS=>take 10 ul of these to 90 ul of cells

FACS Binding:
(1) Washed cells in flask with PBS, aspirate
(2) Added 2-3 mls Accutase, wait ˜10 mins for cells to detach
(3) Resuspended in 10 ml media, count using viability stain
(4) Washed cells 1× in PBS
(5) Resuspended and performed bulk viability dye with Zombie Violet (1:1000), 10 mins at RT, in the dark wash 3×3 mins 1600 RPMs with PBS
(6) Seeded to FACS plate, 50-100,000 cells/well (in hPlasma/PBS (Sigma, P9523-5ML) reconstitute hPlasma powder in 10 ml PBS before use)
(7) Added dilutions of peptides, incubated on ice for 50 mins
(8) Washed 3×3 mins 1600 RPMs with PBS+hPlasma
(9) Added dilution of SA-647 (1:1000), incubated on ice for 30 mins
(10) Washed 3×mins 1600 RPMs with PBS+hPlasma
(11) Read on Cytoflex (find cell using auto; keep PB450 and APC baseline to be below or close to 1000)

EC₅₀ values of compounds of the present disclosure are given in Table 11 below, where EC₅₀ 0 nM<A≤100 nM.

TABLE 11
Peptide binding to FOLR1 expressing cells (IGROV1)

| Compound | EC₅₀ (nM) |
|---|---|
| P-10 | A |
| P-11 | A |

### Example B5-2

### Conjugate Binding to PSMA Expressing Cells

Biotinylated Compounds

LnCap cells, 2e⁶ cells/ml

FACS buffer=1×PBS+1% BSA

SYTOX viability dye
(1) Washed cells with PBS once.
(2) Incubated cells with 5 ml of Accutase at 37 degrees for 2-5 minutes. Gently rocking the flask until the layer of cells pilling off
(3) Added 15 ml of PBS into flask and transferred to a 50 ml conical tube
(4) Spun down 1600 rpm 3 minutes
(5) Resuspended in 2 ml of PBS, and counted cells
(6) Adjusted cell density to 2e6 cells/ml with PBS and then added 100 uL/well to the plate and mixed well
(7) Spun down 1600 rpm 3 minutes
(8) Add 100 uL of compound/well respectively. Prepared serial dilute compound in PBS, 1 uM to 0.01 nM, 1:3 dilution, 10 point curve
(9) Incubated for 50 minutes total
(10) Washed wells with 150 uL PBS and spun down 1600 rpm 3 min and dumped
(11) Added SA-A647, 1:1000 dilution in FACS buffer, 100 ul/well to respective wells
(12) Incubated for 50 min on ice
(13) Washed with 200 uL PBS spun 1600 rpm 3 min dump
(14) Resuspended in 100 uL SYTOX, 1:1000 dilution in FACS buffer
(15) Acquired

EC₅₀ values of compounds of the present disclosure are given in Table 12 below, where EC₅₀ 0 nM<A≤100 nM; 100 nM<B≤1000 nM.

TABLE 12
Peptide binding to PSMA expressing cells (LnCap)

| Compound | EC₅₀ (nM) |
|---|---|
| P-21 | A |
| P-22 | B |

### Example B5-3

### Conjugate Binding to Trop-2 Expressing Cells

Conjugate binding to Trop-2 expressing cells was performed according to Example A5-2 with A431 cells. EC₅₀ values of compounds of the present disclosure are given in Table 13 below, where EC₅₀ 0 nM<A≤100 nM.

TABLE 13
Peptide binding to Trop-2 expressing cells (A431)

| Compound | EC₅₀ (nM) |
|---|---|
| P-40 | A |

The examples and embodiments described herein are for illustrative purposes only and various modifications or changes suggested to persons skilled in the art are to be included within the spirit and purview of this application and scope of the appended claims.

We claim:

1. A radiopharmaceutical conjugate comprising,
(a) a monocyclic binding peptide that binds a cell-surface protein,
wherein the monocyclic binding peptide comprises 8-18 amino acid residues, and
wherein the monocyclic binding peptide is cyclized by a non-disulfide bond;
(b) a metal chelator configured to bind with a radionuclide; and
(c) a linker that covalently attaches the binding peptide to the metal chelator.

2. The radiopharmaceutical conjugate of claim 1, wherein the monocyclic binding peptide consists of 8-14 amino acid residues.

3. The radiopharmaceutical conjugate of claim 1, wherein the monocyclic binding peptide consists of 8-12 amino acid residues.

4. The radiopharmaceutical conjugate of claim 1, wherein the cell-surface protein is an oncofetal antigen, a tight junction protein, a transmembrane glycoprotein, an adhesion protein, a transporter receptor, or a tyrosine kinase receptor (TKR).

5. The radiopharmaceutical conjugate of claim 1, wherein the cell-surface protein is a transmembrane glycoprotein.

6. The radiopharmaceutical conjugate of claim 1, wherein the cell-surface protein is tumor-associated calcium signal transducer 2 (Trop-2).

7. The radiopharmaceutical conjugate of claim 1, wherein the cell-surface protein is nectin cell adhesion molecule 4 (Nectin-4).

8. The radiopharmaceutical conjugate of claim 1, comprising an alpha-emitting radionuclide bound to the metal chelator.

9. The radiopharmaceutical conjugate of claim 8, wherein the alpha-emitting radionuclide is actinium-225.

10. The radiopharmaceutical conjugate of claim 1, wherein the monocyclic binding peptide comprises at least one non-natural amino acid residue.

11. The radiopharmaceutical conjugate of claim 1, wherein the monocyclic binding peptide is cyclized by a ring closing group selected from —(CH₂)ₘ—C(═O)—CH₂—S—(CH₂)ₙ—, —C(═O)—CH₂—S—CH₂—CH₂—, —(CH₂)ₘ—NH—CO—(CH₂)ₙ—, —(CH₂)ₘ—CO—NH—(CH₂)ₙ—, —(CH₂)ₘ—S—(CH₂)ₙ—, —(CH₂)ₘ—CH═CH—(CH₂)ₙ—, —(CH₂)ₘ—NH—(CH₂)ₙ—, —(CH₂)ₘ—S—CH₂-benzene-CH₂—S—(CH₂)ₙ—, —(CH₂)ₘ-triazine-(CH₂)ₙ—, —(CH₂)ₘ-succinimide-S—(CH₂)ₙ—, —C(═O)—CH₂—NH—CH₂—, and —C(═O)—CH₂—O—CH₂—, where m and n are each independently 0 or an integer from 1 to 6.

12. The radiopharmaceutical conjugate of claim 1, wherein the monocyclic binding peptide is cyclized by a ring closing group selected from —C(═O)—CH₂—S—, —C(═O)—CH₂—NH—, and —C(═O)—CH₂—O—.

13. The radiopharmaceutical conjugate of claim 1, wherein the monocyclic binding peptide is cyclized by a ring closing group selected from —C(═O)—CH₂—S—.

14. The radiopharmaceutical conjugate of claim 13, wherein the ring closing group is formed by reacting a cysteine and a chloroacetylated amino acid.

15. The radiopharmaceutical conjugate of claim 1, wherein the monocyclic binding peptide is cyclized via a thioether bond formed between a chloroacetyl group at the N-terminus and a Cys at the C-terminus.

16. A radiopharmaceutical conjugate comprising:
(a) a targeting moiety that comprises a cyclic peptide having a structure of Formula (III); 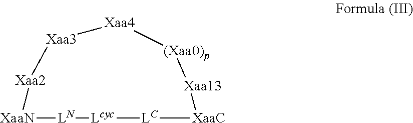
(attachment point to metal chelator not shown); and
(b) a metal chelator configured to bind with a radionuclide; wherein
each of the XaaN, Xaa2, Xaa3, Xaa4, Xaa0, Xaa13, and XaaC is independently an amino acid residue;
p is 0, 1, 2, 3, 4, 5, 6, 7, or 8;
wherein each of the amino acid residues in the cyclic peptide is joined by a peptide bond, provided that XaaN and XaaC is connected through -L^{N}-L^{cyc}-L^{c}-;
L^{N} and L^{C} are each independently optionally substituted C₁-C₆alkylene, optionally substituted C₁-C₆heteroalkylene, or a bond, wherein the alkylene or heteroalkylene is optionally substituted; and
L^{cyc} is a ring closing group comprising a structure selected from —C(═O)—CH₂—S—, —S—, —CH═CH—, —NH—, -succinimide-S—, —C(═O)—CH₂—NH—, and —C(═O)—CH₂—O—.

17. The radiopharmaceutical conjugate of claim 16, wherein the cyclic peptide is monocyclic.

18. The radiopharmaceutical conjugate of claim 16, wherein the cyclic peptide does not contain a di-sulfide bond.

19. The radiopharmaceutical conjugate of claim 16, wherein p is 2, 3, 4, 5, or 6.

20. The radiopharmaceutical conjugate of claim 16, wherein XaaC is cysteine, homocysteine, lysine, homolysine, ornithine, diaminobutric acid, serine, homoserine, threonine, or homothreonine; XaaN is a chloroacetylated amino acid; and L^{cyc} is formed by reacting —C(═O)—CH₂Cl on the XaaN with XaaC.

21. The radiopharmaceutical conjugate of claim 16, wherein the cyclic binding peptide comprises at least one non-natural amino acid residue.


SEQUENCE LISTING (24 sequences)

SEQ ID NO: 1 - protein, 15 aa, synthetic construct
  source 1..15: note = Description of Artificial Sequence: Synthetic peptide
  SITE 5: note = MOD_RES - 2-aminoheptanoic acid
  SITE 6: note = MOD_RES - N-methyl glycine
  SITE 8: note = MOD_RES - N-methyl phenylalanine
  SITE 14: note = MOD_RES - N-methyl phenylalanine
  SITE 1..15: note = Cyclized via 2-(sulfanyl)-ethanyl-1-one between the N terminus of the Ala and the side chain of the Cys
AHYTXGWFRVRWYFC

SEQ ID NO: 2 - protein, 12 aa, synthetic construct
  source 1..12: note = Description of Artificial Sequence: Synthetic peptide
  SITE 1: note = MOD_RES - N-methyl phenylalanine
  SITE 4: note = MOD_RES - N-methyl glycine
  SITE 8: note = MOD_RES - N-methyl 1-naphthylalanine
  SITE 1..12: note = Cyclized via 2-(sulfanyl)-ethanyl-1-one between the N terminus of the Phe and the side chain of the Cys
FSWGYNYXGWWC

SEQ ID NO: 3 - protein, 13 aa, synthetic construct
  source 1..13: note = Description of Artificial Sequence: Synthetic peptide
  SITE 4: note = MOD_RES - N-methyl phenylalanine
  SITE 9: note = MOD_RES - N-methyl tyrosine
  SITE 10: note = MOD_RES - N-methyl tyrosine
  SITE 1..13: note = Cyclized via 2-(sulfanyl)-ethanyl-1-one between the N terminus of the Ala and the side chain of the Cys
ARLFDYDTYYWYC

SEQ ID NO: 4 - protein, 13 aa, synthetic construct
  source 1..13: note = Description of Artificial Sequence: Synthetic peptide
  SITE 3: note = MOD_RES - cyclohexylglycine
  SITE 4: note = MOD_RES - N-methyl phenylalanine
  SITE 6: note = MOD_RES - 3-chlorophenylalanine
  SITE 9: note = MOD_RES - N-methyl tyrosine
  SITE 10: note = MOD_RES - N-methyl tyrosine
  SITE 1..13: note = Cyclized via 2-(sulfanyl)-ethanyl-1-one between the N terminus of the Asp and the side chain of the Cys
DRXFDXDTYYWYC

SEQ ID NO: 5 - protein, 13 aa, synthetic construct
  source 1..13: note = Description of Artificial Sequence: Synthetic peptide
  SITE 3: note = MOD_RES - cyclohexylglycine
  SITE 4: note = MOD_RES - N-methyl phenylalanine
  SITE 9: note = MOD_RES - N-methyl tyrosine
  SITE 10: note = MOD_RES - N-methyl tyrosine
  SITE 1..13: note = Cyclized via 2-(sulfanyl)-ethanyl-1-one between the N terminus of the Asp and the side chain of the Cys
DRXFDYDTYYWYC

SEQ ID NO: 6 - protein, 11 aa, synthetic construct
  source 1..11: note = Description of Artificial Sequence: Synthetic peptide
  SITE 3: note = MOD_RES - N-methyl phenylalanine
  SITE 7: note = MOD_RES - N-methyl tyrosine
  SITE 10: note = MOD_RES - N-methyl tyrosine
  SITE 1..11: note = Cyclized via 2-(sulfanyl)-ethanyl-1-one between the N terminus of the Ala and the side chain of the Cys
AIFYDDYYDYC

SEQ ID NO: 7 - protein, 12 aa, synthetic construct
  source 1..12: note = Description of Artificial Sequence: Synthetic peptide
  SITE 2: note = MOD_RES - 3-phenylphenylalanine
  SITE 8: note = MOD_RES - Cyclobutylalanine
  SITE 10: note = MOD_RES - 3-phenylphenylalanine
  SITE 1..12: note = Cyclized via 2-(sulfanyl)-ethanyl-1-one between the N terminus of the Trp and the side chain of the Cys
WXDVDSDXWXDC

SEQ ID NO: 8 - protein, 10 aa, synthetic construct
  source 1..10: note = Description of Artificial Sequence: Synthetic peptide
  SITE 1: note = MOD_RES - N-methyl phenylalanine
  SITE 8: note = MOD_RES - homophenylalanine
  SITE 1..10: note = Cyclized via 2-(sulfanyl)-ethanyl-1-one between the N terminus of the Phe and the side chain of the Cys
FDDSVWDXYC

SEQ ID NO: 9 - protein, 14 aa, synthetic construct
  source 1..14: note = Description of Artificial Sequence: Synthetic peptide
  SITE 3: note = MOD_RES - 3-chlorophenylalanine
  SITE 7: note = MOD_RES - N-methyl phenylalanine
  SITE 10: note = MOD_RES - 6-chlorotryptaphan
  SITE 12: note = MOD_RES - N-methyl glycine
  SITE 13: note = MOD_RES - 2-aminoheptanoic acid
  SITE 1..14: note = Cyclized via 2-(sulfanyl)-ethanyl-1-one between the N terminus of the Ala and the side chain of the Cys
AVXYRGFTGXVGXC

SEQ ID NO: 10 - protein, 13 aa, synthetic construct
  source 1..13: note = Description of Artificial Sequence: Synthetic peptide
  SITE 1: note = MOD_RES - 18F-Lys - 18-fluorine lysine
KYNDRLPLYISNP

SEQ ID NO: 11 - protein, 5 aa, synthetic construct
  source 1..5: note = Description of Artificial Sequence: Synthetic peptide
EAAAK

SEQ ID NO: 12 - protein, 14 aa, synthetic construct
  source 1..14: note = Description of Artificial Sequence: Synthetic peptide
  VARIANT 2: note = MOD_RES - Ile or Thr
  VARIANT 12: note = MOD_RES - Ala or Glu
  VARIANT 13: note = MOD_RES - Ala, Asp, Val, or Gly
  REGION 1..14: note = See specification as filed for detailed description of substitutions and preferred embodiments
FXDDYGITDYDXXC

SEQ ID NO: 13 - protein, 14 aa, synthetic construct
  SITE 1: note = D-phenylalanine
  SITE 2: note = (S)-2-amino-3-(pyridin-3-yl)propanoic acid
  SITE 5: note = (S)-2-amino-3-(pyridin-3-yl)propanoic acid
  SITE 6: note = N-methyl alanine
  SITE 7: note = N-methyl alanine
  SITE 10: note = N-methyl tyrosine
  SITE 1..14: note = Cyclized via 2-(sulfanyl)-ethanyl-1-one between the N terminus of the Phe and the side chain of the Cys
FXILXAASDYFIIC

SEQ ID NO: 14 - protein, 14 aa, synthetic construct
  SITE 1: note = D-alanine
  SITE 3: note = 4-phenylphenylalanine
  SITE 8: note = 4-phenylphenylalanine
  SITE 13: note = N-methyl phenylalanine
  SITE 1..14: note = Cyclized via 2-(sulfanyl)-ethanyl-1-one between the N terminus of the Ala and the side chain of the Cys
ADXIYPAXSSESFC

SEQ ID NO: 15 - protein, 18 aa, synthetic construct
  SITE 1: note = Bromo acetyl-D-alanine
  SITE 2: note = Aspartic Acid O-tert butyl
  SITE 3: note = 4-phenylphenylalanine
  SITE 5: note = Tert-butyl tyrosine
  SITE 8: note = 4-phenylphenylalanine
  SITE 9: note = Tert-butyl serine
  SITE 10: note = Tert-butyl serine
  SITE 11: note = O-tert butyl glutamic acid
  SITE 12: note = Tert-butyl serine
  SITE 13: note = N-methyl phenylalanine
  SITE 14: note = trityl-cysteine
  SITE 15: note = Beta-alanine
  SITE 16: note = D-lysine-2,2-dimethyl-4,32-dioxo-3,8,11,14,17,20,23 ,26,29,36,39-undecaoxa-5,33-diazahentetracontan-41-oic acid
  SITE 17: note = O-tert butyl gamma-glutamic acid
  SITE 18: note = O-tert butyl gamma-glutamic acid
ADXIYPAXSSESFCXKXX

SEQ ID NO: 16 - protein, 18 aa, synthetic construct
  SITE 1: note = Bromo acetyl D-alanine
  SITE 3: note = 4-phenylphenylalanine
  SITE 8: note = 4-phenylphenylalanine
  SITE 13: note = N-methyl phenylalanine
  SITE 15: note = Beta-alanine
  SITE 16: note = D-lysine-1-amino-27-oxo-3,6,9,12,15,18,21,24,31,34- decaoxa-28-azahexatriacontan-36-oic acid
  SITE 17: note = gamma-glutamic acid
  SITE 18: note = gamma-glutamic acid amide
ADXIYPAXSSESFCXKXX

SEQ ID NO: 17 - protein, 18 aa, synthetic construct
  SITE 1: note = Acetyl D-alanine
  SITE 3: note = 4-phenylphenylalanine
  SITE 8: note = 4-phenylphenylalanine
  SITE 13: note = N-methyl phenylalanine
  SITE 15: note = Beta-alanine
  SITE 16: note = D-lysine-1-amino-27-oxo-3,6,9,12,15,18,21,24,31,34- decaoxa-28-azahexatriacontan-36-oic acid
  SITE 17: note = gamma-glutamic acid
  SITE 18: note = gamma-glutamic acid amide
ADXIYPAXSSESFCXKXX

SEQ ID NO: 18 - protein, 18 aa, synthetic construct
  SITE 1: note = D-alanine
  SITE 3: note = 4-phenylphenylalanine
  SITE 8: note = 4-phenylphenylalanine
  SITE 13: note = N-methyl phenylalanine
  SITE 15: note = Beta-alanine
  SITE 16: note = D-lysine-2,2',2''-(10-(1-carboxy-9,37-dioxo-2,5,12, 15,18,21,24,27,30,33-decaoxa-8,36-diazaoctatriacontan-38-y l)-1,4,7,10-tetraazacyclododecane-1,4,7-triyl)triacetic acid
  SITE 17: note = gamma-glutamic acid
  SITE 18: note = gamma-glutamic acid amide
  SITE 1..14: note = Cyclized via 2-(sulfanyl)-ethanyl-1-one between the N terminus of the Ala and the side chain of the Cys
ADXIYPAXSSESFCXKXX

SEQ ID NO: 19 - protein, 15 aa, synthetic construct
  SITE 3: note = cyclohexylglycine
  SITE 4: note = N-methyl phenylalanine
  SITE 6: note = 3-chlorophenylalanine
  SITE 9: note = N-methyl tyrosine
  SITE 10: note = N-methyl tyrosine
  SITE 14: note = Beta-alanine
  SITE 15: note = D-lysine-2,2',2'',2'''-(1,4,7,10-tetraazacyclododec ane-1,4,7,10-tetrayl)tetraacetic acid amide
  SITE 1..13: note = Cyclized via 2-(sulfanyl)-ethanyl-1-one between the N terminus of the Asp and the side chain of the Cys
DRXFDXDTYYWYCXK

SEQ ID NO: 20 - protein, 15 aa, synthetic construct
  SITE 3: note = cyclohexylglycine
  SITE 4: note = N-methyl phenylalanine
  SITE 9: note = N-methyl tyrosine
  SITE 10: note = N-methyl tyrosine
  SITE 14: note = Beta-alanine
  SITE 15: note = D-lysine-2,2',2'',2'''-(1,4,7,10-tetraazacyclododec ane-1,4,7,10-tetrayl)tetraacetic acid amide
  SITE 1..13: note = Cyclized via 2-(sulfanyl)-ethanyl-1-one between the N terminus of the Asp and the side chain of the Cys
DRXFDYDTYYWYCXK

SEQ ID NO: 21 - protein, 13 aa, synthetic construct
  SITE 2: note = 3-phenylphenylalanine
  SITE 8: note = cyclobutylalanine
  SITE 10: note = 3-phenylphenylalanine
  SITE 13: note = Lysine-2,2',2'',2'''-(1,4,7,10-tetraazacyclododecan e-1,4,7,10-tetrayl)tetraacetic acid amide
  SITE 1..12: note = Cyclized via 2-(sulfanyl)-ethanyl-1-one between the N terminus of the Trp and the side chain of the Cys
WXDVDSDXWXDCK

SEQ ID NO: 22 - protein, 16 aa, synthetic construct
  SITE 1: note = D-phenylalanine
  SITE 2: note = (S)-2-amino-3-(pyridin-3-yl)propanoic acid
  SITE 5: note = (S)-2-amino-3-(pyridin-3-yl)propanoic acid
  SITE 6: note = N-methyl alanine
  SITE 7: note = N-methyl alanine
  SITE 10: note = N-methyl tyrosine
  SITE 15: note = Beta-alanine
  SITE 16: note = D-lysine-2,2',2''-(10-(1-carboxy-9,37-dioxo-2,5,12, 15,18,21,24,27,30,33-decaoxa-8,36-diazaoctatriacontan-38-y l)-1,4,7,10-tetraazacyclododecane-1,4,7-triyl)triacetic acid amide
  SITE 1..14: note = Cyclized via 2-(sulfanyl)-ethanyl-1-one between the N terminus of the Phe and the side chain of the Cys
FXILXAASDYFIICXK

SEQ ID NO: 23 - protein, 14 aa, synthetic construct
  SITE 1: note = D-alanine
  SITE 2: note = N-methyl phenylalanine
  SITE 5: note = (S)-2-aminohexanedioic acid
  SITE 6: note = N-methyl phenylalanine
  SITE 7: note = (2S)-2-amino-3-(1-methyl-1H-indol-3-yl)propanoic acid
  SITE 9: note = (2S)-2-amino-3-(1-methyl-1H-indol-3-yl)propanoic acid
  SITE 13: note = Beta-alanine
  SITE 14: note = D-lysine-2,2',2''-(10-(1-carboxy-9,37-dioxo-2,5,12, 15,18,21,24,27,30,33-decaoxa-8,36-diazaoctatriacontan-38-y l)-1,4,7,10-tetraazacyclododecane-1,4,7-triyl)triacetic acid amide
  SITE 1..12: note = Cyclized via 2-(sulfanyl)-ethanyl-1-one between the N terminus of the Ala and the side chain of the Cys
AFNLXFXVXTECXK

SEQ ID NO: 24 - protein, 16 aa, synthetic construct
  SITE 1: note = D-alanine
  SITE 3: note = 4-phenylphenylalanine
  SITE 8: note = 4-phenylphenylalanine
  SITE 13: note = N-methyl phenylalanine
  SITE 15: note = Beta-alanine
  SITE 16: note = D-lysine-2,2',2'',2'''-(1,4,7,10-tetraazacyclododec ane-1,4,7,10-tetrayl)tetraacetic acid amide
  SITE 1..14: note = Cyclized via 2-(sulfanyl)-ethanyl-1-one between the N terminus of the Ala and the side chain of the Cys
ADXIYPAXSSESFCXK